(19) United States
(12) Patent Application Publication
(10) Pub. No.: US 2023/0242586 A1
(43) Pub. Date: Aug. 3, 2023

(54) RAS INHIBITORS AND USES THEREOF

(71) Applicants: The Regents of the University of California, Oakland, CA (US); The University of Tokyo, Tokyo (JP)

(72) Inventors: Kevan M. Shokat, San Francisco, CA (US); Hiroaki Suga, Tokyo (JP); Ziyang Zhang, San Francisco, CA (US); Rong Gao, Shanghai (CN)

(21) Appl. No.: 17/919,895

(22) PCT Filed: Apr. 23, 2021
(86) PCT No.: PCT/US2021/028874

§ 371 Date: Oct. 19, 2022

Related U.S. Application Data
(60) Provisional application No. 63/014,596, filed on Apr. 23, 2020.

(51) Int. Cl. C07K 7/64; A61P 35/00

(52) U.S. Cl. CPC C07K 7/64; A61P 35/00; A61K 38/00

(57) ABSTRACT

Described herein, inter alia, are Ras inhibitors and uses thereof.

### CROSS-REFERENCES TO RELATED APPLICATIONS

This application is the national stage filing under 35 U.S.C. § 371 of International Application No. PCT/US2021/028874 filed Apr. 23, 2021, which claims the benefit of U.S. Provisional Application No. 63/014,596, filed Apr. 23, 2020, which are incorporated herein by reference in their entirety and for all purposes.

### STATEMENT AS TO RIGHTS TO INVENTIONS MADE UNDER FEDERALLY SPONSORED RESEARCH AND DEVELOPMENT

This invention was made with government support under the Platform Project for Supporting Drug Discovery and Life Science Research Program (Basis for Supporting Innovative Drug Discovery and Life Science Research) of Japan Agency for Medical Research and Development (AMED). The government has certain rights in the invention.

### REFERENCE TO A “SEQUENCE LISTING,” A TABLE, OR A COMPUTER PROGRAM LISTING APPENDIX SUBMITTED AS AN ASCII FILE

The Sequence Listing written in file 048536-684N01US_Sequence_Listing_ST25, created Oct. 10, 2022, 23,084 bytes, machine format IBM-PC, MS Windows operating system, is hereby incorporated by reference.

### BACKGROUND

Missense mutations of the RAS genes (KRAS, HRAS, and NRAS) occur frequently in human cancer and drive oncogenic transformation. Among these, KRAS G12D is the most prevalent point mutation associated with poor clinical outcome. The G12D mutation impairs both intrinsic and GTPase-accelerating protein (GAP)-mediated GTP hydrolysis and liberates K-Ras protein from functional control by GTPase activity. As a result, K-Ras(G12D) is enriched in its GTP-bound, signaling-competent state, given the near 10-fold higher concentration of GTP than GDP inside the cell. Disclosed herein, inter alia, are solutions to these and other problems in the art.

### BRIEF SUMMARY

In an aspect is provided a compound having the formula:

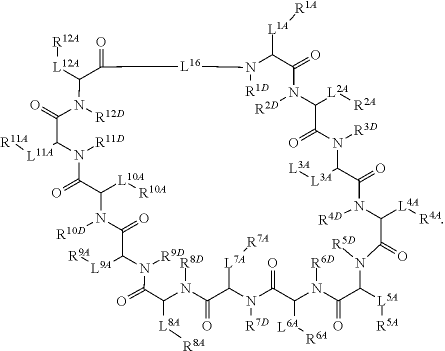

L^{1A}, L^{2A}, L^{3A}, L^{4A}, L^{5A}, L^{6A}, L^{7A}, L^{8A}, L^{9A}, L^{10A}, L^{10A}, and L^{12A} are independently a bond, substituted or unsubstituted alkylene, or substituted or unsubstituted heteroalkylene.

R^{1A}, R^{5A}, and R^{11A} are independently substituted or unsubstituted cycloalkyl, substituted or unsubstituted heterocycloalkyl, substituted or unsubstituted aryl, or substituted or unsubstituted heteroaryl.

R^{2A} and R^{8A} are independently hydrogen, substituted or unsubstituted cycloalkyl, substituted or unsubstituted heterocycloalkyl, substituted or unsubstituted aryl, or substituted or unsubstituted heteroaryl.

R^{3A} is hydrogen, substituted or unsubstituted alkyl, substituted or unsubstituted cycloalkyl, or substituted or unsubstituted aryl.

R^{4A} is hydrogen, —NH₂, —COOH, —CONH₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, substituted or unsubstituted alkyl, substituted or unsubstituted heteroalkyl, or substituted or unsubstituted aryl.

R^{6A} and R^{9A} are independently hydrogen, —CN, —NH₂, —CONH₂, —SO₂NH₂, —NHNH₂, —ONH₂, —NHC(O)NHNH₂, —NHC(O)NH₂, —NHC(NH)NH₂, —NHOH, substituted or unsubstituted alkyl, or substituted or unsubstituted heteroalkyl.

R^{7A} is hydrogen, —NH₂, —COOH, —CONH₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, substituted or unsubstituted alkyl, or substituted or unsubstituted heteroalkyl.

R^{3A} and R^{9A} may optionally be joined to form a covalent linker.

R^{10A} is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —C(O)H, —C(O)OH, —C(O)NH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, -L^{10D}L^{10E}-E, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

L^{10D} is a bond, —S(O)₂—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

L^{10E} is a bond, —S(O)₂—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted heteroalkylene, substituted or unsubstituted heterocycloalkylene, or substituted or unsubstituted heteroarylene.

R^{12A} is hydrogen, —CN, —NH₂, —CONH₂, —SO₂NH₂, —NHNH₂, —ONH₂, —NHC(O)NHNH₂, —NHC(O)NH₂, —NHC(NH)NH₂, —NHOH, substituted or unsubstituted alkyl, substituted or unsubstituted heteroalkyl, or substituted or unsubstituted aryl.

E is an electrophilic moiety.

R^{1D}, R^{2D}, R^{3D}, R^{4D}, R^{5D}, R^{6D}, R^{7D}, R^{8D}, R^{9D}, R^{10D}, R^{11D}, and R^{12D} are independently hydrogen, unsubstituted C₁-C₈ alkyl.

L¹⁶ is a covalent linker.

In an aspect is provided a compound having the formula:

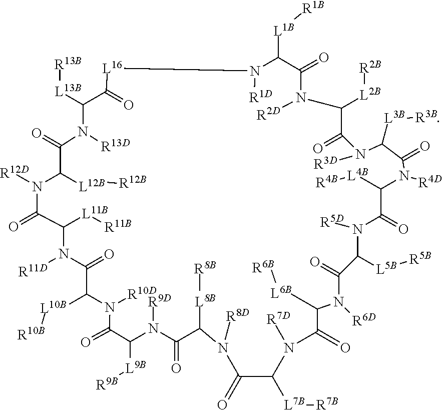

R^{1D}, L^{2D}, R^{3D}, R^{4D}, R^{5D}, R^{6D}, R^{7D}, R^{8D}, R⁷R^{9D}, R^{10D}, R^{11D}, R^{12D}, and L¹⁶ are as described herein, including in embodiments.

L^{1B}, L^{2B}, L^{3B}, L^{4B}, L^{5B}, L^{6B}, L^{7B}, L^{8B}, L^{9B}, L^{10B}, L^{11B}, L^{12B}, and L^{13B} are independently a bond, substituted or unsubstituted alkylene, or substituted or unsubstituted heteroalkylene.

R^{1B}, R^{8B}, and R^{10B} are independently substituted or unsubstituted cycloalkyl, substituted or unsubstituted heterocycloalkyl, substituted or unsubstituted aryl, or substituted or unsubstituted heteroaryl.

R^{2B}, R^{3B}, R^{4B}, R^{9B}, and R^{11B} are independently hydrogen, substituted or unsubstituted alkyl, substituted or unsubstituted cycloalkyl, or substituted or unsubstituted aryl.

R^{5B} is independently hydrogen, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, or substituted or unsubstituted heteroalkyl.

R^{6B} is independently hydrogen, —OH, —COOH, —NO₂, —SO₃H, —OSO₃H, substituted or unsubstituted alkyl, or substituted or unsubstituted heteroalkyl.

R^{7B}, R^{12B}, and R^{13B} independently hydrogen, —NH₂, —CONH₂, —SO₂NH₂, —NHNH₂, —ONH₂, —NHC(O)NHNH₂, —NHC(O)NH₂, —NHC(NH)NH₂, substituted or unsubstituted alkyl, substituted or unsubstituted heteroalkyl, or substituted or unsubstituted heteroaryl.

Two substituents selected from R^{1B}, R^{2B}, R^{3B}, R^{4B}, R^{5B}, R^{6B}, L^{7B}, R^{8B}, R^{9B}, R^{10B}, R^{11B}, R^{12B}, and R^{13B} may optionally be joined to form a covalent linker.

R^{13D} is independently hydrogen or unsubstituted C₁-C₄ alkyl.

In an aspect is provided a compound having the formula:

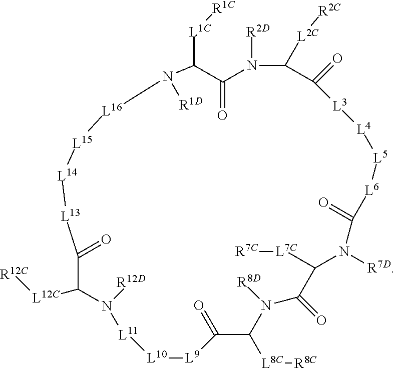

R^{1D}, R^{2D}, R^{3D}, R^{4D}, R^{5D}, R^{6D}, R^{7D}, R^{8D}, R^{9D}, R^{10D}, R^{11D}, R^{12D}, R^{13D}, and L¹⁶ are as described herein, including in embodiments.

L^{1C}, L^{2C}, L^{3C}, L^{4C}, L^{5C}, L^{6C}, L^{7C}, L^{8C}, L^{9C}, L^{10C}, L^{11C}, L^{12C}, L^{13C}, L^{14C}, and L^{15C} are independently a bond, substituted or unsubstituted alkylene, or substituted or unsubstituted heteroalkylene.

R^{1C} is independently substituted or unsubstituted cycloalkyl, substituted or unsubstituted heterocycloalkyl, substituted or unsubstituted aryl, or substituted or unsubstituted heteroaryl.

R^{2C} is independently hydrogen, —OH, —NO₂, —CN, —NH₂, —C(O)OH, —C(O)NH₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, substituted or unsubstituted alkyl, or substituted or unsubstituted heteroalkyl.

L³ is independently a bond or

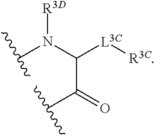

R^{3C} is independently hydrogen, —NH₂, —C(O)OH, —C(O)NH₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

L⁴ is independently a bond or

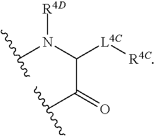

R^{4C} is independently hydrogen, substituted or unsubstituted alkyl, substituted or unsubstituted cycloalkyl, or substituted or unsubstituted aryl.

L⁵ is independently a bond or

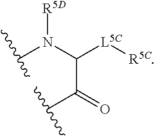

R^{5C} is independently substituted or unsubstituted cycloalkyl, substituted or unsubstituted heterocycloalkyl, substituted or unsubstituted aryl, or substituted or unsubstituted heteroaryl.

L⁶ is independently a bond,

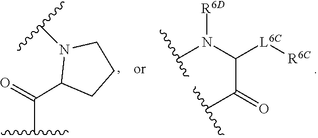

R^{6C} is independently hydrogen, —CN, —NH₂, —C(O)NH₂, —SO₂NH₂, —NHNH₂, —ONH₂, —NHC(O)NHNH₂, —NHC(O)NH₂, —NHC(NH)NH₂, —NHOH, substituted or unsubstituted alkyl, or substituted or unsubstituted heteroalkyl.

R^{7C} and R^{8C} are independently hydrogen, —CN, —NH₂, —C(O)NH₂, —NHNH₂, —ONH₂, —NHC(O)NHNH₂, —NHC(O)NH₂, —NHC(NH)NH₂, —NHOH, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

L⁹ is independently a bond,

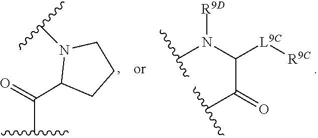

R^{9C} is independently hydrogen, substituted or unsubstituted alkyl, substituted or unsubstituted cycloalkyl, or substituted or unsubstituted aryl.

L¹⁰ is independently a bond,

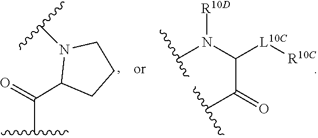

R^{10C} is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

L¹¹ is independently a bond or

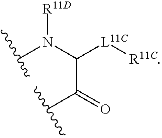

R^{11C} is independently hydrogen, —CN, —OH, —C(O)OH, —NO₂, —SO₃H, —OSO₃H, —NH₂, —C(O)NH₂, —SO₂NH₂, —NHNH₂, —ONH₂, —NHC(O)NHNH₂, —NHC(O)NH₂, —NHC(NH)NH₂, substituted or unsubstituted alkyl, or substituted or unsubstituted heteroalkyl.

R^{12C} is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

L¹³ is independently

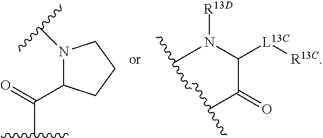

R^{13C} is independently hydrogen, —OH, —NH₂, —C(O)OH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl.

L¹⁴ is independently a bond or

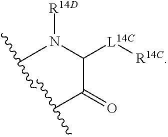

R^{14C} is independently hydrogen, —NH₂, —C(O)OH, —C(O)NH₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

L¹⁵ is independently a bond or

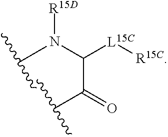

R^{15C} is independently hydrogen, —NH₂, —C(O)OH, —C(O)NH₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Two substituents selected from R^{1C}, R^{2C}, R^{3C}, R^{4C}, R^{5C}, R^{6C}, R^{7C}, R^{8C}, R^{9C}, R^{10C}, R^{11C}, R^{12C}, R^{13C}, R^{14C}, and R^{15C} may optionally be joined to form a covalent linker.

R^{14D} and R^{15D} are independently hydrogen or unsubstituted C₁-C₄ alkyl.

In an aspect is provided a pharmaceutical composition including a compound described herein, or a pharmaceutically acceptable salt thereof, and a pharmaceutically acceptable excipient.

In an aspect is provided a method of treating a cancer in a patient in need of such treatment, the method including administering a therapeutically effective amount of a compound described herein, or a pharmaceutically acceptable salt thereof, to the patient.

In an aspect is provided a method of modulating the activity of a human Ras protein, the method including contacting the human Ras protein with an effective amount of a compound described herein, or a pharmaceutically acceptable salt thereof.

### BRIEF DESCRIPTION OF THE DRAWINGS

FIGS. 1A-1E. Selection of cyclic peptides that selectively bind to State 1 of GTP-bound K-Ras(G12D) from the Random nonstandard Peptide Integrated Discovery (RaPID) mRNA display library. FIG. 1A: Cyclic peptide selection was performed for a total of five rounds using K-Ras(G12D/T35S)·GppNHp as the positive selection target and K-Ras(G12D/T35S)·GDP as the negative selection target. FIG. 1B: Top 20 hits clustered by sequence alignment. One peptide from each cluster (bold typeface) was chosen for further characterization. FIGS. 1C-1E: Structures of three distinct peptides identified from the selection. Bonds in bold indicate the constant regions of the cyclic peptide backbone, including sulfide bridge, the starting amino acid chloroacetyl-D-tyrosine and the ending amino acids cysteine and glycine.

FIGS. 2A-2B. A separate screen using K-Ras(G12D/T35S)·GppNHp as positive selection target and empty beads as negative selection target (FIG. 2A) led to primarily GDP-state selective binders of K-Ras (FIG. 2B). Note that some of the hits do overlap with the results in the primary screen: peptide 2 is identical to KD1, and peptide 20 is identical to KD2.

FIGS. 3A-3D. Cyclic peptides block the interaction of K-Ras(G12D) and effector proteins. FIG. 3A: Illustration of a biochemical assay that detects Ras-Raf interaction by time-resolved fluorescence energy transfer (TR-FRET). FIG. 3B: Cyclic peptides block Ras-Raf interaction with selective for the G12D mutant over wildtype K-Ras. FIG. 3C: Illustration of a biochemical assay that monitors Sos-mediated nucleotide exchange using a fluorescent-GDP analog. FIG. 3D: KD2 and KD17, but not KD1, inhibit Sos-mediated nucleotide exchange of K-Ras(G12D).

FIGS. 4A-4B. Thermal denaturation experiments reveal that KD1, KD2, and KD17 do not increase the melting temperature of K-Ras (G12D).

FIGS. 5A-5D. Crystal structure of KD2 bound to K-Ras(G12D)·GppNHp. FIG. 5A: KD2 binds in the Switch II Groove of K-Ras(G12D)·GppNHp. FIG. 5B: 2Fo-Fc map showing the electron density of KD2, Asp12, and relevant water molecules, contoured at 1.0 σ. FIG. 5C: KD2 forms an intricate hydrogen bond network intramolecularly and intermolecularly with K-Ras. FIG. 5D: Comparison of K-Ras(G12D)·GppNHp·KD2 structure with unliganded K-Ras(G12D)·GppNHp.

FIGS. 6A-6C. Additional views of the KD2·KRas(G12D)·GppNHp cocrystal structure. FIG. 6A: Side view of the α2 helix showing the 40° rotation upon KD2 binding.

FIG. 6B: Comparison of K-Ras(G12D)·GppNHp·KD2 structure with unliganded K-Ras(G12D)·GDP. The conformation of the α2 helix in the KD2-bound structure more resembles that of K-Ras(G12D)·GDP. FIG. 6C: Comparison of K-Ras(G12D)·GppNHp·KD2 structure with H-Ras(M72C)·GppNHp·Compound 3, the latter a previously reported ligand that binds to Ras proteins in the Switch II groove.

FIG. 7. Overlaid HSQC spectra of apo, KD2-bound, and KD17-bound K-Ras(G12D)·GppNHp showing global chemical shift perturbation upon ligand binding.

FIGS. 8A-8D. Substitution of Thr10 in KD2 improves the potency for blocking Ras-Raf interaction. FIG. 8A: Thr10 in KD2 is in proximity with Asp12 of K-Ras(G12D).

FIG. 8B: Structure of KD2 with Thr10 highlighted. FIG. 8C: Thr10 mutants of KD2 are more potent inhibitors of Ras-Raf interaction. FIG. 8D: Thr10 mutants of KD2 do not inhibit wildtype K-Ras-Raf interaction.

FIGS. 9A-9D. Bicyclic variants of KD2 exhibit improved potency for Block Ras-Raf interaction. FIG. 9A: Val4 and Arg9 on KD2 are solvent exposed and participate in neither interaction with K-Ras nor intramolecular interactions. FIG. 9B: Structures of bicyclic variants of KD2. FIG. 9C: Bicyclic variants of KD2 are more potent inhibitors of K-Ras(G12D)-Raf interaction. FIG. 9D: Bicyclic variants of KD2 show improved inhibition of K-Ras(wildtype)-Raf interaction at high concentrations.

FIGS. 10A-10E. KD1, KD2, and KD17 do not exhibit cellular activity, likely due to low cell permeability. FIG. 10A: Treatment of a KRAS(G12D) mutant cell line SW1990 with 10 μM KD1, KD2, KD17, or analogs of KD2 for 24 h does not inhibit Ras signaling. FIG. 10B: Chloroalkane cell penetration assay reveals low cellular permeability of KD2. FIG. 10C: A PROTAC molecule (KD2-Thalidomide) did not induce K-Ras degradation in SW1990 cells. FIG. 10D: Structure of ct-KD2. FIG. 10E: Structure of KD2-Thalidomide.

FIGS. 11A-11B. An electrophile based on KD2 structure is a covalent ligand of K-Ras(G12C) with selectivity for the GTP-state.

FIG. 12. Identifying cyclic peptides of KRAS(G12D) from a mRNA display library. Positive selection: KRAS(G12D/T35S)·GppNHp. Negative selection: KRAS(G12D/T35S)·GDP.

FIG. 13. Three distinct cyclic peptide scaffolds with selectivity for GTP state.

FIG. 14. Cyclic peptides show preferential binding to the GTP-bound state of K-Ras(G12D).

FIG. 15. KD17, but not KD1 or KD2, inhibits Sos mediated nucleotide exchange. Assay conditions: 1 μM KRAS(G12D)·MantGDP; 1 mM GDP; 50 μM compound or DMSO; 1 μM Sos or 5 mM EDTA, 23° C. Average of three measurements.

FIG. 16. KD17, but not KD1 or KD2, inhibits Sos mediated nucleotide exchange. Assay conditions: 1 μM KRAS(G12D)·MantGDP; 1 mM GTP; 50 μM compound or DMSO; 1 μM Sos or 5 mM EDTA, 23° C. Average of three measurements.

FIGS. 17A-17C. Mutations are tolerated, and some improve activity. FIG. 17C: Structures of selected non-natural amino acids.

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di-, and multivalent radicals. The alkyl may include a designated number of carbons (e.g., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds. In embodiments, the alkyl is fully saturated. In embodiments, the alkyl is monounsaturated. In embodiments, the alkyl is polyunsaturated.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene. The term “alkynylene” by itself or as part of another substituent, means, unless otherwise stated, a divalent radical derived from an alkyne. In embodiments, the alkylene is fully saturated. In embodiments, the alkylene is monounsaturated. In embodiments, the alkylene is polyunsaturated. In embodiments, an alkenylene includes one or more double bonds. In embodiments, an alkynylene includes one or more triple bonds.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —S—CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds. In embodiments, the heteroalkyl is fully saturated. In embodiments, the heteroalkyl is monounsaturated. In embodiments, the heteroalkyl is polyunsaturated.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′- and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. The term “heteroalkenylene,” by itself or as part of another substituent, means, unless otherwise stated, a divalent radical derived from a heteroalkene. The term “heteroalkynylene” by itself or as part of another substituent, means, unless otherwise stated, a divalent radical derived from a heteroalkyne. In embodiments, the heteroalkylene is fully saturated. In embodiments, the heteroalkylene is monounsaturated. In embodiments, the heteroalkylene is polyunsaturated. In embodiments, a heteroalkenylene includes one or more double bonds. In embodiments, a heteroalkynylene includes one or more triple bonds.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. In embodiments, the cycloalkyl is fully saturated. In embodiments, the cycloalkyl is monounsaturated. In embodiments, the cycloalkyl is polyunsaturated. In embodiments, the heterocycloalkyl is fully saturated. In embodiments, the heterocycloalkyl is monounsaturated. In embodiments, the heterocycloalkyl is polyunsaturated.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. A bicyclic or multicyclic cycloalkyl ring system refers to multiple rings fused together wherein at least one of the fused rings is a cycloalkyl ring and wherein the multiple rings are attached to the parent molecular moiety through any carbon atom contained within a cycloalkyl ring of the multiple rings.

In embodiments, the term “heterocycloalkyl” means a monocyclic, bicyclic, or a multicyclic heterocycloalkyl ring system. In embodiments, heterocycloalkyl groups are fully saturated. A bicyclic or multicyclic heterocycloalkyl ring system refers to multiple rings fused together wherein at least one of the fused rings is a heterocycloalkyl ring and wherein the multiple rings are attached to the parent molecular moiety through any atom contained within a heterocycloalkyl ring of the multiple rings.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. In embodiments, a fused ring aryl refers to multiple rings fused together wherein at least one of the fused rings is an aryl ring and wherein the multiple rings are attached to the parent molecular moiety through any carbon atom contained within an aryl ring of the multiple rings. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). In embodiments, the term “heteroaryl” includes fused ring heteroaryl groups (i.e., multiple rings fused together wherein at least one of the fused rings is a heteroaromatic ring and wherein the multiple rings are attached to the parent molecular moiety through any atom contained within a heteroaromatic ring of the multiple rings). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzooxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g., substituents for cycloalkyl or heterocycloalkyl rings). Spirocyclic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g., all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker). In embodiments, the alkylarylene group has the formula:

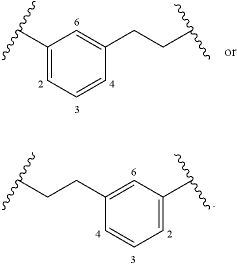 [SMILES: *CCc1cccc(*)c1 ; *CCc1cccc(*)c1]

An alkylarylene moiety may be substituted (e.g., with a substituent group) on the alkylene moiety or the arylene linker (e.g., at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃, —SO₃H, —OSO₃H, —SO₂NH₂, —NHNH₂, —ONH₂, —NHC(O)NHNH₂, substituted or unsubstituted C₁-C₈ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, halogen, —SiR′R″R′″, —OC(O)R′, —C(O)R′, —CO₂R′, —CONR′R″, —OC(O)NR′R″, —NR″C(O)R′, —NR′C(O)NR″R′″, —NR″C(O)₂R′, —NRC(NR′R″R′″)═NR″″, —NRC(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, halogen, —SiR′R″R′″, —OC(O)R′, —C(O)R′, —CO₂R′, —CONR′R″, —OC(O)NR′R″, —NR″C(O)R′, —NR′C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g., cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g., a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), selenium (Se), and silicon (Si). In embodiments, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
   (i) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
      (a) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

In a recited claim or chemical formula description herein, each R substituent or L linker that is described as being “substituted” without reference as to the identity of any chemical moiety that composes the “substituted” group (also referred to herein as an “open substitution” on an R substituent or L linker or an “openly substituted” R substituent or L linker), the recited R substituent or L linker may, in embodiments, be substituted with one or more first substituent groups as defined below.

The first substituent group is denoted with a corresponding first decimal point numbering system such that, for example, R¹ may be substituted with one or more first substituent groups denoted by R^{1.1}, R² may be substituted with one or more first substituent groups denoted by R^{2.1}, R³ may be substituted with one or more first substituent groups denoted by R^{3.1}, R⁴ may be substituted with one or more first substituent groups denoted by R^{4.1}, R⁵ may be substituted with one or more first substituent groups denoted by R^{5.1}, and the like up to or exceeding an R¹⁰⁰ that may be substituted with one or more first substituent groups denoted by R^{100.1}. As a further example, R^{1A} may be substituted with one or more first substituent groups denoted by R^{1A.1}, R^{2A} may be substituted with one or more first substituent groups denoted by R^{2A.1}, R^{3A} may be substituted with one or more first substituent groups denoted by R^{A.1}, R^{4A} may be substituted with one or more first substituent groups denoted by R^{4A.1}, R^{5A} may be substituted with one or more first substituent groups denoted by R^{5A.1} and the like up to or exceeding an R^{100A} may be substituted with one or more first substituent groups denoted by R^{100A.1}. As a further example, L¹ may be substituted with one or more first substituent groups denoted by R^{L1.1}, L² may be substituted with one or more first substituent groups denoted by R^{L2.1}, L³ may be substituted with one or more first substituent groups denoted by R^{L3.1}, L⁴ may be substituted with one or more first substituent groups denoted by R^{L4.1}, L⁵ may be substituted with one or more first substituent groups denoted by R^{L5.1} and the like up to or exceeding an L¹⁰⁰ which may be substituted with one or more first substituent groups denoted by R^{L100.1}. Thus, each numbered R group or L group (alternatively referred to herein as R^{WW} or L^{WW} wherein “WW” represents the stated superscript number of the subject R group or L group) described herein may be substituted with one or more first substituent groups referred to herein generally as R^{WW.1} or R^{LWW.1}, respectively. In turn, each first substituent group (e.g., R^{1.1}, R^{2.1}, R^{3.1}, R^{4.1}, R^{5.1} . . . R^{100.1}; R^{1A.1}, R^{2A.1}, R^{3A.1}, R^{4A.1}, R^{5A.1} . . . R^{100A.1}; R^{L1.1}, R^{L2.1}, R^{L3.1}, R^{L4.1}, R^{L5.1} . . . R^{L100.1}) may be further substituted with one or more second substituent groups (e.g., R^{1.2}, R^{2.2}, R^{3.2}, R^{4.2}, R^{5.2} . . . R^{100.2}; R^{1A.2}, R^{2A.2}, R^{3A.2}, R^{4A.2}, R^{5A.2} . . . R^{100A.2}; R^{L1.2}, R^{L2.2}, R^{L3.2}, R^{L4.2}, R^{L5.2} . . . R^{L100.2}, respectively). Thus, each first substituent group, which may alternatively be represented herein as R^{WW.1} as described above, may be further substituted with one or more second substituent groups, which may alternatively be represented herein as R^{WW.2}.

Finally, each second substituent group (e.g., R^{1.2}, R^{2.2}, R^{3.2}, R^{4.2}, R^{5.2} . . . R^{100.2}; R^{1A.2}, R^{2A.2}, R^{3A.2}, R^{4A.2}, R^{5A.2} . . . R^{100A.2}; R^{L1.2}, R^{L2.2}, R^{L3.2}, R^{L4.2}, R^{L5.2} . . . R^{L100.2}) may be further substituted with one or more third substituent groups (e.g., R^{1.3}, R^{2.3}, R^{3.3}, R^{4.3}, R^{5.3} . . . R^{100.3}; R^{1A.3}, R^{2A.3}, R^{3A.3}, R^{4A.3}, R^{5A.3} . . . R^{100A.3}; R^{L1.3}, R^{L2.3}, R^{L3.3}, R^{L4.3}, R^{L5.3}, R^{L100.3}; respectively). Thus, each second substituent group, which may alternatively be represented herein as R^{WW.2} as described above, may be further substituted with one or more third substituent groups, which may alternatively be represented herein as R^{WW.3}. Each of the first substituent groups may be optionally different. Each of the second substituent groups may be optionally different. Each of the third substituent groups may be optionally different.

Thus, as used herein, R^{WW} represents a substituent recited in a claim or chemical formula description herein which is openly substituted. “WW” represents the stated superscript number of the subject R group (1, 2, 3, 1A, 2A, 3A, 1B, 2B, 3B, etc.). Likewise, L^{WW} is a linker recited in a claim or chemical formula description herein which is openly substituted. Again, “WW” represents the stated superscript number of the subject L group (1, 2, 3, 1A, 2A, 3A, 1B, 2B, 3B, etc.). As stated above, in embodiments, each R^{WW} may be unsubstituted or independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group may be unsubstituted or independently substituted with one or more third substituent groups, referred to herein as R^{WW.3}. Similarly, each L^{WW} linker may be unsubstituted or independently substituted with one or more first substituent groups, referred to herein as R^{LWW.1}; each first substituent group, R^{LWW.1}, may be unsubstituted or independently substituted with one or more second substituent groups, referred to herein as R^{LWW.2}; and each second substituent group may be unsubstituted or independently substituted with one or more third substituent groups, referred to herein as R^{LWW.3}. Each first substituent group is optionally different. Each second substituent group is optionally different. Each third substituent group is optionally different. For example, if R^{WW} is phenyl, the said phenyl group is optionally substituted by one or more R^{WW.1} groups as defined herein below, e.g., when R^{WW.1} is R^{WW.2}-substituted or unsubstituted alkyl, examples of groups so formed include but are not limited to itself optionally substituted by 1 or more R^{WW.2}, which R^{WW.2} is optionally substituted by one or more R^{WW.3}. By way of example when the R^{WW} group is phenyl substituted by R^{WW.1}, which is methyl, the methyl group may be further substituted to form groups including but not limited to:

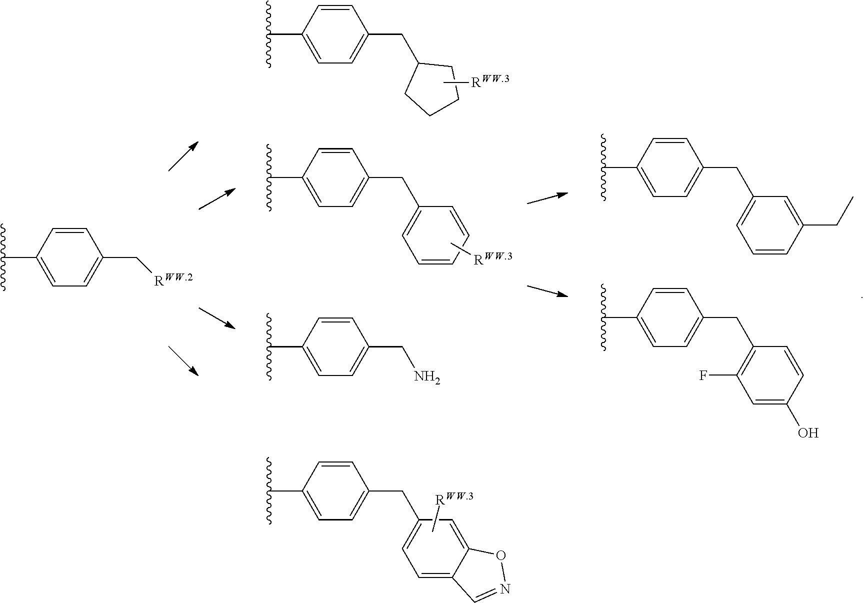

R^{WW.1} is independently oxo, halogen, —CX^{WW.1}₃, —CHX^{WW.1}₂, —CH₂X^{WW.1}, —OCX^{WW.1}₃, —OCH₂X^{WW.1}, —OCHX^{WW.1}2, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.2}-substituted or unsubstituted alkyl (e.g., C₁-C₈, C₁-C₆, C₁-C₄, or C₁-C₂), R^{WW.2}-substituted or unsubstituted heteroalkyl (e.g., 2 to 8 membered, 2 to 6 membered, 4 to 6 membered, 2 to 3 membered, or 4 to 5 membered), R^{WW.2}-substituted or unsubstituted cycloalkyl (e.g., C₃-C₈, C₃-C₆, C₄-C₆, or C₅-C₆), R^{WW.2}-substituted or unsubstituted heterocycloalkyl (e.g., 3 to 8 membered, 3 to 6 membered, 4 to 6 membered, 4 to 5 membered, or 5 to 6 membered), R^{WW.2}-substituted or unsubstituted aryl (e.g., C₆-C₁₂, C₆-C₁₀, or phenyl), or R^{WW.2}-substituted or unsubstituted heteroaryl (e.g., 5 to 12 membered, 5 to 10 membered, 5 to 9 membered, or 5 to 6 membered). In embodiments, R^{WW.1} is independently oxo, halogen, —CX^{WW.1}₃, —CHX^{WW.1}₂, —CH₂X^{WW.1}, —OCX^{WW.1}₃, —OCH₂X^{WW.1}, —OCHX^{WW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NH₄NH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.1} is independently —F, —Cl, —Br, or —I.

R^{WW.2} is independently oxo, halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.3}-substituted or unsubstituted alkyl (e.g., C₁-C₈, C₁-C₆, C₁-C₄, or C₁-C₂), R^{WW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{WW.3}-substituted or unsubstituted heterocycloalkyl (e.g., 3 to 8 membered, 3 to 6 membered, 4 to 6 membered, 4 to 5 membered, or 5 to 6 membered), R^{WW.3}-substituted or unsubstituted aryl (e.g., C₆-C₁₂, C₆-C₁₀, or phenyl), or R^{WW.3}-substituted or unsubstituted heteroaryl (e.g., 5 to 12 membered, 5 to 10 membered, 5 to 9 membered, or 5 to 6 membered). In embodiments, R^{WW.2} is independently oxo, halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NH₄NH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.2} is independently —F, —Cl, —Br, or —I.

R^{WW.3} is independently oxo, halogen, —CX^{WW.3}₃, —CHX^{WW.3}₂, —CH₂X^{WW.3}, —OCX^{WW.3}₃, —OCH₂X^{WW.3}, —OCHX^{WW.3}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.3} is independently —F, —Cl, —Br, or —I.

Where two different R^{WW} substituents are joined together to form an openly substituted ring (e.g., substituted cycloalkyl, substituted heterocycloalkyl, substituted aryl or substituted heteroaryl), in embodiments the openly substituted ring may be independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group, R^{WW.2}, may be unsubstituted or independently substituted with one or more third substituent groups, referred to herein as R^{WW.3}; and each third substituent group, R^{WW.3}, is unsubstituted. Each first substituent group is optionally different. Each second substituent group is optionally different. Each third substituent group is optionally different. In the context of two different R^{WW} substituents joined together to form an openly substituted ring, the “WW” symbol in the R^{WW.1}, R^{WW.2} and R^{WW.3} refers to the designated number of one of the two different R^{WW} substituents. For example, in embodiments where R^{100A} and R^{100B} are optionally joined together to form an openly substituted ring, R^{WW.1} is R^{100A.1}, R^{WW.2} is R^{100A.2}, and R^{WW.3} is R^{100A.3}. Alternatively, in embodiments where R^{100A} and R^{100B} are optionally joined together to form an openly substituted ring, R^{WW.1} is R^{100B.1}, R^{WW.2} is R^{100B.2}, and R^{WW.3} is R^{100B.3}. R^{WW.1}, R^{WW.2} and R^{WW.3} in this paragraph are as defined in the preceding paragraphs.

R^{LWW.1} is independently oxo, halogen, —CX^{LWW.1}₃, —CHX^{LWW.1}₂, —CH₂X^{LWW.1}, —OCX^{LWW.1}₃, —OCH₂X^{LWW.1}, —OCHX^{LWW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{LWW.2}-substituted or unsubstituted alkyl (e.g., C₁-C₈, C₁-C₆, C₁-C₄, or C₁-C₂), R^{LWW.2}-substituted or unsubstituted heteroalkyl (e.g., 2 to 8 membered, 2 to 6 membered, 4 to 6 membered, 2 to 3 membered, or 4 to 5 membered), R^{LWW.2}-substituted or unsubstituted cycloalkyl (e.g., C₃-C₈, C₃-C₆, C₄-C₆, or C₅-C₆), R^{LWW.2}-substituted or unsubstituted heterocycloalkyl (e.g., 3 to 8 membered, 3 to 6 membered, 4 to 6 membered, 4 to 5 membered, or 5 to 6 membered), R^{LWW.2}-substituted or unsubstituted aryl (e.g., C₆-C₁₂, C₆-C₁₀, or phenyl), or R^{LWW.2}-substituted or unsubstituted heteroaryl (e.g., 5 to 12 membered, 5 to 10 membered, 5 to 9 membered, or 5 to 6 membered). In embodiments, R^{LWW.1} is independently oxo, halogen, —CX^{LWW.1}₃, —CHX^{LWW.1}₂, —CH₂X^{LWW.1}, —OCX^{LWW.1}₃, —OCH₂X^{LWW.1}, —OCHX^{LWW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.1} is independently —F, —Cl, —Br, or —I.

R^{LWW.2} is independently oxo, halogen, —CX^{LWW.2}₃, —CHX^{LWW.2}₂, —CH₂X^{LWW.2}—OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{LWW.3}-substituted or unsubstituted alkyl (e.g., C₁-C₈, C₁-C₆, C₁-C₄, or C₁-C₂), R^{LWW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{LWW.3}-substituted or unsubstituted heterocycloalkyl (e.g., 3 to 8 membered, 3 to 6 membered, 4 to 6 membered, 4 to 5 membered, or 5 to 6 membered), R^{LWW.3}-substituted or unsubstituted aryl (e.g., C₆-C₁₂, C₆-C₁₀, or phenyl), or R^{LWW.3}-substituted or unsubstituted heteroaryl (e.g., 5 to 12 membered, 5 to 10 membered, 5 to 9 membered, or 5 to 6 membered). In embodiments, R^{LWW.2} is independently oxo, halogen, —CX^{LWW.2}₃, —CHX^{LWW.2}₂, —CH₂X^{LWW.2}, —OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.2} is independently —F, —Cl, —Br, or —I.

R^{LWW.3} is independently oxo, halogen, —CX^{LWW.3}₃, —CHX^{LWW.3}₂, —CH₂X^{LWW.3}, —OCX^{LWW.3}₃, —OCH₂X^{LWW.3}, —OCHX^{LWW.3}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.3} is independently —F, —Cl, —Br, or —I.

In the event that any R group recited in a claim or chemical formula description set forth herein (R^{WW} substituent) is not specifically defined in this disclosure, then that R group (R^{WW} group) is hereby defined as independently oxo, halogen, —CX^{WW}₃, —CHX^{WW}₂, —CH₂X^{WW}, —OCX^{WW}₃, —OCH₂X^{WW}, —OCHX^{WW}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.1}-substituted or unsubstituted alkyl (e.g., C₁-C₈, C₁-C₆, C₁-C₄, or C₁-C₂), R^{WW.1}-substituted or unsubstituted heteroalkyl (e.g., 2 to 8 membered, 2 to 6 membered, 4 to 6 membered, 2 to 3 membered, or 4 to 5 membered), R^{WW.1}-substituted or unsubstituted cycloalkyl (e.g., C₃-C₈, C₃-C₆, C₄-C₆, or C₅-C₆), R^{WW.1}-substituted or unsubstituted heterocycloalkyl (e.g., 3 to 8 membered, 3 to 6 membered, 4 to 6 membered, 4 to 5 membered, or 5 to 6 membered), R^{WW.1}-substituted or unsubstituted aryl (e.g., C₆-C₁₂, C₆-C₁₀, or phenyl), or R^{WW.1}-substituted or unsubstituted heteroaryl (e.g., 5 to 12 membered, 5 to 10 membered, 5 to 9 membered, or 5 to 6 membered). X^{WW} is independently —F, —Cl, —Br, or —I. Again, “WW” represents the stated superscript number of the subject R group (e.g., 1, 2, 3, 1A, 2A, 3A, 1B, 2B, 3B, etc.). R^{WW.1}, R^{WW.2}, and R^{WW.3} are as defined above.

In the event that any L linker group recited in a claim or chemical formula description set forth herein (i.e., an L^{WW} substituent) is not explicitly defined, then that L group (L^{WW} group) is herein defined as independently a bond, —O—, —NH—, —C(O)—, —C(O)NH—, —NHC(O)—, —NHC(O)NH—, —NHC(NH)NH—, —C(O)O—, —OC(O)—, —S—, —SO₂—, —SO₂NH—, R^{LWW.1}-substituted or unsubstituted alkylene (e.g., C₁-C₈, C₁-C₆, C₁-C₄, or C₁-C₂), R^{LWW.1}-substituted or unsubstituted heteroalkylene (e.g., 2 to 8 membered, 2 to 6 membered, 4 to 6 membered, 2 to 3 membered, or 4 to 5 membered), R^{LWW.1}-substituted or unsubstituted cycloalkylene (e.g., C₃-C₈, C₃-C₆, C₄-C₆, or C₅-C₆), R^{LWW.1}-substituted or unsubstituted heterocycloalkylene (e.g., 3 to 8 membered, 3 to 6 membered, 4 to 6 membered, 4 to 5 membered, or 5 to 6 membered), R^{LWW.1}-substituted or unsubstituted arylene (e.g., C₆-C₁₂, C₆-C₁₀, or phenyl), or R^{LWW.1}-substituted or unsubstituted heteroarylene (e.g., 5 to 12 membered, 5 to 10 membered, 5 to 9 membered, or 5 to 6 membered). Again, “WW” represents the stated superscript number of the subject L group (1, 2, 3, 1A, 2A, 3A, 1B, 2B, 3B, etc.). R^{LWW.1}, as well as R^{LWW.2} and R^{LWW.3} are as defined above.

Certain compounds of the present disclosure possess asymmetric carbon atoms (optical or chiral centers) or double bonds; the enantiomers, racemates, diastereomers, tautomers, geometric isomers, stereoisomeric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

As used herein, the terms “bioconjugate” and “bioconjugate linker” refer to the resulting association between atoms or molecules of bioconjugate reactive groups or bioconjugate reactive moieties. The association can be direct or indirect. For example, a conjugate between a first bioconjugate reactive group (e.g., —NH₂, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g., a first linker of second linker), or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). In embodiments, bioconjugates or bioconjugate linkers are formed using bioconjugate chemistry (i.e., the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g., a sulfhydryl). In embodiments, the first bioconjugate reactive group (e.g., haloacetyl moiety) is covalently attached to the second bioconjugate reactive group (e.g., a sulfhydryl). In embodiments, the first bioconjugate reactive group (e.g., pyridyl moiety) is covalently attached to the second bioconjugate reactive group (e.g., a sulfhydryl). In embodiments, the first bioconjugate reactive group (e.g., —N-hydroxysuccinimide moiety) is covalently attached to the second bioconjugate reactive group (e.g., an amine). In embodiments, the first bioconjugate reactive group (e.g., maleimide moiety) is covalently attached to the second bioconjugate reactive group (e.g., a sulfhydryl). In embodiments, the first bioconjugate reactive group (e.g., -sulfo-N-hydroxysuccinimide moiety) is covalently attached to the second bioconjugate reactive group (e.g., an amine).

Useful bioconjugate reactive moieties used for bioconjugate chemistries herein include, for example: (a) carboxyl groups and various derivatives thereof including, but not limited to, N-hydroxysuccinimide esters, N-hydroxybenzotriazole esters, acid halides, acyl imidazoles, thioesters, p-nitrophenyl esters, alkyl, alkenyl, alkynyl and aromatic esters; (b) hydroxyl groups which can be converted to esters, ethers, aldehydes, etc.; (c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups which are capable of participating in Diels-Alder reactions such as, for example, maleimido or maleimide groups; (e) aldehyde or ketone groups such that subsequent derivatization is possible via formation of carbonyl derivatives such as, for example, imines, hydrazones, semicarbazones or oximes, or via such mechanisms as Grignard addition or alkyllithium addition; (f) sulfonyl halide groups for subsequent reaction with amines, for example, to form sulfonamides; (g) thiol groups, which can be converted to disulfides, reacted with acyl halides, or bonded to metals such as gold, or react with maleimides; (h) amine or sulfhydryl groups (e.g., present in cysteine), which can be, for example, acylated, alkylated or oxidized; (i) alkenes, which can undergo, for example, cycloadditions, acylation, Michael addition, etc.; (j) epoxides, which can react with, for example, amines and hydroxyl compounds; (k) phosphoramidites and other standard functional groups useful in nucleic acid synthesis; (l) metal silicon oxide bonding; (m) metal bonding to reactive phosphorus groups (e.g., phosphines) to form, for example, phosphate diester bonds; (n) azides coupled to alkynes using copper catalyzed cycloaddition click chemistry; and (o) biotin conjugate can react with avidin or streptavidin to form a avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

“Analog,” “analogue,” or “derivative” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an”, as used in herein means one or more. In addition, the phrase “substituted with a[n]”, as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl”, the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13A}, R^{13B}, R^{13C}, R^{13D}, etc., wherein each of R^{13A}, R^{13B}, R^{13C}, R^{13D}, etc. is defined within the scope of the definition of R¹³ and optionally differently.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, propionates, tartrates (e.g., (+)-tartrates, (−)-tartrates, or mixtures thereof including racemic mixtures), succinates, benzoates, and salts with amino acids such as glutamic acid, and quaternary ammonium salts (e.g., methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

A polypeptide, or a cell is “recombinant” when it is artificial or engineered, or derived from or contains an artificial or engineered protein or nucleic acid (e.g., non-natural or not wild type). For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

“Co-administer” is meant that a composition described herein is administered at the same time, just prior to, or just after the administration of one or more additional therapies. The compounds of the invention can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation).

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., Spodoptera) and human cells. Cells may be useful when they are naturally nonadherent or have been treated not to adhere to surfaces, for example by trypsinization.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. For example, the certain methods presented herein successfully treat cancer by decreasing the incidence of cancer and or causing remission of cancer. In some embodiments of the compositions or methods described herein, treating cancer includes slowing the rate of growth or spread of cancer cells, reducing metastasis, or reducing the growth of metastatic tumors. The term “treating” and conjugations thereof, include prevention of an injury, pathology, condition, or disease. In embodiments, treating is preventing. In embodiments, treating does not include preventing. In embodiments, the treating or treatment is no prophylactic treatment.

An “effective amount” is an amount sufficient for a compound to accomplish a stated purpose relative to the absence of the compound (e.g., achieve the effect for which it is administered, treat a disease, reduce enzyme activity, increase enzyme activity, reduce signaling pathway, reduce one or more symptoms of a disease or condition. An example of an “effective amount” is an amount sufficient to contribute to the treatment, prevention, or reduction of a symptom or symptoms of a disease, which could also be referred to as a “therapeutically effective amount” when referred to in this context. A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. An “activity increasing amount,” as used herein, refers to an amount of agonist required to increase the activity of an enzyme relative to the absence of the agonist. A “function increasing amount,” as used herein, refers to the amount of agonist required to increase the function of an enzyme or protein relative to the absence of the agonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity (e.g., signaling pathway) of a protein in the absence of a compound as described herein (including embodiments, examples, figures, or Tables).

“Contacting” is used in accordance with its plain ordinary meaning and refers to the process of allowing at least two distinct species (e.g., chemical compounds including biomolecules, or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be a compound as described herein and a cellular component (e.g., protein, ion, lipid, nucleic acid, nucleotide, amino acid, protein, particle, organelle, cellular compartment, microorganism, virus, lipid droplet, vesicle, small molecule, protein complex, protein aggregate, or macromolecule). In some embodiments contacting includes allowing a compound described herein to interact with a cellular component (e.g., protein, ion, lipid, nucleic acid, nucleotide, amino acid, protein, particle, virus, lipid droplet, organelle, cellular compartment, microorganism, vesicle, small molecule, protein complex, protein aggregate, or macromolecule) that is involved in a signaling pathway.

As defined herein, the term “activation,” “activate,” “activating” and the like in reference to a protein refers to conversion of a protein into a biologically active derivative from an initial inactive or deactivated state. The terms reference activation, or activating, sensitizing, or up-regulating signal transduction or enzymatic activity or the amount of a protein decreased in a disease.

The terms “agonist,” “activator,” “upregulator,” etc. refer to a substance capable of detectably increasing the expression or activity of a given gene or protein. The agonist can increase expression or activity by at least 10%, 20%, 30%, 40%, 50%, 60%, 70%, 80%, 90%, 95%, 96%, 97%, 98%, or 99% in comparison to a control in the absence of the agonist. In certain instances, expression or activity is 1.5-fold, 2-fold, 3-fold, 4-fold, 5-fold, 10-fold or higher than the expression or activity in the absence of the agonist.

As defined herein, the term “inhibition,” “inhibit,” “inhibiting” and the like in reference to a cellular component-inhibitor interaction means negatively affecting (e.g., decreasing) the activity or function of the cellular component (e.g., decreasing the signaling pathway stimulated by a cellular component (e.g., protein, ion, lipid, virus, lipid droplet, nucleic acid, nucleotide, amino acid, protein, particle, organelle, cellular compartment, microorganism, vesicle, small molecule, protein complex, protein aggregate, or macromolecule)), relative to the activity or function of the cellular component in the absence of the inhibitor. In embodiments inhibition means negatively affecting (e.g., decreasing) the concentration or levels of the cellular component relative to the concentration or level of the cellular component in the absence of the inhibitor. In some embodiments, inhibition refers to reduction of a disease or symptoms of disease. In some embodiments, inhibition refers to a reduction in the activity of a signal transduction pathway or signaling pathway (e.g., reduction of a pathway involving the cellular component). Thus, inhibition includes, at least in part, partially or totally blocking stimulation, decreasing, preventing, or delaying activation, or inactivating, desensitizing, or down-regulating the signaling pathway or enzymatic activity or the amount of a cellular component.

The terms “inhibitor,” “repressor,” “antagonist,” or “downregulator” interchangeably refer to a substance capable of detectably decreasing the expression or activity of a given gene or protein. The antagonist can decrease expression or activity by at least 10%, 20%, 30%, 40%, 50%, 60%, 70%, 80%, 90%, 95%, 96%, 97%, 98%, or 99% in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “modulator” refers to a composition that increases or decreases the level of a target molecule or the function of a target molecule or the physical state of the target of the molecule (e.g., a target may be a cellular component (e.g., protein, ion, lipid, virus, lipid droplet, nucleic acid, nucleotide, amino acid, protein, particle, organelle, cellular compartment, microorganism, vesicle, small molecule, protein complex, protein aggregate, or macromolecule)) relative to the absence of the composition.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

“Disease” or “condition” refer to a state of being or health status of a patient or subject capable of being treated with the compounds or methods provided herein. In some embodiments, the disease is a disease related to (e.g., caused by) a cellular component (e.g., protein, ion, lipid, nucleic acid, nucleotide, amino acid, protein, particle, organelle, cellular compartment, microorganism, vesicle, small molecule, protein complex, protein aggregate, or macromolecule). In embodiments, the disease is a cancer.

As used herein, the term “cancer” refers to all types of cancer, neoplasm or malignant tumors found in mammals (e.g., humans), including leukemia, lymphoma, carcinomas and sarcomas. Exemplary cancers that may be treated with a compound or method provided herein include cancer of the thyroid, endocrine system, brain, breast, cervix, colon, head and neck, liver, kidney, lung, non-small cell lung, melanoma, mesothelioma, ovary, sarcoma, stomach, uterus, medulloblastoma, colorectal cancer, or pancreatic cancer. Additional examples include, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

As used herein, the term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cutaneous T-cell lymphoma, peripheral T-cell lymphoma, anaplastic large cell lymphoma, mycosis fungoides, and precursor T-lymphoblastic lymphoma.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungual melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epidermoid carcinoma, carcinoma epitheliale adenoides, exophytic carcinoma, carcinoma ex ulcere, carcinoma fibrosum, gelatinifori carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

The term “drug” is used in accordance with its common meaning and refers to a substance which has a physiological effect (e.g., beneficial effect, is useful for treating a subject) when introduced into or to a subject (e.g., in or on the body of a subject or patient). A drug moiety is a radical of a drug.

A “detectable agent,” “detectable compound,” “detectable label,” or “detectable moiety” is a substance (e.g., element), molecule, or composition detectable by spectroscopic, photochemical, biochemical, immunochemical, chemical, magnetic resonance imaging, or other physical means. For example, detectable agents include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I ¹²⁵I ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g., fluorescent dyes), modified oligonucleotides (e.g., moieties described in PCT/US2015/022063, which is incorporated herein by reference), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monocrystalline iron oxide nanoparticles, monocrystalline iron oxide, nanoparticle contrast agents, liposomes or other delivery vehicles containing Gadolinium chelate (“Gd-chelate”) molecules, Gadolinium, radioisotopes, radionuclides (e.g., carbon-11, nitrogen-13, oxygen-15, fluorine-18, rubidium-82), fluorodeoxyglucose (e.g., fluorine-18 labeled), any gamma ray emitting radionuclides, positron-emitting radionuclide, radiolabeled glucose, radiolabeled water, radiolabeled ammonia, biocolloids, microbubbles (e.g., including microbubble shells including albumin, galactose, lipid, and/or polymers; microbubble gas core including air, heavy gas(es), perfluorocarbon, nitrogen, octafluoropropane, perflexane lipid microsphere, perflutren, etc.), iodinated contrast agents (e.g., iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide.

Radioactive substances (e.g., radioisotopes) that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y. ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac. Paramagnetic ions that may be used as additional imaging agents in accordance with the embodiments of the disclosure include, but are not limited to, ions of transition and lanthanide metals (e.g., metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb, and Lu.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

The compounds described herein can be used in combination with one another, with other active agents known to be useful in treating a disease associated with cells expressing a disease associated cellular component, or with adjunctive agents that may not be effective alone, but may contribute to the efficacy of the active agent.

In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another.

The compounds described herein can be co-administered with conventional neurodegenerative disease treatments including, but not limited to, Parkinson's disease treatments such as levodopa, carbidopa, selegiline, amantadine, donepezil, galanthamine, rivastigmine, tacrine, dopamine agonists (e.g., bromocriptine, pergolide, pramipexole, ropinirole), anticholinergic drugs (e.g., trihexyphenidyl, benztropine, biperiden, procyclidine), and catechol-O-methyl-transferase inhibitors (e.g., tolcapone, entacapone).

The compounds described herein can also be co-administered with conventional anti-inflammatory disease treatments including, but not limited to, analgesics (e.g., acetaminophen, duloxetine), nonsteroidal anti-inflammatory drugs (e.g., aspirin, ibuprofen, naproxen, diclofenac), corticosteroids (e.g., prednisone, betamethasone, cortisone, dexamethasone, hydrocortisone, methylprednisolone, prednisolone), and opioids (e.g., codeine, fentanyl, hydrocodone, hydromorphone, morphine, meperidine, oxycodone).

“Anti-cancer agent” is used in accordance with its plain ordinary meaning and refers to a composition (e.g., compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is an agent identified herein having utility in methods of treating cancer. In some embodiments, an anti-cancer agent is an agent approved by the FDA or similar regulatory agency of a country other than the USA, for treating cancer. In embodiments, an anti-cancer agent is an agent with antineoplastic properties that has not (e.g., yet) been approved by the FDA or similar regulatory agency of a country other than the USA, for treating cancer. Examples of anti-cancer agents include, but are not limited to, MEK (e.g., MEK1, MEK2, or MEK1 and MEK2) inhibitors (e.g., XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethylmelamine, thiotepa), alkyl sulfonates (e.g., busulfan), nitrosoureas (e.g., carmustine, lomustine, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxuridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g., cisplatin, oxaliplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g., U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; elfomithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarine-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; elfomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin I1 (including recombinant interleukin II, or rlL.sub.2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g., Taxol™ (i.e., paclitaxel), Taxotere™, compounds comprising the taxane skeleton, Erbulozole (i.e., R-55104), Dolastatin 10 (i.e., DLS-10 and NSC-376128), Mivobulin isethionate (i.e., as CI-980), Vincristine, NSC-639829, Discodermolide (i.e., as NVP-XX-A-296), ABT-751 (Abbott, i.e., E-7010), Altorhyrtins (e.g., Altorhyrtin A and Altorhyrtin C), Spongistatins (e.g., Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e., LU-103793 and NSC-D-669356), Epothilones (e.g., Epothilone A, Epothilone B, Epothilone C (i.e., desoxyepothilone A or dEpoA), Epothilone D (i.e., KOS-862, dEpoB, and desoxyepothilone B), Epothilone E, Epothilone F, Epothilone B N-oxide, Epothilone A N-oxide, 16-aza-epothilone B, 21-aminoepothilone B (i.e., BMS-310705), 21-hydroxyepothilone D (i.e., Desoxyepothilone F and dEpoF), 26-fluoroepothilone, Auristatin PE (i.e., NSC-654663), Soblidotin (i.e., TZT-1027), LS-4559-P (Pharmacia, i.e., LS-4577), LS-4578 (Pharmacia, i.e., LS-477-P), LS-4477 (Pharmacia), LS-4559 (Pharmacia), RPR-112378 (Aventis), Vincristine sulfate, DZ-3358 (Daiichi), FR-182877 (Fujisawa, i.e., WS-9885B), GS-164 (Takeda), GS-198 (Takeda), KAR-2 (Hungarian Academy of Sciences), BSF-223651 (BASF, i.e., ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (i.e., LY-355703), AC-7739 (Ajinomoto, i.e., AVE-8063A and CS-39.HCl), AC-7700 (Ajinomoto, i.e., AVE-8062, AVE-8062A, CS-39-L-Ser.HCl, and RPR-258062A), Vitilevuamide, Tubulysin A, Canadensol, Centaureidin (i.e., NSC-106969), T-138067 (Tularik, i.e., T-67, TL-138067 and TI-138067), COBRA-1 (Parker Hughes Institute, i.e., DDE-261 and WHI-261), H10 (Kansas State University), H16 (Kansas State University), Oncocidin A1 (i.e., BTO-956 and DIME), DDE-313 (Parker Hughes Institute), Fijianolide B, Laulimalide, SPA-2 (Parker Hughes Institute), SPA-1 (Parker Hughes Institute, i.e., SPIKET-P), 3-IAABU (Cytoskeleton/Mt. Sinai School of Medicine, i.e., MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, i.e., MF-191), TMPN (Arizona State University), Vanadocene acetylacetonate, T-138026 (Tularik), Monsatrol, lnanocine (i.e., NSC-698666), 3-IAABE (Cytoskeleton/Mt. Sinai School of Medicine), A-204197 (Abbott), T-607 (Tuiarik, i.e., T-900607), RPR-115781 (Aventis), Eleutherobins (such as Desmethyleleutherobin, Desaetyleleutherobin, lsoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (i.e., NSCL-96F037), D-68838 (Asta Medica), D-68836 (Asta Medica), Myoseverin B, D-43411 (Zentaris, i.e., D-81862), A-289099 (Abbott), A-318315 (Abbott), HTI-286 (i.e., SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi)), steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethylstilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guerin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to ¹¹¹In, ⁹⁰Y, or ¹³¹I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g., gefitinib (Iressa™), erlotinib (Tarceva™), cetuximab (Erbitux™), lapatinib (Tykerb™) panitumumab (Vectibix™), vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, or the like. A moiety of an anti-cancer agent is a monovalent anti-cancer agent (e.g., a monovalent form of an agent listed above).

In therapeutic use for the treatment of a disease, compound utilized in the pharmaceutical compositions of the present invention may be administered at the initial dosage of about 0.001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the compound or drug being employed. For example, dosages can be empirically determined considering the type and stage of cancer diagnosed in a particular patient. The dose administered to a patient, in the context of the present invention, should be sufficient to affect a beneficial therapeutic response in the patient over time. The size of the dose will also be determined by the existence, nature, and extent of any adverse side effects that accompany the administration of a compound in a particular patient. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired.

The compounds described herein can be used in combination with one another, with other active agents known to be useful in treating cancer or with adjunctive agents that may not be effective alone, but may contribute to the efficacy of the active agent.

The term “associated” or “associated with” in the context of a substance or substance activity or function associated with a disease (e.g., a protein associated disease, disease associated with a cellular component) means that the disease (e.g., cancer) is caused by (in whole or in part), or a symptom of the disease is caused by (in whole or in part) the substance or substance activity or function or the disease or a symptom of the disease may be treated by modulating (e.g., inhibiting or activating) the substance (e.g., cellular component). As used herein, what is described as being associated with a disease, if a causative agent, could be a target for treatment of the disease.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g., by administering a compound or using a method as described herein), results in reduction of the disease or one or more disease symptoms.

The term “electrophilic” as used herein refers to a chemical group that is capable of accepting electron density. An “electrophilic substituent,” “electrophilic chemical moiety,” or “electrophilic moiety” refers to an electron-poor chemical group, substituent, or moiety (monovalent chemical group), which may react with an electron-donating group, such as a nucleophile, by accepting an electron pair or electron density to form a bond. In some embodiments, the electrophilic substituent of the compound is capable of reacting with a cysteine residue. In some embodiments, the electrophilic substituent is capable of forming a covalent bond with a cysteine residue and may be referred to as a “covalent cysteine modifier moiety” or “covalent cysteine modifier substituent.” The covalent bond formed between the electrophilic substituent and the sulfhydryl group of the cysteine may be a reversible or irreversible bond. In some embodiments, the electrophilic substituent of the compound is capable of reacting with a lysine residue. In some embodiments, the electrophilic substituent of the compound is capable of reacting with a serine residue. In some embodiments, the electrophilic substituent of the compound is capable of reacting with a methionine residue.

“Nucleophilic” as used herein refers to a chemical group that is capable of donating electron density.

The term “isolated,” when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide,” and “protein” are used interchangeably herein to refer to a polymer of amino acid residues, wherein the polymer may in embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

The term “protein complex” is used in accordance with its plain ordinary meaning and refers to a protein which is associated with an additional substance (e.g., another protein, protein subunit, or a compound). Protein complexes typically have defined quaternary structure. The association between the protein and the additional substance may be a covalent bond. In embodiments, the association between the protein and the additional substance (e.g., compound) is via non-covalent interactions. In embodiments, a protein complex refers to a group of two or more polypeptide chains. Proteins in a protein complex are linked by non-covalent protein-protein interactions. A non-limiting example of a protein complex is the proteasome.

The term “protein aggregate” is used in accordance with its plain ordinary meaning and refers to an aberrant collection or accumulation of proteins (e.g., misfolded proteins). Protein aggregates are often associated with diseases (e.g., amyloidosis). Typically, when a protein misfolds as a result of a change in the amino acid sequence or a change in the native environment which disrupts normal non-covalent interactions, and the misfolded protein is not corrected or degraded, the unfolded/misfolded protein may aggregate. There are three main types of protein aggregates that may form: amorphous aggregates, oligomers, and amyloid fibrils. In embodiments, protein aggregates are termed aggresomes.

The term “K-Ras” refers to the protein that in humans is encoded by the KRAS gene. The K-Ras protein is a GTPase, which converts guanosine triphosphate to guanosine diphosphate. A mutation in the K-Ras protein (e.g., an amino acid substitution) can result in various malignancies (e.g., lung adenocarcinoma, pancreatic cancer, or colorectal cancer). The term “K-Ras” may refer to the nucleotide sequence or protein sequence of human KRAS (e.g., Entrez 3845, UniProt P01116, RefSeq NM_004985.4, RefSeq NM_033360.3, RefSeq NP_004976.2, or RefSeq NP_203524.1). In embodiments, K-Ras has the following amino acid sequence:

(SEQ ID NO: 1)
MTEYKLVVVGAGGVGKSALTIQLIQNHFVDEYDPTIEDSYRKQVVIDGET
CLLDILDTAGQEEYSAMRDQYMRTGEGFLCVFAINNTKSFEDIHHYREQI
KRVKDSEDVPMVLVGNKCDLPSRTVDTKQAQDLARSYGIPFIETSAKTRQ
RVEDAFYTLVREIRQYRLKKISKEEKTPGCVKIKKCIIM.

The term “H-Ras” refers to the enzyme that in humans is encoded by the HRAS gene. The H-Ras protein is a GTPase, which converts guanosine triphosphate to guanosine diphosphate. Mutations in the H-Ras protein (e.g., an amino acid substitution) can result in various malignancies (e.g., bladder cancer, thyroid cancer, salivary duct carcinoma, epithelial carcinoma, or kidney cancer). The term “H-Ras” may refer to the nucleotide sequence or protein sequence of human HRAS (e.g., Entrez 3265, UniProt P01112, RefSeq NM_001130442.2, RefSeq NM_001318054.1, RefSeq NM_005343.3, RefSeq NM_00176795.4, RefSeq NP_001123914.1, RefSeq NP_001304983.1, RefSeq NP_005334.1, or RefSeq NP_789765.1). In embodiments, H-Ras has the following amino acid sequence:

(SEQ ID NO: 2)
MTEYKLVVVGAGGVGKSALTIQLIQNHFVDEYDPTIEDSYRKQVVIDGET
CLLDILDTAGQEEYSAMRDQYMRTGEGFLCVFAINNTKSFEDIHQYREQI
KRVKDSDDVPMVLVGNKCDLAARTVESRQAQDLARSYGIPYIETSAKTRQ
GVEDAFYTLVREIRQHKLRKLNPPDESGPGCMSCKCVLS.

The term “N-Ras” refers to the enzyme that in humans is encoded by the NRAS gene. The N-Ras protein is a GTPase, which converts guanosine triphosphate to guanosine diphosphate. The term “N-Ras” may refer to the nucleotide sequence or protein sequence of human NRAS (e.g., Entrez 4893, UniProt P01111, RefSeq NM_002524.4, or RefSeq NP_002515.1). In embodiments, N-Ras has the following amino acid sequence:

(SEQ ID NO: 3)
MTEYKLVVVGAGGVGKSALTIQLIQNHFVDEYDPTIEDSYRKQVVIDGET
CLLDILDTAGQEEYSAMRDQYMRTGEGFLCVFAINNSKSFADINLYREQI
KRVKDSDDVPMVLVGNKCDLPTRTVDTKQAHELAKSYGIPFIETSAKTRQ
GVEDAFYTLVREIRQYRMKKLNSSDDGTQGCMGLPCVVM.

The term “Raf” refers to a serine/threonine-specific protein kinase. The Raf kinases participate in the RAS-RAF-MEK-ERK signal transduction cascade. In embodiments, activation of Raf kinases requires interaction with Ras GTPases. In embodiments, Raf is A-Raf (e.g., Entrez 369), B-Raf (e.g., Entrez 673), or c-Raf (e.g., Entrez 5894).

### II. Compounds

In an aspect is provided a compound having the formula:

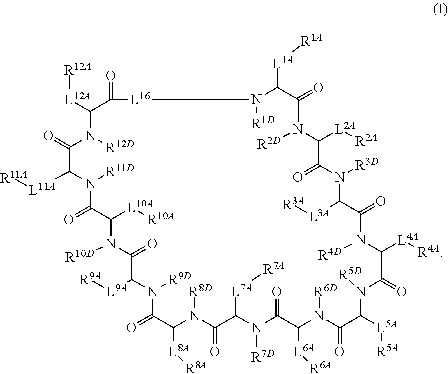

L^{1A}, L^{2A}, L^{3A}, L^{4A}, L^{5A}, L^{6A}, L^{7A}, L^{8A}, L^{9A}, L^{10A}, L^{11A}, and L^{12A} are independently a bond, substituted or unsubstituted alkylene (e.g., C₁-C₈, C₁-C₆, C₁-C₄, or C₁-C₂), or substituted or unsubstituted heteroalkylene (e.g., 2 to 8 membered, 2 to 6 membered, 4 to 6 membered, 2 to 3 membered, or 4 to 5 membered).

R^{1A}, R^{5A}, and R^{11A} are independently substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R^{2A} and R^{8A} are independently hydrogen, substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R^{3A} is hydrogen, substituted or unsubstituted alkyl (e.g., C₁-C₈, C₁-C₆, C₁-C₄, or C₁-C₂), substituted or unsubstituted cycloalkyl (e.g., C₃-C₈, C₃-C₆, C₄-C₆, or C₅-C₆), or substituted or unsubstituted aryl (e.g., C₆-C₁₀ or phenyl).

R^{4A} is hydrogen, —NH₂, —COOH, —CONH₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), or substituted or unsubstituted aryl (e.g., C₆-C₁₀ or phenyl).

R^{6A} and R^{9A} are independently hydrogen, —CN, —NH₂, —CONH₂, —SO₂NH₂, —NHNH₂, —ONH₂, —NHC(O)NHNH₂, —NHC(O)NH₂, —NHC(NH)NH₂, —NHOH, substituted or unsubstituted alkyl (e.g., C₁-C₈, C₁-C₆, C₁-C₄, or C₁-C₂), or substituted or unsubstituted heteroalkyl (e.g., 2 to 8 membered, 2 to 6 membered, 4 to 6 membered, 2 to 3 membered, or 4 to 5 membered).

R^{7A} is hydrogen, —NH₂, —COOH, —CONH₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, substituted or unsubstituted alkyl (e.g., C₁-C₈, C₁-C₆, C₁-C₄, or C₁-C₂), or substituted or unsubstituted heteroalkyl (e.g., 2 to 8 membered, 2 to 6 membered, 4 to 6 membered, 2 to 3 membered, or 4 to 5 membered).

R^{3A} and R^{9A} may optionally be joined to form a covalent linker.

R^{10A} is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —C(O)H, —C(O)OH, —C(O)NH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, -L^{10D}-L^{10E}-E, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

L^{10D} is a bond, —S(O)₂—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

L^{10E} is a bond, —S(O)₂—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted heteroalkylene (e.g., 2 to 8 membered, 2 to 6 membered, 4 to 6 membered, 2 to 3 membered, or 4 to 5 membered), substituted or unsubstituted heterocycloalkylene (e.g., 3 to 8 membered, 3 to 6 membered, 4 to 6 membered, 4 to 5 membered, or 5 to 6 membered), or substituted or unsubstituted heteroarylene (e.g., 5 to 10 membered, 5 to 9 membered, or 5 to 6 membered).

R^{12A} is hydrogen, —CN, —NH₂, —CONH₂, —SO₂NH₂, —NHNH₂, —ONH₂, —NHC(O)NHNH₂, —NHC(O)NH₂, —NHC(NH)NH₂, —NHOH, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), or substituted or unsubstituted aryl (e.g., C₆-C₁₀ or phenyl).

E is an electrophilic moiety.

R^{1D}, R^{2D}, R^{3D}, R^{4D}, R^{5D}, R^{6D}, R^{7D}, R^{8D}, R^{9D}, R^{10D}, R^{11D}, and R^{12D} are independently hydrogen, unsubstituted C₁-C₈ alkyl.

L¹⁶ is a covalent linker.

In embodiments, the compound has the formula:

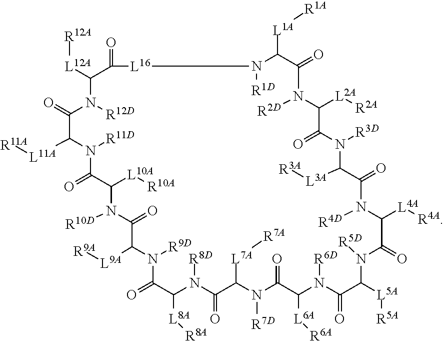

L^{1A}, L^{2A}, L^{3A}, L^{4A}, L^{5A}, L^{6A}, L^{7A}, L^{8A}, L^{9A}, L^{10A}, L^{11A}, and L^{12A} are independently a bond, substituted or unsubstituted alkylene (e.g., C₁-C₈, C₁-C₆, C₁-C₄, or C₁-C₂), or substituted or unsubstituted heteroalkylene (e.g., 2 to 8 membered, 2 to 6 membered, 4 to 6 membered, 2 to 3 membered, or 4 to 5 membered); R^{1A}, R^{2A}, R^{5A}, R^{8A}, and R^{11A} are independently substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{3A} is independently hydrogen, substituted or unsubstituted alkyl (e.g., C₁-C₈, C₁-C₆, C₁-C₄, or C₁-C₂), substituted or unsubstituted cycloalkyl (e.g., C₃-C₈, C₃-C₆, C₄-C₆, or C₅-C₆), or substituted or unsubstituted aryl (e.g., C₆-C₁₀ or phenyl). R^{4A} and R^{7A} are independently hydrogen, —NH₂, —COOH, —CONH₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, substituted or unsubstituted alkyl (e.g., C₁-C₈, C₁-C₆, C₁-C₄, or C₁-C₂), or substituted or unsubstituted heteroalkyl (e.g., 2 to 8 membered, 2 to 6 membered, 4 to 6 membered, 2 to 3 membered, or 4 to 5 membered); R^{6A}, R^{9A}, and R^{12A} are independently hydrogen, —CN, —NH₂, —CONH₂, —SO₂NH₂, —NHNH₂, —ONH₂, —NHC(O)NHNH₂, —NHC(O)NH₂, —NHC(NH)NH₂, —NHOH, substituted or unsubstituted alkyl (e.g., C₁-C₈, C₁-C₆, C₁-C₄, or C₁-C₂), or substituted or unsubstituted heteroalkyl (e.g., 2 to 8 membered, 2 to 6 membered, 4 to 6 membered, 2 to 3 membered, or 4 to 5 membered); R^{3A} and R^{9A} may optionally be joined to form a covalent linker; R^{10A} is independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —C(O)H, —C(O)OH, —C(O)NH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, -L^{10D}-L^{10E}-E, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); L^{10D} is independently a bond, —S(O)₂—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered); L^{10E} is independently a bond, —S(O)₂—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted heteroalkylene (e.g., 2 to 8 membered, 2 to 6 membered, 4 to 6 membered, 2 to 3 membered, or 4 to 5 membered), substituted or unsubstituted heterocycloalkylene (e.g., 3 to 8 membered, 3 to 6 membered, 4 to 6 membered, 4 to 5 membered, or 5 to 6 membered), or substituted or unsubstituted heteroarylene (e.g., 5 to 10 membered, 5 to 9 membered, or 5 to 6 membered); E is an electrophilic moiety; R^{1D}, R^{2D}, R^{3D}, R^{4D}, R^{5D}, R^{6D}, R^{7D}, R^{8D}, R^{9D}, R^{10D}, R^{11D}, and R^{12D} are independently hydrogen or unsubstituted C₁-C₄ alkyl; and L¹⁶ is a covalent linker.

In embodiments, the compound has the formula:

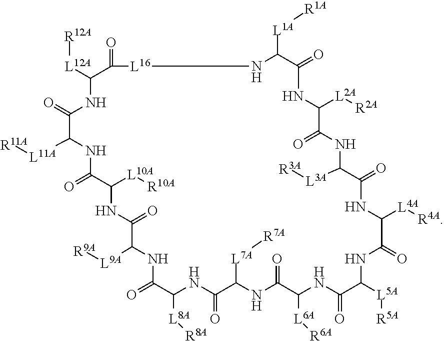

L^{1A}, L^{2A}, L^{3A}, L^{4A}, L^{5A}, L^{6A}, L^{7A}, L^{8A}, L^{9A}, L^{10A}, L^{11A}, L^{12A}, L¹⁶, R^{1A}, R^{2A}, R^{3A}, R^{4A}, R^{5A}, R^{6A}, R^{7A}, R^{8A}, R^{9A}, R^{10A}, R^{11A}, and R^{12A} are as described herein, including in embodiments.

In embodiments, the compound has the formula:

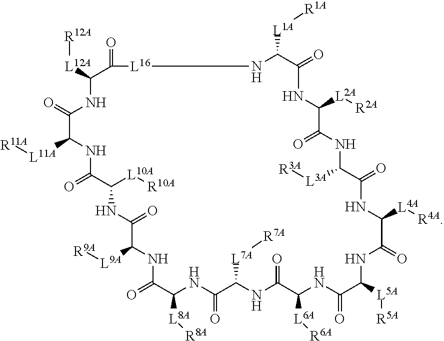

L^{2A}, L^{3A}, L^{4A}, L^{5A}, L^{6A}, L^{7A}, L^{8A}, L^{9A}, L^{10A}, L^{11A}, L^{12A}, L¹⁶, R^{1A}, R^{2A}, R^{3A}, R^{4A}, R^{5A}, R^{6A}, R^{7A}, R^{8A}, R^{9A}, R^{10A}, R^{11A}, and R^{12A} are as described herein, including in embodiments.

In embodiments, a substituted L^{1A} (e.g., substituted alkylene and/or substituted heteroalkylene) is substituted with at least one substituent group, size-limited substituent group, or lower substituent group; wherein if the substituted L^{1A} is substituted with a plurality of groups selected from substituent groups, size-limited substituent groups, and lower substituent groups; each substituent group, size-limited substituent group, and/or lower substituent group may optionally be different. In embodiments, when L^{1A} is substituted, it is substituted with at least one substituent group. In embodiments, when L^{1A} is substituted, it is substituted with at least one size-limited substituent group. In embodiments, when L^{1A} is substituted, it is substituted with at least one lower substituent group.

In embodiments, a substituted L^{2A} (e.g., substituted alkylene and/or substituted heteroalkylene) is substituted with at least one substituent group, size-limited substituent group, or lower substituent group; wherein if the substituted L^{2A} is substituted with a plurality of groups selected from substituent groups, size-limited substituent groups, and lower substituent groups; each substituent group, size-limited substituent group, and/or lower substituent group may optionally be different. In embodiments, when L^{2A} is substituted, it is substituted with at least one substituent group. In embodiments, when L^{2A} is substituted, it is substituted with at least one size-limited substituent group. In embodiments, when L^{2A} is substituted, it is substituted with at least one lower substituent group.

In embodiments, a substituted L^{3A} (e.g., substituted alkylene and/or substituted heteroalkylene) is substituted with at least one substituent group, size-limited substituent group, or lower substituent group; wherein if the substituted L^{3A} is substituted with a plurality of groups selected from substituent groups, size-limited substituent groups, and lower substituent groups; each substituent group, size-limited substituent group, and/or lower substituent group may optionally be different. In embodiments, when L^{3A} is substituted, it is substituted with at least one substituent group. In embodiments, when L^{3A} is substituted, it is substituted with at least one size-limited substituent group. In embodiments, when L^{3A} is substituted, it is substituted with at least one lower substituent group.

In embodiments, a substituted L^{4A} (e.g., substituted alkylene and/or substituted heteroalkylene) is substituted with at least one substituent group, size-limited substituent group, or lower substituent group; wherein if the substituted L^{4A} is substituted with a plurality of groups selected from substituent groups, size-limited substituent groups, and lower substituent groups; each substituent group, size-limited substituent group, and/or lower substituent group may optionally be different. In embodiments, when L^{4A} is substituted, it is substituted with at least one substituent group. In embodiments, when L^{4A} is substituted, it is substituted with at least one size-limited substituent group. In embodiments, when L^{4A} is substituted, it is substituted with at least one lower substituent group.

In embodiments, a substituted L^{5A} (e.g., substituted alkylene and/or substituted heteroalkylene) is substituted with at least one substituent group, size-limited substituent group, or lower substituent group; wherein if the substituted L^{5A} is substituted with a plurality of groups selected from substituent groups, size-limited substituent groups, and lower substituent groups; each substituent group, size-limited substituent group, and/or lower substituent group may optionally be different. In embodiments, when L^{5A} is substituted, it is substituted with at least one substituent group. In embodiments, when L^{5A} is substituted, it is substituted with at least one size-limited substituent group. In embodiments, when L^{5A} is substituted, it is substituted with at least one lower substituent group.

In embodiments, a substituted L^{6A} (e.g., substituted alkylene and/or substituted heteroalkylene) is substituted with at least one substituent group, size-limited substituent group, or lower substituent group; wherein if the substituted L^{6A} is substituted with a plurality of groups selected from substituent groups, size-limited substituent groups, and lower substituent groups; each substituent group, size-limited substituent group, and/or lower substituent group may optionally be different. In embodiments, when L^{6A} is substituted, it is substituted with at least one substituent group. In embodiments, when L^{6A} is substituted, it is substituted with at least one size-limited substituent group. In embodiments, when L^{6A} is substituted, it is substituted with at least one lower substituent group.

In embodiments, a substituted L^{7A} (e.g., substituted alkylene and/or substituted heteroalkylene) is substituted with at least one substituent group, size-limited substituent group, or lower substituent group; wherein if the substituted L^{7A} is substituted with a plurality of groups selected from substituent groups, size-limited substituent groups, and lower substituent groups; each substituent group, size-limited substituent group, and/or lower substituent group may optionally be different. In embodiments, when L^{7A} is substituted, it is substituted with at least one substituent group. In embodiments, when L^{7A} is substituted, it is substituted with at least one size-limited substituent group. In embodiments, when L^{7A} is substituted, it is substituted with at least one lower substituent group.

In embodiments, a substituted L^{8A} (e.g., substituted alkylene and/or substituted heteroalkylene) is substituted with at least one substituent group, size-limited substituent group, or lower substituent group; wherein if the substituted L^{8A} is substituted with a plurality of groups selected from substituent groups, size-limited substituent groups, and lower substituent groups; each substituent group, size-limited substituent group, and/or lower substituent group may optionally be different. In embodiments, when L^{8A} is substituted, it is substituted with at least one substituent group. In embodiments, when L^{8A} is substituted, it is substituted with at least one size-limited substituent group. In embodiments, when L^{8A} is substituted, it is substituted with at least one lower substituent group.

In embodiments, a substituted L^{9A} (e.g., substituted alkylene and/or substituted heteroalkylene) is substituted with at least one substituent group, size-limited substituent group, or lower substituent group; wherein if the substituted L^{9A} is substituted with a plurality of groups selected from substituent groups, size-limited substituent groups, and lower substituent groups; each substituent group, size-limited substituent group, and/or lower substituent group may optionally be different. In embodiments, when L^{9A} is substituted, it is substituted with at least one substituent group. In embodiments, when L^{9A} is substituted, it is substituted with at least one size-limited substituent group. In embodiments, when L^{9A} is substituted, it is substituted with at least one lower substituent group.

In embodiments, a substituted L^{10A} (e.g., substituted alkylene and/or substituted heteroalkylene) is substituted with at least one substituent group, size-limited substituent group, or lower substituent group; wherein if the substituted L^{10A} is substituted with a plurality of groups selected from substituent groups, size-limited substituent groups, and lower substituent groups; each substituent group, size-limited substituent group, and/or lower substituent group may optionally be different. In embodiments, when L^{10A} is substituted, it is substituted with at least one substituent group. In embodiments, when L^{10A} is substituted, it is substituted with at least one size-limited substituent group. In embodiments, when L^{10A} is substituted, it is substituted with at least one lower substituent group.

In embodiments, a substituted L^{11A} (e.g., substituted alkylene and/or substituted heteroalkylene) is substituted with at least one substituent group, size-limited substituent group, or lower substituent group; wherein if the substituted L^{11A} is substituted with a plurality of groups selected from substituent groups, size-limited substituent groups, and lower substituent groups; each substituent group, size-limited substituent group, and/or lower substituent group may optionally be different. In embodiments, when L^{11A} is substituted, it is substituted with at least one substituent group. In embodiments, when L^{11A} is substituted, it is substituted with at least one size-limited substituent group. In embodiments, when L^{11A} is substituted, it is substituted with at least one lower substituent group.

In embodiments, a substituted L^{12A} (e.g., substituted alkylene and/or substituted heteroalkylene) is substituted with at least one substituent group, size-limited substituent group, or lower substituent group; wherein if the substituted L^{12A} is substituted with a plurality of groups selected from substituent groups, size-limited substituent groups, and lower substituent groups; each substituent group, size-limited substituent group, and/or lower substituent group may optionally be different. In embodiments, when L^{12A} is substituted, it is substituted with at least one substituent group. In embodiments, when L^{12A} is substituted, it is substituted with at least one size-limited substituent group. In embodiments, when L^{12A} is substituted, it is substituted with at least one lower substituent group.

In embodiments, a substituted R^{1A} (e.g., substituted cycloalkyl, substituted heterocycloalkyl, substituted aryl, and/or substituted heteroaryl) is substituted with at least one substituent group, size-limited substituent group, or lower substituent group; wherein if the substituted R^{1A} is substituted with a plurality of groups selected from substituent groups, size-limited substituent groups, and lower substituent groups; each substituent group, size-limited substituent group, and/or lower substituent group may optionally be different. In embodiments, when R^{1A} is substituted, it is substituted with at least one substituent group. In embodiments, when R^{1A} is substituted, it is substituted with at least one size-limited substituent group. In embodiments, when R^{1A} is substituted, it is substituted with at least one lower substituent group.

In embodiments, a substituted R^{2A} (e.g., substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2A} is substituted with a plurality of groups selected from substituent groups, size-limited substituent groups, and lower substituent groups; each substituent group, size-limited substituent group, and/or lower substituent group may optionally be different. In embodiments, when R^{2A} is substituted, it is substituted with at least one substituent group. In embodiments, when R^{2A} is substituted, it is substituted with at least one size-limited substituent group. In embodiments, when R^{2A} is substituted, it is substituted with at least one lower substituent group.

In embodiments, a substituted R^{3A} (e.g., substituted alkyl, substituted cycloalkyl, and/or substituted aryl) is substituted with at least one substituent group, size-limited substituent group, or lower substituent group; wherein if the substituted R^{3A} is substituted with a plurality of groups selected from substituent groups, size-limited substituent groups, and lower substituent groups; each substituent group, size-limited substituent group, and/or lower substituent group may optionally be different. In embodiments, when R^{3A} is substituted, it is substituted with at least one substituent group. In embodiments, when R^{3A} is substituted, it is substituted with at least one size-limited substituent group. In embodiments, when R^{3A} is substituted, it is substituted with at least one lower substituent group.

In embodiments, a substituted R^{4A} (e.g., substituted alkyl, substituted heteroalkyl, and/or substituted aryl) is substituted with at least one substituent group, size-limited substituent group, or lower substituent group; wherein if the substituted R^{4A} is substituted with a plurality of groups selected from substituent groups, size-limited substituent groups, and lower substituent groups; each substituent group, size-limited substituent group, and/or lower substituent group may optionally be different. In embodiments, when R^{4A} is substituted, it is substituted with at least one substituent group. In embodiments, when R^{4A} is substituted, it is substituted with at least one size-limited substituent group. In embodiments, when R^{4A} is substituted, it is substituted with at least one lower substituent group.

In embodiments, a substituted R^{5A} (e.g., substituted cycloalkyl, substituted heterocycloalkyl, substituted aryl, and/or substituted heteroaryl) is substituted with at least one substituent group, size-limited substituent group, or lower substituent group; wherein if the substituted R^{5A} is substituted with a plurality of groups selected from substituent groups, size-limited substituent groups, and lower substituent groups; each substituent group, size-limited substituent group, and/or lower substituent group may optionally be different. In embodiments, when R^{5A} is substituted, it is substituted with at least one substituent group. In embodiments, when R^{5A} is substituted, it is substituted with at least one size-limited substituent group. In embodiments, when R^{5A} is substituted, it is substituted with at least one lower substituent group.

In embodiments, a substituted R^{6A} (e.g., substituted alkyl and/or substituted heteroalkyl) is substituted with at least one substituent group, size-limited substituent group, or lower substituent group; wherein if the substituted R^{6A} is substituted with a plurality of groups selected from substituent groups, size-limited substituent groups, and lower substituent groups; each substituent group, size-limited substituent group, and/or lower substituent group may optionally be different. In embodiments, when R^{6A} is substituted, it is substituted with at least one substituent group. In embodiments, when R^{6A} is substituted, it is substituted with at least one size-limited substituent group. In embodiments, when R^{6A} is substituted, it is substituted with at least one lower substituent group.

In embodiments, a substituted R^{7A} (e.g., substituted alkyl and/or substituted heteroalkyl) is substituted with at least one substituent group, size-limited substituent group, or lower substituent group; wherein if the substituted R^{7A} is substituted with a plurality of groups selected from substituent groups, size-limited substituent groups, and lower substituent groups; each substituent group, size-limited substituent group, and/or lower substituent group may optionally be different. In embodiments, when R^{7A} is substituted, it is substituted with at least one substituent group. In embodiments, when R^{7A} is substituted, it is substituted with at least one size-limited substituent group. In embodiments, when R^{7A} is substituted, it is substituted with at least one lower substituent group.

In embodiments, a substituted R^{8A} (e.g., substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{8A} is substituted with a plurality of groups selected from substituent groups, size-limited substituent groups, and lower substituent groups; each substituent group, size-limited substituent group, and/or lower substituent group may optionally be different. In embodiments, when R^{8A} is substituted, it is substituted with at least one substituent group. In embodiments, when R^{8A} is substituted, it is substituted with at least one size-limited substituent group. In embodiments, when R^{8A} is substituted, it is substituted with at least one lower substituent group.

In embodiments, a substituted R^{9A} (e.g., substituted alkyl and/or substituted heteroalkyl) is substituted with at least one substituent group, size-limited substituent group, or lower substituent group; wherein if the substituted R^{9A} is substituted with a plurality of groups selected from substituent groups, size-limited substituent groups, and lower substituent groups; each substituent group, size-limited substituent group, and/or lower substituent group may optionally be different. In embodiments, when R^{9A} is substituted, it is substituted with at least one substituent group. In embodiments, when R^{9A} is substituted, it is substituted with at least one size-limited substituent group. In embodiments, when R^{9A} is substituted, it is substituted with at least one lower substituent group.

In embodiments, a substituted R^{10A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{10A} is substituted with a plurality of groups selected from substituent groups, size-limited substituent groups, and lower substituent groups; each substituent group, size-limited substituent group, and/or lower substituent group may optionally be different. In embodiments, when R^{10A} is substituted, it is substituted with at least one substituent group. In embodiments, when R^{10A} is substituted, it is substituted with at least one size-limited substituent group. In embodiments, when R^{10A} is substituted, it is substituted with at least one lower substituent group.

In embodiments, a substituted L^{10D} (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{10D} is substituted with a plurality of groups selected from substituent groups, size-limited substituent groups, and lower substituent groups; each substituent group, size-limited substituent group, and/or lower substituent group may optionally be different. In embodiments, when L^{10D} is substituted, it is substituted with at least one substituent group. In embodiments, when L^{10D} is substituted, it is substituted with at least one size-limited substituent group. In embodiments, when L^{10D} is substituted, it is substituted with at least one lower substituent group.

In embodiments, a substituted L^{10E} (e.g., substituted heteroalkylene, substituted heterocycloalkylene, and/or substituted heteroarylene) is substituted with at least one substituent group, size-limited substituent group, or lower substituent group; wherein if the substituted L^{10E} is substituted with a plurality of groups selected from substituent groups, size-limited substituent groups, and lower substituent groups; each substituent group, size-limited substituent group, and/or lower substituent group may optionally be different. In embodiments, when L^{10E} is substituted, it is substituted with at least one substituent group. In embodiments, when L^{10E} is substituted, it is substituted with at least one size-limited substituent group. In embodiments, when L^{10E} is substituted, it is substituted with at least one lower substituent group.

In embodiments, a substituted R^{11A} (e.g., substituted cycloalkyl, substituted heterocycloalkyl, substituted aryl, and/or substituted heteroaryl) is substituted with at least one substituent group, size-limited substituent group, or lower substituent group; wherein if the substituted R^{11A} is substituted with a plurality of groups selected from substituent groups, size-limited substituent groups, and lower substituent groups; each substituent group, size-limited substituent group, and/or lower substituent group may optionally be different. In embodiments, when R^{11A} is substituted, it is substituted with at least one substituent group. In embodiments, when R^{11A} is substituted, it is substituted with at least one size-limited substituent group. In embodiments, when R^{11A} is substituted, it is substituted with at least one lower substituent group.

In embodiments, a substituted R^{12A} (e.g., substituted alkyl, substituted heteroalkyl, and/or substituted aryl) is substituted with at least one substituent group, size-limited substituent group, or lower substituent group; wherein if the substituted R^{12A} is substituted with a plurality of groups selected from substituent groups, size-limited substituent groups, and lower substituent groups; each substituent group, size-limited substituent group, and/or lower substituent group may optionally be different. In embodiments, when R^{12A} is substituted, it is substituted with at least one substituent group. In embodiments, when R^{12A} is substituted, it is substituted with at least one size-limited substituent group. In embodiments, when R^{12A} is substituted, it is substituted with at least one lower substituent group.

In embodiments,

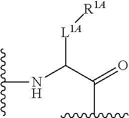

is a divalent form of an unnatural amino acid. In embodiments, L^{1A} is a bond or unsubstituted C₁-C₄ alkylene. In embodiments, R^{1A} is a substituted or unsubstituted aryl, or substituted or unsubstituted heteroaryl. In embodiments,

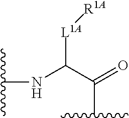

is a divalent form of a natural amino acid. In embodiments,

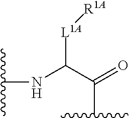

is a divalent form of tyrosine, a divalent form of phenylalanine, or a divalent form of tryptophan.

In embodiments,

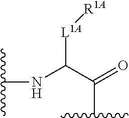

is a divalent form of tyrosine. In embodiments, -L^{1A}-R^{1A} is

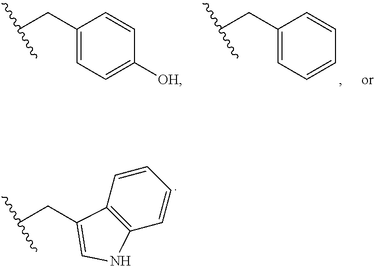 [SMILES: *Cc1ccc(O)cc1 ; *Cc1ccccc1 ; *Cc1c[nH]c2ccccc12]

In embodiments, -L^{1A}-R^{1A} is

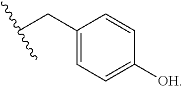 [SMILES: *Cc1ccc(O)cc1]

In embodiments,

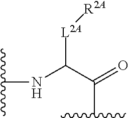

is a divalent form of an unnatural amino acid. In embodiments, L^{2A} is a bond or unsubstituted C₁-C₄ alkylene. In embodiments, R^{2A} is a substituted or unsubstituted aryl, or substituted or unsubstituted heteroaryl. In embodiments,

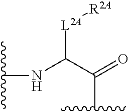

is a divalent form of a natural amino acid. In embodiments,

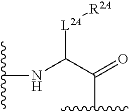

is a divalent form of phenylalanine, a divalent form of tyrosine, or a divalent form of tryptophan.

In embodiments,

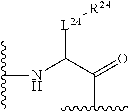

is a divalent form of phenylalanine. In embodiments, -L^{2A}-R^{2A} is

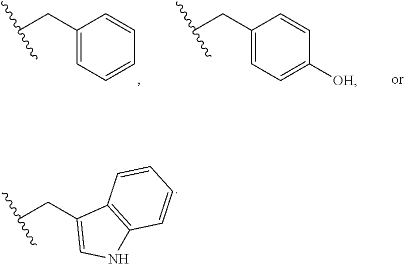 [SMILES: *Cc1ccccc1 ; *Cc1ccc(O)cc1 ; *Cc1c[nH]c2ccccc12]

In embodiments, -L^{2A}-R^{2A} is

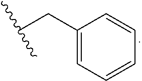 [SMILES: *Cc1ccccc1]

In embodiments,

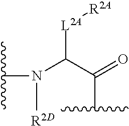

is a divalent form of an unnatural amino acid. In embodiments, L^{2A} is a bond or unsubstituted C₁-C₄ alkylene. In embodiments, R^{2A} is hydrogen, substituted or unsubstituted aryl, or substituted or unsubstituted heteroaryl. In embodiments, R^{2D} is an unsubstituted C₁-C₄ alkyl. In embodiments,

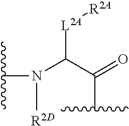

is a divalent form of an unnatural glycine derivative. In embodiments,

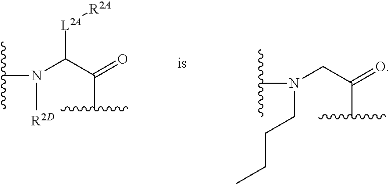

In embodiments,

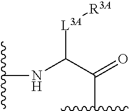

is a divalent form of an unnatural amino acid. In embodiments, L^{3A} is a bond or unsubstituted C₁-C₄ alkylene. In embodiments, R^{3A} is a substituted or unsubstituted alkyl. In embodiments,

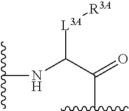

is a divalent form of a natural amino acid. In embodiments,

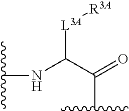

is a divalent form of valine, a divalent form of isoleucine, or a divalent form of leucine. In embodiments,

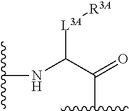

is a divalent form of valine. In embodiments, -L^{3A}-R^{3A} is

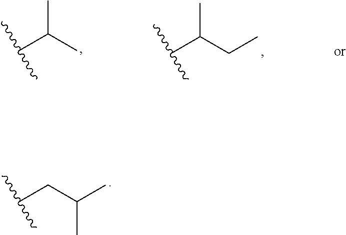

In embodiments, -L^{3A}-R^{3A} is

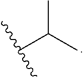 [SMILES: *C(C)C]

In embodiments,

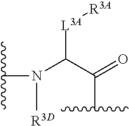

is a divalent form of an unnatural amino acid. In embodiments, L^{3A} is a bond or unsubstituted C₁-C₄ alkylene. In embodiments, R^{3A} is a substituted or unsubstituted aryl. In embodiments, R^{3D} is an unsubstituted C₁-C₄ alkyl. In embodiments,

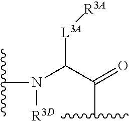

is a divalent form of an unnatural phenylalanine derivative. In embodiments,

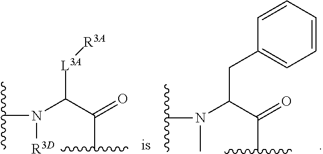

In embodiments,

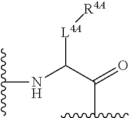

is a divalent form of an unnatural amino acid. In embodiments, L^{4A} is a bond or unsubstituted C₁-C₄ alkylene. In embodiments, R^{4A} is —C(O)NH₂ or substituted or unsubstituted heteroalkyl. In embodiments

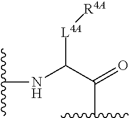

is a divalent form of a natural amino acid. In embodiments,

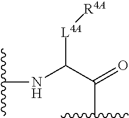

is a divalent form of asparagine or a divalent form of glutamine. In embodiments,

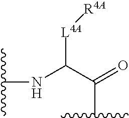

is a divalent form of asparagine. In embodiments, -L^{4A}-R^{4A} is

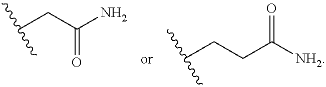 [SMILES: *CC(N)=O ; *CCC(N)=O]

In embodiments, -L^{4A}-R^{4A} is

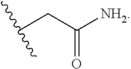 [SMILES: *CC(N)=O]

In embodiments,

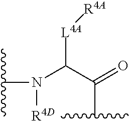

is a divalent form of an unnatural amino acid. In embodiments, L^{4A} is a bond or unsubstituted C₁-C₄ alkylene. In embodiments, R^{4A} is hydrogen, —C(O)NH₂, substituted or unsubstituted alkyl, substituted or unsubstituted heteroalkyl, or substituted or unsubstituted aryl. In embodiments, R^{4D} is an unsubstituted C₁-C₄ alkyl. In embodiments,

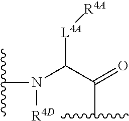

is a divalent form of an unnatural tyrosine derivative. In embodiments,

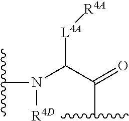

is a divalent form of an unnatural glycine derivative. In embodiments,

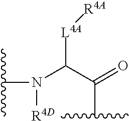

is a divalent form of a 2-aminobutyric acid derivative. In embodiments,

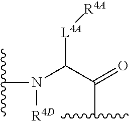

is a divalent form of an unnatural glutamic acid derivative. In embodiments,

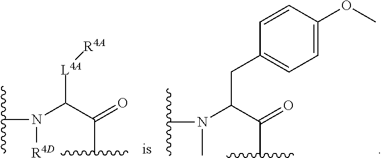

In embodiments,

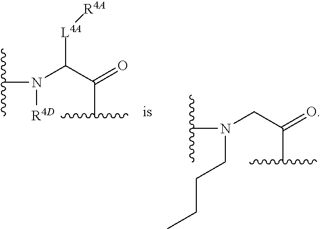

In embodiments,

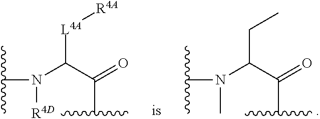

In embodiments,

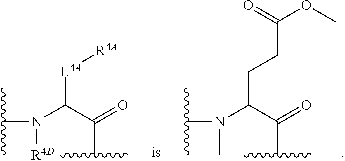

In embodiments,

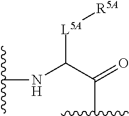

is a divalent form of an unnatural amino acid. In embodiments, L^{5A} is a bond or unsubstituted C₁-C₄ alkylene. In embodiments, R^{5A} is a substituted or unsubstituted aryl, or substituted or unsubstituted heteroaryl. In embodiments, R^{5A} is a halogen-substituted aryl. In embodiments,

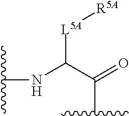

is a divalent form of a natural amino acid. In embodiments,

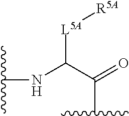

is a divalent form of phenylalanine, a divalent form of tyrosine, or a divalent form of tryptophan. In embodiments,

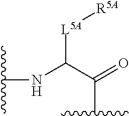

is a divalent form of phenylalanine. In embodiments, -L^{5A}-R^{5A} is

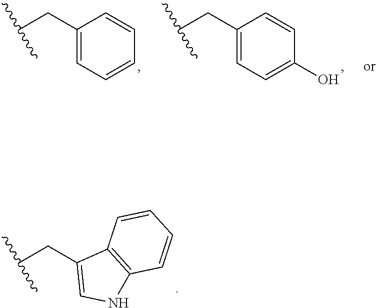

In embodiments, -L^{5A}-R^{5A} is
In embodiments, -L^{5A}-R^{5A} is

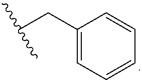 [SMILES: *Cc1ccccc1]

In embodiments, -L^{5A}-R^{5A} is

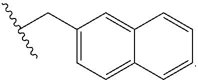 [SMILES: *Cc1ccc2ccccc2c1]

In embodiments, -L^{5A}-R^{5A} is

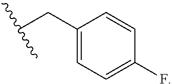 [SMILES: *Cc1ccc(F)cc1]

In embodiments, -L^{5A}-R^{5A} is

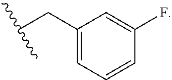 [SMILES: *Cc1cccc(F)c1]

In embodiments,

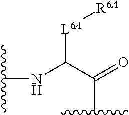

is a divalent form of an unnatural amino acid. In embodiments, L^{6A} is a bond or unsubstituted C₁-C₆ alkylene. In embodiments, R^{6A} is —NH₂, —NHC(NH)NH₂, or substituted or unsubstituted heteroalkyl. In embodiments,

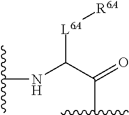

is a divalent form of a natural amino acid. In embodiments,

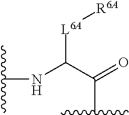

is a divalent form
of arginine or a divalent form of lysine. In embodiments,

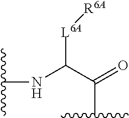

is a divalent form of arginine. In embodiments, -L^{6A}-R^{6A} is

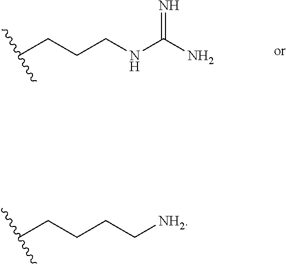 [SMILES: *CCCNC(=N)N ; *CCCCN]

In embodiments, -L^{6A}-R^{6A} is

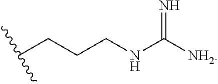 [SMILES: *CCCNC(=N)N]

In embodiments,

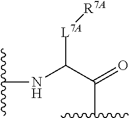

is a divalent form of an unnatural amino acid. In embodiments, L^{7A} is a bond or unsubstituted C₁-C₄ alkylene. In embodiments, R^{7A} is —C(O)NH₂ or substituted or unsubstituted heteroalkyl. In embodiments,

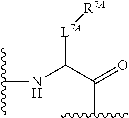

is a divalent form of a natural amino acid. In embodiments,

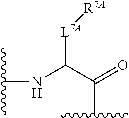

is a divalent form of asparagine or a divalent form of glutamine. In embodiments,

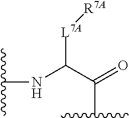

is a divalent form of asparagine. In embodiments, -L^{7A}-R^{7A} is

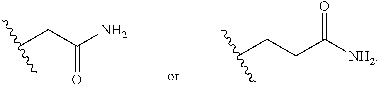 [SMILES: *CC(N)=O ; *CCC(N)=O]

In embodiments, -L^{7A}-R^{7A} is

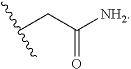 [SMILES: *CC(N)=O]

In embodiments,

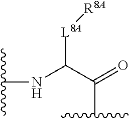

is a divalent form of an unnatural amino acid. In embodiments, L^{8A} is a bond or unsubstituted C₁-C₄ alkylene. In embodiments, R^{8A} is a substituted or unsubstituted aryl, or substituted or unsubstituted heteroaryl. In embodiments,

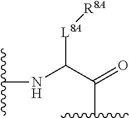

is a divalent form of a natural amino acid. In embodiments,

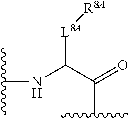

is a divalent form of phenylalanine, a divalent form of tyrosine, or a divalent form of tryptophan.

In embodiments,

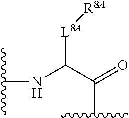

is a divalent form of phenylalanine. In embodiments -L^{8A}-R^{8A} is

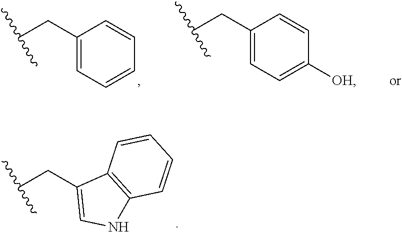 [SMILES: *Cc1ccccc1 ; *Cc1ccc(O)cc1 ; *Cc1c[nH]c2ccccc12]

In embodiments, -L^{8A}-R^{8A} is

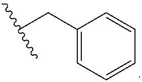 [SMILES: *Cc1ccccc1]

In embodiments, -L^{8A}-R^{8A} is

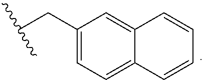 [SMILES: *Cc1ccc2ccccc2c1]

In embodiments,

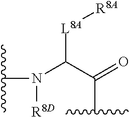

is a divalent form of an unnatural amino acid. In embodiments, L^{8A} is a bond or unsubstituted C₁-C₄ alkylene. In embodiments, R^{8A} is hydrogen, substituted or unsubstituted aryl, or substituted or unsubstituted heteroaryl. In embodiments, R^{8D} is an unsubstituted C₁-C₄ alkyl. In embodiments,

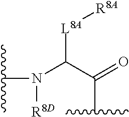

is a divalent form of an unnatural glutamic acid derivative. In embodiments,

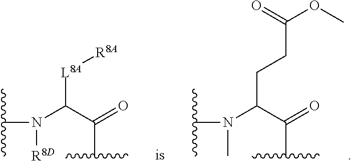

In embodiments,

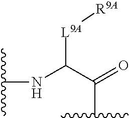

is a divalent form of an unnatural amino acid. In embodiments, L^{9A} is a bond or unsubstituted C₁-C₆ alkylene. In embodiments, R^{9A} is —NH₂, —NHC(NH)NH₂, or substituted or unsubstituted heteroalkyl. In embodiments,

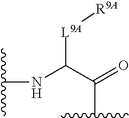

is a divalent form of a natural amino acid. In embodiments,

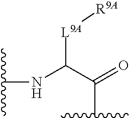

is a divalent form of arginine or a divalent form of lysine. In embodiments,

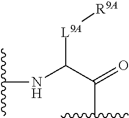

is a divalent form of arginine. In embodiments, -L^{9A}-R^{9A} is

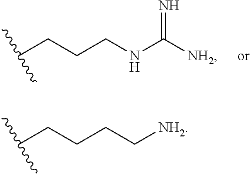 [SMILES: *CCCNC(=N)N ; *CCCCN]

In embodiments, -L^{9A}-R^{9A} is

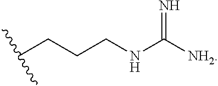 [SMILES: *CCCNC(=N)N]

In embodiments,

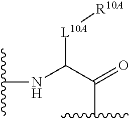

is a divalent form of an unnatural amino acid. In embodiments,

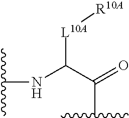

is a divalent form of 2,3-diaminoproprionic acid (Dap). In embodiments,

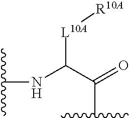

is a divalent form of citrulline (Cit). In embodiments, L^{10A} is a bond or unsubstituted C₁-C₄ alkylene. In embodiments, R^{10A} is —OH, —NH₂, —NHC(O)NH₂, —NHC(NH)NH₂, substituted or unsubstituted alkyl, substituted or unsubstituted heterocycloalkyl, or substituted or unsubstituted heteroaryl. In embodiments, R^{10A} is —OH, —NH₂, —NHC(O)NH₂, —NHC(NH)NH₂, substituted or unsubstituted alkyl, substituted or unsubstituted heterocycloalkyl, substituted or unsubstituted aryl, or substituted or unsubstituted heteroaryl. In embodiments, -L^{10A}-R^{10A} is

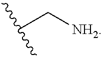 [SMILES: *CN]

In embodiments, -L^{10A}-R^{10A} is

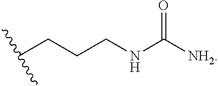 [SMILES: *CCCNC(N)=O]

In embodiments, -L^{10A}-R^{10A} is

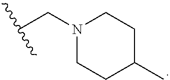 [SMILES: *CN1CCC(C)CC1]

In embodiments,

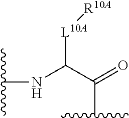

is a divalent form of a natural amino acid. In embodiments,

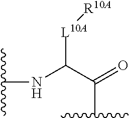

is a divalent form of threonine, a divalent form of histidine, a divalent form of lysine, or a divalent form of arginine. In embodiments,

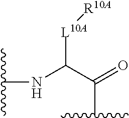

is a divalent form of threonine. In embodiments,

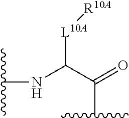

is a divalent form of histidine. In embodiments,

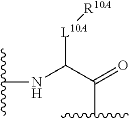

is a divalent form of lysine. In embodiments,

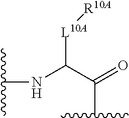

is a divalent form of arginine. In embodiments, -L^{10A}-R^{10A} is

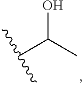 [SMILES: *C(C)O]

In embodiments, -L^{10A}-R^{10A} is

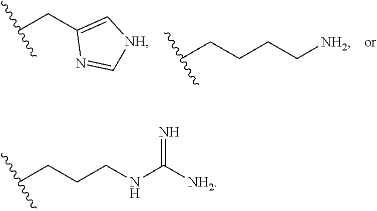 [SMILES: *Cc1c[nH]cn1 ; *CCCCN ; *CCCNC(=N)N]

In embodiments, -L^{10A}-R^{10A} is

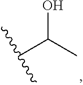 [SMILES: *C(C)O]

In embodiments, -L^{10A}-R^{10A} is

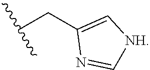 [SMILES: *Cc1c[nH]cn1]

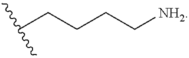 [SMILES: *CCCCN]

In embodiments, -L^{10A}-R^{10A} is

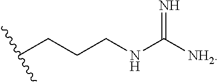 [SMILES: *CCCNC(=N)N]

In embodiments, -L^{10A}-R^{10A} is

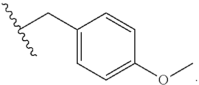 [SMILES: *Cc1ccc(OC)cc1]

In embodiments,

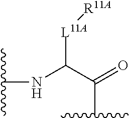

is a divalent form of an unnatural amino acid. In embodiments, L^{11A} is a bond or unsubstituted C₁-C₄ alkylene. In embodiments, R^{11A} is a substituted or unsubstituted aryl, or substituted or unsubstituted heteroaryl. In embodiments,

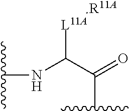

is a divalent form of a natural amino acid. In embodiments,

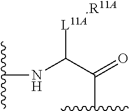

is a divalent form of phenylalanine, a divalent form of tyrosine, or a divalent form of tryptophan. In embodiments,

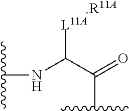

is a divalent form of phenylalanine. In embodiments, -L^{11A}-R^{11A} is

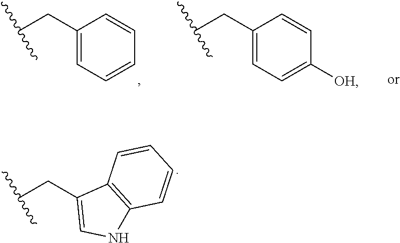 [SMILES: *Cc1ccccc1 ; *Cc1ccc(O)cc1 ; *Cc1c[nH]c2ccccc12]

In embodiments, -L^{11A}-R^{11A} is

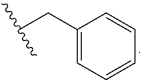 [SMILES: *Cc1ccccc1]

In embodiments,

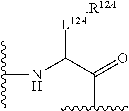

is a divalent form of an unnatural amino acid. In embodiments, L^{12A} is a bond or unsubstituted C₁-C₆ alkylene. In embodiments, R^{12A} is —NH₂, —NHC(NH)NH₂, or substituted or unsubstituted heteroalkyl. In embodiments,

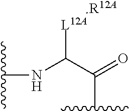

is a divalent form of a natural amino acid. In embodiments,

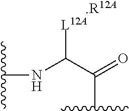

is a divalent form of arginine or a divalent form of lysine. In embodiments,

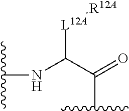

is a divalent form of arginine. In embodiments, -L^{12A}-R^{12A} is

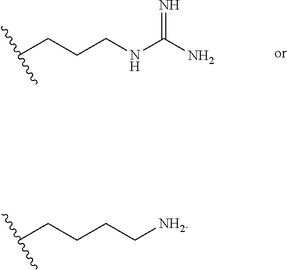 [SMILES: *CCCNC(=N)N ; *CCCCN]

In embodiments, -L^{12A}-R^{12A} is

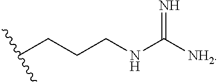 [SMILES: *CCCNC(=N)N]

In embodiments,

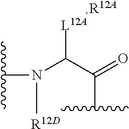

is a divalent form of an unnatural amino acid. In embodiments, L^{12A} is a bond or unsubstituted C₁-C₆ alkylene. In embodiments, R^{12A} is —NH₂, —NHC(NH)NH₂, substituted or unsubstituted heteroalkyl, or substituted or unsubstituted aryl. In embodiments, R^{12D} is an unsubstituted C₁-C₄ alkyl. In embodiments,

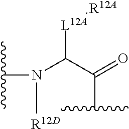

is a divalent form of an unnatural phenylalanine derivative. In embodiments,

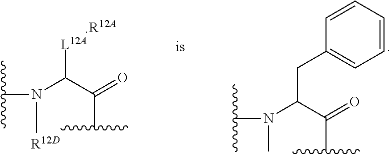

In embodiments, R^{10A} is independently hydrogen, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, or substituted or unsubstituted heteroalkyl.

In embodiments, R^{10A} is -L^{10D}-L^{10E}-E. L^{10D}, L^{10E}, and E are as described herein, including in embodiments.

In embodiments, L^{10D} is independently a bond, —NH—, —O—, —C(O)—, —C(O)NH—, —NHC(O)NH—, —NHC(NH)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L^{10D} is independently a bond. In embodiments, L^{10D} is independently —NH—. In embodiments, L^{10D} is independently —O—. In embodiments, L^{10D} is independently —C(O)—. In embodiments, L^{10D} is independently —C(O)NH—. In embodiments, L^{10D} is independently —NHC(O)NH—. In embodiments, L^{10D} is independently substituted or unsubstituted alkylene. In embodiments, L^{10D} is independently substituted or unsubstituted heteroalkylene. In embodiments, L^{10D} is independently substituted or unsubstituted heterocycloalkylene. In embodiments, L^{10D} is independently substituted or unsubstituted arylene. In embodiments, L^{10D} is independently substituted or unsubstituted heteroarylene.

In embodiments, L^{10E} is independently a bond, —NH—, —O—, —C(O)—, —C(O)NH—, —NHC(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L^{10E} is independently a bond. In embodiments, L^{10E} is independently —NH—. In embodiments, L^{10E} is independently —O—. In embodiments, L^{10E} is independently —C(O)—. In embodiments, L^{10E} is independently —C(O)NH—. In embodiments, L^{10E} is independently —NHC(O)NH—. In embodiments, L^{10E} is independently substituted or unsubstituted alkylene. In embodiments, L^{10E} is independently substituted or unsubstituted heteroalkylene. In embodiments, L^{10E} is independently substituted or unsubstituted heterocycloalkylene. In embodiments, L^{10E} is independently substituted or unsubstituted arylene. In embodiments, L^{10E} is independently substituted or unsubstituted heteroarylene.

In embodiments, E is an electrophilic moiety capable of forming a covalent bond with a cysteine, aspartate, lysine, arginine, histidine, leucine, tyrosine, methionine, serine, or glutamate residue.

In embodiments, E is an electrophilic moiety as described in Mukherjee et al. Curr. Opin. Chem. Biol. 44, 30-38 (2018), which is incorporated herein by reference in its entirety and for all purposes. In embodiments, E is an electrophilic moiety as described in Gehringer et al. J. Med. Chem. 62, 5673-5724 (2019), which is incorporated herein by reference in its entirety and for all purposes.

In embodiments, E is —SH, —SSR²⁶,

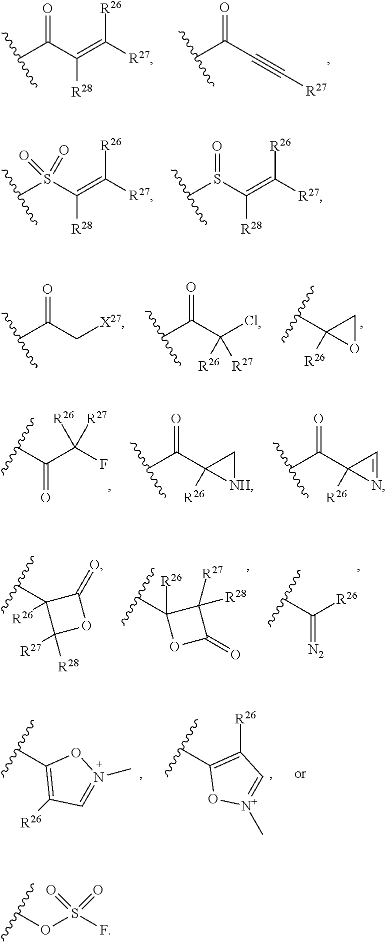

R²⁶, R²⁷, and R²⁸ are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —C(O)H, —C(O)OH, —C(O)NH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

X²⁷ is independently —F, —Cl, —Br, or —I.

In embodiments, a substituted R²⁶ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R²⁶ is substituted with a plurality of groups selected from substituent groups, size-limited substituent groups, and lower substituent groups; each substituent group, size-limited substituent group, and/or lower substituent group may optionally be different. In embodiments, when R²⁶ is substituted, it is substituted with at least one substituent group. In embodiments, when R²⁶ is substituted, it is substituted with at least one size-limited substituent group. In embodiments, when R²⁶ is substituted, it is substituted with at least one lower substituent group.

In embodiments, a substituted R²⁷ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R²⁷ is substituted with a plurality of groups selected from substituent groups, size-limited substituent groups, and lower substituent groups; each substituent group, size-limited substituent group, and/or lower substituent group may optionally be different. In embodiments, when R²⁷ is substituted, it is substituted with at least one substituent group. In embodiments, when R²⁷ is substituted, it is substituted with at least one size-limited substituent group. In embodiments, when R²⁷ is substituted, it is substituted with at least one lower substituent group.

In embodiments, a substituted R²⁸ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R²⁸ is substituted with a plurality of groups selected from substituent groups, size-limited substituent groups, and lower substituent groups; each substituent group, size-limited substituent group, and/or lower substituent group may optionally be different. In embodiments, when R²⁸ is substituted, it is substituted with at least one substituent group. In embodiments, when R²⁸ is substituted, it is substituted with at least one size-limited substituent group. In embodiments, when R²⁸ is substituted, it is substituted with at least one lower substituent group.

In embodiments, E is

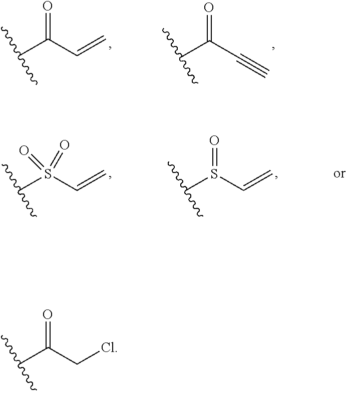

In embodiments, -L^{10A}-R^{10A} is

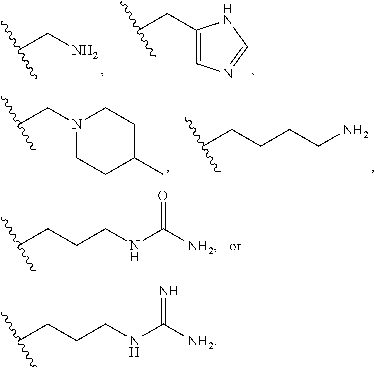

In embodiments, R^{3A} and R^{9A} are joined to form a bioconjugate linker.

In embodiments, R^{3A} and R^{9A} are joined to form a covalent linker having the formula -L^{18A}-L^{18B}-L^{18C}-L^{18D}-L^{18E}-L^{18F}-.

L^{18A}, L^{18B}, L^{18C}, L^{18D}, L^{18E}, and L^{18F} are independently bond, —SS—, —S(O)₂—, —OS(O)₂—, —S(O)₂O—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

In embodiments, a substituted L^{18A} (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{18A} is substituted with a plurality of groups selected from substituent groups, size-limited substituent groups, and lower substituent groups; each substituent group, size-limited substituent group, and/or lower substituent group may optionally be different. In embodiments, when L^{18A} is substituted, it is substituted with at least one substituent group. In embodiments, when L^{18A} is substituted, it is substituted with at least one size-limited substituent group. In embodiments, when L^{18A} is substituted, it is substituted with at least one lower substituent group.

In embodiments, a substituted L^{18B} (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{18B} is substituted with a plurality of groups selected from substituent groups, size-limited substituent groups, and lower substituent groups; each substituent group, size-limited substituent group, and/or lower substituent group may optionally be different. In embodiments, when L^{18B} is substituted, it is substituted with at least one substituent group. In embodiments, when L^{18B} is substituted, it is substituted with at least one size-limited substituent group. In embodiments, when L^{18B} is substituted, it is substituted with at least one lower substituent group.

In embodiments, a substituted L^{18C} (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{18C} is substituted with a plurality of groups selected from substituent groups, size-limited substituent groups, and lower substituent groups; each substituent group, size-limited substituent group, and/or lower substituent group may optionally be different. In embodiments, when L^{18C} is substituted, it is substituted with at least one substituent group. In embodiments, when L^{18C} is substituted, it is substituted with at least one size-limited substituent group. In embodiments, when L^{18C} is substituted, it is substituted with at least one lower substituent group.

In embodiments, a substituted L^{18D} (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{18D} is substituted with a plurality of groups selected from substituent groups, size-limited substituent groups, and lower substituent groups; each substituent group, size-limited substituent group, and/or lower substituent group may optionally be different. In embodiments, when L^{18D} is substituted, it is substituted with at least one substituent group. In embodiments, when L^{18D} is substituted, it is substituted with at least one size-limited substituent group. In embodiments, when L^{18D} is substituted, it is substituted with at least one lower substituent group.

In embodiments, a substituted L^{18E} (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{18E} is substituted with a plurality of groups selected from substituent groups, size-limited substituent groups, and lower substituent groups; each substituent group, size-limited substituent group, and/or lower substituent group may optionally be different. In embodiments, when L^{18E} is substituted, it is substituted with at least one substituent group. In embodiments, when L^{18E} is substituted, it is substituted with at least one size-limited substituent group. In embodiments, when L^{18E} is substituted, it is substituted with at least one lower substituent group.

In embodiments, a substituted L^{18F} (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{18F} is substituted with a plurality of groups selected from substituent groups, size-limited substituent groups, and lower substituent groups; each substituent group, size-limited substituent group, and/or lower substituent group may optionally be different. In embodiments, when L^{18F} is substituted, it is substituted with at least one substituent group. In embodiments, when L^{18F} is substituted, it is substituted with at least one size-limited substituent group. In embodiments, when L^{18F} is substituted, it is substituted with at least one lower substituent group.

In embodiments, L^{18A} is independently a bond or unsubstituted C₁-C₄ alkyl.

In embodiments, L^{18B} is independently —SS— or unsubstituted heteroarylene. In embodiments, L^{18B} is independently —SS—. In embodiments, L^{18B} is independently an unsubstituted 3 to 8 membered heteroarylene. In embodiments, L^{18B} is independently an unsubstituted triazolylene. In embodiments, L^{18B} is independently

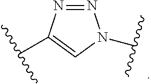 [SMILES: *c1cn(*)nn1]

In embodiments, L^{18C} is independently a bond or unsubstituted C₁-C₄ alkyl.

In embodiments, L^{18D} is independently a bond or unsubstituted C₁-C₄ alkyl.

In embodiments, L^{18E} is independently a bond or unsubstituted C₁-C₄ alkyl.

In embodiments, L^{18F} is independently a bond or unsubstituted C₁-C₄ alkyl.

In embodiments, R^{3A} and R^{9A} are joined to form

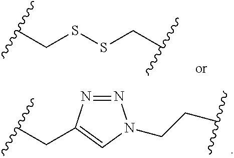 [SMILES: *CSSC* ; *CCn1cc(C*)nn1]

In embodiments, R^{3A} and R^{9A} are joined to form

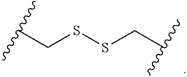 [SMILES: *CSSC*]

In embodiments, R^{3A} and R^{9A} are joined to form

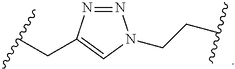 [SMILES: *CCn1cc(C*)nn1]

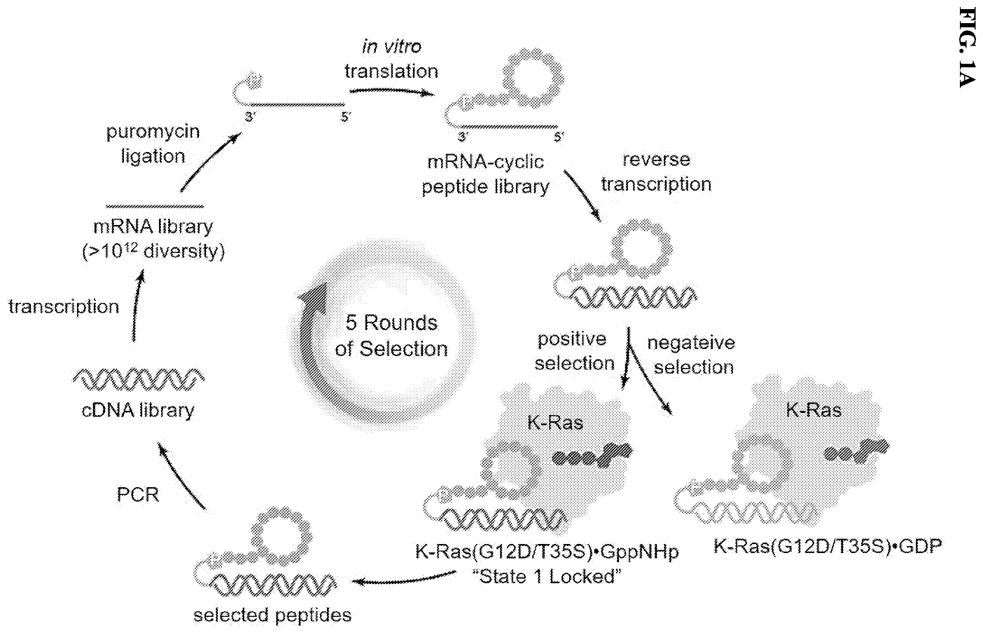

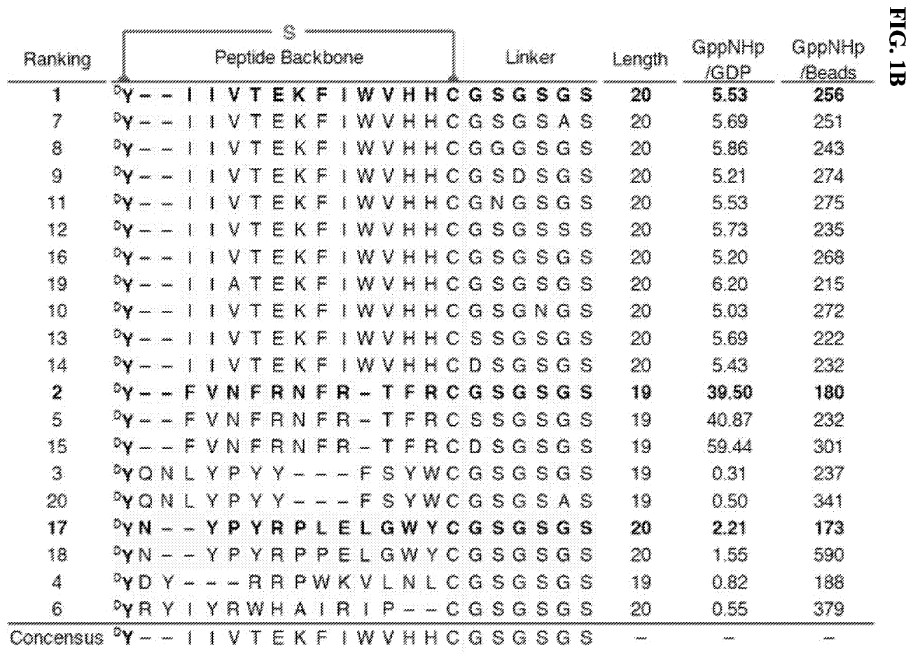

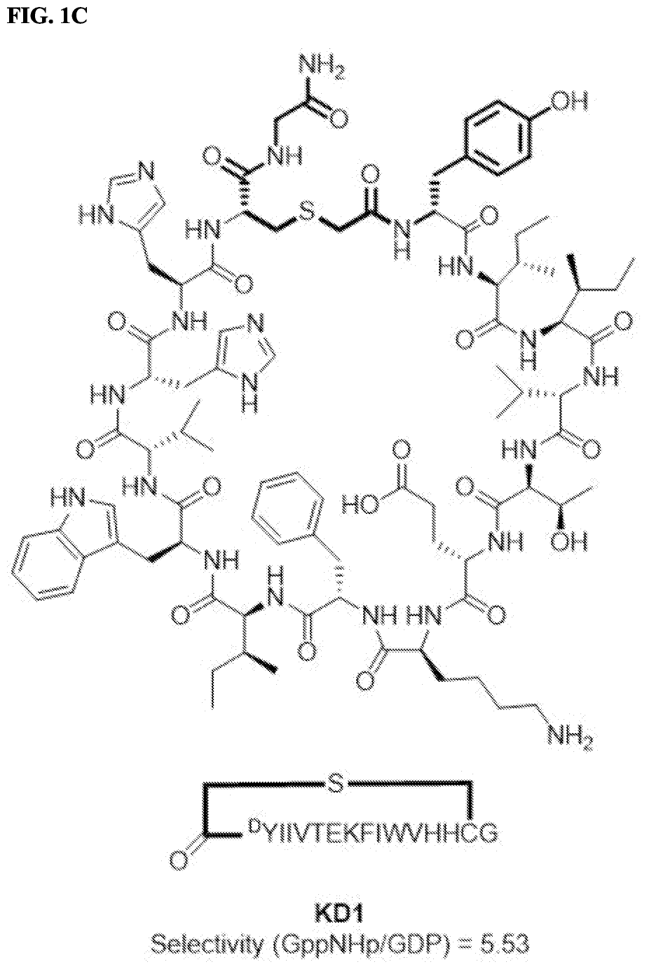

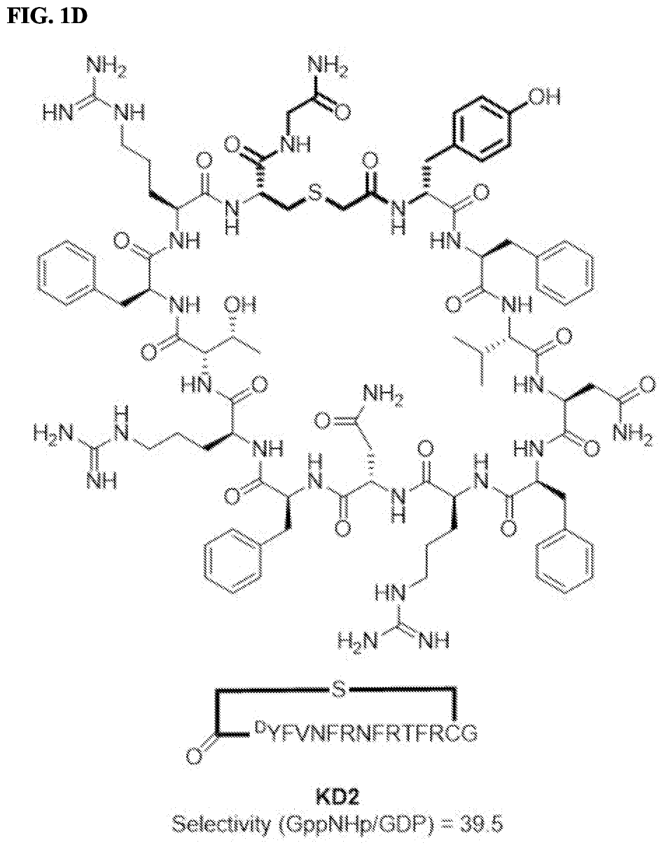

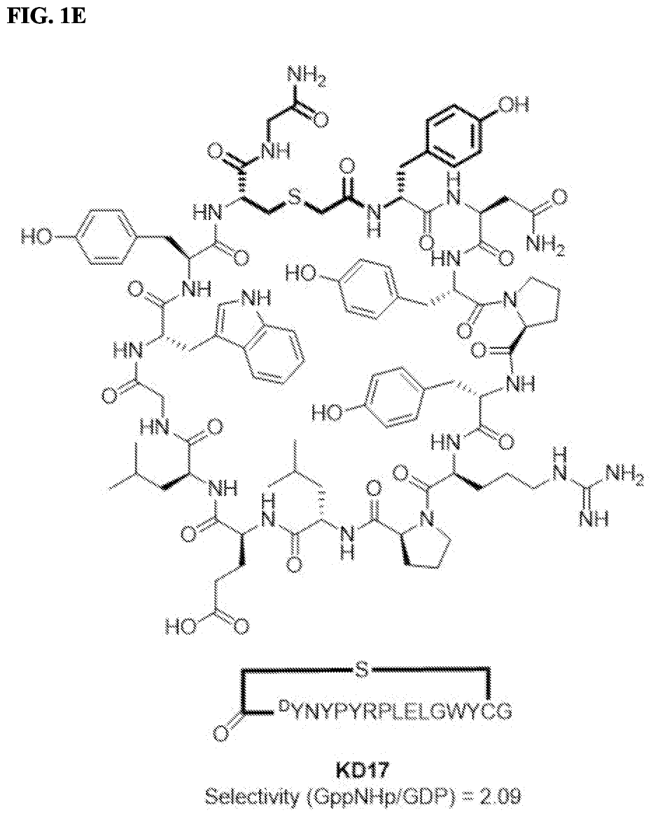

In embodiments, the compound of formula (I) is a peptide of FIG. 1B. In embodiments, the compound of formula (I) is peptide 2, 5, or 15 of FIG. 1B. For example, for peptide 2 of FIG. 1B,

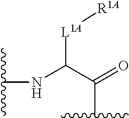

is a divalent form of D-tyrosine;

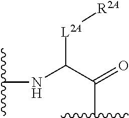

is a divalent form of phenylalanine;

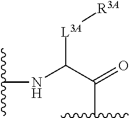

is a divalent form of valine;

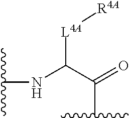

is a divalent form of asparagine;

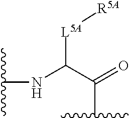

is a divalent form of phenylalanine;

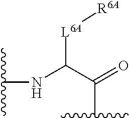

is a divalent form of arginine;

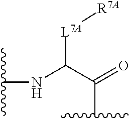

is divalent form of asparagine;

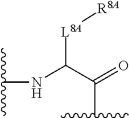

is a divalent form of phenylalanine;

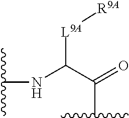

is a divalent form of
arginine;

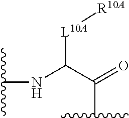

is a divalent form of threonine;

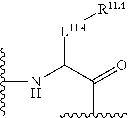

is a divalent form of phenylalanine; and

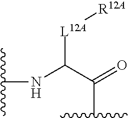

is a divalent form of arginine. Where the compound of formula (I) is peptide 5 or 15 of FIG. 1B, the same exemplification would apply. For example, for peptide 5 of FIG. 1B,

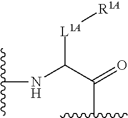

is a divalent form of D-tyrosine (with the same exemplification of the remaining amino acids). And for example, for peptide 15 of FIG. 1B,

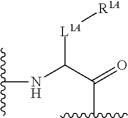

is a divalent form of D-tyrosine (with the same exemplification of the remaining amino acids).

In embodiments, the compound has the formula:

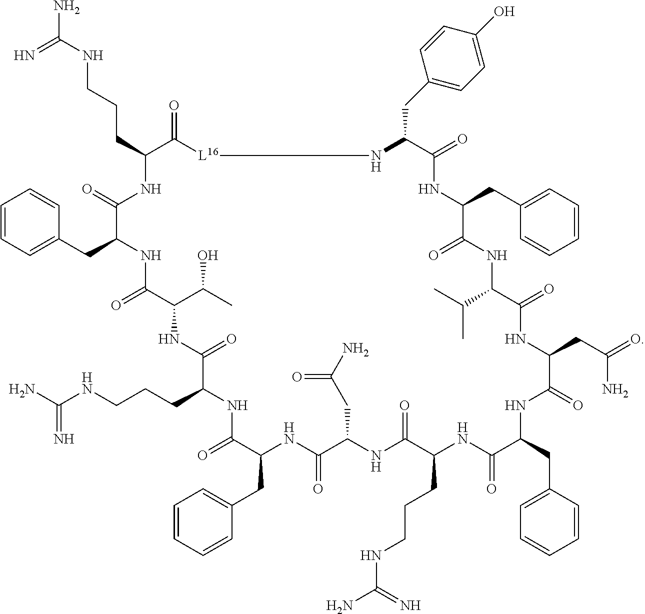

L¹⁶ is as described herein, including in embodiments.

In an aspect is provided a compound having the formula:

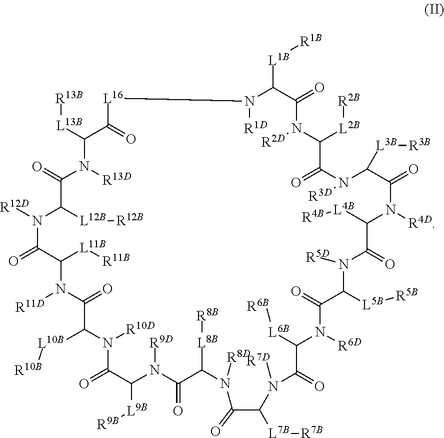

R^{1D}, R^{2D}, R^{3D}, R^{4D}, R^{5D}, R^{6D}, R^{7D}, R^{8D}, R^{9D}, R^{10D}, R^{11D}, R^{12D}, and L¹⁶ are as described herein, including in embodiments.

L^{1B}, L^{2B}, L^{3B}, L^{4B}, L^{5B}, L^{6B}, L^{7B}, L^{8B}, L^{9B}, L^{10B}, L^{11B}, L^{12B}, and L^{13B} are independently a bond, substituted or unsubstituted alkylene (e.g., C₁-C₈, C₁-C₆, C₁-C₄, or C₁-C₂), or substituted or unsubstituted heteroalkylene (e.g., 2 to 8 membered, 2 to 6 membered, 4 to 6 membered, 2 to 3 membered, or 4 to 5 membered).

R^{1B}, R^{8B}, and R^{10B} are independently substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R^{2B}, R^{3B}, R^{4B}, R^{9B}, and R^{11B} are independently hydrogen, substituted or unsubstituted alkyl (e.g., C₁-C₈, C₁-C₆, C₁-C₄, or C₁-C₂), substituted or unsubstituted cycloalkyl (e.g., C₃-C₈, C₃-C₆, C₄-C₆, or C₅-C₆), or substituted or unsubstituted aryl (e.g., C₆-C₁₀ or phenyl).

R^{5B} is independently hydrogen, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), or substituted or unsubstituted heteroalkyl (e.g., 2 to 8 membered, 2 to 6 membered, 4 to 6 membered, 2 to 3 membered, or 4 to 5 membered).

R^{6B} is independently hydrogen, —OH, —COOH, —NO₂, —SO₃H, —OSO₃H, substituted or unsubstituted alkyl (e.g., C₁-C₈, C₁-C₆, C₁-C₄, or C₁-C₂), or substituted or unsubstituted heteroalkyl (e.g., 2 to 8 membered, 2 to 6 membered, 4 to 6 membered, 2 to 3 membered, or 4 to 5 membered).

R^{7B}, R^{12B}, and R^{13B} are independently hydrogen, —NH₂, —CONH₂, —SO₂NH₂, —NHNH₂, —ONH₂, —NHC(O)NHNH₂, —NHC(O)NH₂, —NHC(NH)NH₂, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), or substituted or unsubstituted heteroaryl (e.g., 5 to 10 membered, 5 to 9 membered, or 5 to 6 membered).

Two substituents selected from R^{1B}, R^{2B}, R^{3B}, R^{4B}, R^{5B}, R^{6B}, L^{7B}, R^{8B}, R^{9B}, R^{10B}, R^{11B}, R^{12B}, and R^{13B} may optionally be joined to form a covalent linker.

R^{13D} is independently hydrogen or unsubstituted C₁-C₄ alkyl.

In embodiments, the compound has the formula:

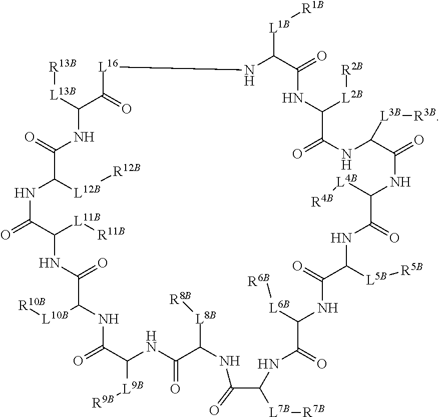

L^{1B}, L^{2B}, L^{3B}, L^{4B}, L^{5B}, L^{6B}, L^{7B}, L^{8B}, L^{9B}, L^{10B}, L^{11B}, L^{12B}, L^{13B}, L¹⁶, R^{1B}, R^{2B}, R^{3B}, R^{4B}, R^{5B}, R^{6B}, R^{7B}, R^{8B}, R^{9B}, R^{10B}, R^{11B}, R^{12B}, and R^{13B} are as described herein, including in embodiments.

In embodiments, the compound has the formula:

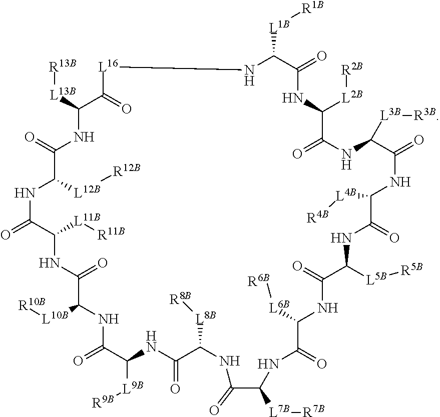

L^{1B}, L^{2B}, L^{3B}, L^{4B}, L^{5B}, L^{6B}, L^{7B}, L^{8B}, L^{9B}, L^{10B}, L^{11B}, L^{12B}, L^{13B}, L¹⁶, R^{1B}, R^{2B}, R^{3B}, R^{4B}, R^{5B}, R^{6B}, R^{7B}, R^{8B}, R^{9B}, R^{10B}, R^{11B}, R^{12B}, and R^{13B} are as described herein, including in embodiments.

In embodiments, a substituted L^{1B} (e.g., substituted alkylene and/or substituted heteroalkylene) is substituted with at least one substituent group, size-limited substituent group, or lower substituent group; wherein if the substituted L^{1B} is substituted with a plurality of groups selected from substituent groups, size-limited substituent groups, and lower substituent groups; each substituent group, size-limited substituent group, and/or lower substituent group may optionally be different. In embodiments, when L^{1B} is substituted, it is substituted with at least one substituent group. In embodiments, when L^{1B} is substituted, it is substituted with at least one size-limited substituent group. In embodiments, when L^{1B} is substituted, it is substituted with at least one lower substituent group.

In embodiments, a substituted L^{2B} (e.g., substituted alkylene and/or substituted heteroalkylene) is substituted with at least one substituent group, size-limited substituent group, or lower substituent group; wherein if the substituted L^{2B} is substituted with a plurality of groups selected from substituent groups, size-limited substituent groups, and lower substituent groups; each substituent group, size-limited substituent group, and/or lower substituent group may optionally be different. In embodiments, when L^{2B} is substituted, it is substituted with at least one substituent group. In embodiments, when L^{2B} is substituted, it is substituted with at least one size-limited substituent group. In embodiments, when L^{2B} is substituted, it is substituted with at least one lower substituent group.

In embodiments, a substituted L^{3B} (e.g., substituted alkylene and/or substituted heteroalkylene) is substituted with at least one substituent group, size-limited substituent group, or lower substituent group; wherein if the substituted L^{3B} is substituted with a plurality of groups selected from substituent groups, size-limited substituent groups, and lower substituent groups; each substituent group, size-limited substituent group, and/or lower substituent group may optionally be different. In embodiments, when L^{3B} is substituted, it is substituted with at least one substituent group. In embodiments, when L^{3B} is substituted, it is substituted with at least one size-limited substituent group. In embodiments, when L^{3B} is substituted, it is substituted with at least one lower substituent group.

In embodiments, a substituted L^{4B} (e.g., substituted alkylene and/or substituted heteroalkylene) is substituted with at least one substituent group, size-limited substituent group, or lower substituent group; wherein if the substituted L^{4B} is substituted with a plurality of groups selected from substituent groups, size-limited substituent groups, and lower substituent groups; each substituent group, size-limited substituent group, and/or lower substituent group may optionally be different. In embodiments, when L^{4B} is substituted, it is substituted with at least one substituent group. In embodiments, when L^{4B} is substituted, it is substituted with at least one size-limited substituent group. In embodiments, when L^{4B} is substituted, it is substituted with at least one lower substituent group.

In embodiments, a substituted L^{5B} (e.g., substituted alkylene and/or substituted heteroalkylene) is substituted with at least one substituent group, size-limited substituent group, or lower substituent group; wherein if the substituted L^{5B} is substituted with a plurality of groups selected from substituent groups, size-limited substituent groups, and lower substituent groups; each substituent group, size-limited substituent group, and/or lower substituent group may optionally be different. In embodiments, when L^{5B} is substituted, it is substituted with at least one substituent group. In embodiments, when L^{5B} is substituted, it is substituted with at least one size-limited substituent group. In embodiments, when L^{5B} is substituted, it is substituted with at least one lower substituent group.

In embodiments, a substituted L^{6B} (e.g., substituted alkylene and/or substituted heteroalkylene) is substituted with at least one substituent group, size-limited substituent group, or lower substituent group; wherein if the substituted L^{6B} is substituted with a plurality of groups selected from substituent groups, size-limited substituent groups, and lower substituent groups; each substituent group, size-limited substituent group, and/or lower substituent group may optionally be different. In embodiments, when L^{6B} is substituted, it is substituted with at least one substituent group. In embodiments, when L^{6B} is substituted, it is substituted with at least one size-limited substituent group. In embodiments, when L^{6B} is substituted, it is substituted with at least one lower substituent group.

In embodiments, a substituted L^{7B} (e.g., substituted alkylene and/or substituted heteroalkylene) is substituted with at least one substituent group, size-limited substituent group, or lower substituent group; wherein if the substituted L^{7B} is substituted with a plurality of groups selected from substituent groups, size-limited substituent groups, and lower substituent groups; each substituent group, size-limited substituent group, and/or lower substituent group may optionally be different. In embodiments, when L^{7B} is substituted, it is substituted with at least one substituent group. In embodiments, when L^{7B} is substituted, it is substituted with at least one size-limited substituent group. In embodiments, when L^{7B} is substituted, it is substituted with at least one lower substituent group.

In embodiments, a substituted L^{8B} (e.g., substituted alkylene and/or substituted heteroalkylene) is substituted with at least one substituent group, size-limited substituent group, or lower substituent group; wherein if the substituted L^{8B} is substituted with a plurality of groups selected from substituent groups, size-limited substituent groups, and lower substituent groups; each substituent group, size-limited substituent group, and/or lower substituent group may optionally be different. In embodiments, when L^{8B} is substituted, it is substituted with at least one substituent group. In embodiments, when L^{8B} is substituted, it is substituted with at least one size-limited substituent group. In embodiments, when L^{8B} is substituted, it is substituted with at least one lower substituent group.

In embodiments, a substituted L^{9B} (e.g., substituted alkylene and/or substituted heteroalkylene) is substituted with at least one substituent group, size-limited substituent group, or lower substituent group; wherein if the substituted L^{9B} is substituted with a plurality of groups selected from substituent groups, size-limited substituent groups, and lower substituent groups; each substituent group, size-limited substituent group, and/or lower substituent group may optionally be different. In embodiments, when L^{9B} is substituted, it is substituted with at least one substituent group. In embodiments, when L^{9B} is substituted, it is substituted with at least one size-limited substituent group. In embodiments, when L^{9B} is substituted, it is substituted with at least one lower substituent group.

In embodiments, a substituted L^{10B} (e.g., substituted alkylene and/or substituted heteroalkylene) is substituted with at least one substituent group, size-limited substituent group, or lower substituent group; wherein if the substituted L^{10B} is substituted with a plurality of groups selected from substituent groups, size-limited substituent groups, and lower substituent groups; each substituent group, size-limited substituent group, and/or lower substituent group may optionally be different. In embodiments, when L^{10B} is substituted, it is substituted with at least one substituent group. In embodiments, when L^{10B} is substituted, it is substituted with at least one size-limited substituent group. In embodiments, when L^{10B} is substituted, it is substituted with at least one lower substituent group.

In embodiments, a substituted L^{11B} (e.g., substituted alkylene and/or substituted heteroalkylene) is substituted with at least one substituent group, size-limited substituent group, or lower substituent group; wherein if the substituted L^{11B} is substituted with a plurality of groups selected from substituent groups, size-limited substituent groups, and lower substituent groups; each substituent group, size-limited substituent group, and/or lower substituent group may optionally be different. In embodiments, when L^{11B} is substituted, it is substituted with at least one substituent group. In embodiments, when L^{11B} is substituted, it is substituted with at least one size-limited substituent group. In embodiments, when L^{11B} is substituted, it is substituted with at least one lower substituent group.

In embodiments, a substituted L^{12B} (e.g., substituted alkylene and/or substituted heteroalkylene) is substituted with at least one substituent group, size-limited substituent group, or lower substituent group; wherein if the substituted L^{12B} is substituted with a plurality of groups selected from substituent groups, size-limited substituent groups, and lower substituent groups; each substituent group, size-limited substituent group, and/or lower substituent group may optionally be different. In embodiments, when L^{12B} is substituted, it is substituted with at least one substituent group. In embodiments, when L^{12B} is substituted, it is substituted with at least one size-limited substituent group. In embodiments, when L^{12B} is substituted, it is substituted with at least one lower substituent group.

In embodiments, a substituted L^{13B} (e.g., substituted alkylene and/or substituted heteroalkylene) is substituted with at least one substituent group, size-limited substituent group, or lower substituent group; wherein if the substituted L^{13B} is substituted with a plurality of groups selected from substituent groups, size-limited substituent groups, and lower substituent groups; each substituent group, size-limited substituent group, and/or lower substituent group may optionally be different. In embodiments, when L^{13B} is substituted, it is substituted with at least one substituent group. In embodiments, when L^{13B} is substituted, it is substituted with at least one size-limited substituent group. In embodiments, when L^{13B} is substituted, it is substituted with at least one lower substituent group.

In embodiments, a substituted R^{1B} (e.g., substituted cycloalkyl, substituted heterocycloalkyl, substituted aryl, and/or substituted heteroaryl) is substituted with at least one substituent group, size-limited substituent group, or lower substituent group; wherein if the substituted R^{1B} is substituted with a plurality of groups selected from substituent groups, size-limited substituent groups, and lower substituent groups; each substituent group, size-limited substituent group, and/or lower substituent group may optionally be different. In embodiments, when R^{1B} is substituted, it is substituted with at least one substituent group. In embodiments, when R^{1B} is substituted, it is substituted with at least one size-limited substituent group. In embodiments, when R^{1B} is substituted, it is substituted with at least one lower substituent group.

In embodiments, a substituted R^{2B} (e.g., substituted alkyl, substituted cycloalkyl, and/or substituted aryl) is substituted with at least one substituent group, size-limited substituent group, or lower substituent group; wherein if the substituted R^{2B} is substituted with a plurality of groups selected from substituent groups, size-limited substituent groups, and lower substituent groups; each substituent group, size-limited substituent group, and/or lower substituent group may optionally be different. In embodiments, when R^{2B} is substituted, it is substituted with at least one substituent group. In embodiments, when R^{2B} is substituted, it is substituted with at least one size-limited substituent group. In embodiments, when R^{2B} is substituted, it is substituted with at least one lower substituent group.

In embodiments, a substituted R^{3B} (e.g., substituted alkyl, substituted cycloalkyl, and/or substituted aryl) is substituted with at least one substituent group, size-limited substituent group, or lower substituent group; wherein if the substituted R^{3B} is substituted with a plurality of groups selected from substituent groups, size-limited substituent groups, and lower substituent groups; each substituent group, size-limited substituent group, and/or lower substituent group may optionally be different. In embodiments, when R^{3B} is substituted, it is substituted with at least one substituent group. In embodiments, when R^{3B} is substituted, it is substituted with at least one size-limited substituent group. In embodiments, when R^{3B} is substituted, it is substituted with at least one lower substituent group.

In embodiments, a substituted R^{4B} (e.g., substituted alkyl, substituted cycloalkyl, and/or substituted aryl) is substituted with at least one substituent group, size-limited substituent group, or lower substituent group; wherein if the substituted R^{4B} is substituted with a plurality of groups selected from substituent groups, size-limited substituent groups, and lower substituent groups; each substituent group, size-limited substituent group, and/or lower substituent group may optionally be different. In embodiments, when R^{4B} is substituted, it is substituted with at least one substituent group. In embodiments, when R^{4B} is substituted, it is substituted with at least one size-limited substituent group. In embodiments, when R^{4B} is substituted, it is substituted with at least one lower substituent group.

In embodiments, a substituted R^{5B} (e.g., substituted alkyl and/or substituted heteroalkyl) is substituted with at least one substituent group, size-limited substituent group, or lower substituent group; wherein if the substituted R^{5B} is substituted with a plurality of groups selected from substituent groups, size-limited substituent groups, and lower substituent groups; each substituent group, size-limited substituent group, and/or lower substituent group may optionally be different. In embodiments, when R^{5B} is substituted, it is substituted with at least one substituent group. In embodiments, when R^{5B} is substituted, it is substituted with at least one size-limited substituent group. In embodiments, when R^{5B} is substituted, it is substituted with at least one lower substituent group.

In embodiments, a substituted R^{6B} (e.g., substituted alkyl and/or substituted heteroalkyl) is substituted with at least one substituent group, size-limited substituent group, or lower substituent group; wherein if the substituted R^{6B} is substituted with a plurality of groups selected from substituent groups, size-limited substituent groups, and lower substituent groups; each substituent group, size-limited substituent group, and/or lower substituent group may optionally be different. In embodiments, when R^{6B} is substituted, it is substituted with at least one substituent group. In embodiments, when R^{6B} is substituted, it is substituted with at least one size-limited substituent group. In embodiments, when R^{6B} is substituted, it is substituted with at least one lower substituent group.

In embodiments, a substituted R^{7B} (e.g., substituted alkyl, substituted heteroalkyl, and/or substituted heteroaryl) is substituted with at least one substituent group, size-limited substituent group, or lower substituent group; wherein if the substituted R^{7B} is substituted with a plurality of groups selected from substituent groups, size-limited substituent groups, and lower substituent groups; each substituent group, size-limited substituent group, and/or lower substituent group may optionally be different. In embodiments, when R^{7B} is substituted, it is substituted with at least one substituent group. In embodiments, when R^{7B} is substituted, it is substituted with at least one size-limited substituent group. In embodiments, when R^{7B} is substituted, it is substituted with at least one lower substituent group.

In embodiments, a substituted R^{8B} (e.g., substituted cycloalkyl, substituted heterocycloalkyl, substituted aryl, and/or substituted heteroaryl) is substituted with at least one substituent group, size-limited substituent group, or lower substituent group; wherein if the substituted R^{8B} is substituted with a plurality of groups selected from substituent groups, size-limited substituent groups, and lower substituent groups; each substituent group, size-limited substituent group, and/or lower substituent group may optionally be different. In embodiments, when R^{8B} is substituted, it is substituted with at least one substituent group. In embodiments, when R^{8B} is substituted, it is substituted with at least one size-limited substituent group. In embodiments, when R^{8B} is substituted, it is substituted with at least one lower substituent group.

In embodiments, a substituted R^{9B} (e.g., substituted alkyl, substituted cycloalkyl, and/or substituted aryl) is substituted with at least one substituent group, size-limited substituent group, or lower substituent group; wherein if the substituted R^{9B} is substituted with a plurality of groups selected from substituent groups, size-limited substituent groups, and lower substituent groups; each substituent group, size-limited substituent group, and/or lower substituent group may optionally be different. In embodiments, when R^{9B} is substituted, it is substituted with at least one substituent group. In embodiments, when R^{9B} is substituted, it is substituted with at least one size-limited substituent group. In embodiments, when R^{9B} is substituted, it is substituted with at least one lower substituent group.

In embodiments, a substituted R^{10B} (e.g., substituted cycloalkyl, substituted heterocycloalkyl, substituted aryl, and/or substituted heteroaryl) is substituted with at least one substituent group, size-limited substituent group, or lower substituent group; wherein if the substituted R^{10B} is substituted with a plurality of groups selected from substituent groups, size-limited substituent groups, and lower substituent groups; each substituent group, size-limited substituent group, and/or lower substituent group may optionally be different. In embodiments, when R^{10B} is substituted, it is substituted with at least one substituent group. In embodiments, when R^{10B} is substituted, it is substituted with at least one size-limited substituent group. In embodiments, when R^{10B} is substituted, it is substituted with at least one lower substituent group.

In embodiments, a substituted R^{11B} (e.g., substituted alkyl, substituted cycloalkyl, and/or substituted aryl) is substituted with at least one substituent group, size-limited substituent group, or lower substituent group; wherein if the substituted R^{11B} is substituted with a plurality of groups selected from substituent groups, size-limited substituent groups, and lower substituent groups; each substituent group, size-limited substituent group, and/or lower substituent group may optionally be different. In embodiments, when R^{11B} is substituted, it is substituted with at least one substituent group. In embodiments, when R^{11B} is substituted, it is substituted with at least one size-limited substituent group. In embodiments, when R^{11B} is substituted, it is substituted with at least one lower substituent group.

In embodiments, a substituted R^{12B} (e.g., substituted alkyl, substituted heteroalkyl, and/or substituted heteroaryl) is substituted with at least one substituent group, size-limited substituent group, or lower substituent group; wherein if the substituted R^{12B} is substituted with a plurality of groups selected from substituent groups, size-limited substituent groups, and lower substituent groups; each substituent group, size-limited substituent group, and/or lower substituent group may optionally be different. In embodiments, when R^{12B} is substituted, it is substituted with at least one substituent group. In embodiments, when R^{12B} is substituted, it is substituted with at least one size-limited substituent group. In embodiments, when R^{12B} is substituted, it is substituted with at least one lower substituent group.

In embodiments, a substituted R^{13B} (e.g., substituted alkyl, substituted heteroalkyl, and/or substituted heteroaryl) is substituted with at least one substituent group, size-limited substituent group, or lower substituent group; wherein if the substituted R^{13B} is substituted with a plurality of groups selected from substituent groups, size-limited substituent groups, and lower substituent groups; each substituent group, size-limited substituent group, and/or lower substituent group may optionally be different. In embodiments, when R^{13B} is substituted, it is substituted with at least one substituent group. In embodiments, when R^{13B} is substituted, it is substituted with at least one size-limited substituent group. In embodiments, when R^{13B} is substituted, it is substituted with at least one lower substituent group.

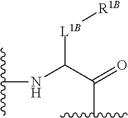

In embodiments, H is a divalent form of an unnatural amino acid. In embodiments, L^{1B} is a bond or unsubstituted C₁-C₄ alkylene. In embodiments, R^{1B} is substituted or unsubstituted aryl, or substituted or unsubstituted heteroaryl. In embodiments,

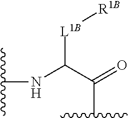

is a divalent form of a natural amino acid. In embodiments,

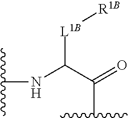

is a divalent form of tyrosine, a divalent form of phenylalanine, or a divalent form of tryptophan.

In embodiments,

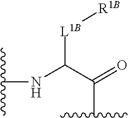

is a divalent form of tyrosine. In embodiments, -L^{1B}-R^{1B} is

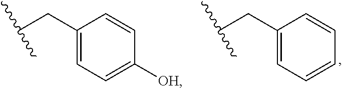 [SMILES: *Cc1ccc(O)cc1 ; *Cc1ccccc1]

In embodiments, -L^{1B}-R^{1B} is

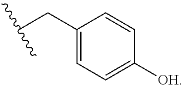 [SMILES: *Cc1ccc(O)cc1]

In embodiments,

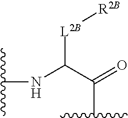

is a divalent form of an unnatural amino acid. In embodiments, L^{2B} is a bond or unsubstituted C₁-C₄ alkylene. In embodiments, R^{2B} is a substituted or unsubstituted alkyl. In embodiments,

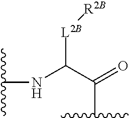

is a divalent form of a natural amino acid. In embodiments,

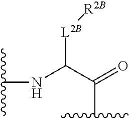

is a divalent form of isoleucine, a divalent form of leucine, or a divalent form of valine. In embodiments,

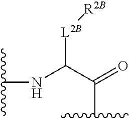

is a divalent form of isoleucine. In embodiments, -L^{2B}-R^{2B} is

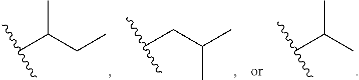

In embodiments, -L^{2B}-R^{2B} is

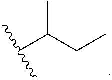 [SMILES: *C(C)CC]

In embodiments,

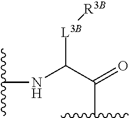

is a divalent form of an unnatural amino acid. In embodiments, L^{3B} is a bond or unsubstituted C₁-C₄ alkylene. In embodiments, R^{3B} is a substituted or unsubstituted alkyl. In embodiments,

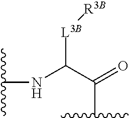

is a divalent form of a natural amino acid. In embodiments,

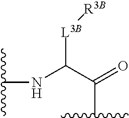

is a divalent form of isoleucine, a divalent form of leucine, or a divalent form of valine. In embodiments,

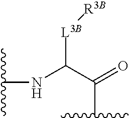

is a divalent form of isoleucine. In embodiments, -L^{3B}-R^{3B} is

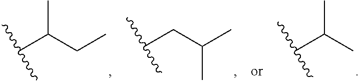

In embodiments, -L^{3B}-R^{3B} is

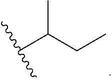 [SMILES: *C(C)CC]

In embodiments,

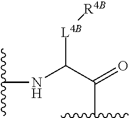

is a divalent form of an unnatural amino acid. In embodiments, L^{4B} is a bond or unsubstituted C₁-C₄ alkylene. In embodiments, R^{4B} is a substituted or unsubstituted alkyl. In embodiments,

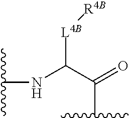

is a divalent form of a natural amino acid. In embodiments,

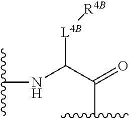

is a divalent form of valine, a divalent form of isoleucine, a divalent form of leucine, or a divalent form of alanine. In embodiments,

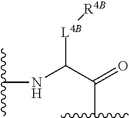

is a divalent form of valine. In embodiments,

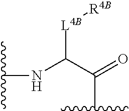

is a divalent form of alanine. In embodiments, -L^{4B}-R^{4B} is

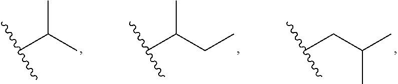

or —CH₃. In embodiments, -L^{4B}-R^{4B} is

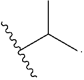 [SMILES: *C(C)C]

In embodiments, -L^{4B}-R^{4B} is —CH₃.

In embodiments,

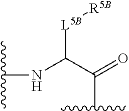

is a divalent form of an unnatural amino acid. In embodiments, L^{5B} is a bond or unsubstituted C₁-C₄ alkylene. In embodiments, R^{5B} is —OH or substituted or unsubstituted alkyl. In embodiments,

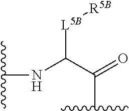

is a divalent form of a natural amino acid. In embodiments,

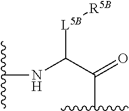

is a divalent form of threonine or a divalent form of serine. In embodiments,

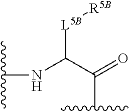

is a divalent form of threonine. In embodiments, -L^{5B}-R^{5B} is

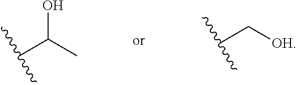

In embodiments, -L^{5B}-R^{5B} is

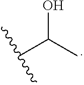 [SMILES: *C(C)O]

In embodiments,

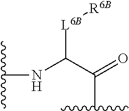

is a divalent form of an unnatural amino acid. In embodiments, L^{6B} is a bond or unsubstituted C₁-C₄ alkylene. In embodiments, R^{6B} is —C(O)OH or substituted or unsubstituted heteroalkyl. In embodiments,

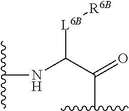

is a divalent form of a natural amino acid. In embodiments,

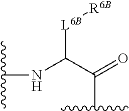

is a divalent form of glutamic acid or a divalent form of aspartic acid. In embodiments,

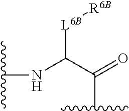

is a divalent form of glutamic acid. In embodiments, -L^{6B}-R^{6B} is

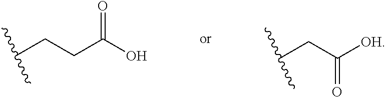 [SMILES: *CCC(=O)O ; *CC(=O)O]

In embodiments, -L^{6B}-R^{6B} is

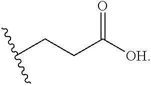 [SMILES: *CCC(=O)O]

In embodiments,

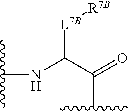

is a divalent form of an unnatural amino acid. In embodiments, L^{7B} is a bond or unsubstituted C₁-C₆ alkylene. In embodiments, R^{7B} is —NH₂, —NHC(NH)NH₂, substituted or unsubstituted heteroalkyl, or substituted or unsubstituted heteroaryl. In embodiments,

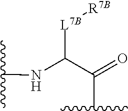

is a divalent form of a natural amino acid. In embodiments,

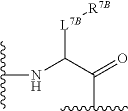

is a divalent form of lysine, a divalent form of arginine, or a divalent form of histidine. In embodiments,

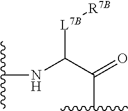

is a divalent form of lysine. In embodiments, -L^{7B}-R^{7B} is

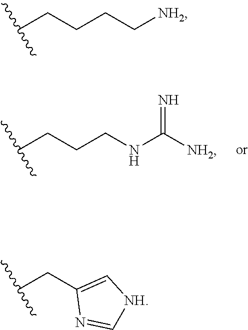 [SMILES: *CCCCN ; *CCCNC(=N)N ; *Cc1c[nH]cn1]

In embodiments, -L^{7B}-R^{7B} is

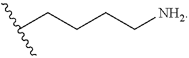 [SMILES: *CCCCN]

In embodiments,

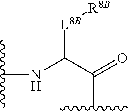

is a divalent form of an unnatural amino acid. In embodiments, L⁸B is a bond or unsubstituted C₁-C₄ alkylene. In embodiments, R^{8B} is a substituted or unsubstituted aryl, or substituted or unsubstituted heteroaryl. In embodiments,

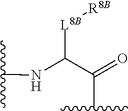

is a divalent form of a natural amino acid. In embodiments,

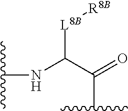

is a divalent form of phenylalanine, a divalent form of tyrosine, or a divalent form of tryptophan.

In embodiments,

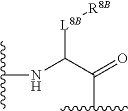

is a divalent form of phenylalanine. In embodiments, -L^{8B}-R^{8B} is

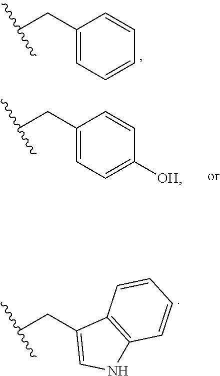

In embodiments, -L^{8B}-R^{8B} is

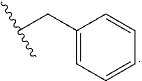 [SMILES: *Cc1ccccc1]

In embodiments,

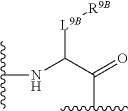

is a divalent form of an unnatural amino acid. In embodiments, L^{9B} is a bond or unsubstituted C₁-C₄ alkylene. In embodiments, R^{9B} is a substituted or unsubstituted alkyl. In embodiments,

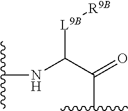

is a divalent form of a natural amino acid. In embodiments,

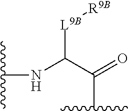

is a divalent form of isoleucine, a divalent form of leucine, or a divalent form of valine. In embodiments,

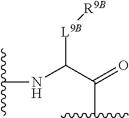

is a divalent form of isoleucine. In embodiments, -L^{9B}-R^{9B} is

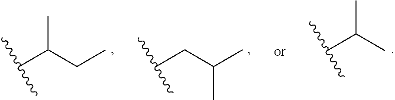

In embodiments, -L^{9B}-R^{9B} is

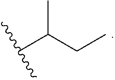 [SMILES: *C(C)CC]

In embodiments,

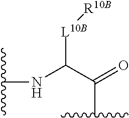

is a divalent form of an unnatural amino acid. In embodiments, L^{10B} is a bond or unsubstituted C₁-C₄ alkylene. In embodiments, R^{10B} is a substituted or unsubstituted aryl, or substituted or unsubstituted heteroaryl. In embodiments,

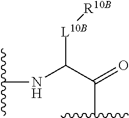

is a divalent form of a natural amino acid. In embodiments,

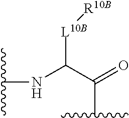

is a divalent form of tryptophan, a divalent form of phenylalanine, or a divalent form of tyrosine. In embodiments,

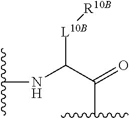

is a divalent form of tryptophan. In embodiments, -L^{10B}-R^{10B} is

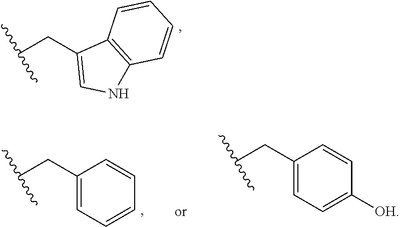 [SMILES: *Cc1c[nH]c2ccccc12 ; *Cc1ccccc1 ; *Cc1ccc(O)cc1]

In embodiments, -L^{10B}-R^{10B} is

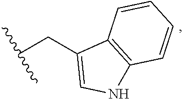 [SMILES: *Cc1c[nH]c2ccccc12]

In embodiments,

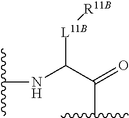

is a divalent form of an unnatural amino acid. In embodiments, L^{11B} is a bond or unsubstituted C₁-C₄ alkylene. In embodiments, R^{11B} is a substituted or unsubstituted alkyl. In embodiments,

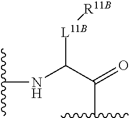

is a divalent form of a natural amino acid. In embodiments,

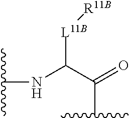

is a divalent form of valine, a divalent form of isoleucine, or a divalent form of leucine. In embodiments,

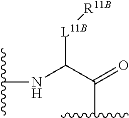

is a divalent form of vane. In embodiments, -L^{11B}-L^{11B} is

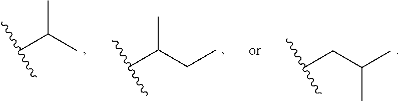

In embodiments, -L^{11B}-R^{11B} is

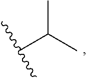 [SMILES: *C(C)C]

In embodiments

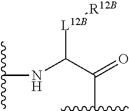

is a divalent form of an unnatural amino acid. In embodiments, L^{12B} is a bond or unsubstituted C₁-C₆ alkylene. In embodiments, R^{12B} is —NH₂, —NHC(NH)NH₂, substituted or unsubstituted heteroalkyl, or substituted or unsubstituted heteroaryl. In embodiments,

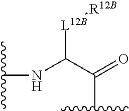

is a divalent form of a natural amino acid. In embodiments,

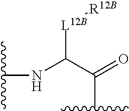

is a divalent form of histidine, a divalent form of lysine, or a divalent form of arginine. In embodiments,

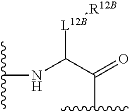

is a divalent form of histidine. In embodiments, -L^{12B}-R^{12B} is

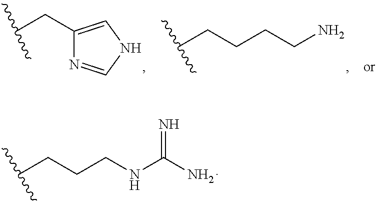 [SMILES: *Cc1c[nH]cn1 ; *CCCCN ; *CCCNC(=N)N]

In embodiments, -L^{12B}-R^{12B} is

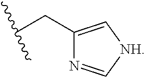 [SMILES: *Cc1c[nH]cn1]

In embodiments,

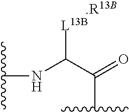

is a divalent form of an unnatural amino acid. In embodiments, L^{13B} is a bond or unsubstituted C₁-C₆ alkylene. In embodiments, R^{13B} is —NH₂, —NHC(NH)NH₂, substituted or unsubstituted heteroalkyl, or substituted or unsubstituted heteroaryl. In embodiments,

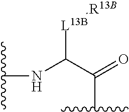

is a divalent form of a natural amino acid. In embodiments,

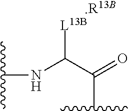

is a divalent form of histidine, a divalent form of lysine, or a divalent form of arginine. In embodiments,

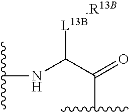

is a divalent form of histidine. In embodiments, -L^{13B}-L^{13B} is

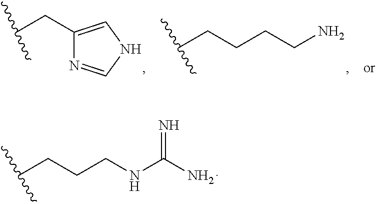 [SMILES: *Cc1c[nH]cn1 ; *CCCCN ; *CCCNC(=N)N]

In embodiments, -L^{13B}-R^{13B} is

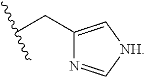 [SMILES: *Cc1c[nH]cn1]

In embodiments, two substituents selected from R^{1B}, R^{2B}, R^{3B}, R^{4B}, R^{5B}, R^{6B}, L^{7B}, R^{8B}, R^{9B}, R^{10B}, R^{11B}, R^{12B}, and R^{13B} may optionally be joined to form a bioconjugate linker. In embodiments, two substituents selected from R^{1B}, R^{2B}, R^{3B}, R^{4B}, R^{5B}, R^{6B}, L^{7B}, R^{8B}, R^{9B}, R^{10B}, R^{11B}, R^{12B}, and R^{13B} may optionally be joined to form a covalent linker having the formula -L^{18A}-L^{18B}-L^{18C}-L^{18D}-L^{18E}-L^{18F}-, L^{18A}, L^{18B}, L^{18C}, L^{18D}, L^{18E}, and L^{18F} are as described herein, including in embodiments.

In embodiments, the compound of formula (II) is a peptide of FIG. 1B. In embodiments, the compound of formula (II) is peptide 1, 7, 8, 9, 10, 11, 12, 13, 14, 16, or 19 of FIG. 1B. For example, for peptide 1 of FIG. 1B,

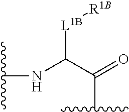

is a divalent form of D-tyrosine;

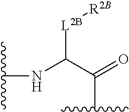

is a divalent form of isoleucine;

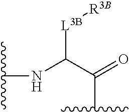

is a divalent form of isoleucine;

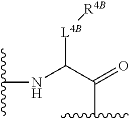

is a divalent form of valine;

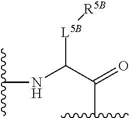

is a divalent form of threonine;

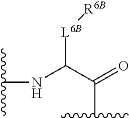

is a divalent form of glutamic acid;

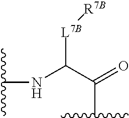

is a divalent form of lysine;

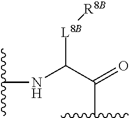

is a divalent form of phenylalanine;

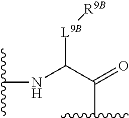

is a divalent form of isoleucine;

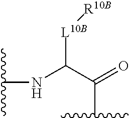

is a divalent form of tryptophan;

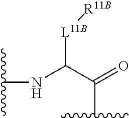

is a divalent form of valine;

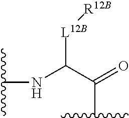

is a divalent form of histidine; and

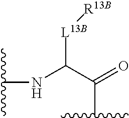

is a divalent form of histidine. Where the compound of formula (II) is peptide 7, 8, 9, 10, 11, 12, 13, 14, 16, or 19 of FIG. 1B, the same exemplification would apply. For example, for peptide 7 of FIG. 1B,

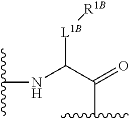

is a divalent form of D-tyrosine (with the same exemplification of the remaining amino acids). For example, for peptide 8 of FIG. 1B,

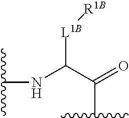

is a divalent form of D-tyrosine (with the same exemplification of the remaining amino acids).

For example, for peptide 9 of FIG. 1B,

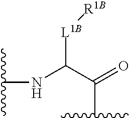

is a divalent form of D-tyrosine (with the same exemplification of the remaining amino acids). For example, for peptide 10 of FIG. 1B,

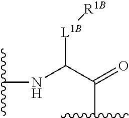

is a divalent form of D-tyrosine (with the same exemplification of the remaining amino acids). For example, for peptide 11 of FIG. 1B,

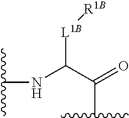

is a divalent form of D-tyrosine (with the same exemplification of the remaining amino acids). For example, for peptide 12 of FIG. 1B,

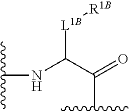

is a divalent form of D-tyrosine (with the same exemplification of the remaining amino acids). For example, for peptide 13 of FIG. 1B,

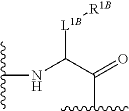

is a divalent form of D-tyrosine (with the same exemplification of the remaining amino acids). For example, for peptide 14 of FIG. 1B,

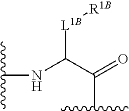

is a divalent form of D-tyrosine (with the same exemplification of the remaining amino acids). For example, for peptide 16 of FIG. 1B,

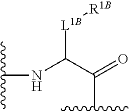

is a divalent form of D-tyrosine (with the same exemplification of the remaining amino acids). For example, for peptide 19 of FIG. 1B,

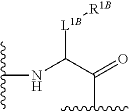

is a divalent form of D-tyrosine (with the same exemplification of the remaining amino acids).

In embodiments, the compound has the formula:

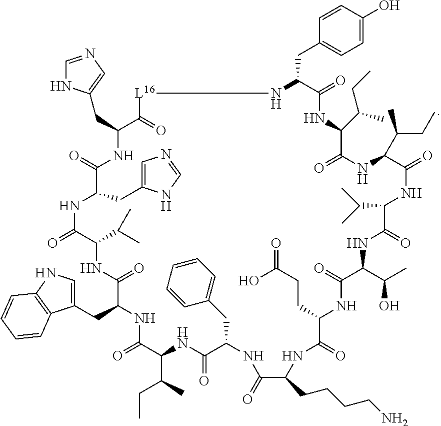

L¹⁶ is as described herein, including in embodiments.

In an aspect is provided a compound having the formula:

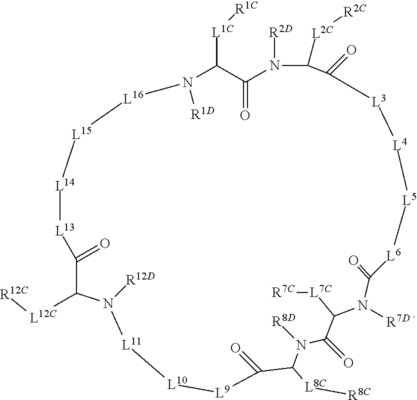

R^{1D}, R^{2D}, R^{3D}, R^{4D}, R^{5D}, R^{6D}, R^{7D}, R^{8D}, R^{9D}, R^{10D}, R^{11D}, R^{12D}, R^{13D}, and L¹⁶ are as described herein, including in embodiments.

In an aspect is provided a compound having the formula:

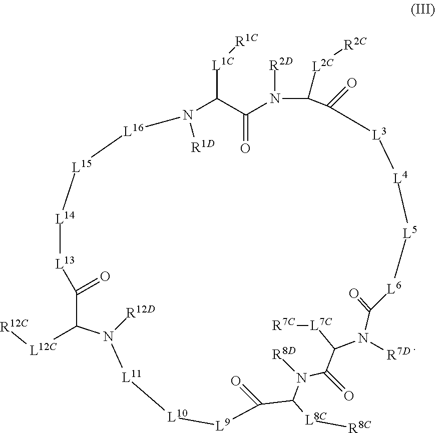

R^{1D}, R^{2D}, R^{3D}, R^{4D}, R^{5D}, R^{6D}, R^{7D}, R^{8D}, R^{9D}, R^{10D}, R^{11D}, R^{12D}, R^{13D}, and L¹⁶ are as described herein, including in embodiments.

L^{1C}, L^{2C}, L^{3C}, L^{4C}, L^{5C}, L^{6C}, L^{7C}, L^{8C}, L^{9C}, L^{10C}, L^{11C}, L^{12C}, L^{13C}, L^{14C}, and L^{15C} are independently a bond, substituted or unsubstituted alkylene (e.g., C₁-C₈, C₁-C₆, C₁-C₄, or C₁-C₂), or substituted or unsubstituted heteroalkylene (e.g., 2 to 8 membered, 2 to 6 membered, 4 to 6 membered, 2 to 3 membered, or 4 to 5 membered).

R^{1C} is independently substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R^{2C} is independently hydrogen, —OH, —NO₂, —CN, —NH₂, —C(O)OH, —C(O)NH₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, substituted or unsubstituted alkyl (e.g., C₁-C₈, C₁-C₆, C₁-C₄, or C₁-C₂), or substituted or unsubstituted heteroalkyl (e.g., 2 to 8 membered, 2 to 6 membered, 4 to 6 membered, 2 to 3 membered, or 4 to 5 membered).

L³ is independently a bond or

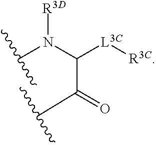

R^{3C} is independently hydrogen, —NH₂, —C(O)OH, —C(O)NH₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

L⁴ is independently a bond or

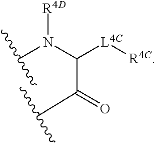

R^{4C} is independently hydrogen, substituted or unsubstituted alkyl (e.g., C₁-C₈, C₁-C₆, C₁-C₄, or C₁-C₂), substituted or unsubstituted cycloalkyl (e.g., C₃-C₈, C₃-C₆, C₄-C₆, or C₅-C₆), or substituted or unsubstituted aryl (e.g., C₆-C₁₀ or phenyl).

L⁵ is independently a bond or

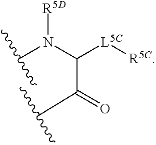

R^{5C} is independently substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

L⁶ is independently a bond,

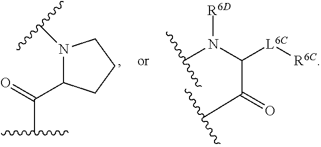

R^{6C} is independently hydrogen, —CN, —NH₂, —C(O)NH₂, —SO₂NH₂, —NHNH₂, —ONH₂, —NHC(O)NHNH₂, —NHC(O)NH₂, —NHC(NH)NH₂, —NHOH, substituted or unsubstituted alkyl (e.g., C₁-C₈, C₁-C₆, C₁-C₄, or C₁-C₂), or substituted or unsubstituted heteroalkyl (e.g., 2 to 8 membered, 2 to 6 membered, 4 to 6 membered, 2 to 3 membered, or 4 to 5 membered).

R^{7C} and R^{8C} are independently hydrogen, —CN, —NH₂, —C(O)NH₂, —NHNH₂, —ONH₂, —NHC(O)NHNH₂, —NHC(O)NH₂, —NHC(NH)NH₂, —NHOH, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

L⁹ is independently a bond,

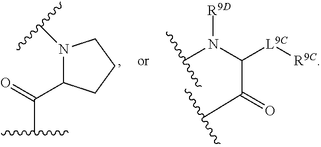

R^{9C} is independently hydrogen, substituted or unsubstituted alkyl (e.g., C₁-C₈, C₁-C₆, C₁-C₄, or C₁-C₂), substituted or unsubstituted cycloalkyl (e.g., C₃-C₈, C₃-C₆, C₄-C₆, or C₅-C₆), or substituted or unsubstituted aryl (e.g., C₆-C₁₀ or phenyl).

L¹⁰ is independently a bond,

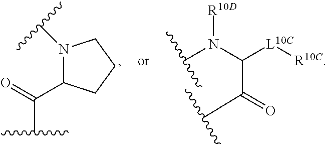

R^{10C} is independently hydrogen, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

L¹¹ is independently a bond or

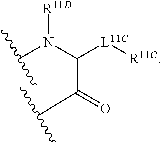

R^{11C} is independently hydrogen, —CN, —OH, —C(O)OH, —NO₂, —SO₃H, —OSO₃H, —NH₂, —C(O)NH₂, —SO₂NH₂, —NHNH₂, —ONH₂, —NHC(O)NHNH₂, —NHC(O)NH₂, —NHC(NH)NH₂, substituted or unsubstituted alkyl (e.g., C₁-C₈, C₁-C₆, C₁-C₄, or C₁-C₂), or substituted or unsubstituted heteroalkyl (e.g., 2 to 8 membered, 2 to 6 membered, 4 to 6 membered, 2 to 3 membered, or 4 to 5 membered).

R^{12C} is independently hydrogen, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

L¹³ is independently

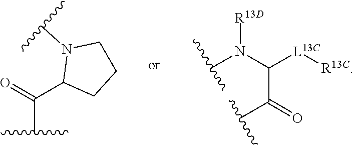

R^{13C} is independently hydrogen, —OH, —NH₂, —C(O)OH, —C(O)NH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), or substituted or unsubstituted aryl (e.g., C₆-C₁₀ or phenyl).

L¹⁴ is independently a bond or

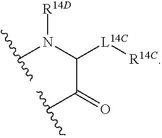

R^{14C} is independently hydrogen, —NH₂, —C(O)OH, —C(O)NH₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

L¹⁵ is independently a bond or

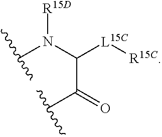

R^{15C} is independently hydrogen, —NH₂, —C(O)OH, —C(O)NH₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

Two substituents selected from R^{1C}, R^{2C}, R^{3C}, R^{4C}, R^{5C}, R^{6C}, R^{7C}, R^{8C}, R^{9C}, R^{10C}, R^{11C}, R^{12C}, R^{13C}, R^{14C}, and R^{15C} may optionally be joined to form a covalent linker.

R^{14D} and R^{15D} are independently hydrogen or unsubstituted C₁-C₄ alkyl.

In embodiments, the compound has the formula:

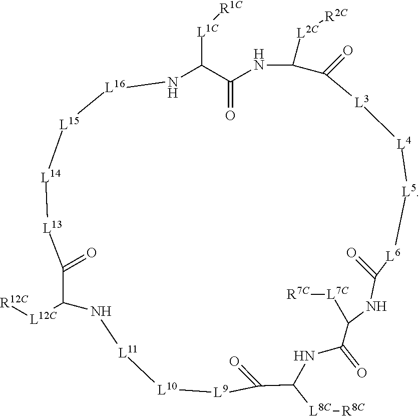

L^{1C}, L^{2C}, L³, L⁴, L⁵, L⁶, L^{7C}, L^{8C}, L⁹, L¹⁰, L¹¹, L^{12C}, L¹³, L¹⁴, L¹⁵, L¹⁶, R^{1C}, R^{2C}, R^{7C}, R^{8C}, and R^{12C} are as described herein, including in embodiments.

In embodiments, the compound has the formula:

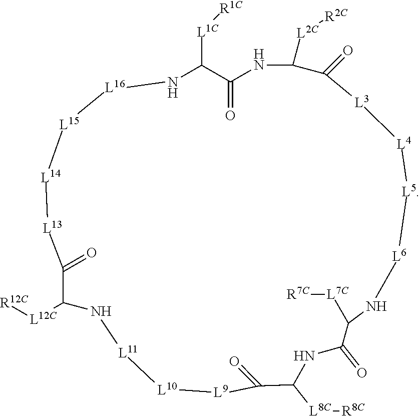

L^{1C}, L^{2C}, L³, L⁴, L⁵, L⁶, L^{7C}, L^{8C}, L⁹, L¹⁰, L¹¹, L^{12C}, L¹³, L¹⁴, L¹⁵, L¹⁶, R^{1C}, R^{2C}, R^{7C}, R^{8C}, and R^{12C} are as described herein, including in embodiments.

In embodiments, the compound has the formula:

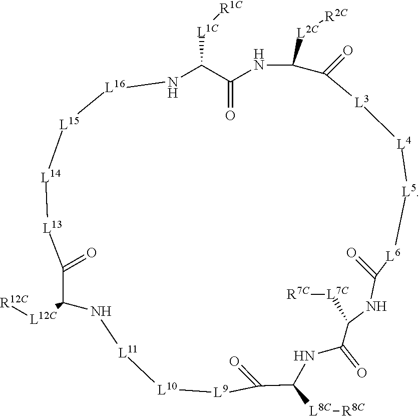

L^{1C}, L^{2C}, L³, L⁴, L⁵, L⁶, L^{7C}, L^{8C}, L⁹, L¹⁰, L¹¹, L^{12C}, L¹³, L¹⁴, L¹⁵, L¹⁶, R^{1C}, R^{2C}, R^{7C}, R^{8C}, and R^{12C} are as described herein, including in embodiments.

In embodiments, the compound has the formula:

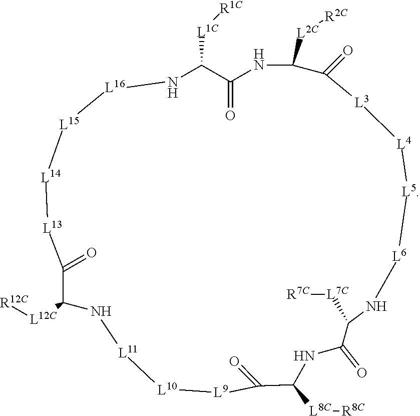

L^{1C}, L^{2C}, L³, L⁴, L⁵, L⁶, L^{7C}, L^{8C}, L⁹, L¹⁰, L¹¹, L^{12C}, L¹³, L¹⁴, L¹⁵, L¹⁶, R^{1C}, R^{2C}, R^{7C}, R^{8C}, and R^{12C} are as described herein, including in embodiments.

In embodiments, a substituted L^{1C} (e.g., substituted alkylene and/or substituted heteroalkylene) is substituted with at least one substituent group, size-limited substituent group, or lower substituent group; wherein if the substituted L^{1C} is substituted with a plurality of groups selected from substituent groups, size-limited substituent groups, and lower substituent groups; each substituent group, size-limited substituent group, and/or lower substituent group may optionally be different. In embodiments, when L^{1C} is substituted, it is substituted with at least one substituent group. In embodiments, when L^{1C} is substituted, it is substituted with at least one size-limited substituent group. In embodiments, when L^{1C} is substituted, it is substituted with at least one lower substituent group.

In embodiments, a substituted L^{2C} (e.g., substituted alkylene and/or substituted heteroalkylene) is substituted with at least one substituent group, size-limited substituent group, or lower substituent group; wherein if the substituted L^{2C} is substituted with a plurality of groups selected from substituent groups, size-limited substituent groups, and lower substituent groups; each substituent group, size-limited substituent group, and/or lower substituent group may optionally be different. In embodiments, when L^{2C} is substituted, it is substituted with at least one substituent group. In embodiments, when L^{2C} is substituted, it is substituted with at least one size-limited substituent group. In embodiments, when L^{2C} is substituted, it is substituted with at least one lower substituent group.

In embodiments, a substituted L^{3C} (e.g., substituted alkylene and/or substituted heteroalkylene) is substituted with at least one substituent group, size-limited substituent group, or lower substituent group; wherein if the substituted L^{3C} is substituted with a plurality of groups selected from substituent groups, size-limited substituent groups, and lower substituent groups; each substituent group, size-limited substituent group, and/or lower substituent group may optionally be different. In embodiments, when L^{3C} is substituted, it is substituted with at least one substituent group. In embodiments, when L^{3C} is substituted, it is substituted with at least one size-limited substituent group. In embodiments, when L^{3C} is substituted, it is substituted with at least one lower substituent group.

In embodiments, a substituted L^{4C} (e.g., substituted alkylene and/or substituted heteroalkylene) is substituted with at least one substituent group, size-limited substituent group, or lower substituent group; wherein if the substituted L^{4C} is substituted with a plurality of groups selected from substituent groups, size-limited substituent groups, and lower substituent groups; each substituent group, size-limited substituent group, and/or lower substituent group may optionally be different. In embodiments, when L^{4C} is substituted, it is substituted with at least one substituent group. In embodiments, when L^{4C} is substituted, it is substituted with at least one size-limited substituent group. In embodiments, when L^{4C} is substituted, it is substituted with at least one lower substituent group.

In embodiments, a substituted L^{5C} (e.g., substituted alkylene and/or substituted heteroalkylene) is substituted with at least one substituent group, size-limited substituent group, or lower substituent group; wherein if the substituted L^{5C} is substituted with a plurality of groups selected from substituent groups, size-limited substituent groups, and lower substituent groups; each substituent group, size-limited substituent group, and/or lower substituent group may optionally be different. In embodiments, when L^{5C} is substituted, it is substituted with at least one substituent group. In embodiments, when L^{5C} is substituted, it is substituted with at least one size-limited substituent group. In embodiments, when L^{5C} is substituted, it is substituted with at least one lower substituent group.

In embodiments, a substituted L^{6C} (e.g., substituted alkylene and/or substituted heteroalkylene) is substituted with at least one substituent group, size-limited substituent group, or lower substituent group; wherein if the substituted L^{6C} is substituted with a plurality of groups selected from substituent groups, size-limited substituent groups, and lower substituent groups; each substituent group, size-limited substituent group, and/or lower substituent group may optionally be different. In embodiments, when L^{6C} is substituted, it is substituted with at least one substituent group. In embodiments, when L^{6C} is substituted, it is substituted with at least one size-limited substituent group. In embodiments, when L^{6C} is substituted, it is substituted with at least one lower substituent group.

In embodiments, a substituted L^{7C} (e.g., substituted alkylene and/or substituted heteroalkylene) is substituted with at least one substituent group, size-limited substituent group, or lower substituent group; wherein if the substituted L^{7C} is substituted with a plurality of groups selected from substituent groups, size-limited substituent groups, and lower substituent groups; each substituent group, size-limited substituent group, and/or lower substituent group may optionally be different. In embodiments, when L^{7C} is substituted, it is substituted with at least one substituent group. In embodiments, when L^{7C} is substituted, it is substituted with at least one size-limited substituent group. In embodiments, when L^{7C} is substituted, it is substituted with at least one lower substituent group.

In embodiments, a substituted L^{8C} (e.g., substituted alkylene and/or substituted heteroalkylene) is substituted with at least one substituent group, size-limited substituent group, or lower substituent group; wherein if the substituted L^{8C} is substituted with a plurality of groups selected from substituent groups, size-limited substituent groups, and lower substituent groups; each substituent group, size-limited substituent group, and/or lower substituent group may optionally be different. In embodiments, when L^{8C} is substituted, it is substituted with at least one substituent group. In embodiments, when L^{8C} is substituted, it is substituted with at least one size-limited substituent group. In embodiments, when L^{8C} is substituted, it is substituted with at least one lower substituent group.

In embodiments, a substituted L^{9C} (e.g., substituted alkylene and/or substituted heteroalkylene) is substituted with at least one substituent group, size-limited substituent group, or lower substituent group; wherein if the substituted L^{9C} is substituted with a plurality of groups selected from substituent groups, size-limited substituent groups, and lower substituent groups; each substituent group, size-limited substituent group, and/or lower substituent group may optionally be different. In embodiments, when L^{9C} is substituted, it is substituted with at least one substituent group. In embodiments, when L^{9C} is substituted, it is substituted with at least one size-limited substituent group. In embodiments, when L^{9C} is substituted, it is substituted with at least one lower substituent group.

In embodiments, a substituted L^{10C} (e.g., substituted alkylene and/or substituted heteroalkylene) is substituted with at least one substituent group, size-limited substituent group, or lower substituent group; wherein if the substituted L^{10C} is substituted with a plurality of groups selected from substituent groups, size-limited substituent groups, and lower substituent groups; each substituent group, size-limited substituent group, and/or lower substituent group may optionally be different. In embodiments, when L^{10C} is substituted, it is substituted with at least one substituent group. In embodiments, when L^{10C} is substituted, it is substituted with at least one size-limited substituent group. In embodiments, when L^{10C} is substituted, it is substituted with at least one lower substituent group.

In embodiments, a substituted L^{11C} (e.g., substituted alkylene and/or substituted heteroalkylene) is substituted with at least one substituent group, size-limited substituent group, or lower substituent group; wherein if the substituted L^{11C} is substituted with a plurality of groups selected from substituent groups, size-limited substituent groups, and lower substituent groups; each substituent group, size-limited substituent group, and/or lower substituent group may optionally be different. In embodiments, when L^{11C} is substituted, it is substituted with at least one substituent group. In embodiments, when L^{11C} is substituted, it is substituted with at least one size-limited substituent group. In embodiments, when L^{11C} is substituted, it is substituted with at least one lower substituent group.

In embodiments, a substituted L^{12C} (e.g., substituted alkylene and/or substituted heteroalkylene) is substituted with at least one substituent group, size-limited substituent group, or lower substituent group; wherein if the substituted L^{12C} is substituted with a plurality of groups selected from substituent groups, size-limited substituent groups, and lower substituent groups; each substituent group, size-limited substituent group, and/or lower substituent group may optionally be different. In embodiments, when L^{12C} is substituted, it is substituted with at least one substituent group. In embodiments, when L^{12C} is substituted, it is substituted with at least one size-limited substituent group. In embodiments, when L^{12C} is substituted, it is substituted with at least one lower substituent group.

In embodiments, a substituted L^{13C} (e.g., substituted alkylene and/or substituted heteroalkylene) is substituted with at least one substituent group, size-limited substituent group, or lower substituent group; wherein if the substituted L^{13C} is substituted with a plurality of groups selected from substituent groups, size-limited substituent groups, and lower substituent groups; each substituent group, size-limited substituent group, and/or lower substituent group may optionally be different. In embodiments, when L^{13C} is substituted, it is substituted with at least one substituent group. In embodiments, when L^{13C} is substituted, it is substituted with at least one size-limited substituent group. In embodiments, when L^{13C} is substituted, it is substituted with at least one lower substituent group.

In embodiments, a substituted L^{14C} (e.g., substituted alkylene and/or substituted heteroalkylene) is substituted with at least one substituent group, size-limited substituent group, or lower substituent group; wherein if the substituted L^{14C} is substituted with a plurality of groups selected from substituent groups, size-limited substituent groups, and lower substituent groups; each substituent group, size-limited substituent group, and/or lower substituent group may optionally be different. In embodiments, when L^{14C} is substituted, it is substituted with at least one substituent group. In embodiments, when L^{14C} is substituted, it is substituted with at least one size-limited substituent group. In embodiments, when L^{14C} is substituted, it is substituted with at least one lower substituent group.

In embodiments, a substituted L^{15C} (e.g., substituted alkylene and/or substituted heteroalkylene) is substituted with at least one substituent group, size-limited substituent group, or lower substituent group; wherein if the substituted L^{15C} is substituted with a plurality of groups selected from substituent groups, size-limited substituent groups, and lower substituent groups; each substituent group, size-limited substituent group, and/or lower substituent group may optionally be different. In embodiments, when L^{15C} is substituted, it is substituted with at least one substituent group. In embodiments, when L^{15C} is substituted, it is substituted with at least one size-limited substituent group. In embodiments, when L^{15C} is substituted, it is substituted with at least one lower substituent group.

In embodiments, a substituted R^{1C} (e.g., substituted cycloalkyl, substituted heterocycloalkyl, substituted aryl, and/or substituted heteroaryl) is substituted with at least one substituent group, size-limited substituent group, or lower substituent group; wherein if the substituted R^{1C} is substituted with a plurality of groups selected from substituent groups, size-limited substituent groups, and lower substituent groups; each substituent group, size-limited substituent group, and/or lower substituent group may optionally be different. In embodiments, when R^{1C} is substituted, it is substituted with at least one substituent group. In embodiments, when R^{1C} is substituted, it is substituted with at least one size-limited substituent group. In embodiments, when R^{1C} is substituted, it is substituted with at least one lower substituent group.

In embodiments, a substituted R^{2C} (e.g., substituted alkyl and/or substituted heteroalkyl) is substituted with at least one substituent group, size-limited substituent group, or lower substituent group; wherein if the substituted R^{2C} is substituted with a plurality of groups selected from substituent groups, size-limited substituent groups, and lower substituent groups; each substituent group, size-limited substituent group, and/or lower substituent group may optionally be different. In embodiments, when R^{2C} is substituted, it is substituted with at least one substituent group. In embodiments, when R^{2C} is substituted, it is substituted with at least one size-limited substituent group. In embodiments, when R^{2C} is substituted, it is substituted with at least one lower substituent group.

In embodiments, a substituted R^{3C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3C} is substituted with a plurality of groups selected from substituent groups, size-limited substituent groups, and lower substituent groups; each substituent group, size-limited substituent group, and/or lower substituent group may optionally be different. In embodiments, when R^{3C} is substituted, it is substituted with at least one substituent group. In embodiments, when R^{3C} is substituted, it is substituted with at least one size-limited substituent group. In embodiments, when R^{3C} is substituted, it is substituted with at least one lower substituent group.

In embodiments, a substituted R^{4C} (e.g., substituted alkyl, substituted cycloalkyl, and/or substituted aryl) is substituted with at least one substituent group, size-limited substituent group, or lower substituent group; wherein if the substituted R^{4C} is substituted with a plurality of groups selected from substituent groups, size-limited substituent groups, and lower substituent groups; each substituent group, size-limited substituent group, and/or lower substituent group may optionally be different. In embodiments, when R^{4C} is substituted, it is substituted with at least one substituent group. In embodiments, when R^{4C} is substituted, it is substituted with at least one size-limited substituent group. In embodiments, when R^{4C} is substituted, it is substituted with at least one lower substituent group.

In embodiments, a substituted R^{5C} (e.g., substituted cycloalkyl, substituted heterocycloalkyl, substituted aryl, and/or substituted heteroaryl) is substituted with at least one substituent group, size-limited substituent group, or lower substituent group; wherein if the substituted R^{5C} is substituted with a plurality of groups selected from substituent groups, size-limited substituent groups, and lower substituent groups; each substituent group, size-limited substituent group, and/or lower substituent group may optionally be different. In embodiments, when R^{5C} is substituted, it is substituted with at least one substituent group. In embodiments, when R^{5C} is substituted, it is substituted with at least one size-limited substituent group. In embodiments, when R^{5C} is substituted, it is substituted with at least one lower substituent group.

In embodiments, a substituted R^{6C} (e.g., substituted alkyl and/or substituted heteroalkyl) is substituted with at least one substituent group, size-limited substituent group, or lower substituent group; wherein if the substituted R^{6C} is substituted with a plurality of groups selected from substituent groups, size-limited substituent groups, and lower substituent groups; each substituent group, size-limited substituent group, and/or lower substituent group may optionally be different. In embodiments, when R^{6C} is substituted, it is substituted with at least one substituent group. In embodiments, when R^{6C} is substituted, it is substituted with at least one size-limited substituent group. In embodiments, when R^{6C} is substituted, it is substituted with at least one lower substituent group.

In embodiments, a substituted R^{7C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{7C} is substituted with a plurality of groups selected from substituent groups, size-limited substituent groups, and lower substituent groups; each substituent group, size-limited substituent group, and/or lower substituent group may optionally be different. In embodiments, when R^{7C} is substituted, it is substituted with at least one substituent group. In embodiments, when R^{7C} is substituted, it is substituted with at least one size-limited substituent group. In embodiments, when R^{7C} is substituted, it is substituted with at least one lower substituent group.

In embodiments, a substituted R^{8C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{8C} is substituted with a plurality of groups selected from substituent groups, size-limited substituent groups, and lower substituent groups; each substituent group, size-limited substituent group, and/or lower substituent group may optionally be different. In embodiments, when R^{8C} is substituted, it is substituted with at least one substituent group. In embodiments, when R^{8C} is substituted, it is substituted with at least one size-limited substituent group. In embodiments, when R^{8C} is substituted, it is substituted with at least one lower substituent group.

In embodiments, a substituted R^{9C} (e.g., substituted alkyl, substituted cycloalkyl, and/or substituted aryl) is substituted with at least one substituent group, size-limited substituent group, or lower substituent group; wherein if the substituted R^{9C} is substituted with a plurality of groups selected from substituent groups, size-limited substituent groups, and lower substituent groups; each substituent group, size-limited substituent group, and/or lower substituent group may optionally be different. In embodiments, when R^{9C} is substituted, it is substituted with at least one substituent group. In embodiments, when R^{9C} is substituted, it is substituted with at least one size-limited substituent group. In embodiments, when R^{9C} is substituted, it is substituted with at least one lower substituent group.

In embodiments, a substituted R^{10C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{10C} is substituted with a plurality of groups selected from substituent groups, size-limited substituent groups, and lower substituent groups; each substituent group, size-limited substituent group, and/or lower substituent group may optionally be different. In embodiments, when R^{10C} is substituted, it is substituted with at least one substituent group. In embodiments, when R^{10C} is substituted, it is substituted with at least one size-limited substituent group. In embodiments, when R^{10C} is substituted, it is substituted with at least one lower substituent group.

In embodiments, a substituted R^{11C} (e.g., substituted alkyl and/or substituted heteroalkyl) is substituted with at least one substituent group, size-limited substituent group, or lower substituent group; wherein if the substituted R^{11C} is substituted with a plurality of groups selected from substituent groups, size-limited substituent groups, and lower substituent groups; each substituent group, size-limited substituent group, and/or lower substituent group may optionally be different. In embodiments, when R^{11C} is substituted, it is substituted with at least one substituent group. In embodiments, when R^{11C} is substituted, it is substituted with at least one size-limited substituent group. In embodiments, when R^{11C} is substituted, it is substituted with at least one lower substituent group.

In embodiments, a substituted R^{12C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{12C} is substituted with a plurality of groups selected from substituent groups, size-limited substituent groups, and lower substituent groups; each substituent group, size-limited substituent group, and/or lower substituent group may optionally be different. In embodiments, when R^{12C} is substituted, it is substituted with at least one substituent group. In embodiments, when R^{12C} is substituted, it is substituted with at least one size-limited substituent group. In embodiments, when R^{12C} is substituted, it is substituted with at least one lower substituent group.

In embodiments, a substituted R^{13C} (e.g., substituted alkyl, substituted heteroalkyl, substituted cycloalkyl, and/or substituted aryl) is substituted with at least one substituent group, size-limited substituent group, or lower substituent group; wherein if the substituted R^{13C} is substituted with a plurality of groups selected from substituent groups, size-limited substituent groups, and lower substituent groups; each substituent group, size-limited substituent group, and/or lower substituent group may optionally be different. In embodiments, when R^{13C} is substituted, it is substituted with at least one substituent group. In embodiments, when R^{13C} is substituted, it is substituted with at least one size-limited substituent group. In embodiments, when R^{13C} is substituted, it is substituted with at least one lower substituent group.

In embodiments, a substituted R^{14C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{14C} is substituted with a plurality of groups selected from substituent groups, size-limited substituent groups, and lower substituent groups; each substituent group, size-limited substituent group, and/or lower substituent group may optionally be different. In embodiments, when R^{14C} is substituted, it is substituted with at least one substituent group. In embodiments, when R^{14C} is substituted, it is substituted with at least one size-limited substituent group. In embodiments, when R^{14C} is substituted, it is substituted with at least one lower substituent group.

In embodiments, a substituted R^{15C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{15C} is substituted with a plurality of groups selected from substituent groups, size-limited substituent groups, and lower substituent groups; each substituent group, size-limited substituent group, and/or lower substituent group may optionally be different. In embodiments, when R^{15C} is substituted, it is substituted with at least one substituent group. In embodiments, when R^{15C} is substituted, it is substituted with at least one size-limited substituent group. In embodiments, when R^{15C} is substituted, it is substituted with at least one lower substituent group.

In embodiments,

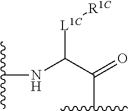

is a divalent form of an unnatural amino acid. In embodiments, L^{1C} is a bond or unsubstituted C₁-C₄ alkylene. In embodiments, R^{1C} is a substituted or unsubstituted aryl, or substituted or unsubstituted heteroaryl. In embodiments,

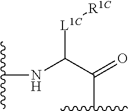

is a divalent form of a natural amino acid. In embodiments,

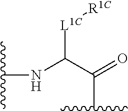

is a divalent form of tyrosine, a divalent form of phenylalanine, or a divalent form of tryptophan.

In embodiments,

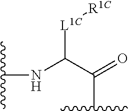

is a divalent form of tyrosine. In embodiments, -L^{1C}-R^{1C} is

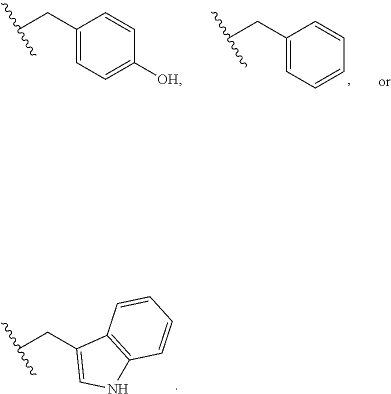 [SMILES: *Cc1ccc(O)cc1 ; *Cc1ccccc1 ; *Cc1c[nH]c2ccccc12]

In embodiments, -L^{1C}-R^{1C} is

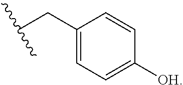 [SMILES: *Cc1ccc(O)cc1]

In embodiments,

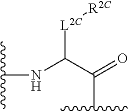

is a divalent form of an unnatural amino acid. In embodiments, L^{2C} is a bond or unsubstituted C₁-C₆ alkylene. In embodiments, R^{2C} is —C(O)OH, —C(O)NH₂, —NHC(NH)NH₂, or substituted or unsubstituted heteroalkyl. In embodiments,

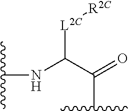

is a divalent form of a natural amino acid. In embodiments,

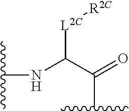

is a divalent form of asparagine, a divalent form of glutamine, a divalent form of aspartic acid, or a divalent form of arginine. In embodiments,

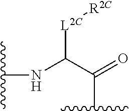

is a divalent form of asparagine. In embodiments,

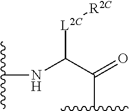

is a divalent form of glutamine. In embodiments,

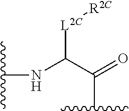

is a divalent form of aspartic acid. In embodiments,

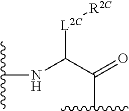

is a divalent form of arginine. In embodiments, -L^{2C}-R^{2C} is

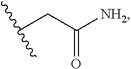 [SMILES: *CC(N)=O]

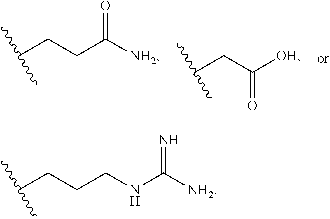 [SMILES: *CCC(N)=O ; *CC(=O)O ; *CCCNC(=N)N]

In embodiments, -L^{2C}-R^{2C} is

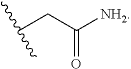 [SMILES: *CC(N)=O]

In embodiments, -L^{2C}-R^{2C} is

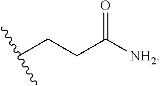 [SMILES: *CCC(N)=O]

In embodiments, -L^{2C}-R^{2C} is

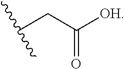 [SMILES: *CC(=O)O]

In embodiments, -L^{2C}-R^{2C} is

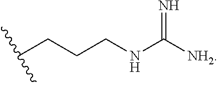 [SMILES: *CCCNC(=N)N]

In embodiments, L³ is independently a bond or

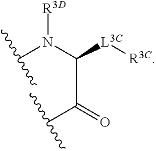

In embodiments, L³ is independently a bond or

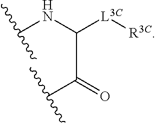

In embodiments, L³ is independently a bond or

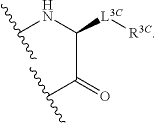

In embodiments, L³ is independently a bond. In embodiments, L³ is independently

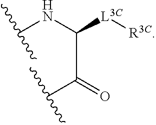

In embodiments,

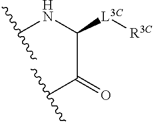

is a divalent form of an unnatural amino acid. In embodiments, L^{3C} is a bond or unsubstituted C₁-C₄ alkylene. In embodiments, R^{3C} is a —C(O)NH₂, substituted or unsubstituted aryl, or substituted or unsubstituted heteroaryl. In embodiments,

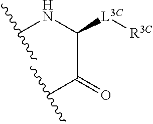

is a divalent form of a natural amino acid. In embodiments,

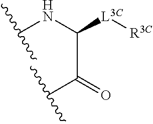

is a divalent form of tyrosine, a divalent form of phenylalanine, a divalent form of tryptophan, a divalent form of asparagine, or a divalent form of glutamine. In embodiments,

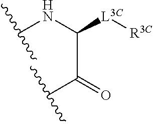

is a divalent form of tyrosine. In embodiments,

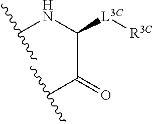

is a divalent form of asparagine. In embodiments, -L^{3C}-R^{3C} is

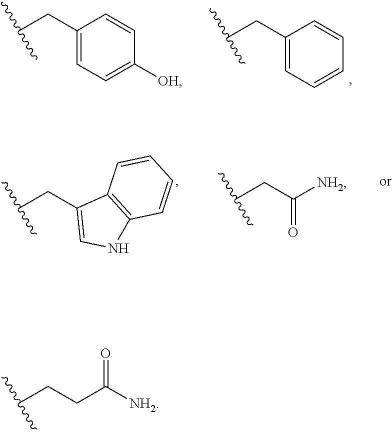 [SMILES: *Cc1ccc(O)cc1 ; *Cc1ccccc1 ; *Cc1c[nH]c2ccccc12 ; *CC(N)=O ; *CCC(N)=O]

In embodiments, -L^{3C}-R^{3C} is

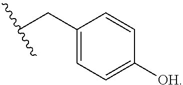 [SMILES: *Cc1ccc(O)cc1]

In embodiments, -L^{3C}-R^{3C} is

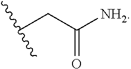 [SMILES: *CC(N)=O]

In embodiments, L⁴ is independently a bond or

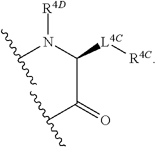

In embodiments, L⁴ is independently a bond or

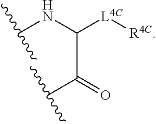

In embodiments, L⁴ is independently a bond or

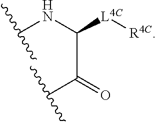

In embodiments, L⁴ is independently a bond. In embodiments, L⁴ is independently

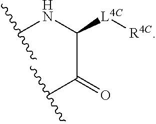

In embodiments,

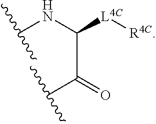

is a divalent form of an unnatural amino acid. In embodiments, L^{4C} is a bond or unsubstituted C₁-C₄ alkylene. In embodiments, R^{4C} is a substituted or unsubstituted alkyl. In embodiments,

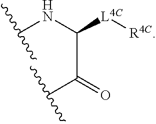

is a divalent form of a natural amino acid. In embodiments,

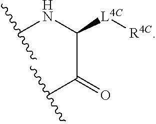

is a divalent form of leucine, a divalent form of isoleucine, or a divalent form of valine. In embodiments,

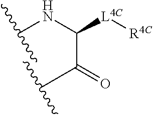

is a divalent form of leucine. In embodiments,

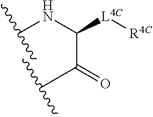

is a divalent form of isoleucine. In embodiments, -L^{4C}-R^{4C} is

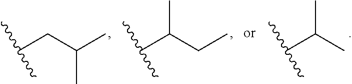

In embodiments, -L^{4C}-R^{4C} is

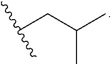 [SMILES: *CC(C)C]

In embodiments, -L^{4C}-R^{4C} is

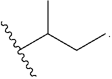 [SMILES: *C(C)CC]

In embodiments, L⁵ is independently a bond or

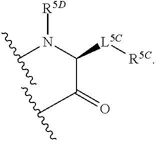

In embodiments, L⁵ is independently a bond or

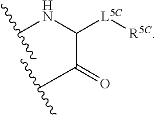

In embodiments, L⁵ is independently a bond or

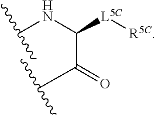

In embodiments, L⁵ is independently a bond. In embodiments, L⁵ is independently

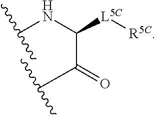

In embodiments,

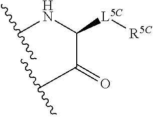

is a divalent form of an unnatural amino acid. In embodiments, L^{5C} is a bond or unsubstituted C₁-C₄ alkylene. In embodiments, R^{5C} is a substituted or unsubstituted aryl, or substituted or unsubstituted heteroaryl. In embodiments,

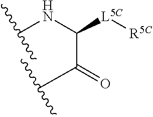

is a divalent form of a natural amino acid. In embodiments,

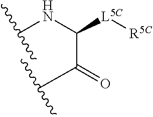

is a divalent form of tyrosine, a divalent form of phenylalanine, or a divalent form of tryptophan. In embodiments,

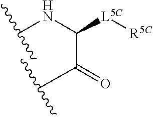

is a divalent form of tyrosine. In embodiments, -L^{5C}-R^{5C} is

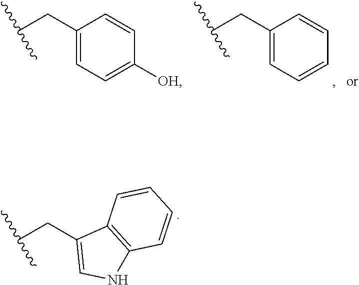 [SMILES: *Cc1ccc(O)cc1 ; *Cc1ccccc1 ; *Cc1c[nH]c2ccccc12]

In embodiments, -L^{5C}-R^{5C} is OH.

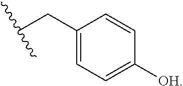 [SMILES: *Cc1ccc(O)cc1]

In embodiments, L⁶ is independently a bond,

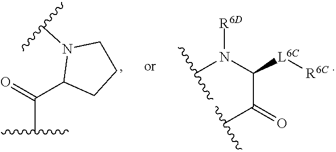

In embodiments, L⁶ is independently a bond,

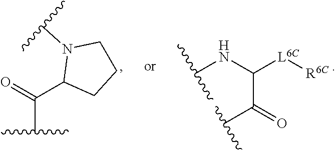

In embodiments, L⁶ is independently a bond,

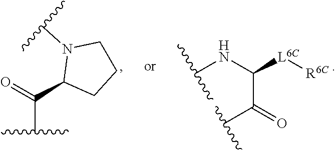

In embodiments, L⁶ is independently a bond. In embodiments, L⁶ is independently

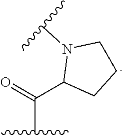 [SMILES: *C(=O)C1CCCN1*]

In embodiments, L⁶ is independently

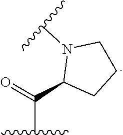 [SMILES: *C(=O)[C@@H]1CCCN1*]

In embodiments, L⁶ is independently a divalent form of proline. In embodiments, L⁶ is independently

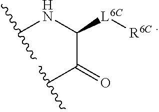

In embodiments,

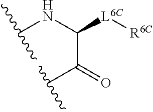

is a divalent form of an unnatural amino acid. In embodiments, L^{6C} is a bond or unsubstituted C₁-C₆ alkylene. In embodiments, R^{6C} is —NHC(NH)NH₂ or substituted or unsubstituted heteroalkyl. In embodiments,

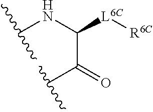

is a divalent form of a natural amino acid. In embodiments,

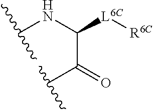

is a divalent form of arginine. In embodiments, -L^{6C}-R^{6C} is

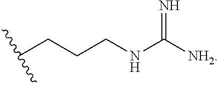 [SMILES: *CCCNC(=N)N]

In embodiments,

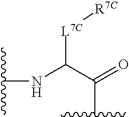

is a divalent form of an unnatural amino acid. In embodiments, L^{7C} is a bond or unsubstituted C₁-C₄ alkylene. In embodiments, R^{7C} is a substituted or unsubstituted aryl, or substituted or unsubstituted heteroaryl. In embodiments,

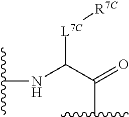

is a divalent form of a natural amino acid. In embodiments,

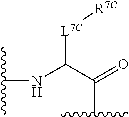

is a divalent form of tyrosine, a divalent form of phenylalanine, or a divalent form of tryptophan.

In embodiments,

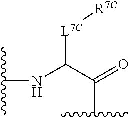

is a divalent form of tyrosine. In embodiments,

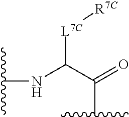

is a divalent form of tryptophan. In embodiments, -L^{7C}-R^{7C} is

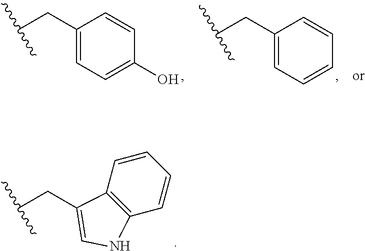 [SMILES: *Cc1ccc(O)cc1 ; *Cc1ccccc1 ; *Cc1c[nH]c2ccccc12]

In embodiments, -L^{7C}-R^{7C} is

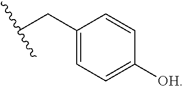 [SMILES: *Cc1ccc(O)cc1]

In embodiments, -L^{7C}-R^{7C} is

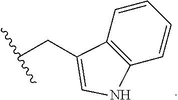 [SMILES: *Cc1c[nH]c2ccccc12]

In embodiments,

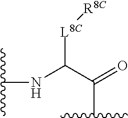

is a divalent form of an unnatural amino acid. In embodiments, L^{8C} is a bond or unsubstituted C₁-C₆ alkylene. In embodiments, R^{8C} is —NH₂, —NHC(NH)NH₂, substituted or unsubstituted heteroalkyl, substituted or unsubstituted aryl, or substituted or unsubstituted heteroaryl. In embodiments,

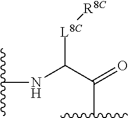

is a divalent form of a natural amino acid. In embodiments,

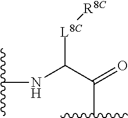

is a divalent form of arginine, a divalent form of histidine, a divalent form of lysine, a divalent form of phenylalanine, or a divalent form of tyrosine. In embodiments,

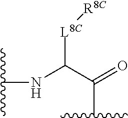

is a divalent form of arginine. In embodiments,

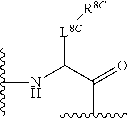

is a divalent form of histidine. In embodiments,

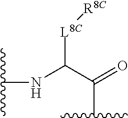

is a divalent form of tyrosine. In embodiments, -L^{8C}-R^{8C} is

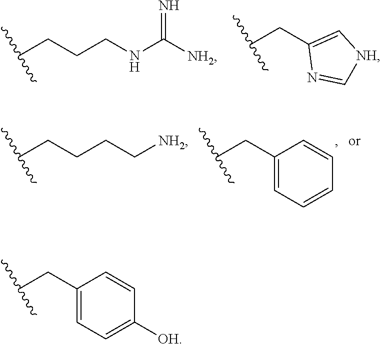 [SMILES: *CCCNC(=N)N ; *Cc1c[nH]cn1 ; *CCCCN ; *Cc1ccccc1 ; *Cc1ccc(O)cc1]

In embodiments, -L^{8C}-R^{8C} is

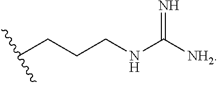 [SMILES: *CCCNC(=N)N]

In embodiments, -L^{8C}-R^{8C} is

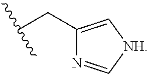 [SMILES: *Cc1c[nH]cn1]

In embodiments, -L^{8C}-R^{8C} is

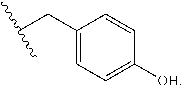 [SMILES: *Cc1ccc(O)cc1]

In embodiments, L⁹ is independently a bond,

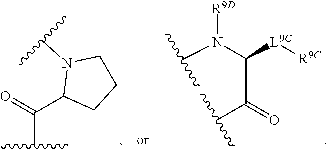

In embodiments, L⁹ is independently a bond,

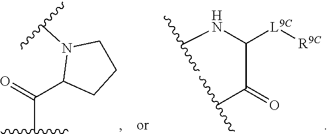

In embodiments, L⁹ is independently a bond,

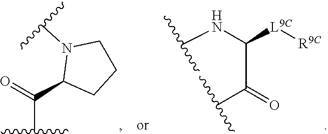

In embodiments, L⁹ is independently a bond. In embodiments, L⁹ is independently

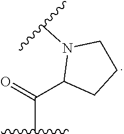 [SMILES: *C(=O)C1CCCN1*]

In embodiments, L⁹ is independently

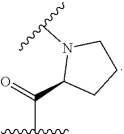 [SMILES: *C(=O)[C@@H]1CCCN1*]

In embodiments, L⁹ is independently

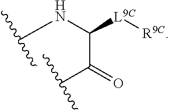

In embodiments,

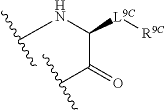

is a divalent form of an unnatural amino acid. In embodiments,

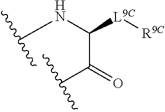

is a divalent form of a natural amino acid.

In embodiments, L¹⁰ is independently a bond,

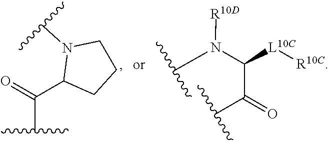

In embodiments, L¹⁰ is independently a bond,

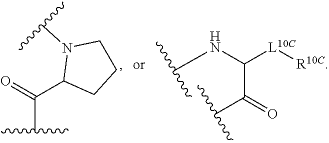

In embodiments, L¹⁰ is independently a bond,

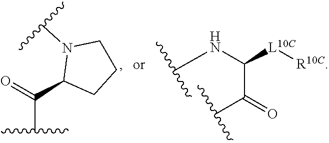

In embodiments, L¹⁰ is independently a bond. In embodiments, L¹⁰ is independently

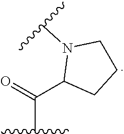 [SMILES: *C(=O)C1CCCN1*]

In embodiments, L¹⁰ is independently

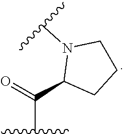 [SMILES: *C(=O)[C@@H]1CCCN1*]

In embodiments, L¹⁰ is independently

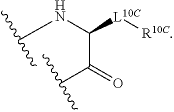

In embodiments

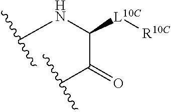

is a divalent form of an unnatural amino acid. In embodiments, L^{10C} is a bond or unsubstituted C₁-C₄ alkylene. In embodiments, R^{10C} is a substituted or unsubstituted alkyl. In embodiments,

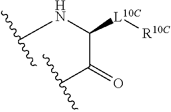

is a divalent form of a natural amino acid. In embodiments,

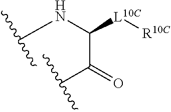

is a divalent form of alanine, a divalent form of valine, a divalent form of leucine, or a divalent form of isoleucine. In embodiments,

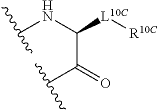

is a divalent form of alanine. In embodiments,

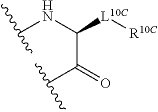

is a divalent form of valine. In embodiments,

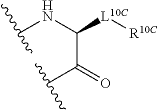

is a divalent form of leucine. In embodiments, -L^{10C}-R^{10C} is —CH₃,

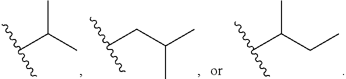

In embodiments, -L^{10C}-R^{10C} is —CH₃. In embodiments, -L^{10C}-R^{10C} is

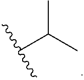 [SMILES: *C(C)C]

In embodiments, -L^{10C}-R^{10C} is

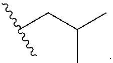 [SMILES: *CC(C)C]

In embodiments, L¹¹ is independently a bond or

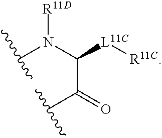

In embodiments, L¹¹ is independently a bond or

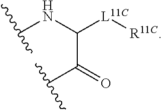

In embodiments, L¹¹ is independently a bond or

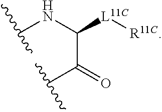

In embodiments, L¹¹ is independently a bond. In embodiments,

L¹¹ is independently

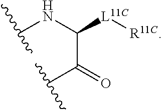

In embodiments,

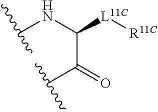

is a divalent form of an unnatural amino acid. In embodiments, L^{11C} is a bond or unsubstituted C₁-C₄ alkylene. In embodiments, R^{11C} is —C(O)OH or substituted or unsubstituted heteroalkyl. In embodiments,

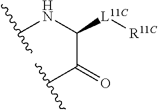

is a divalent form of a natural amino acid. In embodiments,

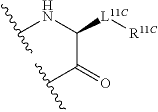

is a divalent form of glutamic acid or a divalent form of aspartic acid. In embodiments,

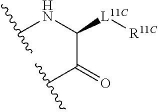

is a divalent form of glutamic acid. In embodiments, -L^{11C}-R^{11C} is

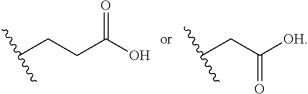 [SMILES: *CCC(=O)O ; *CC(=O)O]

In embodiments, -L^{11C}-R^{11C} is

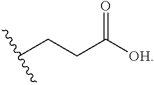 [SMILES: *CCC(=O)O]

In embodiments,

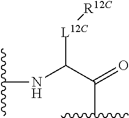

is a divalent form of an unnatural amino acid. In embodiments, L^{12C} is a bond or unsubstituted C₁-C₄ alkylene. In embodiments, R^{12C} is a substituted or unsubstituted alkyl or a substituted or unsubstituted aryl. In embodiments,

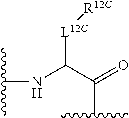

is a divalent form of a natural amino acid. In embodiments,

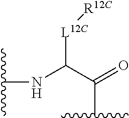

is a divalent form of leucine, a divalent form of isoleucine, a divalent form of valine, a divalent form of phenylalanine, or a divalent form of tyrosine. In embodiments,

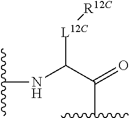

is a divalent form of leucine. In embodiments,

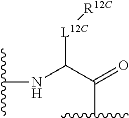

is a divalent form of isoleucine. In embodiments,

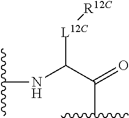

is a divalent form of phenylalanine. In embodiments, -L^{12C}-R^{12C} is

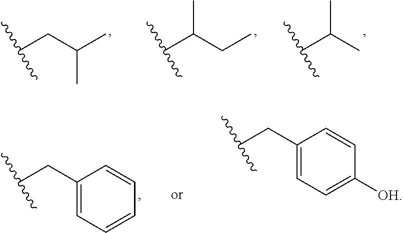

In embodiments, -L^{12C}-R^{12C} is

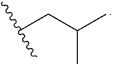 [SMILES: *CC(C)C]

In embodiments, -L^{12C}-R^{12C} is

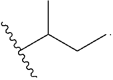 [SMILES: *C(C)CC]

In embodiments, -L^{12C}-R^{12C} is

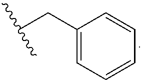 [SMILES: *Cc1ccccc1]

In embodiments, L¹³ is independently

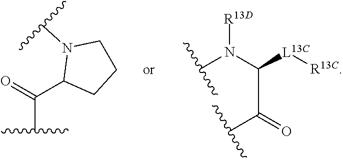

In embodiments, L¹³ is independently

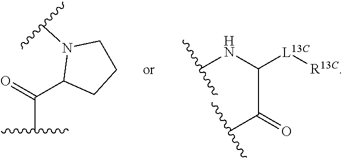

In embodiments, L¹³ is independently

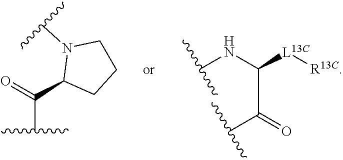

In embodiments, L¹³ is independently

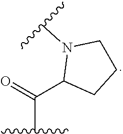 [SMILES: *C(=O)C1CCCN1*]

In embodiments, L¹³ is independently

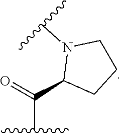 [SMILES: *C(=O)[C@@H]1CCCN1*]

In embodiments, L¹³ is independently

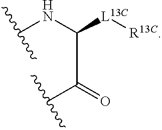

In embodiments, L^{13C} is a bond or unsubstituted C₁-C₄ alkylene. In embodiments, R^{13C} is hydrogen, —OH, or substituted or unsubstituted alkyl. In embodiments,

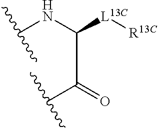

is a divalent form of a natural amino acid. In embodiments,

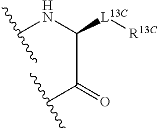

is a divalent form of glycine, a divalent form of alanine, a divalent form of leucine, or a divalent form of serine. In embodiments,

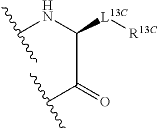

is a divalent form of glycine. In embodiments,

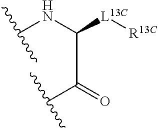

is a divalent form of leucine. In embodiments,

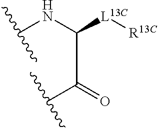

is a divalent form of serine. In embodiments, -L^{13C}-R^{13C} is —H, —CH₃,

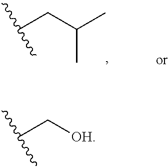

In embodiments, -L^{13C}-R^{13C} is —H. In embodiments, -L^{13C}-R^{13C} is

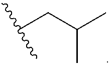 [SMILES: *CC(C)C]

In embodiments, -L^{13C}-R^{13C} is

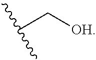 [SMILES: *CO]

In embodiments, L¹⁴ is independently a bond or

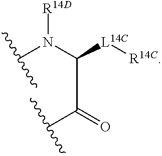

In embodiments, L¹⁴ is independently a bond or

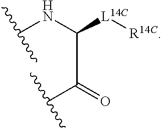

In embodiments, L¹⁴ is independently a bond or

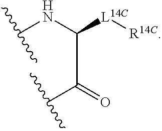

In embodiments, L¹⁴ is independently a bond. In embodiments, L¹⁴ is independently

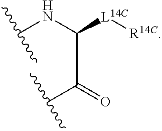

In embodiments,

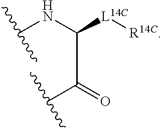

is a divalent form of an unnatural amino acid. In embodiments, L^{14C} is a bond or unsubstituted C₁-C₄ alkylene. In embodiments, R^{14C} is a substituted or unsubstituted aryl, or substituted or unsubstituted heteroaryl. In embodiments,

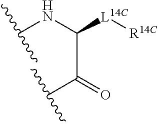

is a divalent form of a natural amino acid. In embodiments,

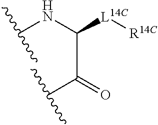

is a divalent form of tryptophan, a divalent form of phenylalanine, or a divalent form of tyrosine. In embodiments,

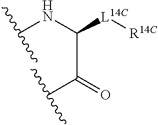

is a divalent form of tryptophan. In embodiments,

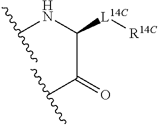

is a divalent form of tyrosine. In embodiments, -L^{14C}-R^{14C} is

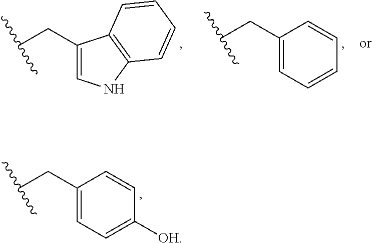 [SMILES: *Cc1c[nH]c2ccccc12 ; *Cc1ccccc1 ; *Cc1ccc(O)cc1]

In embodiments, -L^{14C}-R^{14C} is

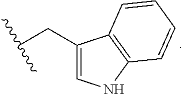 [SMILES: *Cc1c[nH]c2ccccc12]

In embodiments, -L^{14C}-R^{14C} is

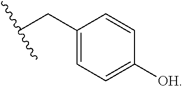 [SMILES: *Cc1ccc(O)cc1]

In embodiments, L¹⁵ is independently a bond or

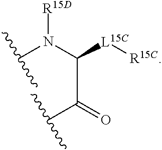

In embodiments, L¹⁵ is independently a bond or

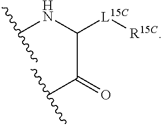

In embodiments, L¹⁵ is independently a bond or

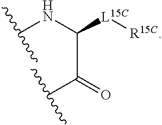

In embodiments, L¹⁵ is independently a bond. In embodiments, L¹⁵ is independently

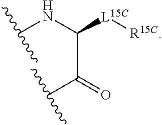

In embodiments,

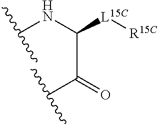

is a divalent form of an unnatural amino acid. In embodiments, L^{15C} is a bond or unsubstituted C₁-C₄ alkylene. In embodiments, R^{15C} is a substituted or unsubstituted aryl, or substituted or unsubstituted heteroaryl. In embodiments,

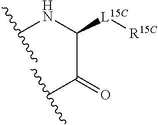

is a divalent form of a natural amino acid. In embodiments,

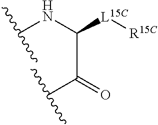

is a divalent form of tyrosine, a divalent form of phenylalanine, or a divalent form of tryptophan. In embodiments,

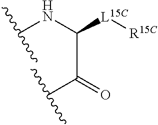

is a divalent form of tyrosine. In embodiments,

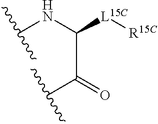

is a divalent form of tryptophan. In embodiments, -L^{15C}-R^{15C} is

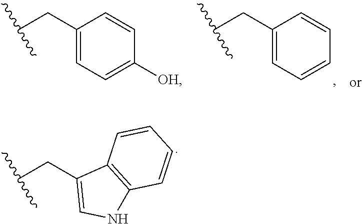 [SMILES: *Cc1ccc(O)cc1 ; *Cc1ccccc1 ; *Cc1c[nH]c2ccccc12]

In embodiments, -L^{15C}-R^{15C} is

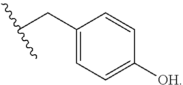 [SMILES: *Cc1ccc(O)cc1]

In embodiments, -L^{15C}-R^{15C} is

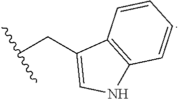 [SMILES: *Cc1c[nH]c2ccccc12]

In embodiments, two substituents selected from R^{1C}, R^{2C}, R^{3C}, R^{4C}, R^{5C}, R^{6C}, R^{7C}, R^{8C}, R^{9C}, R^{10C}, R^{11C}, R^{12C}, R^{13C}, R^{14C}, and R^{15C} may optionally be joined to form a bioconjugate linker. In embodiments, two substituents selected from R^{1C}, R^{2C}, R^{3C}, R^{4C}, R^{5C}, R^{6C}, R^{7C}, R^{8C}, R^{9C}, R^{10C}, R^{11C}, R^{12C}, R^{13C}, R^{14C}, and R^{15C} may optionally be joined to form a covalent linker having the formula -L^{18A}-L^{18B}-L^{18C}-L^{18D}-L^{18E}-L^{18F}; L^{18A}, L^{18B}, L^{18C}, L^{18D}, L^{18E}, and L^{18F} are as described herein, including in embodiments.

In embodiments, the compound of formula (III) is a peptide of FIG. 1B. In embodiments, the compound of formula (III) is peptide 3, 4, 6, 17, 18, or 20 of FIG. 1B. For example, for peptide 17 of FIG. 1B,

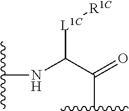

is a divalent form of D-tyrosine;

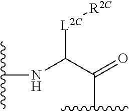

is a divalent form of asparagine; L³ and L⁴ are a bond;

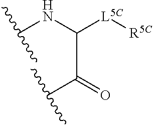

is a divalent form of tyrosine; L⁶ is

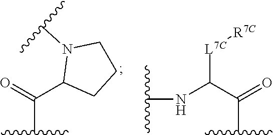

is a divalent form of tyrosine;

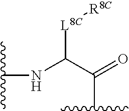

is a divalent form of arginine; L⁹ is

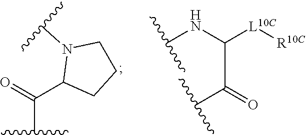

is a divalent form of leucine;

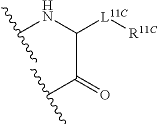

is a divalent form of glutamic acid;

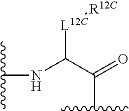

is a divalent form of leucine;

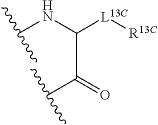

is a divalent form of glycine;

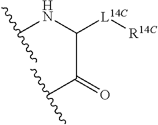

is a divalent form of tryptophan; and

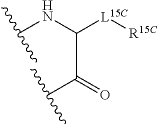

is a divalent form of tyrosine. Where the compound of formula (III) is peptide 3, 4, 6, 18, or 20 of FIG. 1B, the same exemplification would apply. For example, for peptide 3 of FIG. 1B,

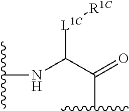

is a divalent form of D-tyrosine (with the same exemplification of the remaining amino acids).

For example, for peptide 4 of FIG. 1B,

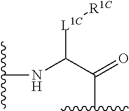

is a divalent form of D-tyrosine (with the same exemplification of the remaining amino acids). For example, for peptide 6 of FIG. 1B,

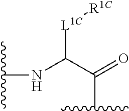

is a divalent form of D-tyrosine (with the same exemplification of the remaining amino acids). For example, for peptide 18 of FIG. 1B,

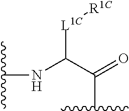

is a divalent form of D-tyrosine (with the same exemplification of the remaining amino acids). For example, for peptide 20 of FIG. 1B,

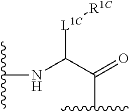

is a divalent form of D-tyrosine (with the same exemplification of the remaining amino acids).

In embodiments, the compound has the formula:

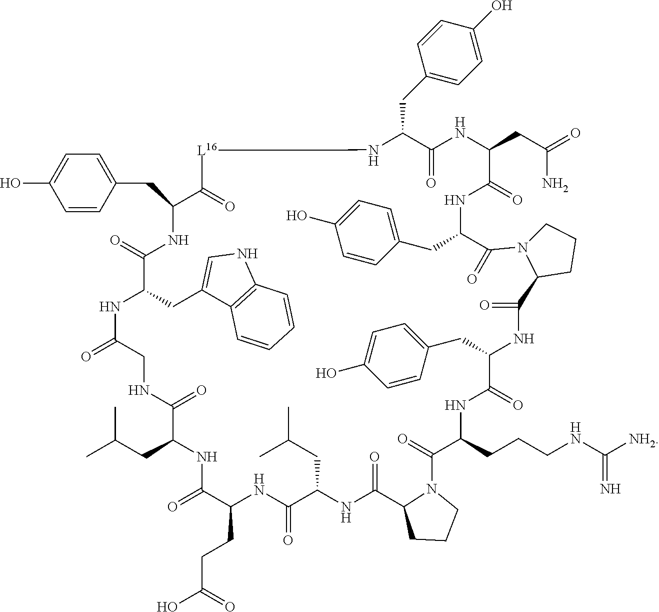

L¹⁶ is as described herein, including in embodiments.

In an aspect is provided a compound having the formula:

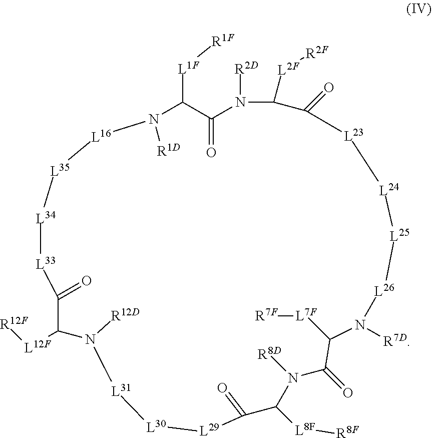

R^{1D}, R^{2D}, R^{3D}, R^{4D}, R^{5D}, R^{6D}, R^{7D}, R^{8D}, R^{9D}, R^{10D}, R^{11D}, R^{12D}, R^{13D}, R^{14D}, R^{15D}, and L¹⁶ are as described herein, including in embodiments.

L^{1F}, L^{2F}, L^{3F}, L^{4F}, L^{5F}, L^{6F}, L^{7F}, L^{8F}, L^{9F}, L^{10F}, L^{11F}, L^{12F}, L^{13F}, L^{14F}, and L^{15F} are independently a bond, substituted or unsubstituted alkylene (e.g., C₁-C₈, C₁-C₆, C₁-C₄, or C₁-C₂), or substituted or unsubstituted heteroalkylene (e.g., 2 to 8 membered, 2 to 6 membered, 4 to 6 membered, 2 to 3 membered, or 4 to 5 membered).

R^{1F} is substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R^{2F} is hydrogen, —OH, —NO₂, —CN, —NH₂, —C(O)OH, —C(O)NH₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), or substituted or unsubstituted aryl (e.g., C₆-C₁₀ or phenyl).

L²³ is a bond or

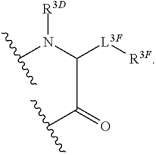

R^{3F} is hydrogen, —NH₂, —C(O)OH, —C(O)NH₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

L²⁴ is a bond or

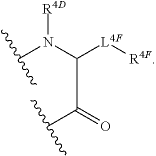

R^{4F} is hydrogen, —NH₂, —C(O)OH, —C(O)NH₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), or substituted or unsubstituted aryl (e.g., C₆-C₁₀ or phenyl).

L²⁵ is a bond or

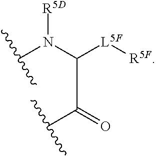

R^{5F} is —OH, —NO₂, —CN, —NH₂, —C(O)OH, —C(O)NH₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, substituted or unsubstituted alkyl (e.g., C₁-C₈, C₁-C₆, C₁-C₄, or C₁-C₂), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

L²⁶ is a bond,

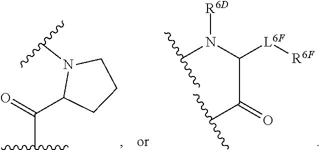

R^{6F} is hydrogen, —OH, —NO₂, —CN, —NH₂, —C(O)OH, —C(O)NH₂, —SH—, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, substituted or unsubstituted alkyl (e.g., C₁-C₈, C₁-C₆, C₁-C₄, or C₁-C₂), or substituted or unsubstituted heteroalkyl (e.g., 2 to 8 membered, 2 to 6 membered, 4 to 6 membered, 2 to 3 membered, or 4 to 5 membered).

R^{7F} and R^{8F} are independently hydrogen, —OH, —NO₂, —CN, —NH₂, —C(O)OH, —C(O)NH₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

L²⁹ is a bond,

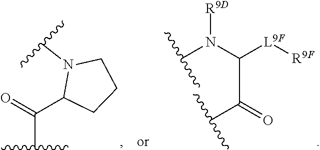

R^{9F} is hydrogen, —OH, —NO₂, —CN, —NH₂, —C(O)OH, —C(O)NH₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, substituted or unsubstituted alkyl (e.g., C₁-C₈, C₁-C₆, C₁-C₄, or C₁-C₂), substituted or unsubstituted cycloalkyl (e.g., C₃-C₈, C₃-C₆, C₄-C₆, or C₅-C₆), substituted or unsubstituted aryl (e.g., C₆-C₁₀ or phenyl), or substituted or unsubstituted heteroaryl (e.g., 5 to 10 membered, 5 to 9 membered, or 5 to 6 membered).

L³⁰ is a bond,

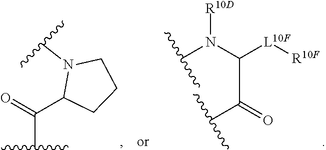

R^{10F} is hydrogen, —OH, —NO₂, —CN, —NH₂, —C(O)OH, —C(O)NH₂, —SH, SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

L³¹ is a bond,

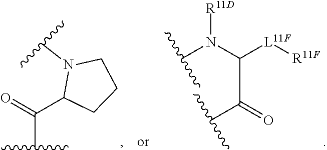

R^{11F} is hydrogen, —CN, —OH, —C(O)OH, —NO₂, —SO₃H, —OSO₃H, —NH₂, —C(O)NH₂, —SO₂NH₂, —NHNH₂, —ONH₂, —NHC(O)NHNH₂, —NHC(O)NH₂, —NHC(NH)NH₂, substituted or unsubstituted alkyl (e.g., C₁-C₈, C₁-C₆, C₁-C₄, or C₁-C₂), or substituted or unsubstituted heteroalkyl (e.g., 2 to 8 membered, 2 to 6 membered, 4 to 6 membered, 2 to 3 membered, or 4 to 5 membered).

R^{12F} is hydrogen, —OH, —NO₂, —CN, —NH₂, —C(O)OH, —C(O)NH₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

L³³ is

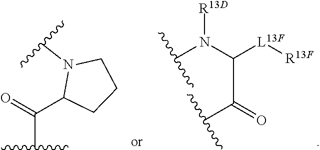

R^{13F} is hydrogen, —OH, —NH₂, —C(O)OH, —C(O)NH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

L³⁴ is a bond or

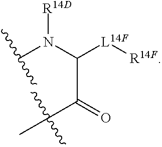

R^{14F} is hydrogen, —NH₂, —C(O)OH, —C(O)NH₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

L³⁵ is a bond or

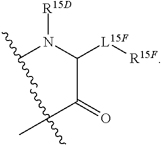

R^{15F} is hydrogen, —NH₂, —C(O)OH, —C(O)NH₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

Two substituents selected from R^{1F}, R^{2F}, R^{3F}, R^{4F}, R^{5F}, R^{6F}, R^{7F}, R^{8F}, R^{9F}, R^{10F}, R^{11F}, R^{12F}, R^{13F}, R^{14F}, and R^{15F} may optionally be joined to form a covalent linker.

In embodiments, the compound has the formula:

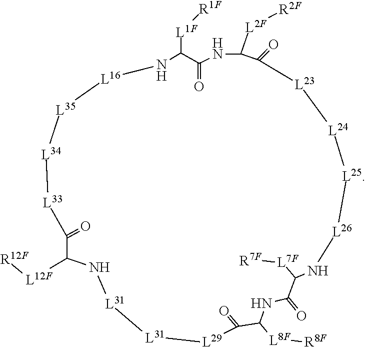

L^{1F}, L^{2F}, L²³, L²⁴, L²⁵, L²⁶, L^{7F}, L^{8F}, L²⁹, L³⁰, L³¹, L^{12F}, L³³, L³⁴, L³⁵, L¹⁶, R^{1F}, R^{2F}, R^{7F}, R^{8F}, and R^{12F} are as described herein, including in embodiments.

In embodiments, the compound has the formula:

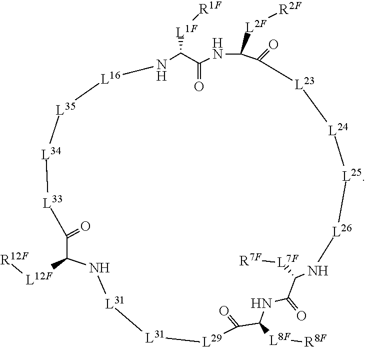

L^{1F}, L^{2F}, L²³, L²⁴, L²⁵, L²⁶, L^{7F}, L^{8F}, L²⁹, L³⁰, L³¹, L^{12F}, L³³, L³⁴, L³⁵, L¹⁶, R^{1F}, R^{2F}, R^{7F}, R^{8F}, and R^{12F} are as described herein, including in embodiments.

In embodiments, a substituted L^{1F} (e.g., substituted alkylene and/or substituted heteroalkylene) is substituted with at least one substituent group, size-limited substituent group, or lower substituent group; wherein if the substituted L^{1F} is substituted with a plurality of groups selected from substituent groups, size-limited substituent groups, and lower substituent groups; each substituent group, size-limited substituent group, and/or lower substituent group may optionally be different. In embodiments, when L^{1F} is substituted, it is substituted with at least one substituent group. In embodiments, when L^{1F} is substituted, it is substituted with at least one size-limited substituent group. In embodiments, when L^{1F} is substituted, it is substituted with at least one lower substituent group.

In embodiments, a substituted L^{2F} (e.g., substituted alkylene and/or substituted heteroalkylene) is substituted with at least one substituent group, size-limited substituent group, or lower substituent group; wherein if the substituted L^{2F} is substituted with a plurality of groups selected from substituent groups, size-limited substituent groups, and lower substituent groups; each substituent group, size-limited substituent group, and/or lower substituent group may optionally be different. In embodiments, when L^{2F} is substituted, it is substituted with at least one substituent group. In embodiments, when L^{2F} is substituted, it is substituted with at least one size-limited substituent group. In embodiments, when L^{2F} is substituted, it is substituted with at least one lower substituent group.

In embodiments, a substituted L^{3F} (e.g., substituted alkylene and/or substituted heteroalkylene) is substituted with at least one substituent group, size-limited substituent group, or lower substituent group; wherein if the substituted L^{3F} is substituted with a plurality of groups selected from substituent groups, size-limited substituent groups, and lower substituent groups; each substituent group, size-limited substituent group, and/or lower substituent group may optionally be different. In embodiments, when L^{3F} is substituted, it is substituted with at least one substituent group. In embodiments, when L^{3F} is substituted, it is substituted with at least one size-limited substituent group. In embodiments, when L^{3F} is substituted, it is substituted with at least one lower substituent group.

In embodiments, a substituted L^{4F} (e.g., substituted alkylene and/or substituted heteroalkylene) is substituted with at least one substituent group, size-limited substituent group, or lower substituent group; wherein if the substituted L^{4F} is substituted with a plurality of groups selected from substituent groups, size-limited substituent groups, and lower substituent groups; each substituent group, size-limited substituent group, and/or lower substituent group may optionally be different. In embodiments, when L^{4F} is substituted, it is substituted with at least one substituent group. In embodiments, when L^{4F} is substituted, it is substituted with at least one size-limited substituent group. In embodiments, when L^{4F} is substituted, it is substituted with at least one lower substituent group.

In embodiments, a substituted L^{5F} (e.g., substituted alkylene and/or substituted heteroalkylene) is substituted with at least one substituent group, size-limited substituent group, or lower substituent group; wherein if the substituted L^{5F} is substituted with a plurality of groups selected from substituent groups, size-limited substituent groups, and lower substituent groups; each substituent group, size-limited substituent group, and/or lower substituent group may optionally be different. In embodiments, when L^{5F} is substituted, it is substituted with at least one substituent group. In embodiments, when L^{5F} is substituted, it is substituted with at least one size-limited substituent group. In embodiments, when L^{5F} is substituted, it is substituted with at least one lower substituent group.

In embodiments, a substituted L^{6F} (e.g., substituted alkylene and/or substituted heteroalkylene) is substituted with at least one substituent group, size-limited substituent group, or lower substituent group; wherein if the substituted L^{6F} is substituted with a plurality of groups selected from substituent groups, size-limited substituent groups, and lower substituent groups; each substituent group, size-limited substituent group, and/or lower substituent group may optionally be different. In embodiments, when L^{6F} is substituted, it is substituted with at least one substituent group. In embodiments, when L^{6F} is substituted, it is substituted with at least one size-limited substituent group. In embodiments, when L^{6F} is substituted, it is substituted with at least one lower substituent group.

In embodiments, a substituted L^{7F} (e.g., substituted alkylene and/or substituted heteroalkylene) is substituted with at least one substituent group, size-limited substituent group, or lower substituent group; wherein if the substituted L^{7F} is substituted with a plurality of groups selected from substituent groups, size-limited substituent groups, and lower substituent groups; each substituent group, size-limited substituent group, and/or lower substituent group may optionally be different. In embodiments, when L^{7F} is substituted, it is substituted with at least one substituent group. In embodiments, when L^{7F} is substituted, it is substituted with at least one size-limited substituent group. In embodiments, when L^{7F} is substituted, it is substituted with at least one lower substituent group.

In embodiments, a substituted L^{8F} (e.g., substituted alkylene and/or substituted heteroalkylene) is substituted with at least one substituent group, size-limited substituent group, or lower substituent group; wherein if the substituted L^{8F} is substituted with a plurality of groups selected from substituent groups, size-limited substituent groups, and lower substituent groups; each substituent group, size-limited substituent group, and/or lower substituent group may optionally be different. In embodiments, when L^{8F} is substituted, it is substituted with at least one substituent group. In embodiments, when L^{8F} is substituted, it is substituted with at least one size-limited substituent group. In embodiments, when L^{8F} is substituted, it is substituted with at least one lower substituent group.

In embodiments, a substituted L^{9F} (e.g., substituted alkylene and/or substituted heteroalkylene) is substituted with at least one substituent group, size-limited substituent group, or lower substituent group; wherein if the substituted L^{9F} is substituted with a plurality of groups selected from substituent groups, size-limited substituent groups, and lower substituent groups; each substituent group, size-limited substituent group, and/or lower substituent group may optionally be different. In embodiments, when L^{9F} is substituted, it is substituted with at least one substituent group. In embodiments, when L^{9F} is substituted, it is substituted with at least one size-limited substituent group. In embodiments, when L^{9F} is substituted, it is substituted with at least one lower substituent group.

In embodiments, a substituted L^{10F} (e.g., substituted alkylene and/or substituted heteroalkylene) is substituted with at least one substituent group, size-limited substituent group, or lower substituent group; wherein if the substituted L^{10F} is substituted with a plurality of groups selected from substituent groups, size-limited substituent groups, and lower substituent groups; each substituent group, size-limited substituent group, and/or lower substituent group may optionally be different. In embodiments, when L^{10F} is substituted, it is substituted with at least one substituent group. In embodiments, when L^{10F} is substituted, it is substituted with at least one size-limited substituent group. In embodiments, when L^{10F} is substituted, it is substituted with at least one lower substituent group.

In embodiments, a substituted L^{11F} (e.g., substituted alkylene and/or substituted heteroalkylene) is substituted with at least one substituent group, size-limited substituent group, or lower substituent group; wherein if the substituted L^{11F} is substituted with a plurality of groups selected from substituent groups, size-limited substituent groups, and lower substituent groups; each substituent group, size-limited substituent group, and/or lower substituent group may optionally be different. In embodiments, when L^{11F} is substituted, it is substituted with at least one substituent group. In embodiments, when L^{11F} is substituted, it is substituted with at least one size-limited substituent group. In embodiments, when L^{11F} is substituted, it is substituted with at least one lower substituent group.

In embodiments, a substituted L^{12F} (e.g., substituted alkylene and/or substituted heteroalkylene) is substituted with at least one substituent group, size-limited substituent group, or lower substituent group; wherein if the substituted L^{12F} is substituted with a plurality of groups selected from substituent groups, size-limited substituent groups, and lower substituent groups; each substituent group, size-limited substituent group, and/or lower substituent group may optionally be different. In embodiments, when L^{12F} is substituted, it is substituted with at least one substituent group. In embodiments, when L^{12F} is substituted, it is substituted with at least one size-limited substituent group. In embodiments, when L^{12F} is substituted, it is substituted with at least one lower substituent group.

In embodiments, a substituted L^{13F} (e.g., substituted alkylene and/or substituted heteroalkylene) is substituted with at least one substituent group, size-limited substituent group, or lower substituent group; wherein if the substituted L^{13F} is substituted with a plurality of groups selected from substituent groups, size-limited substituent groups, and lower substituent groups; each substituent group, size-limited substituent group, and/or lower substituent group may optionally be different. In embodiments, when L^{13F} is substituted, it is substituted with at least one substituent group. In embodiments, when L^{13F} is substituted, it is substituted with at least one size-limited substituent group. In embodiments, when L^{13F} is substituted, it is substituted with at least one lower substituent group.

In embodiments, a substituted L^{14F} (e.g., substituted alkylene and/or substituted heteroalkylene) is substituted with at least one substituent group, size-limited substituent group, or lower substituent group; wherein if the substituted L^{14F} is substituted with a plurality of groups selected from substituent groups, size-limited substituent groups, and lower substituent groups; each substituent group, size-limited substituent group, and/or lower substituent group may optionally be different. In embodiments, when L^{14F} is substituted, it is substituted with at least one substituent group. In embodiments, when L^{14F} is substituted, it is substituted with at least one size-limited substituent group. In embodiments, when L^{14F} is substituted, it is substituted with at least one lower substituent group.

In embodiments, a substituted L^{15F} (e.g., substituted alkylene and/or substituted heteroalkylene) is substituted with at least one substituent group, size-limited substituent group, or lower substituent group; wherein if the substituted L^{15F} is substituted with a plurality of groups selected from substituent groups, size-limited substituent groups, and lower substituent groups; each substituent group, size-limited substituent group, and/or lower substituent group may optionally be different. In embodiments, when L^{15F} is substituted, it is substituted with at least one substituent group. In embodiments, when L^{15F} is substituted, it is substituted with at least one size-limited substituent group. In embodiments, when L^{15F} is substituted, it is substituted with at least one lower substituent group.

In embodiments, a substituted R^{1F} (e.g., substituted cycloalkyl, substituted heterocycloalkyl, substituted aryl, and/or substituted heteroaryl) is substituted with at least one substituent group, size-limited substituent group, or lower substituent group; wherein if the substituted R^{1F} is substituted with a plurality of groups selected from substituent groups, size-limited substituent groups, and lower substituent groups; each substituent group, size-limited substituent group, and/or lower substituent group may optionally be different. In embodiments, when R^{1F} is substituted, it is substituted with at least one substituent group. In embodiments, when R^{1F} is substituted, it is substituted with at least one size-limited substituent group. In embodiments, when R^{1F} is substituted, it is substituted with at least one lower substituent group.

In embodiments, a substituted R^{2F} (e.g., substituted alkyl, substituted heteroalkyl, and/or substituted aryl) is substituted with at least one substituent group, size-limited substituent group, or lower substituent group; wherein if the substituted R^{2F} is substituted with a plurality of groups selected from substituent groups, size-limited substituent groups, and lower substituent groups; each substituent group, size-limited substituent group, and/or lower substituent group may optionally be different. In embodiments, when R^{2F} is substituted, it is substituted with at least one substituent group. In embodiments, when R^{2F} is substituted, it is substituted with at least one size-limited substituent group. In embodiments, when R^{2F} is substituted, it is substituted with at least one lower substituent group.

In embodiments, a substituted R^{3F} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3F} is substituted with a plurality of groups selected from substituent groups, size-limited substituent groups, and lower substituent groups; each substituent group, size-limited substituent group, and/or lower substituent group may optionally be different. In embodiments, when R^{3F} is substituted, it is substituted with at least one substituent group. In embodiments, when R^{3F} is substituted, it is substituted with at least one size-limited substituent group. In embodiments, when R^{3F} is substituted, it is substituted with at least one lower substituent group.

In embodiments, a substituted R^{4F} (e.g., substituted alkyl, substituted heteroalkyl, substituted cycloalkyl, and/or substituted aryl) is substituted with at least one substituent group, size-limited substituent group, or lower substituent group; wherein if the substituted R^{4F} is substituted with a plurality of groups selected from substituent groups, size-limited substituent groups, and lower substituent groups; each substituent group, size-limited substituent group, and/or lower substituent group may optionally be different. In embodiments, when R^{4F} is substituted, it is substituted with at least one substituent group. In embodiments, when R^{4F} is substituted, it is substituted with at least one size-limited substituent group. In embodiments, when R^{4F} is substituted, it is substituted with at least one lower substituent group.

In embodiments, a substituted R^{5F} (e.g., substituted alkyl, substituted cycloalkyl, substituted heterocycloalkyl, substituted aryl, and/or substituted heteroaryl) is substituted with at least one substituent group, size-limited substituent group, or lower substituent group; wherein if the substituted R^{5F} is substituted with a plurality of groups selected from substituent groups, size-limited substituent groups, and lower substituent groups; each substituent group, size-limited substituent group, and/or lower substituent group may optionally be different. In embodiments, when R^{5F} is substituted, it is substituted with at least one substituent group. In embodiments, when R^{5F} is substituted, it is substituted with at least one size-limited substituent group. In embodiments, when R^{5F} is substituted, it is substituted with at least one lower substituent group.

In embodiments, a substituted R^{6F} (e.g., substituted alkyl and/or substituted heteroalkyl) is substituted with at least one substituent group, size-limited substituent group, or lower substituent group; wherein if the substituted R^{6F} is substituted with a plurality of groups selected from substituent groups, size-limited substituent groups, and lower substituent groups; each substituent group, size-limited substituent group, and/or lower substituent group may optionally be different. In embodiments, when R^{6F} is substituted, it is substituted with at least one substituent group. In embodiments, when R^{6F} is substituted, it is substituted with at least one size-limited substituent group. In embodiments, when R^{6F} is substituted, it is substituted with at least one lower substituent group.

In embodiments, a substituted R^{7F} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{7F} is substituted with a plurality of groups selected from substituent groups, size-limited substituent groups, and lower substituent groups; each substituent group, size-limited substituent group, and/or lower substituent group may optionally be different. In embodiments, when R^{7F} is substituted, it is substituted with at least one substituent group. In embodiments, when R^{7F} is substituted, it is substituted with at least one size-limited substituent group. In embodiments, when R^{7F} is substituted, it is substituted with at least one lower substituent group.

In embodiments, a substituted R^{8F} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{8F} is substituted with a plurality of groups selected from substituent groups, size-limited substituent groups, and lower substituent groups; each substituent group, size-limited substituent group, and/or lower substituent group may optionally be different. In embodiments, when R^{8F} is substituted, it is substituted with at least one substituent group. In embodiments, when R^{8F} is substituted, it is substituted with at least one size-limited substituent group. In embodiments, when R^{8F} is substituted, it is substituted with at least one lower substituent group.

In embodiments, a substituted R^{9F} (e.g., substituted alkyl, substituted cycloalkyl, substituted aryl, and/or substituted heteroaryl) is substituted with at least one substituent group, size-limited substituent group, or lower substituent group; wherein if the substituted R^{9F} is substituted with a plurality of groups selected from substituent groups, size-limited substituent groups, and lower substituent groups; each substituent group, size-limited substituent group, and/or lower substituent group may optionally be different. In embodiments, when R^{9F} is substituted, it is substituted with at least one substituent group. In embodiments, when R^{9F} is substituted, it is substituted with at least one size-limited substituent group. In embodiments, when R^{9F} is substituted, it is substituted with at least one lower substituent group.

In embodiments, a substituted R^{10F} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{10F} is substituted with a plurality of groups selected from substituent groups, size-limited substituent groups, and lower substituent groups; each substituent group, size-limited substituent group, and/or lower substituent group may optionally be different. In embodiments, when R^{10F} is substituted, it is substituted with at least one substituent group. In embodiments, when R^{10F} is substituted, it is substituted with at least one size-limited substituent group. In embodiments, when R^{10F} is substituted, it is substituted with at least one lower substituent group.

In embodiments, a substituted R^{11F} (e.g., substituted alkyl and/or substituted heteroalkyl) is substituted with at least one substituent group, size-limited substituent group, or lower substituent group; wherein if the substituted R^{11F} is substituted with a plurality of groups selected from substituent groups, size-limited substituent groups, and lower substituent groups; each substituent group, size-limited substituent group, and/or lower substituent group may optionally be different. In embodiments, when R^{11F} is substituted, it is substituted with at least one substituent group. In embodiments, when R^{11F} is substituted, it is substituted with at least one size-limited substituent group. In embodiments, when R^{11F} is substituted, it is substituted with at least one lower substituent group.

In embodiments, a substituted R^{12F} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{12F} is substituted with a plurality of groups selected from substituent groups, size-limited substituent groups, and lower substituent groups; each substituent group, size-limited substituent group, and/or lower substituent group may optionally be different. In embodiments, when R^{12F} is substituted, it is substituted with at least one substituent group. In embodiments, when R^{12F} is substituted, it is substituted with at least one size-limited substituent group. In embodiments, when R^{12F} is substituted, it is substituted with at least one lower substituent group.

In embodiments, a substituted R^{13F} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{13F} is substituted with a plurality of groups selected from substituent groups, size-limited substituent groups, and lower substituent groups; each substituent group, size-limited substituent group, and/or lower substituent group may optionally be different. In embodiments, when R^{13F} is substituted, it is substituted with at least one substituent group. In embodiments, when R^{13F} is substituted, it is substituted with at least one size-limited substituent group. In embodiments, when R^{13F} is substituted, it is substituted with at least one lower substituent group.

In embodiments, a substituted R^{14F} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{14F} is substituted with a plurality of groups selected from substituent groups, size-limited substituent groups, and lower substituent groups; each substituent group, size-limited substituent group, and/or lower substituent group may optionally be different. In embodiments, when R^{14F} is substituted, it is substituted with at least one substituent group. In embodiments, when R^{14F} is substituted, it is substituted with at least one size-limited substituent group. In embodiments, when R^{14F} is substituted, it is substituted with at least one lower substituent group.

In embodiments, a substituted R^{15F} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{15F} is substituted with a plurality of groups selected from substituent groups, size-limited substituent groups, and lower substituent groups; each substituent group, size-limited substituent group, and/or lower substituent group may optionally be different. In embodiments, when R^{15F} is substituted, it is substituted with at least one substituent group. In embodiments, when R^{15F} is substituted, it is substituted with at least one size-limited substituent group. In embodiments, when R^{15F} is substituted, it is substituted with at least one lower substituent group.

In embodiments,

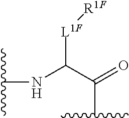

is a divalent form of an unnatural amino acid. In embodiments,

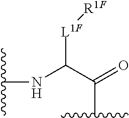

is a divalent form of a natural amino acid.

In embodiments,

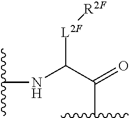

is a divalent form of an unnatural amino acid. In embodiments,

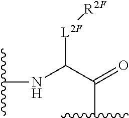

is a divalent form of a natural amino acid.

In embodiments,

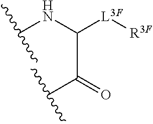

is a divalent form of an unnatural amino acid. In embodiments,

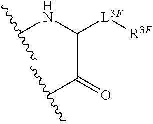

is a divalent form of a natural amino acid.

In embodiments,

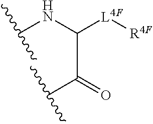

is a divalent form of an unnatural amino acid. In embodiments,

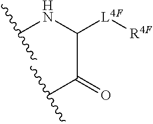

is a divalent form of a natural amino acid.

In embodiments,

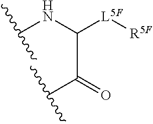

is a divalent form of an unnatural amino acid. In embodiments,

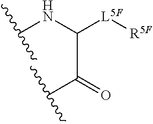

is a divalent form of a natural amino acid.

In embodiments,

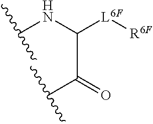

is a divalent form of an unnatural amino acid. In embodiments,

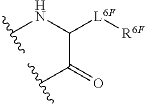

is a divalent form of a natural amino acid.

In embodiments,

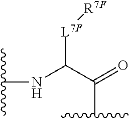

is a divalent form of an unnatural amino acid. In embodiments,

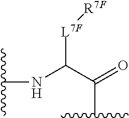

is a divalent form of a natural amino acid.

In embodiments,

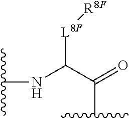

is a divalent form of an unnatural amino acid. In embodiments,

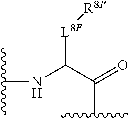

is a divalent form of a natural amino acid.

In embodiments,

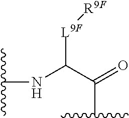

is a divalent form of an unnatural amino acid. In embodiments,

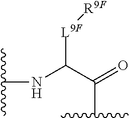

is a divalent form of a natural amino acid.

In embodiments,

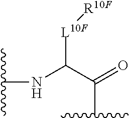

is a divalent form of an unnatural amino acid. In embodiments,

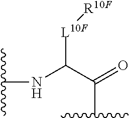

is a divalent form of a natural amino acid.

In embodiments,

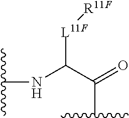

is a divalent form of an unnatural amino acid. In embodiments,

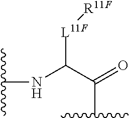

is a divalent form of a natural amino acid.

In embodiments,

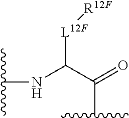

is a divalent form of an unnatural amino acid. In embodiments,

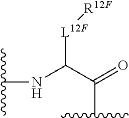

is a divalent form of a natural amino acid.

In embodiments,

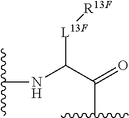

is a divalent form of an unnatural amino acid. In embodiments,

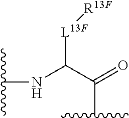

is a divalent form of a natural amino acid.

In embodiments,

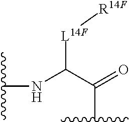

is a divalent form of an unnatural amino acid. In embodiments,

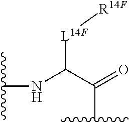

is a divalent form of a natural amino acid.

In embodiments,

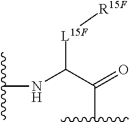

is a divalent form of an unnatural amino acid. In embodiments,

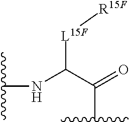

is a divalent form of a natural amino acid.

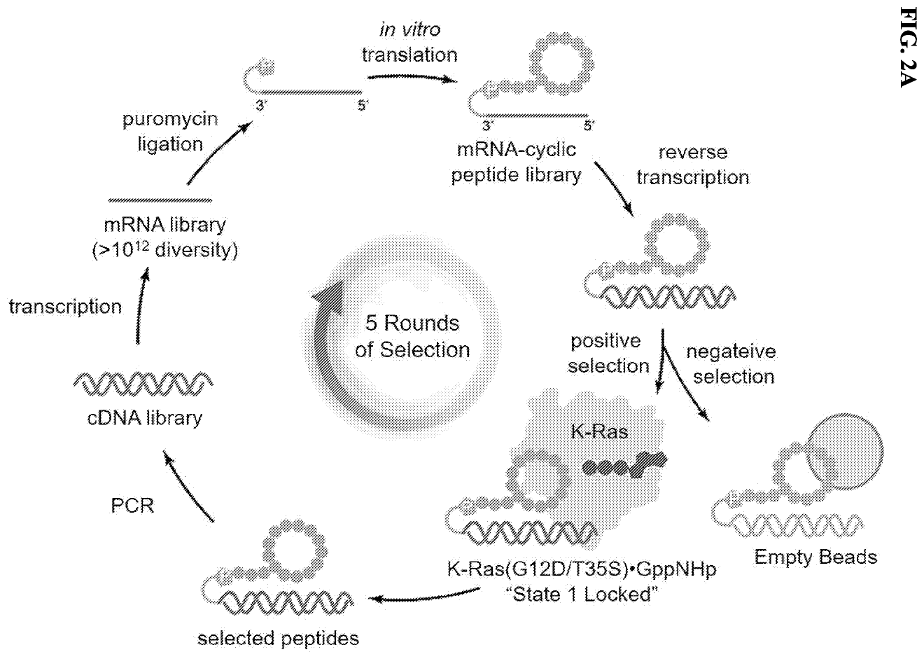

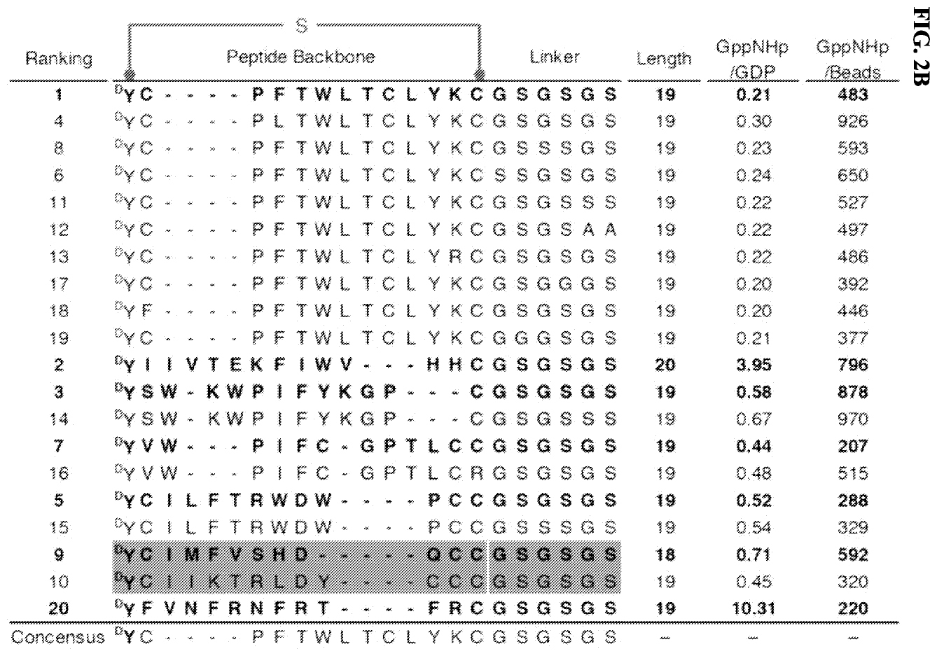

In embodiments, the compound of formula (IV) is a peptide of FIG. 2B. In embodiments, the compound of formula (IV) is peptide 1, 2, 3, 4, 5, 6, 7, 8, 9, 10, 11, 12, 13, 14, 15, 16, 17, 18, 19, or 20 of FIG. 2B. For example, for peptide 1 of FIG. 2B,

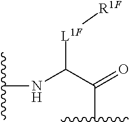

is a divalent form of D-tyrosine;

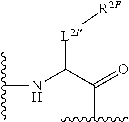

is a divalent form of cysteine;
L²³, L²⁴, and L²⁵ are a bond; L²⁶ is

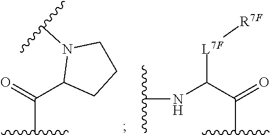

is a divalent form of phenylalanine;

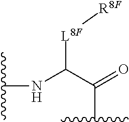

is a divalent form of threonine;

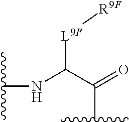

is a divalent form of tryptophan;

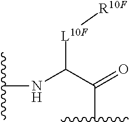

is a divalent form of leucine;

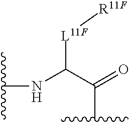

is a divalent form of threonine;

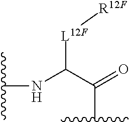

is a divalent form of cysteine;

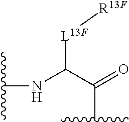

is a divalent form of leucine;

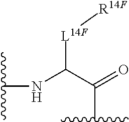

is a divalent form of tyrosine; and

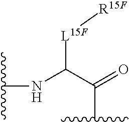

is a divalent form of lysine. Where the compound of formula (IV) is peptide 2, 3, 4, 5, 6, 7, 8, 9, 10, 11, 12, 13, 14, 15, 16, 17, 18, 19, or 20 of FIG. 2B, the same exemplification would apply. For example, for peptide 2 of FIG. 2B,

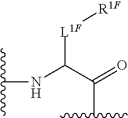

is a divalent form of D-tyrosine (with the same exemplification of the remaining amino acids). For example, for peptide 3 of FIG. 2B,

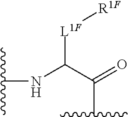

is a divalent form of D-tyrosine (with the same exemplification of the remaining amino acids). For example, for peptide 4 of FIG. 2B,

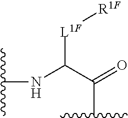

is a divalent form of D-tyrosine (with the same exemplification of the remaining amino acids). For example, for peptide 5 of FIG. 2B,

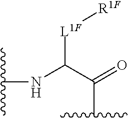

is a divalent form of D-tyrosine (with the same exemplification of the remaining amino acids). For example, for peptide 6 of FIG. 2B,

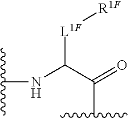

is a divalent form of D-tyrosine (with the same exemplification of the remaining amino acids). For example, for peptide 7 of FIG. 2B,

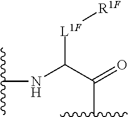

is a divalent form of D-tyrosine (with the same exemplification of the remaining amino acids). For example, for peptide 8 of FIG. 2B,

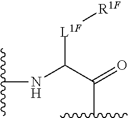

is a divalent form of D-tyrosine (with the same exemplification of the remaining amino acids). For example, for peptide 9 of FIG. 2B,

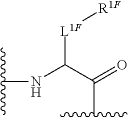

is a divalent form of D-tyrosine (with the same exemplification of the remaining amino acids). For example, for peptide 10 of FIG. 2B,

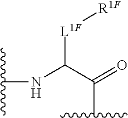

is a divalent form of D-tyrosine (with the same exemplification of the remaining amino acids). For example, for peptide 11 of FIG. 2B,

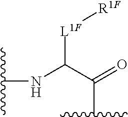

is a divalent form of D-tyrosine (with the same exemplification of the remaining amino acids). For example, for peptide 12 of FIG. 2B,

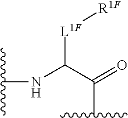

is a divalent form of D-tyrosine (with the same exemplification of the remaining amino acids). For example, for peptide 13 of FIG. 2B,

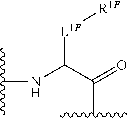

is a divalent form of D-tyrosine (with the same exemplification of the remaining amino acids). For example, for peptide 14 of FIG. 2B,

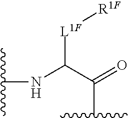

is a divalent form of D-tyrosine (with the same exemplification of the remaining amino acids). For example, for peptide 15 of FIG. 2B,

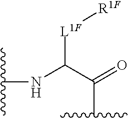

is a divalent form of D-tyrosine (with the same exemplification of the remaining amino acids). For example, for peptide 16 of FIG. 2B,

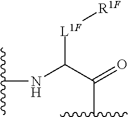

is a divalent form of D-tyrosine (with the same exemplification of the remaining amino acids). For example, for peptide 17 of FIG. 2B,

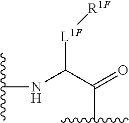

is a divalent form of D-tyrosine (with the same exemplification of the remaining amino acids). For example, for peptide 18 of FIG. 2B,

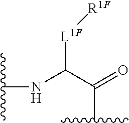

is a divalent form of D-tyrosine (with the same exemplification of the remaining amino acids). For example, for peptide 19 of FIG. 2B,

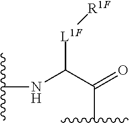

is a divalent form of D-tyrosine (with the same exemplification of the remaining amino acids). For example, for peptide 20 of FIG. 2B,

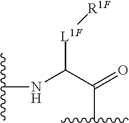

is a divalent form of D-tyrosine (with the same exemplification of the remaining amino acids).

In embodiments, R^{1D} is independently hydrogen. In embodiments, R^{1D} is independently unsubstituted methyl. In embodiments, R^{2D} is independently hydrogen. In embodiments, R^{2D} is independently unsubstituted methyl. In embodiments, R^{3D} is independently hydrogen. In embodiments, R^{3D} is independently unsubstituted methyl. In embodiments, R^{4D} is independently hydrogen. In embodiments, R^{4D} is independently unsubstituted methyl. In embodiments, R^{5D} is independently hydrogen. In embodiments, R^{5D} is independently unsubstituted methyl. In embodiments, R^{6D} is independently hydrogen. In embodiments, R^{6D} is independently unsubstituted methyl. In embodiments, R^{7D} is independently hydrogen. In embodiments, R^{7D} is independently unsubstituted methyl. In embodiments, R^{8D} is independently hydrogen. In embodiments, R^{8D} is independently unsubstituted methyl. In embodiments, R^{9D} is independently hydrogen. In embodiments, R^{9D} is independently unsubstituted methyl. In embodiments, R^{10D} is independently hydrogen. In embodiments, R^{10D} is independently unsubstituted methyl. In embodiments, R^{1D} is independently hydrogen. In embodiments, R^{1D} is independently unsubstituted methyl. In embodiments, R^{12D} is independently hydrogen. In embodiments, R^{12D} is independently unsubstituted methyl. In embodiments, R^{13D} is independently hydrogen. In embodiments, R^{13D} is independently unsubstituted methyl. In embodiments, R^{14D} is independently hydrogen. In embodiments, R^{14D} is independently unsubstituted methyl. In embodiments, R^{15D} is independently hydrogen. In embodiments, R^{15D} is independently unsubstituted methyl.

In embodiments, R^{1D} is independently hydrogen. In embodiments, R^{1D} is independently unsubstituted methyl. In embodiments, R^{1D} is independently unsubstituted ethyl. In embodiments, R^{1D} is independently unsubstituted propyl. In embodiments, R^{1D} is independently unsubstituted butyl. In embodiments, R^{1D} is independently unsubstituted pentyl. In embodiments, R^{1D} is independently unsubstituted isopentyl. In embodiments, R^{2D} is independently hydrogen. In embodiments, R^{2D} is independently unsubstituted methyl. In embodiments, R^{2D} is independently unsubstituted ethyl. In embodiments, R^{2D} is independently unsubstituted propyl. In embodiments, R^{2D} is independently unsubstituted butyl. In embodiments, R^{2D} is independently unsubstituted pentyl. In embodiments, R^{2D} is independently unsubstituted isopentyl. In embodiments, R^{3D} is independently hydrogen. In embodiments, R^{3D} is independently unsubstituted methyl. In embodiments, R^{3D} is independently unsubstituted ethyl. In embodiments, R^{3D} is independently unsubstituted propyl. In embodiments, R^{3D} is independently unsubstituted butyl. In embodiments, R^{3D} is independently unsubstituted pentyl. In embodiments, R^{3D} is independently unsubstituted isopentyl. In embodiments, R^{4D} is independently hydrogen. In embodiments, R^{4D} is independently unsubstituted methyl. In embodiments, R^{4D} is independently unsubstituted ethyl. In embodiments, R^{4D} is independently unsubstituted propyl. In embodiments, R^{4D} is independently unsubstituted butyl. In embodiments, R^{4D} is independently unsubstituted pentyl. In embodiments, R^{4D} is independently unsubstituted isopentyl. In embodiments, R^{5D} is independently hydrogen. In embodiments, R^{5D} is independently unsubstituted methyl. In embodiments, R^{5D} is independently unsubstituted ethyl. In embodiments, R^{5D} is independently unsubstituted propyl. In embodiments, R^{5D} is independently unsubstituted butyl. In embodiments, R^{5D} is independently unsubstituted pentyl. In embodiments, R^{5D} is independently unsubstituted isopentyl. In embodiments, R^{6D} is independently hydrogen. In embodiments, R^{6D} is independently unsubstituted methyl. In embodiments, R^{6D} is independently unsubstituted ethyl. In embodiments, R^{6D} is independently unsubstituted propyl. In embodiments, R^{6D} is independently unsubstituted butyl. In embodiments, R^{6D} is independently unsubstituted pentyl. In embodiments, R^{6D} is independently unsubstituted isopentyl. In embodiments, R^{7D} is independently hydrogen. In embodiments, R^{7D} is independently unsubstituted methyl. In embodiments, R^{7D} is independently unsubstituted ethyl. In embodiments, R^{7D} is independently unsubstituted propyl. In embodiments, R^{7D} is independently unsubstituted butyl. In embodiments, R^{7D} is independently unsubstituted pentyl. In embodiments, R^{7D} is independently unsubstituted isopentyl. In embodiments, R^{8D} is independently hydrogen. In embodiments, R^{8D} is independently unsubstituted methyl. In embodiments, R^{8D} is independently unsubstituted ethyl. In embodiments, R^{8D} is independently unsubstituted propyl. In embodiments, R^{8D} is independently unsubstituted butyl. In embodiments, R^{8D} is independently unsubstituted pentyl. In embodiments, R^{8D} is independently unsubstituted isopentyl. In embodiments, R^{9D} is independently hydrogen. In embodiments, R^{9D} is independently unsubstituted methyl. In embodiments, R^{9D} is independently unsubstituted ethyl. In embodiments, R^{9D} is independently unsubstituted propyl. In embodiments, R^{9D} is independently unsubstituted butyl. In embodiments, R^{9D} is independently unsubstituted pentyl. In embodiments, R^{9D} is independently unsubstituted isopentyl. In embodiments, R^{10D} is independently hydrogen. In embodiments, R^{10D} is independently unsubstituted methyl. In embodiments, R^{10D} is independently unsubstituted ethyl. In embodiments, R^{10D} is independently unsubstituted propyl. In embodiments, R^{10D} is independently unsubstituted butyl. In embodiments, R^{10D} is independently unsubstituted pentyl. In embodiments, R^{10D} is independently unsubstituted isopentyl. In embodiments, R^{11D} is independently hydrogen. In embodiments, R^{11D} is independently unsubstituted methyl. In embodiments, R^{11D} is independently unsubstituted ethyl. In embodiments, R^{11D} is independently unsubstituted propyl. In embodiments, R^{11D} is independently unsubstituted butyl. In embodiments, R^{11D} is independently unsubstituted pentyl. In embodiments, R^{11D} is independently unsubstituted isopentyl. In embodiments, R^{12D} is independently hydrogen. In embodiments, R^{12D} is independently unsubstituted methyl. In embodiments, R^{12D} is independently unsubstituted ethyl. In embodiments, R^{12D} is independently unsubstituted propyl. In embodiments, R^{12D} is independently unsubstituted butyl. In embodiments, R^{12D} is independently unsubstituted pentyl. In embodiments, R^{12D} is independently unsubstituted isopentyl. In embodiments, R^{13D} is independently hydrogen. In embodiments, R^{13D} is independently unsubstituted methyl. In embodiments, R^{13D} is independently unsubstituted ethyl. In embodiments, R^{13D} is independently unsubstituted propyl. In embodiments, R^{13D} is independently unsubstituted butyl. In embodiments, R^{13D} is independently unsubstituted pentyl. In embodiments, R^{13D} is independently unsubstituted isopentyl. In embodiments, R^{14D} is independently hydrogen. In embodiments, R^{14D} is independently unsubstituted methyl. In embodiments, R^{14D} is independently unsubstituted ethyl. In embodiments, R^{14D} is independently unsubstituted propyl. In embodiments, R^{14D} is independently unsubstituted butyl. In embodiments, R^{14D} is independently unsubstituted pentyl. In embodiments, R^{14D} is independently unsubstituted isopentyl. In embodiments, R^{15D} is independently hydrogen. In embodiments, R^{15D} is independently unsubstituted methyl. In embodiments, R^{15D} is independently unsubstituted ethyl. In embodiments, R^{15D} is independently unsubstituted propyl. In embodiments, R^{15D} is independently unsubstituted butyl. In embodiments, R^{15D} is independently unsubstituted pentyl. In embodiments, R^{15D} is independently unsubstituted isopentyl.

In embodiments, L¹⁶ is a bioconjugate linker. In embodiments, L¹⁶ is a substituted or unsubstituted divalent amino acid. In embodiments, L¹⁶ is a substituted or unsubstituted divalent δ-amino acid.

In embodiments, a substituted L¹⁶ (e.g., substituted divalent amino acid and/or substituted divalent δ-amino acid) is substituted with at least one substituent group, size-limited substituent group, or lower substituent group; wherein if the substituted L¹⁶ is substituted with a plurality of groups selected from substituent groups, size-limited substituent groups, and lower substituent groups; each substituent group, size-limited substituent group, and/or lower substituent group may optionally be different. In embodiments, when L¹⁶ is substituted, it is substituted with at least one substituent group. In embodiments, when L¹⁶ is substituted, it is substituted with at least one size-limited substituent group. In embodiments, when L¹⁶ is substituted, it is substituted with at least one lower substituent group.

In embodiments, L¹⁶ is -L^{16A}-L^{16B}-L^{16C}-L^{16D}-L^{16E}-L^{16F}-.

L^{16A}, L^{16B}, L^{16C}, L^{16D}, L^{16E}, and L^{16F} are independently bond, —SS—, —S(O)₂—, —OS(O)₂—, —S(O)₂O—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

In embodiments, a substituted L^{16A} (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{16A} is substituted with a plurality of groups selected from substituent groups, size-limited substituent groups, and lower substituent groups; each substituent group, size-limited substituent group, and/or lower substituent group may optionally be different. In embodiments, when L^{16A} is substituted, it is substituted with at least one substituent group. In embodiments, when L^{16A} is substituted, it is substituted with at least one size-limited substituent group. In embodiments, when L^{16A} is substituted, it is substituted with at least one lower substituent group.

In embodiments, a substituted L^{16B} (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{16B} is substituted with a plurality of groups selected from substituent groups, size-limited substituent groups, and lower substituent groups; each substituent group, size-limited substituent group, and/or lower substituent group may optionally be different. In embodiments, when L^{16B} is substituted, it is substituted with at least one substituent group. In embodiments, when L^{16B} is substituted, it is substituted with at least one size-limited substituent group. In embodiments, when L^{16B} is substituted, it is substituted with at least one lower substituent group.

In embodiments, a substituted L^{16C} (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{16C} is substituted with a plurality of groups selected from substituent groups, size-limited substituent groups, and lower substituent groups; each substituent group, size-limited substituent group, and/or lower substituent group may optionally be different. In embodiments, when L^{16C} is substituted, it is substituted with at least one substituent group. In embodiments, when L^{16C} is substituted, it is substituted with at least one size-limited substituent group. In embodiments, when L^{16C} is substituted, it is substituted with at least one lower substituent group.

In embodiments, a substituted L^{16D} (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{16D} is substituted with a plurality of groups selected from substituent groups, size-limited substituent groups, and lower substituent groups; each substituent group, size-limited substituent group, and/or lower substituent group may optionally be different. In embodiments, when L^{16D} is substituted, it is substituted with at least one substituent group. In embodiments, when L^{16D} is substituted, it is substituted with at least one size-limited substituent group. In embodiments, when L^{16D} is substituted, it is substituted with at least one lower substituent group.

In embodiments, a substituted L^{16E} (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{16E} is substituted with a plurality of groups selected from substituent groups, size-limited substituent groups, and lower substituent groups; each substituent group, size-limited substituent group, and/or lower substituent group may optionally be different. In embodiments, when L^{16E} is substituted, it is substituted with at least one substituent group. In embodiments, when L^{16E} is substituted, it is substituted with at least one size-limited substituent group. In embodiments, when L^{16E} is substituted, it is substituted with at least one lower substituent group.

In embodiments, a substituted L^{16F} (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{16F} is substituted with a plurality of groups selected from substituent groups, size-limited substituent groups, and lower substituent groups; each substituent group, size-limited substituent group, and/or lower substituent group may optionally be different. In embodiments, when L^{16F} is substituted, it is substituted with at least one substituent group. In embodiments, when L^{16F} is substituted, it is substituted with at least one size-limited substituent group. In embodiments, when L^{16F} is substituted, it is substituted with at least one lower substituent group.

In embodiments, L^{16A} is independently bond, —SS—, substituted or unsubstituted alkylene, substituted or unsubstituted heteroalkylene, or substituted or unsubstituted heteroarylene. In embodiments, L^{16A} is independently bond. In embodiments, L^{16A} is independently —SS—. In embodiments, L^{16A} is independently substituted or unsubstituted C₁-C₄ alkylene. In embodiments, L^{16A} is independently substituted or unsubstituted 2 to 6 membered heteroalkylene. In embodiments, L^{16A} is independently substituted or unsubstituted 3 to 6 membered heteroarylene. In embodiments, L^{16A} is independently unsubstituted triazolylene. In embodiments, L^{16A} is independently

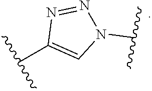 [SMILES: *c1cn(*)nn1]

In embodiments, L^{16B} is independently bond, —SS—, substituted or unsubstituted alkylene, substituted or unsubstituted heteroalkylene, or substituted or unsubstituted heteroarylene. In embodiments, L^{16B} is independently bond. In embodiments, L^{16B} is independently —SS—. In embodiments, L^{16B} is independently substituted or unsubstituted C₁-C₄ alkylene. In embodiments, L^{16B} is independently substituted or unsubstituted 2 to 6 membered heteroalkylene. In embodiments, L^{16B} is independently substituted or unsubstituted 3 to 6 membered heteroarylene. In embodiments, L^{16B} is independently unsubstituted triazolylene. In embodiments, L^{16B} is independently

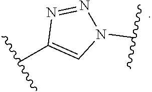 [SMILES: *c1cn(*)nn1]

In embodiments, L^{16C} is independently bond, —SS—, substituted or unsubstituted alkylene, substituted or unsubstituted heteroalkylene, or substituted or unsubstituted heteroarylene. In embodiments, L^{16C} is independently bond. In embodiments, L^{16C} is independently —SS—. In embodiments, L^{16C} is independently substituted or unsubstituted C₁-C₄ alkylene. In embodiments, L^{16C} is independently substituted or unsubstituted 2 to 6 membered heteroalkylene. In embodiments, L^{16C} is independently substituted or unsubstituted 3 to 6 membered heteroarylene. In embodiments, L^{16C} is independently unsubstituted triazolylene. In embodiments, L^{16C} is independently

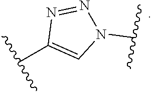 [SMILES: *c1cn(*)nn1]

In embodiments, L^{16D} is independently bond, —SS—, substituted or unsubstituted alkylene, substituted or unsubstituted heteroalkylene, or substituted or unsubstituted heteroarylene. In embodiments, L^{16D} is independently bond. In embodiments, L^{16D} is independently —SS—. In embodiments, L^{16D} is independently substituted or unsubstituted C₁-C₄ alkylene. In embodiments, L^{16D} is independently substituted or unsubstituted 2 to 6 membered heteroalkylene. In embodiments, L^{16D} is independently substituted or unsubstituted 3 to 6 membered heteroarylene. In embodiments, L^{16D} is independently unsubstituted triazolylene. In embodiments, L^{16D} is independently

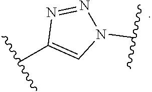 [SMILES: *c1cn(*)nn1]

In embodiments, L^{16E} is independently bond, —SS—, substituted or unsubstituted alkylene, substituted or unsubstituted heteroalkylene, or substituted or unsubstituted heteroarylene. In embodiments, L^{16E} is independently bond. In embodiments, L^{16E} is independently —SS—. In embodiments, L^{16E} is independently substituted or unsubstituted C₁-C₄ alkylene. In embodiments, L^{16E} is independently substituted or unsubstituted 2 to 6 membered heteroalkylene. In embodiments, L^{16E} is independently substituted or unsubstituted 3 to 6 membered heteroarylene. In embodiments, L^{16E} is independently unsubstituted triazolylene. In embodiments, L^{16E} is independently

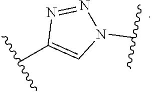 [SMILES: *c1cn(*)nn1]

In embodiments, L^{16F} is independently bond, —SS—, substituted or unsubstituted alkylene, substituted or unsubstituted heteroalkylene, or substituted or unsubstituted heteroarylene. In embodiments, L^{16F} is independently bond. In embodiments, L^{16F} is independently —SS—. In embodiments, L^{16F} is independently substituted or unsubstituted C₁-C₄ alkylene. In embodiments, L^{16F} is independently substituted or unsubstituted 2 to 6 membered heteroalkylene. In embodiments, L^{16F} is independently substituted or unsubstituted 3 to 6 membered heteroarylene. In embodiments, L^{16F} is independently unsubstituted triazolylene. In embodiments, L^{16F} is independently

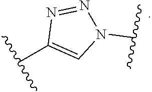 [SMILES: *c1cn(*)nn1]

In embodiments, -L^{16B}-L^{16C}-L^{16D}- is —SS—,

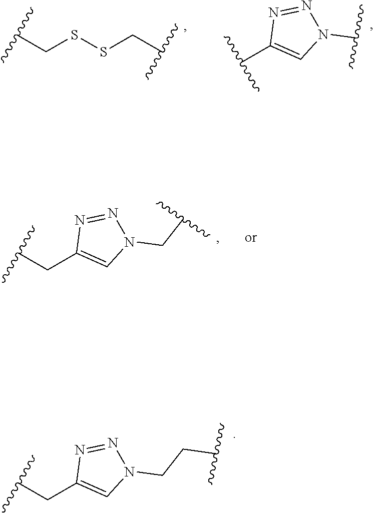 [SMILES: *CSSC* ; *c1cn(*)nn1 ; *Cc1cn(C*)nn1 ; *CCn1cc(C*)nn1]

In embodiments, L¹⁶ is —NH-L^{16B}-L^{16C}-L^{16D}-L^{16E}-C(O)—.

In embodiments, L^{16B} is

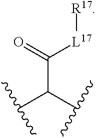

L¹⁷ is -L^{17A}-L^{17B}-L^{17C}-L^{17D}-L^{17E}-L^{17F}-.

L^{17A}, L^{17B}, L^{17C}, L^{17D}, L^{17E}, and L^{17F} are independently bond, —SS—, —S(O)₂—, —OS(O)₂—, —S(O)₂O—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), or substituted or unsubstituted heteroarylene (e.g., 5 to 10 membered, 5 to 9 membered, or 5 to 6 membered).

R¹⁷ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —C(O)H, —C(O)OH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), substituted or unsubstituted heteroaryl (e.g., 5 to 10 membered, 5 to 9 membered, or 5 to 6 membered), a monovalent nucleic acid, a monovalent protein, a detectable moiety, or a drug moiety.

In embodiments, L¹⁶ is a bond,

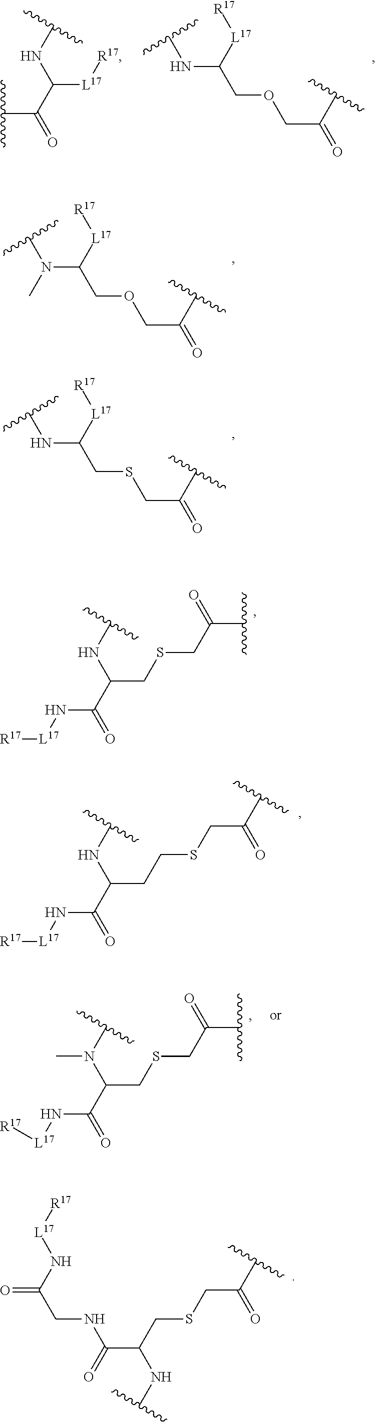

L¹⁷ and R¹⁷ are as described herein, including in embodiments. In embodiments, L¹⁶ is a bond. In embodiments, L¹⁶ is

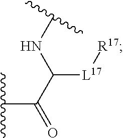

L¹⁷ and R¹⁷ are as described herein, including in embodiments. In embodiments, L¹⁶ is

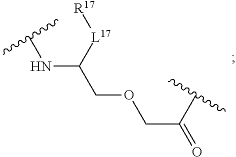

L¹⁷ and R¹⁷ are as described herein, including in embodiments. In embodiments, L¹⁶ is

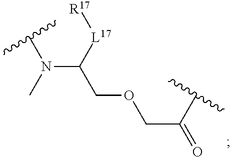

L¹⁷ and R¹⁷ are as described herein, including in embodiments. In embodiments, L¹⁶ is

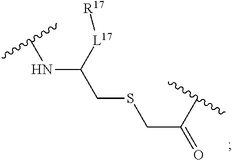

L¹⁷ and R¹⁷ are as described herein, including in embodiments. In embodiments, L¹⁶ is

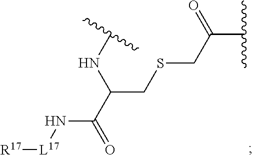

L¹⁷ and R¹⁷ are as described herein, including in embodiments. In embodiments, L¹⁶ is

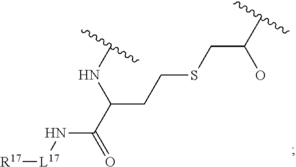

L¹⁷ and R¹⁷ are as described herein, including in embodiments. In embodiments, L¹⁶ is

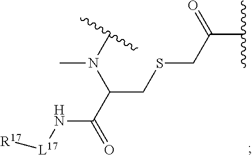

L¹⁷ and R¹⁷ are as described herein, including in embodiments.

In embodiments, L¹⁶ is

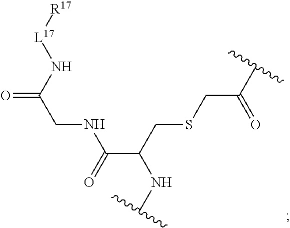

L¹⁷ and R¹⁷ are as described herein, including in embodiments.

In embodiments, a substituted L^{17A} (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{17A} is substituted with a plurality of groups selected from substituent groups, size-limited substituent groups, and lower substituent groups; each substituent group, size-limited substituent group, and/or lower substituent group may optionally be different. In embodiments, when L^{17A} is substituted, it is substituted with at least one substituent group. In embodiments, when L^{17A} is substituted, it is substituted with at least one size-limited substituent group. In embodiments, when L^{17A} is substituted, it is substituted with at least one lower substituent group.

In embodiments, a substituted L^{17B} (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{17B} is substituted with a plurality of groups selected from substituent groups, size-limited substituent groups, and lower substituent groups; each substituent group, size-limited substituent group, and/or lower substituent group may optionally be different. In embodiments, when L^{17B} is substituted, it is substituted with at least one substituent group. In embodiments, when L^{17B} is substituted, it is substituted with at least one size-limited substituent group. In embodiments, when L^{17B} is substituted, it is substituted with at least one lower substituent group.

In embodiments, a substituted L^{17C} (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{17C} is substituted with a plurality of groups selected from substituent groups, size-limited substituent groups, and lower substituent groups; each substituent group, size-limited substituent group, and/or lower substituent group may optionally be different. In embodiments, when L^{17C} is substituted, it is substituted with at least one substituent group. In embodiments, when L^{17C} is substituted, it is substituted with at least one size-limited substituent group. In embodiments, when L^{17C} is substituted, it is substituted with at least one lower substituent group.

In embodiments, a substituted L^{17D} (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{17D} is substituted with a plurality of groups selected from substituent groups, size-limited substituent groups, and lower substituent groups; each substituent group, size-limited substituent group, and/or lower substituent group may optionally be different. In embodiments, when L^{17D} is substituted, it is substituted with at least one substituent group. In embodiments, when L^{17D} is substituted, it is substituted with at least one size-limited substituent group. In embodiments, when L^{17D} is substituted, it is substituted with at least one lower substituent group.

In embodiments, a substituted L^{17E} (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{17E} is substituted with a plurality of groups selected from substituent groups, size-limited substituent groups, and lower substituent groups; each substituent group, size-limited substituent group, and/or lower substituent group may optionally be different. In embodiments, when L^{17E} is substituted, it is substituted with at least one substituent group. In embodiments, when L^{17E} is substituted, it is substituted with at least one size-limited substituent group. In embodiments, when L^{17E} is substituted, it is substituted with at least one lower substituent group.

In embodiments, a substituted L^{17F} (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L^{17F} is substituted with a plurality of groups selected from substituent groups, size-limited substituent groups, and lower substituent groups; each substituent group, size-limited substituent group, and/or lower substituent group may optionally be different. In embodiments, when L^{17F} is substituted, it is substituted with at least one substituent group. In embodiments, when L^{17F} is substituted, it is substituted with at least one size-limited substituent group. In embodiments, when L^{17F} is substituted, it is substituted with at least one lower substituent group.

In embodiments, L^{17A} is a bond, unsubstituted alkylene, or unsubstituted heteroalkylene. In embodiments, L^{17A} is a bond, unsubstituted C₁-C₆ alkylene, or unsubstituted 2 to 6 membered heteroalkylene. In embodiments, L^{17A} is a bond. In embodiments, L^{17A} is unsubstituted C₁-C₆ alkylene. In embodiments, L^{17A} is unsubstituted 2 to 6 membered heteroalkylene. In embodiments, L^{17A} is

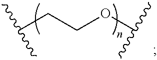

n is independently an integer from 1 to 100. In embodiments, n is independently an integer from 1 to 5.

In embodiments, L^{17B} is a bond, —NHC(O)—, —C(O)NH—, substituted or unsubstituted alkylene, or substituted or unsubstituted heteroalkylene. In embodiments, L^{17B} is a bond, —NHC(O)—, —C(O)NH—, substituted or unsubstituted C₁-C₆ alkylene, or substituted or unsubstituted 2 to 6 membered heteroalkylene. In embodiments, L^{17B} is a bond. In embodiments, L^{17B} is —NHC(O)—. In embodiments, L^{17B} is —C(O)NH—. In embodiments, L^{17B} is substituted or unsubstituted C₁-C₆ alkylene. In embodiments, L^{17B} is substituted or unsubstituted 2 to 6 membered heteroalkylene. In embodiments, L^{17B} is

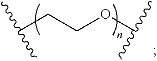

n is independently an integer from 1 to 100. In embodiments, n is independently an integer from 1 to 5.

In embodiments, L^{17C} is a bond, unsubstituted alkylene, or unsubstituted heteroalkylene. In embodiments, L^{17C} is a bond, unsubstituted C₁-C₆ alkylene, or unsubstituted 2 to 6 membered heteroalkylene. In embodiments, L^{17C} is a bond. In embodiments, L^{17C} is unsubstituted C₁-C₆ alkylene. In embodiments, L^{17C} is unsubstituted 2 to 6 membered heteroalkylene. In embodiments, L¹⁷c is

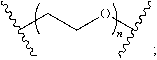

n is independently an integer from 1 to 100. In embodiments, n is independently an integer from 1 to 5.

In embodiments, L^{17D} is a bond, —O—, unsubstituted alkylene, or unsubstituted heteroalkylene. In embodiments, L^{17D} is a bond, —O—, unsubstituted C₁-C₈ alkylene, or unsubstituted 2 to 6 membered heteroalkylene. In embodiments, L^{17D} is a bond. In embodiments, L^{17D} is —O—. In embodiments, L^{17D} is unsubstituted C₁-C₈ alkylene. In embodiments, L^{17D} is unsubstituted 2 to 6 membered heteroalkylene.

In embodiments, L^{17E} is a bond, unsubstituted alkylene, or unsubstituted heteroalkylene. In embodiments, L^{17E} is a bond, unsubstituted C₁-C₈ alkylene, or unsubstituted 2 to 6 membered heteroalkylene. In embodiments, L^{17E} is a bond. In embodiments, L^{17E} is unsubstituted C₁-C₈ alkylene. In embodiments, L^{17E} is unsubstituted 2 to 6 membered heteroalkylene.

In embodiments, L^{17F} is a bond, unsubstituted alkylene, or unsubstituted heteroalkylene. In embodiments, L^{17F} is a bond, unsubstituted C₁-C₈ alkylene, or unsubstituted 2 to 6 membered heteroalkylene. In embodiments, L^{17F} is a bond. In embodiments, L^{17F} is unsubstituted C₁-C₈ alkylene. In embodiments, L^{17F} is unsubstituted 2 to 6 membered heteroalkylene.

In embodiments, n is independently 1. In embodiments, n is independently 2. In embodiments, n is independently 3. In embodiments, n is independently 4. In embodiments, n is independently 5.

In embodiments, L¹⁷ is a divalent form of puromycin. In embodiments, L¹⁷ is -L^{7A}-(divalent form of puromycin)-L^{17E}-L^{17F}-; L^{7A}, L^{17E}, and L^{17F} are as described herein, including in embodiments. In embodiments, L¹⁷ is

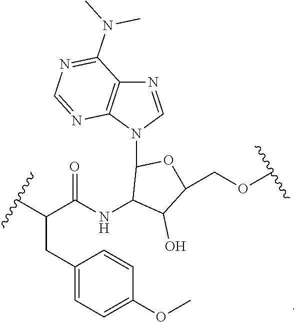 [SMILES: *OCC1OC(n2cnc3c(N(C)C)ncnc32)C(NC(=O)C(*)Cc2ccc(OC)cc2)C1O]

In embodiments, L¹⁷ is

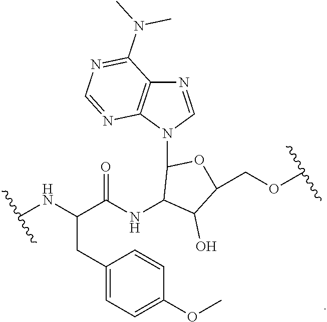 [SMILES: *NC(Cc1ccc(OC)cc1)C(=O)NC1C(O)C(CO*)OC1n1cnc2c(N(C)C)ncnc21]

In embodiments, L¹⁷ is

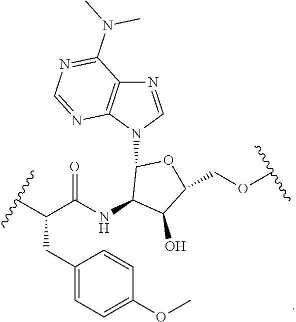 [SMILES: *OC[C@H]1O[C@@H](n2cnc3c(N(C)C)ncnc32)[C@H](NC(=O)[C@@H](*)Cc2ccc(OC)cc2)[C@@H]1O]

In embodiments, L¹⁷ is

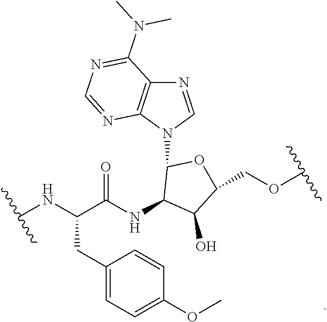 [SMILES: *N[C@@H](Cc1ccc(OC)cc1)C(=O)N[C@@H]1[C@H](O)[C@@H](CO*)O[C@H]1n1cnc2c(N(C)C)ncnc21]

In embodiments, -L^{17B}-L^{17C}-L^{17D}- is a divalent form of puromycin. In embodiments, -L^{17B}-L^{17C}-L^{17D}- is

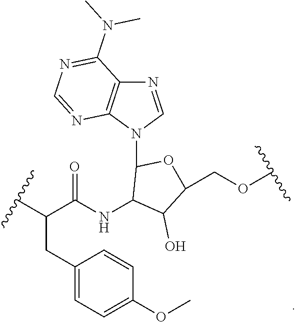 [SMILES: *OCC1OC(n2cnc3c(N(C)C)ncnc32)C(NC(=O)C(*)Cc2ccc(OC)cc2)C1O]

In embodiments, -L^{17B}-L^{17C}-L^{17D}- is

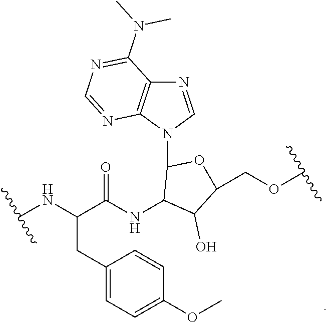 [SMILES: *NC(Cc1ccc(OC)cc1)C(=O)NC1C(O)C(CO*)OC1n1cnc2c(N(C)C)ncnc21]

In embodiments, -L^{17B}-L^{17C}-L^{17D}- is

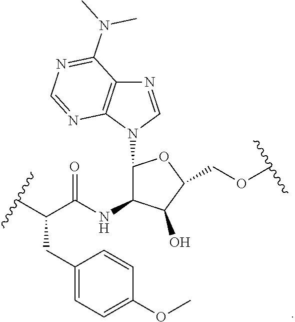 [SMILES: *OC[C@H]1O[C@@H](n2cnc3c(N(C)C)ncnc32)[C@H](NC(=O)[C@@H](*)Cc2ccc(OC)cc2)[C@@H]1O]

In embodiments, -L^{17B}, L^{17C}-L^{17D}- is

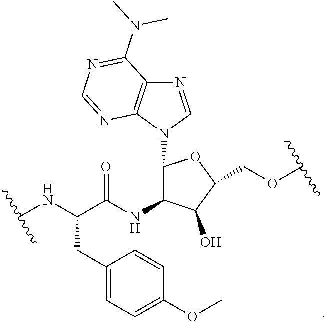 [SMILES: *N[C@@H](Cc1ccc(OC)cc1)C(=O)N[C@@H]1[C@H](O)[C@@H](CO*)O[C@H]1n1cnc2c(N(C)C)ncnc21]

In embodiments, a substituted R¹⁷ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁷ is substituted with a plurality of groups selected from substituent groups, size-limited substituent groups, and lower substituent groups; each substituent group, size-limited substituent group, and/or lower substituent group may optionally be different. In embodiments, when R¹⁷ is substituted, it is substituted with at least one substituent group. In embodiments, when R¹⁷ is substituted, it is substituted with at least one size-limited substituent group. In embodiments, when R¹⁷ is substituted, it is substituted with at least one lower substituent group.

In embodiments, R¹⁷ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —C(O)H, —C(O)OH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), substituted or unsubstituted heteroaryl (e.g., 5 to 10 membered, 5 to 9 membered, or 5 to 6 membered), a monovalent nucleic acid, a monovalent protein, or a detectable moiety.

In embodiments, R¹⁷ is hydrogen. In embodiments, R¹⁷ is —F. In embodiments, R¹⁷ is —Cl. In embodiments, R¹⁷ is —Br. In embodiments, R¹⁷ is —I. In embodiments, R¹⁷ is —CCl₃. In embodiments, R¹⁷ is —CBr₃. In embodiments, R¹⁷ is —CF₃. In embodiments, R¹⁷ is —CI₃. In embodiments, R¹⁷ is —CHCl₂. In embodiments, R¹⁷ is —CHBr₂. In embodiments, R¹⁷ is —CHF₂. In embodiments, R¹⁷ is —CHI₂. In embodiments, R¹⁷ is —CH₂Cl. In embodiments, R¹⁷ is —CH₂Br. In embodiments, R¹⁷ is —CH₂F. In embodiments, R¹⁷ is —CH₂I. In embodiments, R¹⁷ is —OH. In embodiments, R¹⁷ is —NH₂. In embodiments, R¹⁷ is substituted or unsubstituted alkyl. In embodiments, R¹⁷ is substituted or unsubstituted heteroalkyl. In embodiments, R¹⁷ is substituted or unsubstituted cycloalkyl. In embodiments, R¹⁷ is substituted or unsubstituted heterocycloalkyl. In embodiments, R¹⁷ is substituted or unsubstituted aryl. In embodiments, R¹⁷ is substituted or unsubstituted heteroaryl. In embodiments, R¹⁷ is a monovalent nucleic acid. In embodiments, R¹⁷ is a monovalent protein. In embodiments, R¹⁷ is a detectable moiety. In embodiments, R¹⁷ is a drug moiety. In embodiments, R¹⁷ is a monovalent form of thalidomide.

In embodiments, -L¹⁷-R¹⁷ is

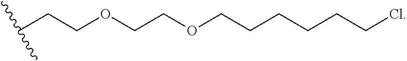 [SMILES: *CCOCCOCCCCCCCl]

In embodiments, -L¹⁷-R¹⁷ is

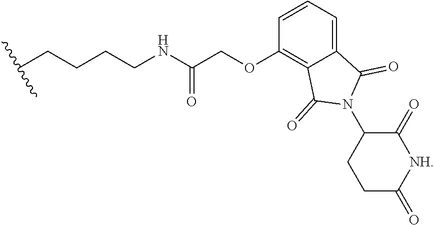 [SMILES: *CCCCNC(=O)COc1cccc2c1C(=O)N(C1CCC(=O)NC1=O)C2=O]

In embodiments, L¹⁶ is —SS—,

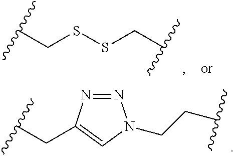 [SMILES: *CSSC* ; *CCn1cc(C*)nn1]

In embodiments, L¹⁶ is a bond,

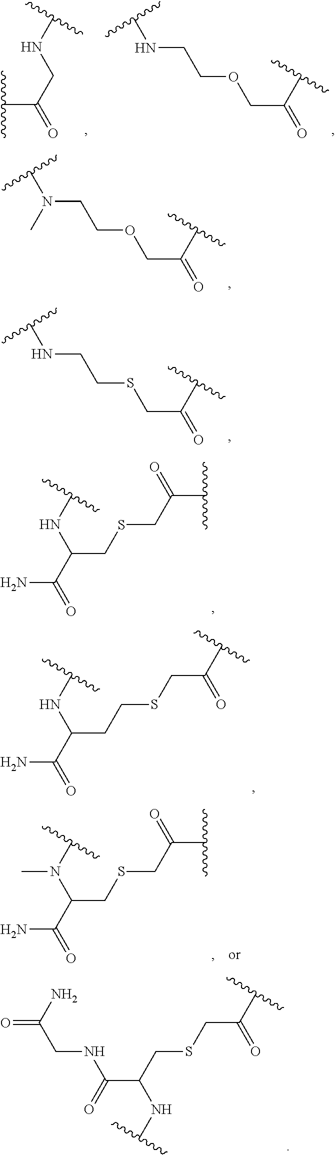

In embodiments, L¹⁶ is a bond. In embodiments, L¹⁶ is

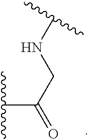 [SMILES: *NCC(*)=O]

In embodiments, L¹⁶ is

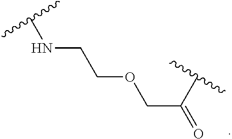 [SMILES: *NCCOCC(*)=O]

In embodiments, L¹⁶ is

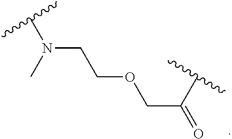 [SMILES: *C(=O)COCCN(*)C]

In embodiments, L¹⁶ is

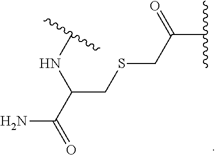 [SMILES: *NC(CSCC(*)=O)C(N)=O]

In embodiments, L¹⁶ is

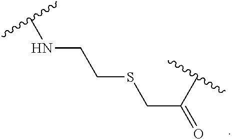 [SMILES: *NCCSCC(*)=O]

In embodiments, L¹⁶ is

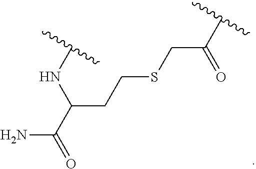 [SMILES: *NC(CCSCC(*)=O)C(N)=O]

In embodiments, L¹⁶ is

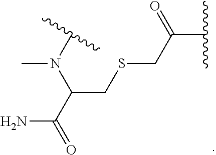 [SMILES: *C(=O)CSCC(C(N)=O)N(*)C]

In embodiments, L¹⁶ is

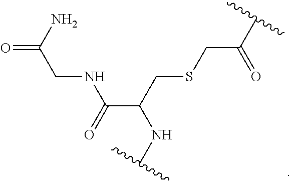 [SMILES: *NC(CSCC(*)=O)C(=O)NCC(N)=O]

In embodiments, the compound has the formula:

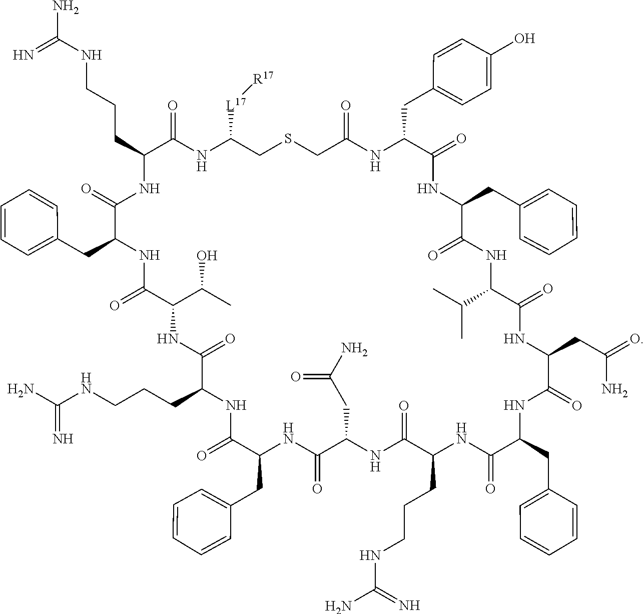

L¹⁷ and R¹⁷ are as described herein, including in embodiments.

In embodiments, the compound has the formula:

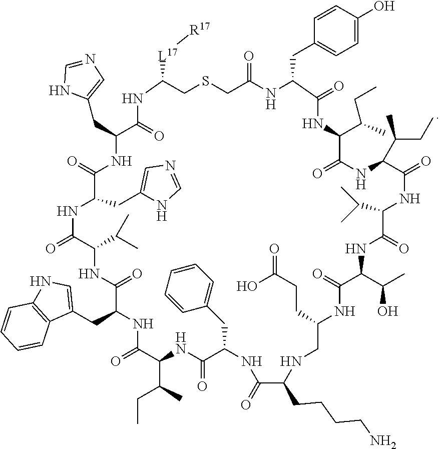

L¹⁷ and R¹⁷ are as described herein, including in embodiments.

In embodiments, the compound has the formula:

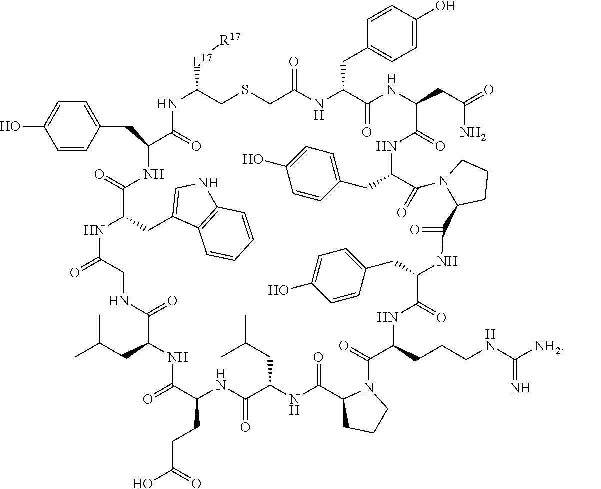

L¹⁷ and R¹⁷ are as described herein, including in embodiments.

In embodiments, the compound has the formula:

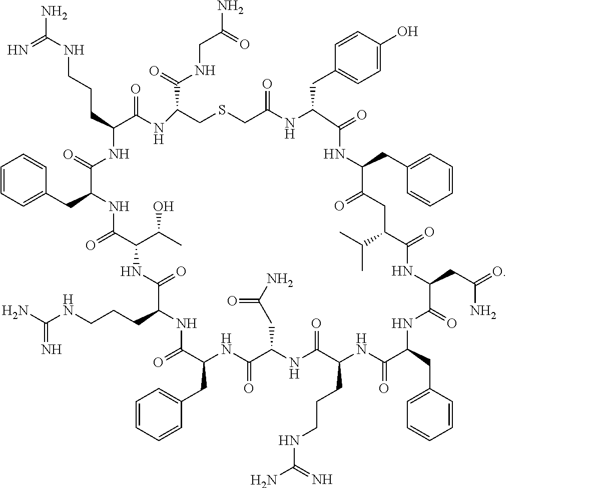 [SMILES: CC(C)[C@@H]1CC(=O)[C@H](Cc2ccccc2)NC(=O)[C@@H](Cc2ccc(O)cc2)NC(=O)CSC[C@@H](C(=O)NCC(N)=O)NC(=O)[C@H](CCCNC(=N)N)NC(=O)[C@H](Cc2ccccc2)NC(=O)[C@H]([C@@H](C)O)NC(=O)[C@H](CCCNC(=N)N)NC(=O)[C@H](Cc2ccccc2)NC(=O)[C@H](CC(N)=O)NC(=O)[C@H](CCCNC(=N)N)NC(=O)[C@H](Cc2ccccc2)NC(=O)[C@H](CC(N)=O)NC1=O]

In embodiments, the compound has the formula:

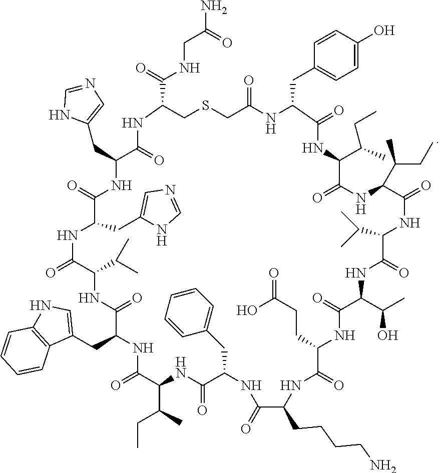 [SMILES: CC[C@H](C)[C@@H]1NC(=O)[C@H](Cc2ccccc2)NC(=O)[C@H](CCCCN)NC(=O)[C@H](CCC(=O)O)NC(=O)[C@H]([C@@H](C)O)NC(=O)[C@H](C(C)C)NC(=O)[C@H]([C@@H](C)CC)NC(=O)[C@H]([C@@H](C)CC)NC(=O)[C@@H](Cc2ccc(O)cc2)NC(=O)CSC[C@@H](C(=O)NCC(N)=O)NC(=O)[C@H](Cc2cnc[nH]2)NC(=O)[C@H](Cc2cnc[nH]2)NC(=O)[C@H](C(C)C)NC(=O)[C@H](Cc2c[nH]c3ccccc23)NC1=O]

In embodiments, the compound has the formula:

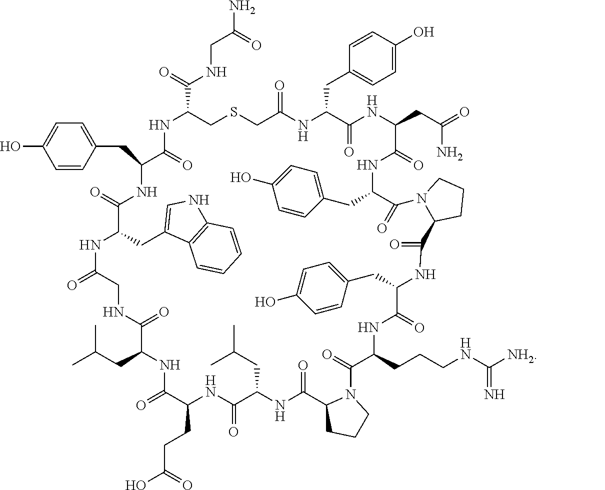 [SMILES: CC(C)C[C@@H]1NC(=O)[C@H](CCC(=O)O)NC(=O)[C@H](CC(C)C)NC(=O)[C@@H]2CCCN2C(=O)[C@H](CCCNC(=N)N)NC(=O)[C@H](Cc2ccc(O)cc2)NC(=O)[C@@H]2CCCN2C(=O)[C@H](Cc2ccc(O)cc2)NC(=O)[C@H](CC(N)=O)NC(=O)[C@@H](Cc2ccc(O)cc2)NC(=O)CSC[C@@H](C(=O)NCC(N)=O)NC(=O)[C@H](Cc2ccc(O)cc2)NC(=O)[C@H](Cc2c[nH]c3ccccc23)NC(=O)CNC1=O]

In embodiments, the compound has the formula:

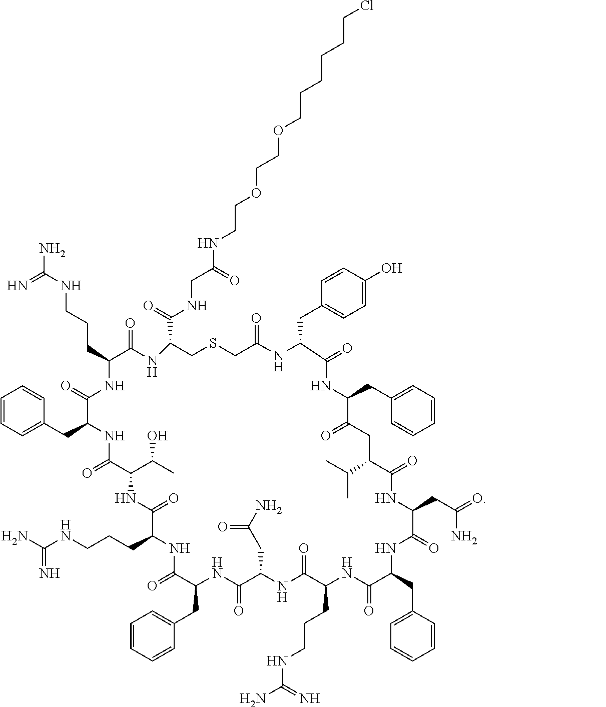 [SMILES: CC(C)[C@@H]1CC(=O)[C@H](Cc2ccccc2)NC(=O)[C@@H](Cc2ccc(O)cc2)NC(=O)CSC[C@@H](C(=O)NCC(=O)NCCOCCOCCCCCCCl)NC(=O)[C@H](CCCNC(=N)N)NC(=O)[C@H](Cc2ccccc2)NC(=O)[C@H]([C@@H](C)O)NC(=O)[C@H](CCCNC(=N)N)NC(=O)[C@H](Cc2ccccc2)NC(=O)[C@H](CC(N)=O)NC(=O)[C@H](CCCNC(=N)N)NC(=O)[C@H](Cc2ccccc2)NC(=O)[C@H](CC(N)=O)NC1=O]

In embodiments, the compound has the formula:

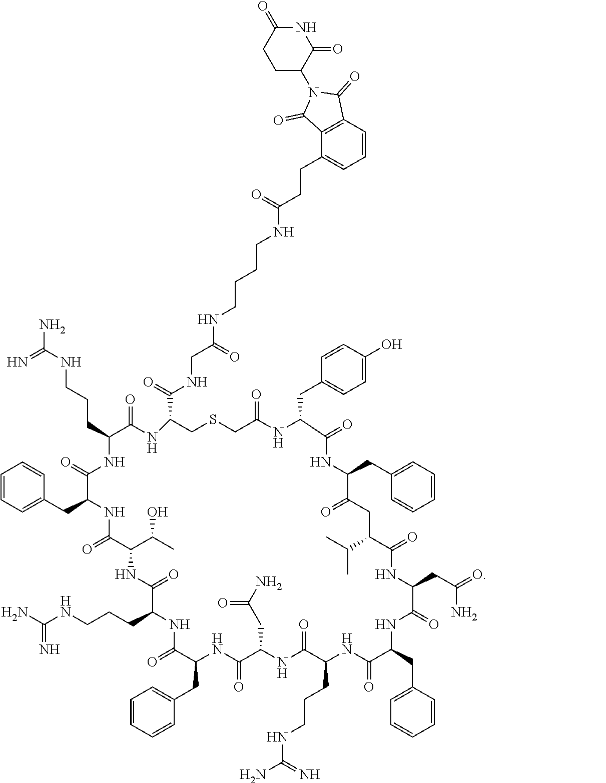 [SMILES: CC(C)[C@@H]1CC(=O)[C@H](Cc2ccccc2)NC(=O)[C@@H](Cc2ccc(O)cc2)NC(=O)CSC[C@@H](C(=O)NCC(=O)NCCCCNC(=O)CCc2cccc3c2C(=O)N(C2CCC(=O)NC2=O)C3=O)NC(=O)[C@H](CCCNC(=N)N)NC(=O)[C@H](Cc2ccccc2)NC(=O)[C@H]([C@@H](C)O)NC(=O)[C@H](CCCNC(=N)N)NC(=O)[C@H](Cc2ccccc2)NC(=O)[C@H](CC(N)=O)NC(=O)[C@H](CCCNC(=N)N)NC(=O)[C@H](Cc2ccccc2)NC(=O)[C@H](CC(N)=O)NC1=O]

In embodiments, when R^{1A} is substituted, R^{1A} is substituted with one or more first substituent groups denoted by R^{1A.1} as explained in the definitions section above in the description of “first substituent group(s)”. In embodiments, when an R^{1A.1} substituent group is substituted, the R^{1A.1} substituent group is substituted with one or more second substituent groups denoted by R^{1A.2} as explained in the definitions section above in the description of “first substituent group(s)”. In embodiments, when an R^{1A.2} substituent group is substituted, the R^{1A.2} substituent group is substituted with one or more third substituent groups denoted by R^{1A.3} as explained in the definitions section above in the description of “first substituent group(s)”. In the above embodiments, R^{1A}, R^{1A.1}, R^{1A.2}, and R^{1A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1A}, R^{1A.1}, R^{1A.2} and R^{1A.3}, respectively.

In embodiments, when R^{1B} is substituted, R^{1B} is substituted with one or more first substituent groups denoted by R^{1B.1} as explained in the definitions section above in the description of “first substituent group(s)”. In embodiments, when an R^{1B.1} substituent group is substituted, the R^{1B.1} substituent group is substituted with one or more second substituent groups denoted by R^{1B.2} as explained in the definitions section above in the description of “first substituent group(s)”. In embodiments, when an R^{1B.2} substituent group is substituted, the R^{1B.2} substituent group is substituted with one or more third substituent groups denoted by R^{1B.3} as explained in the definitions section above in the description of “first substituent group(s)”. In the above embodiments, R^{1B}, R^{1B.1}, R^{1B.2}, and R^{1B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1B}, R^{1B.1}, R^{1B.2}, and R^{1B.3}, respectively.

In embodiments, when R^{1C} is substituted, R^{1C} is substituted with one or more first substituent groups denoted by R^{1C.1} as explained in the definitions section above in the description of “first substituent group(s)”. In embodiments, when an R^{1C.1} substituent group is substituted, the R^{1C.1} substituent group is substituted with one or more second substituent groups denoted by R^{1C.2} as explained in the definitions section above in the description of “first substituent group(s)”. In embodiments, when an R^{1C.2} substituent group is substituted, the R^{1C.2} substituent group is substituted with one or more third substituent groups denoted by R^{1C.3} as explained in the definitions section above in the description of “first substituent group(s)”. In the above embodiments, R^{1C}, R^{1C.1}, R^{1C.2}, and R^{1C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1C}, R^{1C.1}, R^{1C.2}, and R^{1C.3}, respectively.

In embodiments, when R^{1F} is substituted, R^{1F} is substituted with one or more first substituent groups denoted by R^{1F.1} as explained in the definitions section above in the description of “first substituent group(s)”. In embodiments, when an R^{1F.1} substituent group is substituted, the R^{1F.1} substituent group is substituted with one or more second substituent groups denoted by R^{1F.2} as explained in the definitions section above in the description of “first substituent group(s)”. In embodiments, when an R^{1F.2} substituent group is substituted, the R^{1F.2} substituent group is substituted with one or more third substituent groups denoted by R^{1F.3} as explained in the definitions section above in the description of “first substituent group(s)”. In the above embodiments, R^{1F}, R^{1F.1}, R^{1F.2}, and R^{1F.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1F}, R^{1F.1}, R^{1F.2}, and R^{1F.3}, respectively.

In embodiments, when R^{2A} is substituted, R^{2A} is substituted with one or more first substituent groups denoted by R^{2A.1} as explained in the definitions section above in the description of “first substituent group(s)”. In embodiments, when an R^{2A.1} substituent group is substituted, the R^{2A.1} substituent group is substituted with one or more second substituent groups denoted by R^{2A.2} as explained in the definitions section above in the description of “first substituent group(s)”. In embodiments, when an R^{2A.2} substituent group is substituted, the R^{2A.2} substituent group is substituted with one or more third substituent groups denoted by R^{2A.3} as explained in the definitions section above in the description of “first substituent group(s)”. In the above embodiments, R^{2A}, R^{2A.1}, R^{2A.2}, and R^{2A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2A}, R^{2A.1}, R^{2A.2}, and R^{2A.3}, respectively.

In embodiments, when R^{2B} is substituted, R^{2B} is substituted with one or more first substituent groups denoted by R^{2B.1} as explained in the definitions section above in the description of “first substituent group(s)”. In embodiments, when an R^{2B.1} substituent group is substituted, the R^{2B.1} substituent group is substituted with one or more second substituent groups denoted by R^{2B.2} as explained in the definitions section above in the description of “first substituent group(s)”. In embodiments, when an R^{2B.2} substituent group is substituted, the R^{2B.2} substituent group is substituted with one or more third substituent groups denoted by R^{2B.3} as explained in the definitions section above in the description of “first substituent group(s)”. In the above embodiments, R^{2B}, R^{2B.1}, R^{2B.2}, and R^{2B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2B}, R^{2B.1}, R^{2B.2}, and R^{2B.3}, respectively.

In embodiments, when R^{2C} is substituted, R^{2C} is substituted with one or more first substituent groups denoted by R^{2C.1} as explained in the definitions section above in the description of “first substituent group(s)”. In embodiments, when an R^{2C.1} substituent group is substituted, the R^{2C.1} substituent group is substituted with one or more second substituent groups denoted by R^{2C.2} as explained in the definitions section above in the description of “first substituent group(s)”. In embodiments, when an R^{2C.2} substituent group is substituted, the R^{2C.2} substituent group is substituted with one or more third substituent groups denoted by R^{2C.3} as explained in the definitions section above in the description of “first substituent group(s)”. In the above embodiments, R^{2C}, R^{2C.1}, R^{2C.2}, and R^{2C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2C}, R^{2C.1}, R^{2C.2}, and R^{2C.3}, respectively.

In embodiments, when R^{2F} is substituted, R^{2F} is substituted with one or more first substituent groups denoted by R^{2F.1} as explained in the definitions section above in the description of “first substituent group(s)”. In embodiments, when an R^{2F.1} substituent group is substituted, the R^{2F.1} substituent group is substituted with one or more second substituent groups denoted by R^{2F.2} as explained in the definitions section above in the description of “first substituent group(s)”. In embodiments, when an R^{2F.2} substituent group is substituted, the R^{2F.2} substituent group is substituted with one or more third substituent groups denoted by R^{2F.3} as explained in the definitions section above in the description of “first substituent group(s)”. In the above embodiments, R^{2F}, R^{2F.1}, R^{2F.2}, and R^{2F.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2F}, R^{2F.1}, R^{2F.2}, and R^{2F.3}, respectively.

In embodiments, when R^{3A} is substituted, R^{3A} is substituted with one or more first substituent groups denoted by R^{3A.1} as explained in the definitions section above in the description of “first substituent group(s)”. In embodiments, when an R^{3A.1} substituent group is substituted, the R^{3A.1} substituent group is substituted with one or more second substituent groups denoted by R^{3A.2} as explained in the definitions section above in the description of “first substituent group(s)”. In embodiments, when an R^{3A.2} substituent group is substituted, the R^{3A.2} substituent group is substituted with one or more third substituent groups denoted by R^{3A.3} as explained in the definitions section above in the description of “first substituent group(s)”. In the above embodiments, R^{3A}, R^{3A.1}, R^{3A.2}, and R^{3A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3A}, R^{3A.1}, R^{3A.2} and R^{3A.3}, respectively.

In embodiments, when R^{3B} is substituted, R^{3B} is substituted with one or more first substituent groups denoted by R^{3B.1} as explained in the definitions section above in the description of “first substituent group(s)”. In embodiments, when an R^{3B.1} substituent group is substituted, the R^{3B.1} substituent group is substituted with one or more second substituent groups denoted by R^{3B.2} as explained in the definitions section above in the description of “first substituent group(s)”. In embodiments, when an R^{3B.2} substituent group is substituted, the R^{3B.2} substituent group is substituted with one or more third substituent groups denoted by R^{3B.3} as explained in the definitions section above in the description of “first substituent group(s)”. In the above embodiments, R^{3B}, R^{3B.1}, R^{3B.2}, and R^{3B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3B}, R^{3B.1}, R^{3B.2}, and R^{3B.3}, respectively.

In embodiments, when R^{3C} is substituted, R^{3C} is substituted with one or more first substituent groups denoted by R^{3C.1} as explained in the definitions section above in the description of “first substituent group(s)”. In embodiments, when an R^{3C.1} substituent group is substituted, the R^{3C.1} substituent group is substituted with one or more second substituent groups denoted by R^{3C.2} as explained in the definitions section above in the description of “first substituent group(s)”. In embodiments, when an R^{3C.2} substituent group is substituted, the R^{3C.2} substituent group is substituted with one or more third substituent groups denoted by R^{3C.3} as explained in the definitions section above in the description of “first substituent group(s)”. In the above embodiments, R^{3C}, R^{3C.1}, R^{3C.2}, and R^{3C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3C}, R^{3C.1}, R^{3C.2}, and R^{3C.3}, respectively.

In embodiments, when R^{3F} is substituted, R^{3F} is substituted with one or more first substituent groups denoted by R^{3F.1} as explained in the definitions section above in the description of “first substituent group(s)”. In embodiments, when an R^{3F.1} substituent group is substituted, the R^{3F.1} substituent group is substituted with one or more second substituent groups denoted by R^{3F.2} as explained in the definitions section above in the description of “first substituent group(s)”. In embodiments, when an R^{3F.2} substituent group is substituted, the R^{3F.2} substituent group is substituted with one or more third substituent groups denoted by R^{3F.3} as explained in the definitions section above in the description of “first substituent group(s)”. In the above embodiments, R^{3F}, R^{3F.1}, R^{3F.2}, and R^{3F.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{3F}, R^{3F.1}, R^{3F.2}, and R^{3F.3}, respectively.

In embodiments, when R^{4A} is substituted, R^{4A} is substituted with one or more first substituent groups denoted by R^{4A.1} as explained in the definitions section above in the description of “first substituent group(s)”. In embodiments, when an R^{4A.1} substituent group is substituted, the R^{4A.1} substituent group is substituted with one or more second substituent groups denoted by R^{4A.2} as explained in the definitions section above in the description of “first substituent group(s)”. In embodiments, when an R^{4A.2} substituent group is substituted, the R^{4A.2} substituent group is substituted with one or more third substituent groups denoted by R^{4A.3} as explained in the definitions section above in the description of “first substituent group(s)”. In the above embodiments, R^{4A}, R^{4A.1}, R^{4A.2}, and R^{4A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{4A}, R^{4A.1}, R^{4A.2}, and R^{4A.3}, respectively.

In embodiments, when R^{4B} is substituted, R^{4B} is substituted with one or more first substituent groups denoted by R^{4B.1} as explained in the definitions section above in the description of “first substituent group(s)”. In embodiments, when an R^{4B.1} substituent group is substituted, the R^{4B.1} substituent group is substituted with one or more second substituent groups denoted by R^{4B.2} as explained in the definitions section above in the description of “first substituent group(s)”. In embodiments, when an R^{4B.2} substituent group is substituted, the R^{4B.2} substituent group is substituted with one or more third substituent groups denoted by R^{4B.3} as explained in the definitions section above in the description of “first substituent group(s)”. In the above embodiments, R^{4B}, R^{4B.1}, R^{4B.2}, and R^{4B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{4B}, R^{4B.1}, R^{4B.2}, and R^{4B.3}, respectively.

In embodiments, when R^{4C} is substituted, R^{4C} is substituted with one or more first substituent groups denoted by R^{4C.1} as explained in the definitions section above in the description of “first substituent group(s)”. In embodiments, when an R^{4C.1} substituent group is substituted, the R^{4C.1} substituent group is substituted with one or more second substituent groups denoted by R^{4C.2} as explained in the definitions section above in the description of “first substituent group(s)”. In embodiments, when an R^{4C.2} substituent group is substituted, the R^{4C.2} substituent group is substituted with one or more third substituent groups denoted by R^{4C.3} as explained in the definitions section above in the description of “first substituent group(s)”. In the above embodiments, R^{4C}, R^{4C.1}, R^{4C.2}, and R^{4C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{4C}, R^{4C.1}, R^{4C.2}, and R^{4C.3}, respectively.

In embodiments, when R^{4F} is substituted, R^{4F} is substituted with one or more first substituent groups denoted by R^{4F.1} as explained in the definitions section above in the description of “first substituent group(s)”. In embodiments, when an R^{4F.1} substituent group is substituted, the R^{4F.1} substituent group is substituted with one or more second substituent groups denoted by R^{4F.2} as explained in the definitions section above in the description of “first substituent group(s)”. In embodiments, when an R^{4F.2} substituent group is substituted, the R^{4F.2} substituent group is substituted with one or more third substituent groups denoted by R^{4F.3} as explained in the definitions section above in the description of “first substituent group(s)”. In the above embodiments, R^{4F}, R^{4F.1}, R^{4F.2}, and R^{4F.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{4F}, R^{4F.1}, R^{4F.2}, and R^{4F.3}, respectively.

In embodiments, when R^{5A} is substituted, R^{5A} is substituted with one or more first substituent groups denoted by R^{5A.1} as explained in the definitions section above in the description of “first substituent group(s)”. In embodiments, when an R^{5A.1} substituent group is substituted, the R^{5A.1} substituent group is substituted with one or more second substituent groups denoted by R^{5A.2} as explained in the definitions section above in the description of “first substituent group(s)”. In embodiments, when an R^{5A.2} substituent group is substituted, the R^{5A.2} substituent group is substituted with one or more third substituent groups denoted by R^{5A.3} as explained in the definitions section above in the description of “first substituent group(s)”. In the above embodiments, R^{5A}, R^{5A.1}, R^{5A.2}, and R^{5A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{5A}, R^{5A.1}, R^{5A.2} and R^{5A.3}, respectively.

In embodiments, when R^{5B} is substituted, R^{5B} is substituted with one or more first substituent groups denoted by R^{5B.1} as explained in the definitions section above in the description of “first substituent group(s)”. In embodiments, when an R^{5B.1} substituent group is substituted, the R^{5B.1} substituent group is substituted with one or more second substituent groups denoted by R^{5B.2} as explained in the definitions section above in the description of “first substituent group(s)”. In embodiments, when an R^{5B.2} substituent group is substituted, the R^{5B.2} substituent group is substituted with one or more third substituent groups denoted by R^{5B.3} as explained in the definitions section above in the description of “first substituent group(s)”. In the above embodiments, R^{5B}, R^{5B.1}, R^{5B.2}, and R^{5B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{5B}, R^{5B.1}, R^{5B.2}, and R^{5B.3}, respectively.

In embodiments, when R^{5C} is substituted, R^{5C} is substituted with one or more first substituent groups denoted by R^{5C.1} as explained in the definitions section above in the description of “first substituent group(s)”. In embodiments, when an R^{5C.1} substituent group is substituted, the R^{5C.1} substituent group is substituted with one or more second substituent groups denoted by R^{5C.2} as explained in the definitions section above in the description of “first substituent group(s)”. In embodiments, when an R^{5C.2} substituent group is substituted, the R^{5C.2} substituent group is substituted with one or more third substituent groups denoted by R^{5C.3} as explained in the definitions section above in the description of “first substituent group(s)”. In the above embodiments, R^{5C}, R^{5C.1}, R^{5C.2}, and R^{5C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{5C}, R^{5C.1}, R^{5C.2}, and R^{5C.3}, respectively.

In embodiments, when R^{5F} is substituted, R^{5F} is substituted with one or more first substituent groups denoted by R^{5F.1} as explained in the definitions section above in the description of “first substituent group(s)”. In embodiments, when an R^{5F.1} substituent group is substituted, the R^{5F.1} substituent group is substituted with one or more second substituent groups denoted by R^{5F.2} as explained in the definitions section above in the description of “first substituent group(s)”. In embodiments, when an R^{5F.2} substituent group is substituted, the R^{5F.2} substituent group is substituted with one or more third substituent groups denoted by R^{5F.3} as explained in the definitions section above in the description of “first substituent group(s)”. In the above embodiments, R^{5F}, R^{5F.1}, R^{5F.2}, and R^{5F.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{5F}, R^{5F.1}, R^{5F.2}, and R^{5F.3}, respectively.

In embodiments, when R^{6A} is substituted, R^{6A} is substituted with one or more first substituent groups denoted by R^{6A.1} as explained in the definitions section above in the description of “first substituent group(s)”. In embodiments, when an R^{6A.1} substituent group is substituted, the R^{6A.1} substituent group is substituted with one or more second substituent groups denoted by R^{6A.2} as explained in the definitions section above in the description of “first substituent group(s)”. In embodiments, when an R^{6A.2} substituent group is substituted, the R^{6A.2} substituent group is substituted with one or more third substituent groups denoted by R^{6A.3} as explained in the definitions section above in the description of “first substituent group(s)”. In the above embodiments, R^{6A}, R^{6A.1}, R^{6A.2}, and R^{6A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{6A}, R^{6A.1}, R^{6A.2}, and R^{6A.3}, respectively.

In embodiments, when R^{6B} is substituted, R^{6B} is substituted with one or more first substituent groups denoted by R^{6B.1} as explained in the definitions section above in the description of “first substituent group(s)”. In embodiments, when an R^{6B.1} substituent group is substituted, the R^{6B.1} substituent group is substituted with one or more second substituent groups denoted by R^{6B.2} as explained in the definitions section above in the description of “first substituent group(s)”. In embodiments, when an R^{6B.2} substituent group is substituted, the R^{6B.2} substituent group is substituted with one or more third substituent groups denoted by R^{6B.3} as explained in the definitions section above in the description of “first substituent group(s)”. In the above embodiments, R^{6B}, R^{6B.1}, R^{6B.2}, and R^{6B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{6B}, R^{6B.1}, R^{6B.2}, and R^{6B.3}, respectively.

In embodiments, when R^{6C} is substituted, R^{6C} is substituted with one or more first substituent groups denoted by R^{6C.1} as explained in the definitions section above in the description of “first substituent group(s)”. In embodiments, when an R^{6C.1} substituent group is substituted, the R^{6C.1} substituent group is substituted with one or more second substituent groups denoted by R^{6C.2} as explained in the definitions section above in the description of “first substituent group(s)”. In embodiments, when an R^{6C.2} substituent group is substituted, the R^{6C.2} substituent group is substituted with one or more third substituent groups denoted by R^{6C.3} as explained in the definitions section above in the description of “first substituent group(s)”. In the above embodiments, R^{6C}, R^{6C.1}, R^{6C.2}, and R^{6C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{6C}, R^{6C.1}, R^{6C.2}, and R^{6C.3}, respectively.

In embodiments, when R^{6F} is substituted, R^{6F} is substituted with one or more first substituent groups denoted by R^{6F.1} as explained in the definitions section above in the description of “first substituent group(s)”. In embodiments, when an R^{6F.1} substituent group is substituted, the R^{6F.1} substituent group is substituted with one or more second substituent groups denoted by R^{6F.2} as explained in the definitions section above in the description of “first substituent group(s)”. In embodiments, when an R^{6F.2} substituent group is substituted, the R^{6F.2} substituent group is substituted with one or more third substituent groups denoted by R^{6F.3} as explained in the definitions section above in the description of “first substituent group(s)”. In the above embodiments, R^{6F}, R^{6F.1}, R^{6F.2}, and R^{6F.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{6F}, R^{6F.1}, R^{6F.2}, and R^{6F.3}, respectively.

In embodiments, when R^{7A} is substituted, R^{7A} is substituted with one or more first substituent groups denoted by R^{7A.1} as explained in the definitions section above in the description of “first substituent group(s)”. In embodiments, when an R^{7A.1} substituent group is substituted, the R^{7A.1} substituent group is substituted with one or more second substituent groups denoted by R^{7A.2} as explained in the definitions section above in the description of “first substituent group(s)”. In embodiments, when an R^{7A.2} substituent group is substituted, the R^{7A.2} substituent group is substituted with one or more third substituent groups denoted by R^{7A.3} as explained in the definitions section above in the description of “first substituent group(s)”. In the above embodiments, R^{7A}, R^{7A.1}, R^{7A.2}, and R^{7A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{7A}, R^{7A.1}, R^{7A.2}, and R^{7A.3}, respectively.

In embodiments, when R^{7B} is substituted, R^{7B} is substituted with one or more first substituent groups denoted by R^{7B.1} as explained in the definitions section above in the description of “first substituent group(s)”. In embodiments, when an R^{7B.1} substituent group is substituted, the R^{7B.1} substituent group is substituted with one or more second substituent groups denoted by R^{7B.2} as explained in the definitions section above in the description of “first substituent group(s)”. In embodiments, when an R^{7B.2} substituent group is substituted, the R^{7B.2} substituent group is substituted with one or more third substituent groups denoted by R^{7B.3} as explained in the definitions section above in the description of “first substituent group(s)”. In the above embodiments, R^{7B}, R^{7B.1}, R^{7B.2}, and R^{7B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{7B}, R^{7B.1}, R^{7B.2}, and R^{7B.3}, respectively.

In embodiments, when R^{7C} is substituted, R^{7C} is substituted with one or more first substituent groups denoted by R^{7C.1} as explained in the definitions section above in the description of “first substituent group(s)”. In embodiments, when an R^{7C.1} substituent group is substituted, the R^{7C.1} substituent group is substituted with one or more second substituent groups denoted by R^{7C.2} as explained in the definitions section above in the description of “first substituent group(s)”. In embodiments, when an R^{7C.2} substituent group is substituted, the R^{7C.2} substituent group is substituted with one or more third substituent groups denoted by R^{7C.3} as explained in the definitions section above in the description of “first substituent group(s)”. In the above embodiments, R^{7C}, R^{7C.1}, R^{7C.2}, and R^{7C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{7C}, R^{7C.1}, R^{7C.2}, and R^{7C.3}, respectively.

In embodiments, when R^{7F} is substituted, R^{7F} is substituted with one or more first substituent groups denoted by R^{7F.1} as explained in the definitions section above in the description of “first substituent group(s)”. In embodiments, when an R^{7F.1} substituent group is substituted, the R^{7F.1} substituent group is substituted with one or more second substituent groups denoted by R^{7F.2} as explained in the definitions section above in the description of “first substituent group(s)”. In embodiments, when an R^{7F.2} substituent group is substituted, the R^{7F.2} substituent group is substituted with one or more third substituent groups denoted by R^{7F.3} as explained in the definitions section above in the description of “first substituent group(s)”. In the above embodiments, R^{7F}, R^{7F.1}, R^{7F.2}, and R^{7F.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{7F}, R^{7F.1}, R^{7F.2}, and R^{7F.3}, respectively.

In embodiments, when R^{8A} is substituted, R^{8A} is substituted with one or more first substituent groups denoted by R^{8A.1} as explained in the definitions section above in the description of “first substituent group(s)”. In embodiments, when an R^{8A.1} substituent group is substituted, the R^{8A.1} substituent group is substituted with one or more second substituent groups denoted by R^{8A.2} as explained in the definitions section above in the description of “first substituent group(s)”. In embodiments, when an R^{8A.2} substituent group is substituted, the R^{8A.2} substituent group is substituted with one or more third substituent groups denoted by R^{8A.3} as explained in the definitions section above in the description of “first substituent group(s)”. In the above embodiments, R^{8A}, R^{8A.1}, R^{8A.2}, and R^{8A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{8A}, R^{8A.1}, R^{8A.2}, and R^{8A.3}, respectively.

In embodiments, when R^{8B} is substituted, R^{8B} is substituted with one or more first substituent groups denoted by R^{8B.1} as explained in the definitions section above in the description of “first substituent group(s)”. In embodiments, when an R^{8B.1} substituent group is substituted, the R^{8B.1} substituent group is substituted with one or more second substituent groups denoted by R^{8B.2} as explained in the definitions section above in the description of “first substituent group(s)”. In embodiments, when an R^{8B.2} substituent group is substituted, the R^{8B.2} substituent group is substituted with one or more third substituent groups denoted by R^{8B.3} as explained in the definitions section above in the description of “first substituent group(s)”. In the above embodiments, R^{8B}, R^{8B.1}, R^{8B.2}, and R^{8B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{8B}, R^{8B.1}, R^{8B.2}, and R^{8B.3}, respectively.

In embodiments, when R^{8C} is substituted, R^{5C} is substituted with one or more first substituent groups denoted by R^{8C.1} as explained in the definitions section above in the description of “first substituent group(s)”. In embodiments, when an R^{8C.1} substituent group is substituted, the R^{8C.1} substituent group is substituted with one or more second substituent groups denoted by R^{8C.2} as explained in the definitions section above in the description of “first substituent group(s)”. In embodiments, when an R^{8C.2} substituent group is substituted, the R^{8C.2} substituent group is substituted with one or more third substituent groups denoted by R^{8C.3} as explained in the definitions section above in the description of “first substituent group(s)”. In the above embodiments, R^{8C}, R^{8C.1}, R^{8C.2}, and R^{8C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{8C}, R^{8C.1}, R^{8C.2}, and R^{8C.3}, respectively.

In embodiments, when R^{8F} is substituted, R^{8F} is substituted with one or more first substituent groups denoted by R^{8F.1} as explained in the definitions section above in the description of “first substituent group(s)”. In embodiments, when an R^{8F.1} substituent group is substituted, the R^{8F.1} substituent group is substituted with one or more second substituent groups denoted by R^{8F.2} as explained in the definitions section above in the description of “first substituent group(s)”. In embodiments, when an R^{8F.2} substituent group is substituted, the R^{8F.2} substituent group is substituted with one or more third substituent groups denoted by R^{8F.3} as explained in the definitions section above in the description of “first substituent group(s)”. In the above embodiments, R^{8F}, R^{8F.1}, R^{8F.2}, and R^{8F.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{8F}, R^{8F.1}, R^{8F.2}, and R^{8F.3}, respectively.

In embodiments, when R^{9A} is substituted, R^{9A} is substituted with one or more first substituent groups denoted by R^{9A.1} as explained in the definitions section above in the description of “first substituent group(s)”. In embodiments, when an R^{9A.1} substituent group is substituted, the R^{9A.1} substituent group is substituted with one or more second substituent groups denoted by R^{9A.2} as explained in the definitions section above in the description of “first substituent group(s)”. In embodiments, when an R^{9A.2} substituent group is substituted, the R^{9A.2} substituent group is substituted with one or more third substituent groups denoted by R^{9A.3} as explained in the definitions section above in the description of “first substituent group(s)”. In the above embodiments, R^{9A}, R^{9A.1}, R^{9A.2}, and R^{9A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{9A}, R^{9A.1}, R^{9A.2} and R^{9A.3}, respectively.

In embodiments, when R^{9B} is substituted, R^{9B} is substituted with one or more first substituent groups denoted by R^{9B.1} as explained in the definitions section above in the description of “first substituent group(s)”. In embodiments, when an R^{9B.1} substituent group is substituted, the R^{9B.1} substituent group is substituted with one or more second substituent groups denoted by R^{9B.2} as explained in the definitions section above in the description of “first substituent group(s)”. In embodiments, when an R^{9B.2} substituent group is substituted, the R^{9B.2} substituent group is substituted with one or more third substituent groups denoted by R^{9B.3} as explained in the definitions section above in the description of “first substituent group(s)”. In the above embodiments, R^{9B}, R^{9B.1}, R^{9B.2}, and R^{9B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{9B}, R^{9B.1}, R^{9B.2}, and R^{9B.3}, respectively.

In embodiments, when R^{9C} is substituted, R^{9C} is substituted with one or more first substituent groups denoted by R^{9C.1} as explained in the definitions section above in the description of “first substituent group(s)”. In embodiments, when an R^{9C.1} substituent group is substituted, the R^{9C.1} substituent group is substituted with one or more second substituent groups denoted by R^{9C.2} as explained in the definitions section above in the description of “first substituent group(s)”. In embodiments, when an R^{9C.2} substituent group is substituted, the R^{9C.2} substituent group is substituted with one or more third substituent groups denoted by R^{9C.3} as explained in the definitions section above in the description of “first substituent group(s)”. In the above embodiments, R^{9C}, R^{9C.1}, R^{9C.2}, and R^{9C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{9C}, R^{9C.1}, R^{9C.2}, and R^{9C.3}, respectively.

In embodiments, when R^{9F} is substituted, R^{9F} is substituted with one or more first substituent groups denoted by R^{9F.1} as explained in the definitions section above in the description of “first substituent group(s)”. In embodiments, when an R^{9F.1} substituent group is substituted, the R^{9F.1} substituent group is substituted with one or more second substituent groups denoted by R^{9F.2} as explained in the definitions section above in the description of “first substituent group(s)”. In embodiments, when an R^{9F.2} substituent group is substituted, the R^{9F.2} substituent group is substituted with one or more third substituent groups denoted by R^{9F.3} as explained in the definitions section above in the description of “first substituent group(s)”. In the above embodiments, R^{9F}, R^{9F.1}, R^{9F.2}, and R^{9F.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{9F}, R^{9F.1}, R^{9F.2}, and R^{9F.3}, respectively.

In embodiments, when R^{10A} is substituted, R^{10A} is substituted with one or more first substituent groups denoted by R^{10A.1} as explained in the definitions section above in the description of “first substituent group(s)”. In embodiments, when an R^{10A.1} substituent group is substituted, the R^{10A.1} substituent group is substituted with one or more second substituent groups denoted by R^{10A.2} as explained in the definitions section above in the description of “first substituent group(s)”. In embodiments, when an R^{10A.2} substituent group is substituted, the R^{10A.2} substituent group is substituted with one or more third substituent groups denoted by R^{10A.3} as explained in the definitions section above in the description of “first substituent group(s)”. In the above embodiments, R^{10A}, R^{10A.1}, R^{10A.2}, and R^{10A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{10A}, R^{10A.1}, R^{10A.2}, and R^{10A.3}, respectively.

In embodiments, when R^{10B} is substituted, R^{10B} is substituted with one or more first substituent groups denoted by R^{10B.1} as explained in the definitions section above in the description of “first substituent group(s)”. In embodiments, when an R^{10B} substituent group is substituted, the R^{10B.1} substituent group is substituted with one or more second substituent groups denoted by R^{10B.2} as explained in the definitions section above in the description of “first substituent group(s)”. In embodiments, when an R^{10B.2} substituent group is substituted, the R^{10B.2} substituent group is substituted with one or more third substituent groups denoted by R^{10B.3} as explained in the definitions section above in the description of “first substituent group(s)”. In the above embodiments, R^{10B}, R^{10B.1}, R^{10B.2}, and R^{10B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{10B}, R^{10B.1}, R^{10B.2}, and R^{10B.3}, respectively.

In embodiments, when R^{10C} is substituted, R^{10C} is substituted with one or more first substituent groups denoted by R^{10C.1} as explained in the definitions section above in the description of “first substituent group(s)”. In embodiments, when an R^{10C.1} substituent group is substituted, the R^{10C.1} substituent group is substituted with one or more second substituent groups denoted by R^{10C.2} as explained in the definitions section above in the description of “first substituent group(s)”. In embodiments, when an R^{10C.2} substituent group is substituted, the R^{10C.2} substituent group is substituted with one or more third substituent groups denoted by R^{10C.3} as explained in the definitions section above in the description of “first substituent group(s)”. In the above embodiments, R^{10C}, R^{10C.1}, R^{10C.2}, and R^{10C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{10C}, R^{10C.1}, R^{10C.2}, and R^{10C.3}, respectively.

In embodiments, when R^{10F} is substituted, R^{10F} is substituted with one or more first substituent groups denoted by R^{10F.1} as explained in the definitions section above in the description of “first substituent group(s)”. In embodiments, when an R^{10F.1} substituent group is substituted, the R^{10F.1} substituent group is substituted with one or more second substituent groups denoted by R^{10F.2} as explained in the definitions section above in the description of “first substituent group(s)”. In embodiments, when an R^{10F.2} substituent group is substituted, the R^{10F.2} substituent group is substituted with one or more third substituent groups denoted by R^{10F.3} as explained in the definitions section above in the description of “first substituent group(s)”. In the above embodiments, R^{10F}, R^{10F.1}, R^{10F.2}, and R^{10F.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{10F}, R^{10F.1}, R^{10F.2}, and R^{10F.3}, respectively.

In embodiments, when R^{11A} is substituted, R^{11A} is substituted with one or more first substituent groups denoted by R^{11A} as explained in the definitions section above in the description of “first substituent group(s)”. In embodiments, when an R^{11A.1} substituent group is substituted, the R^{11A.1} substituent group is substituted with one or more second substituent groups denoted by R^{11A.2} as explained in the definitions section above in the description of “first substituent group(s)”. In embodiments, when an R^{11A.2} substituent group is substituted, the R^{11A.2} substituent group is substituted with one or more third substituent groups denoted by R^{11A.3} as explained in the definitions section above in the description of “first substituent group(s)”. In the above embodiments, R^{11A}, R^{11A.1}, R^{11A.2}, and R^{11A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{11A}, R^{11A.1}, R^{11A.2}, and R^{11A.3}, respectively.

In embodiments, when R^{11B} is substituted, R^{11B} is substituted with one or more first substituent groups denoted by R^{11B.1} as explained in the definitions section above in the description of “first substituent group(s)”. In embodiments, when an R^{11B.1} substituent group is substituted, the R^{11B.1} substituent group is substituted with one or more second substituent groups denoted by R^{11B.2} as explained in the definitions section above in the description of “first substituent group(s)”. In embodiments, when an R^{11B.2} substituent group is substituted, the R^{11B.2} substituent group is substituted with one or more third substituent groups denoted by R^{11B.3} as explained in the definitions section above in the description of “first substituent group(s)”. In the above embodiments, R^{11B}, R^{11B.1}, R^{11B.2}, and R^{11B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{11B}, R^{11B.1}, R^{11B.2}, and R^{11B.3}, respectively.

In embodiments, when R^{11C} is substituted, R^{11C} is substituted with one or more first substituent groups denoted by R^{11C.1} as explained in the definitions section above in the description of “first substituent group(s)”. In embodiments, when an R^{11C.1} substituent group is substituted, the R^{11C.1} substituent group is substituted with one or more second substituent groups denoted by R^{11C.2} as explained in the definitions section above in the description of “first substituent group(s)”. In embodiments, when an R^{11C.2} substituent group is substituted, the R^{11C.2} substituent group is substituted with one or more third substituent groups denoted by R^{11C.3} as explained in the definitions section above in the description of “first substituent group(s)”. In the above embodiments, R^{11C}, R^{11C.1}, R^{11C.2}, and R^{11C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{11C}, R^{11C.1}, R^{11C.2}, and R^{11C.3}, respectively.

In embodiments, when R^{11F} is substituted, R^{11F} is substituted with one or more first substituent groups denoted by R^{11F.1} as explained in the definitions section above in the description of “first substituent group(s)”. In embodiments, when an R^{11F.1} substituent group is substituted, the R^{11F.1} substituent group is substituted with one or more second substituent groups denoted by R^{11F.2} as explained in the definitions section above in the description of “first substituent group(s)”. In embodiments, when an R^{11F.2} substituent group is substituted, the R^{11F.2} substituent group is substituted with one or more third substituent groups denoted by R^{11F.3} as explained in the definitions section above in the description of “first substituent group(s)”. In the above embodiments, R^{11F}, R^{11F.1}, R^{11F.2}, and R^{11F.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{11F}, R^{11F.1}, R^{11F.2}, and R^{11F.3}, respectively.

In embodiments, when R^{12A} is substituted, R^{12A} is substituted with one or more first substituent groups denoted by R^{12A.1} as explained in the definitions section above in the description of “first substituent group(s)”. In embodiments, when an R^{12A.1} substituent group is substituted, the R^{12A.1} substituent group is substituted with one or more second substituent groups denoted by R^{12A.2} as explained in the definitions section above in the description of “first substituent group(s)”. In embodiments, when an R^{12A.2} substituent group is substituted, the R^{12A.2} substituent group is substituted with one or more third substituent groups denoted by R^{12A.3} as explained in the definitions section above in the description of “first substituent group(s)”. In the above embodiments, R^{12A}, R^{12A.1}, R^{12A.2}, and R^{12A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{12A}, R^{12A.1}, R^{12A.2}, and R^{12A.3}, respectively.

In embodiments, when R^{12B} is substituted, R^{12B} is substituted with one or more first substituent groups denoted by R^{12B.1} as explained in the definitions section above in the description of “first substituent group(s)”. In embodiments, when an R^{12B.1} substituent group is substituted, the R^{12B.1} substituent group is substituted with one or more second substituent groups denoted by R^{12B.2} as explained in the definitions section above in the description of “first substituent group(s)”. In embodiments, when an R^{12B.2} substituent group is substituted, the R^{12B.2} substituent group is substituted with one or more third substituent groups denoted by R^{12B.3} as explained in the definitions section above in the description of “first substituent group(s)”. In the above embodiments, R^{12B}, R^{12B.1}, R^{12B.2}, and R^{12B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{12B}, R^{12B.1}, R^{12B.2}, and R^{12B.3}, respectively.

In embodiments, when R^{12C} is substituted, R^{12C} is substituted with one or more first substituent groups denoted by R^{12C.1} as explained in the definitions section above in the description of “first substituent group(s)”. In embodiments, when an R^{12C.1} substituent group is substituted, the R^{12C.1} substituent group is substituted with one or more second substituent groups denoted by R^{12C.2} as explained in the definitions section above in the description of “first substituent group(s)”. In embodiments, when an R^{12C.2} substituent group is substituted, the R^{12C.2} substituent group is substituted with one or more third substituent groups denoted by R^{12C.3} as explained in the definitions section above in the description of “first substituent group(s)”. In the above embodiments, R^{12C}, R^{12C.1}, R^{12C.2}, and R^{12C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{12C}, R^{12C.1}, R^{12C.2}, and R^{12C.3}, respectively.

In embodiments, when R^{12F} is substituted, R^{12F} is substituted with one or more first substituent groups denoted by R^{12F.1} as explained in the definitions section above in the description of “first substituent group(s)”. In embodiments, when an R^{12F.1} substituent group is substituted, the R^{12F.1} substituent group is substituted with one or more second substituent groups denoted by R^{12F.2} as explained in the definitions section above in the description of “first substituent group(s)”. In embodiments, when an R^{12F.2} substituent group is substituted, the R^{12F.2} substituent group is substituted with one or more third substituent groups denoted by R^{12F.3} as explained in the definitions section above in the description of “first substituent group(s)”. In the above embodiments, R^{12F}, R^{12F.1}, R^{12F.2}, and R^{12F.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{12F}, R^{12F.1}, R^{12F.2}, and R^{12F.3}, respectively.

In embodiments, when R^{13B} is substituted, R^{13B} is substituted with one or more first substituent groups denoted by R^{13B.1} as explained in the definitions section above in the description of “first substituent group(s)”. In embodiments, when an R^{13B.1} substituent group is substituted, the R^{13B.1} substituent group is substituted with one or more second substituent groups denoted by R^{13B.2} as explained in the definitions section above in the description of “first substituent group(s)”. In embodiments, when an R^{13B.2} substituent group is substituted, the R^{13B.2} substituent group is substituted with one or more third substituent groups denoted by R^{13B.3} as explained in the definitions section above in the description of “first substituent group(s)”. In the above embodiments, R^{13B}, R^{13B.1}, R^{13B.2}, and R^{13B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{13B}, R^{13B.1}, R^{13B.2}, and R^{13B.3}, respectively.

In embodiments, when R^{13C} is substituted, R^{13C} is substituted with one or more first substituent groups denoted by R^{13C.1} as explained in the definitions section above in the description of “first substituent group(s)”. In embodiments, when an R^{13C.1} substituent group is substituted, the R^{13C.1} substituent group is substituted with one or more second substituent groups denoted by R^{13C.2} as explained in the definitions section above in the description of “first substituent group(s)”. In embodiments, when an R^{13C.2} substituent group is substituted, the R^{13C.2} substituent group is substituted with one or more third substituent groups denoted by R^{13C.3} as explained in the definitions section above in the description of “first substituent group(s)”. In the above embodiments, R^{13C}, R^{13C.1}, R^{13C.2}, and R^{13C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{13C}, R^{13C.1}, R^{13C.2}, and R^{13C.3}, respectively.

In embodiments, when R^{13F} is substituted, R^{13F} is substituted with one or more first substituent groups denoted by R^{3F.1} as explained in the definitions section above in the description of “first substituent group(s)”. In embodiments, when an R^{3F.1} substituent group is substituted, the R^{3F.1} substituent group is substituted with one or more second substituent groups denoted by R^{13F.2} as explained in the definitions section above in the description of “first substituent group(s)”. In embodiments, when an R^{13F.2} substituent group is substituted, the R^{13F.2} substituent group is substituted with one or more third substituent groups denoted by R^{13F.3} as explained in the definitions section above in the description of “first substituent group(s)”. In the above embodiments, R^{13F}, R^{13F.1}, R^{13F.2}, and R^{13F.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{13F}, R^{13F.1}, R^{13F.2}, and R^{13F.3}, respectively.

In embodiments, when R^{14C} is substituted, R^{14C} is substituted with one or more first substituent groups denoted by R^{14C.1} as explained in the definitions section above in the description of “first substituent group(s)”. In embodiments, when an R^{14C.1} substituent group is substituted, the R^{14C.1} substituent group is substituted with one or more second substituent groups denoted by R^{14C.2} as explained in the definitions section above in the description of “first substituent group(s)”. In embodiments, when an R^{14C.2} substituent group is substituted, the R^{14C.2} substituent group is substituted with one or more third substituent groups denoted by R^{14C.3} as explained in the definitions section above in the description of “first substituent group(s)”. In the above embodiments, R^{14C}, R^{14C.1}, R^{14C.2}, and R^{14C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{14C}, R^{14C.1}, R^{14C.2}, and R^{14C.3}, respectively.

In embodiments, when R^{14F} is substituted, R^{14F} is substituted with one or more first substituent groups denoted by R^{14F.1} as explained in the definitions section above in the description of “first substituent group(s)”. In embodiments, when an R^{14F.1} substituent group is substituted, the R^{14F.1} substituent group is substituted with one or more second substituent groups denoted by R^{14F.2} as explained in the definitions section above in the description of “first substituent group(s)”. In embodiments, when an R^{14F.2} substituent group is substituted, the R^{14F.2} substituent group is substituted with one or more third substituent groups denoted by R^{14F.3} as explained in the definitions section above in the description of “first substituent group(s)”. In the above embodiments, R^{14F}, R^{14F.1}, R^{14F.2}, and R^{14F.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{14F}, R^{14F.1}, R^{14F.2}, and R^{14F.3}, respectively.

In embodiments, when R^{15C} is substituted, R^{15C} is substituted with one or more first substituent groups denoted by R^{15C.1} as explained in the definitions section above in the description of “first substituent group(s)”. In embodiments, when an R^{15C.1} substituent group is substituted, the R^{15C.1} substituent group is substituted with one or more second substituent groups denoted by R^{15C.2} as explained in the definitions section above in the description of “first substituent group(s)”. In embodiments, when an R^{15C.2} substituent group is substituted, the R^{15C.2} substituent group is substituted with one or more third substituent groups denoted by R^{15C.3} as explained in the definitions section above in the description of “first substituent group(s)”. In the above embodiments, R^{15C}, R^{15C.1}, R^{15C.2}, and R^{15C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{15C}, R^{15C.1}, R^{15C.2}, and R^{15C.3}, respectively.

In embodiments, when R^{15F} is substituted, R^{15F} is substituted with one or more first substituent groups denoted by R^{15F.1} as explained in the definitions section above in the description of “first substituent group(s)”. In embodiments, when an R^{15F.1} substituent group is substituted, the R^{15F.1} substituent group is substituted with one or more second substituent groups denoted by R^{15F.2} as explained in the definitions section above in the description of “first substituent group(s)”. In embodiments, when an R^{15F.2} substituent group is substituted, the R^{15F.2} substituent group is substituted with one or more third substituent groups denoted by R^{15F.3} as explained in the definitions section above in the description of “first substituent group(s)”. In the above embodiments, R^{15F}, R^{15F.1}, R^{15F.2}, and R^{15F.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{15F}, R^{15F.1}, R^{15F.2}, and R^{15F.3}, respectively.

In embodiments, when R¹⁷ is substituted, R¹⁷ is substituted with one or more first substituent groups denoted by R^{17.1} as explained in the definitions section above in the description of “first substituent group(s)”. In embodiments, when an R^{17.1} substituent group is substituted, the R^{17.1} substituent group is substituted with one or more second substituent groups denoted by R^{17.2} as explained in the definitions section above in the description of “first substituent group(s)”. In embodiments, when an R^{17.2} substituent group is substituted, the R^{17.2} substituent group is substituted with one or more third substituent groups denoted by R^{17.3} as explained in the definitions section above in the description of “first substituent group(s)”. In the above embodiments, R¹⁷, R^{17.1}, R^{17.2}, and R^{17.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹⁷, R^{17.1}, R^{17.2}, and R^{17.3}, respectively.

In embodiments, when R²⁶ is substituted, R²⁶ is substituted with one or more first substituent groups denoted by R^{26.1} as explained in the definitions section above in the description of “first substituent group(s)”. In embodiments, when an R^{26.1} substituent group is substituted, the R^{26.1} substituent group is substituted with one or more second substituent groups denoted by R^{26.2} as explained in the definitions section above in the description of “first substituent group(s)”. In embodiments, when an R^{26.2} substituent group is substituted, the R^{26.2} substituent group is substituted with one or more third substituent groups denoted by R^{26.3} as explained in the definitions section above in the description of “first substituent group(s)”. In the above embodiments, R²⁶, R^{26.1}, R^{26.2}, and R^{26.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R²⁶, R^{26.1}, R^{26.2}, and R^{26.3}, respectively.

In embodiments, when R²⁷ is substituted, R²⁷ is substituted with one or more first substituent groups denoted by R^{27.1} as explained in the definitions section above in the description of “first substituent group(s)”. In embodiments, when an R^{27.1} substituent group is substituted, the R^{27.1} substituent group is substituted with one or more second substituent groups denoted by R^{27.2} as explained in the definitions section above in the description of “first substituent group(s)”. In embodiments, when an R^{27.2} substituent group is substituted, the R^{27.2} substituent group is substituted with one or more third substituent groups denoted by R^{27.3} as explained in the definitions section above in the description of “first substituent group(s)”. In the above embodiments, R²⁷, R^{27.1}, R^{27.2}, and R^{27.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R²⁷, R^{27.1}, R^{27.2}, and R^{27.3}, respectively.

In embodiments, when R²⁸ is substituted, R²⁸ is substituted with one or more first substituent groups denoted by R^{28.1} as explained in the definitions section above in the description of “first substituent group(s)”. In embodiments, when an R^{28.1} substituent group is substituted, the R^{28.1} substituent group is substituted with one or more second substituent groups denoted by R^{28.2} as explained in the definitions section above in the description of “first substituent group(s)”. In embodiments, when an R^{28.2} substituent group is substituted, the R^{28.2} substituent group is substituted with one or more third substituent groups denoted by R^{28.3} as explained in the definitions section above in the description of “first substituent group(s)”. In the above embodiments, R²⁸, R^{28.1}, R^{28.2}, and R^{28.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R²⁸, R^{28.1}, R^{28.2}, and R^{28.3}, respectively.

In embodiments, when L^{1A} is substituted, L^{1A} is substituted with one or more first substituent groups denoted by R^{L1A.1} as explained in the definitions section above in the description of “first substituent group(s)”. In embodiments, when an R^{L1A.1} substituent group is substituted, the R^{L1A.1} substituent group is substituted with one or more second substituent groups denoted by R^{L1A.2} as explained in the definitions section above in the description of “first substituent group(s)”. In embodiments, when an R^{L1A.2} substituent group is substituted, the R^{L1A.2} substituent group is substituted with one or more third substituent groups denoted by R^{L1A.3} as explained in the definitions section above in the description of “first substituent group(s)”. In the above embodiments, L^{1A}, R^{L1A.1}, R^{L1A.2}, and R^{L1A.3} have values corresponding to the values of L^{WW}, R^{LWW.1}, R^{LWW.2} and R^{LWW.3}, respectively, as explained in the definitions section above in the description of “first substituent group(s)”, wherein L^{WW}, R^{LWW.1}, R^{LWW.2}, and R^{LWW.3} are L^{1A}, R^{L1A.1}, R^{L1A.2} and R^{L1A.3}, respectively.

In embodiments, when L^{1B} is substituted, L^{1B} is substituted with one or more first substituent groups denoted by R^{L1B.1} as explained in the definitions section above in the description of “first substituent group(s)”. In embodiments, when an R^{L1B.1} substituent group is substituted, the R^{L1B.1} substituent group is substituted with one or more second substituent groups denoted by R^{L1B.2} as explained in the definitions section above in the description of “first substituent group(s)”. In embodiments, when an R^{L1B.2} substituent group is substituted, the R^{L1B.2} substituent group is substituted with one or more third substituent groups denoted by R^{L1B.3} as explained in the definitions section above in the description of “first substituent group(s)”. In the above embodiments, L^{1B}, R^{L1B.1}, R^{L1B.2}, and R^{L1B.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{1B}, R^{L1B.1}, R^{L1B.2}, and R^{L1B.3}, respectively.

In embodiments, when LC is substituted, L^{1C} is substituted with one or more first substituent groups denoted by R^{L1C.1} as explained in the definitions section above in the description of “first substituent group(s)”. In embodiments, when an R^{L1C.1} substituent group is substituted, the R^{L1C.1} substituent group is substituted with one or more second substituent groups denoted by R^{L1C.2} as explained in the definitions section above in the description of “first substituent group(s)”. In embodiments, when an R^{L1C.2} substituent group is substituted, the R^{L1C.2} substituent group is substituted with one or more third substituent groups denoted by R^{L1C.3} as explained in the definitions section above in the description of “first substituent group(s)”. In the above embodiments, L^{1C}, R^{L1C.1}, R^{L1C.2}, and R^{L1C.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{1C}, R^{L1C.1}, R^{L1C.2}, and R^{L1C.3}, respectively.

In embodiments, when L^{1F} is substituted, L^{1F} is substituted with one or more first substituent groups denoted by R^{L1F.1} as explained in the definitions section above in the description of “first substituent group(s)”. In embodiments, when an R^{L1F.1} substituent group is substituted, the R^{L1F.1} substituent group is substituted with one or more second substituent groups denoted by R^{L1F.2} as explained in the definitions section above in the description of “first substituent group(s)”. In embodiments, when an R^{L1F.2} substituent group is substituted, the R^{L1F.2} substituent group is substituted with one or more third substituent groups denoted by R^{L1F.3} as explained in the definitions section above in the description of “first substituent group(s)”. In the above embodiments, L^{1F}, R^{L1F.1}, R^{L1F.2}, and R^{L1F.3} have values corresponding to the values of L^{WW}, R^{LWW.1}, R^{LWW.2} and R^{LWW.3}, respectively, as explained in the definitions section above in the description of “first substituent group(s)”, wherein L^{WW}, R^{LWW.1}, R^{LWW.2}, and R^{LWW.3} are L^{1F}, R^{L1F.1}, R^{L1F.2}, and R^{L1F.3}, respectively.

In embodiments, when L^{2A} is substituted, L^{2A} is substituted with one or more first substituent groups denoted by R^{L2A.1} as explained in the definitions section above in the description of “first substituent group(s)”. In embodiments, when an R^{L2A.1} substituent group is substituted, the R^{L2A.1} substituent group is substituted with one or more second substituent groups denoted by R^{L2A.2} as explained in the definitions section above in the description of “first substituent group(s)”. In embodiments, when an R^{L2A.2} substituent group is substituted, the R^{L2A.2} substituent group is substituted with one or more third substituent groups denoted by R^{L2A.3} as explained in the definitions section above in the description of “first substituent group(s)”. In the above embodiments, L^{2A}, R^{L2A.1}, R^{L2A.2}, and R^{L2A.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{2A}, R^{L2A.1}, R^{L2A.2}, and R^{L2A.3}, respectively.

In embodiments, when L^{2B} is substituted, L^{2B} is substituted with one or more first substituent groups denoted by R^{L2B.1} as explained in the definitions section above in the description of “first substituent group(s)”. In embodiments, when an R^{L2B.1} substituent group is substituted, the R^{L2B.1} substituent group is substituted with one or more second substituent groups denoted by R^{L2B.2} as explained in the definitions section above in the description of “first substituent group(s)”. In embodiments, when an R^{L2B.2} substituent group is substituted, the R^{L2B.2} substituent group is substituted with one or more third substituent groups denoted by R^{L2B.3} as explained in the definitions section above in the description of “first substituent group(s)”. In the above embodiments, L^{2B}, R^{L2B.1}, R^{L2B.2}, and R^{L2B.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{2B}, R^{L2B.1}, R^{L2B.2}, and R^{L2B.3}, respectively.

In embodiments, when L^{2C} is substituted, L^{2C} is substituted with one or more first substituent groups denoted by R^{L2C.1} as explained in the definitions section above in the description of “first substituent group(s)”. In embodiments, when an R^{L2C.1} substituent group is substituted, the R^{L2C.1} substituent group is substituted with one or more second substituent groups denoted by R^{L2C.2} as explained in the definitions section above in the description of “first substituent group(s)”. In embodiments, when an R^{L2C.2} substituent group is substituted, the R^{L2C.2} substituent group is substituted with one or more third substituent groups denoted by R^{L2C.3} as explained in the definitions section above in the description of “first substituent group(s)”. In the above embodiments, L^{2C}, R^{L2C.1}, R^{L2C.2}, and R^{L2C.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{2C}, R^{L2C.1}, R^{L2C.2}, and R^{L2C.3}, respectively.

In embodiments, when L^{2F} is substituted, L^{2F} is substituted with one or more first substituent groups denoted by R^{L2F.1} as explained in the definitions section above in the description of “first substituent group(s)”. In embodiments, when an R^{L2F.1} substituent group is substituted, the R^{L2F.1} substituent group is substituted with one or more second substituent groups denoted by R^{L2F.2} as explained in the definitions section above in the description of “first substituent group(s)”. In embodiments, when an R^{L2F.2} substituent group is substituted, the R^{L2F.2} substituent group is substituted with one or more third substituent groups denoted by R^{L2F.3} as explained in the definitions section above in the description of “first substituent group(s)”. In the above embodiments, L^{2F}, R^{L2F.1}, R^{L2F.2}, and R^{L2F.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{2F}, R^{L2F.1}, R^{L2F.2}, and R^{L2F.3}, respectively.

In embodiments, when L^{3A} is substituted, L^{3A} is substituted with one or more first substituent groups denoted by R^{L3A.1} as explained in the definitions section above in the description of “first substituent group(s)”. In embodiments, when an R^{L3A.1} substituent group is substituted, the R^{L3A.1} substituent group is substituted with one or more second substituent groups denoted by R^{L3A.2} as explained in the definitions section above in the description of “first substituent group(s)”. In embodiments, when an R^{L3A.2} substituent group is substituted, the R^{L3A.2} substituent group is substituted with one or more third substituent groups denoted by R^{L3A.3} as explained in the definitions section above in the description of “first substituent group(s)”. In the above embodiments, L^{3A}, R^{L3A.1}, R^{L3A.2}, and R^{L3A.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{3A}, R^{L3A.1}, R^{L3A.2} and R^{L3A.3}, respectively.

In embodiments, when L^{3B} is substituted, L^{3B} is substituted with one or more first substituent groups denoted by R^{L3B.1} as explained in the definitions section above in the description of “first substituent group(s)”. In embodiments, when an R^{L3B.1} substituent group is substituted, the R^{L3B.1} substituent group is substituted with one or more second substituent groups denoted by R^{L3B.2} as explained in the definitions section above in the description of “first substituent group(s)”. In embodiments, when an R^{L3B.2} substituent group is substituted, the R^{L3B.2} substituent group is substituted with one or more third substituent groups denoted by R^{L3B.3} as explained in the definitions section above in the description of “first substituent group(s)”. In the above embodiments, L^{3B}, R^{L3B.1}, R^{L3B.2}, and R^{L3B.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{3B}, R^{L3B.1}, R^{L3B.2}, and R^{L3B.3}, respectively.

In embodiments, when L^{3C} is substituted, L^{3C} is substituted with one or more first substituent groups denoted by R^{L3C.1} as explained in the definitions section above in the description of “first substituent group(s)”. In embodiments, when an R^{L3C.1} substituent group is substituted, the R^{L3C.1} substituent group is substituted with one or more second substituent groups denoted by R^{L3C.2} as explained in the definitions section above in the description of “first substituent group(s)”. In embodiments, when an R^{L3C.2} substituent group is substituted, the R^{L3C.2} substituent group is substituted with one or more third substituent groups denoted by R^{L3C.3} as explained in the definitions section above in the description of “first substituent group(s)”. In the above embodiments, L^{3C}, R^{L3C.1}, R^{L3C.2}, and R^{L3C.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{3C}, R^{L3C.1}, R^{L3C.2}, and R^{L3C.3}, respectively.

In embodiments, when L^{3F} is substituted, L^{3F} is substituted with one or more first substituent groups denoted by R^{L3F.1} as explained in the definitions section above in the description of “first substituent group(s)”. In embodiments, when an R^{L3F.1} substituent group is substituted, the R^{L3F.1} substituent group is substituted with one or more second substituent groups denoted by R^{L3F.2} as explained in the definitions section above in the description of “first substituent group(s)”. In embodiments, when an R^{L3F.2} substituent group is substituted, the R^{L3F.2} substituent group is substituted with one or more third substituent groups denoted by R^{L3F.3} as explained in the definitions section above in the description of “first substituent group(s)”. In the above embodiments, L^{3F}, R^{L3F.1}, R^{L3F.2}, and R^{L3F.3} have values corresponding to the values of L^{WW}, R^{LWW.1}, R^{LWW.2} and R^{LWW.3}, respectively, as explained in the definitions section above in the description of “first substituent group(s)”, wherein L^{WW}, R^{LWW.1}, R^{LWW.2}, and R^{LWW.3} are L^{3F}, R^{L3F.1}, R^{L3F.2}, and R^{L3F.3}, respectively.

In embodiments, when L^{4A} is substituted, L^{4A} is substituted with one or more first substituent groups denoted by R^{L4A.1} as explained in the definitions section above in the description of “first substituent group(s)”. In embodiments, when an R^{L4A.1} substituent group is substituted, the R^{L4A.1} substituent group is substituted with one or more second substituent groups denoted by R^{L4A.2} as explained in the definitions section above in the description of “first substituent group(s)”. In embodiments, when an R^{L4A.2} substituent group is substituted, the R^{L4A.2} substituent group is substituted with one or more third substituent groups denoted by R^{L4A.3} as explained in the definitions section above in the description of “first substituent group(s)”. In the above embodiments, L^{4A}, R^{L4A.1}, R^{L4A.2}, and R^{L4A.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{4A}, R^{L4A.1}, R^{L4A.2}, and R^{L4A.3}, respectively.

In embodiments, when L^{4B} is substituted, L^{4B} is substituted with one or more first substituent groups denoted by R^{L4B.1} as explained in the definitions section above in the description of “first substituent group(s)”. In embodiments, when an R^{L4B.1} substituent group is substituted, the R^{L4B.1} substituent group is substituted with one or more second substituent groups denoted by R^{L4B.2} as explained in the definitions section above in the description of “first substituent group(s)”. In embodiments, when an R^{L4B.2} substituent group is substituted, the R^{L4B.2} substituent group is substituted with one or more third substituent groups denoted by R^{L4B.3} as explained in the definitions section above in the description of “first substituent group(s)”. In the above embodiments, L^{4B}, R^{L4B.1}, R^{L4B.2}, and R^{L4B.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{4B}, R^{L4B.1}, R^{L4B.2}, and R^{L4B.3}, respectively.

In embodiments, when L^{4C} is substituted, L^{4C} is substituted with one or more first substituent groups denoted by R^{L4C.1} as explained in the definitions section above in the description of “first substituent group(s)”. In embodiments, when an R^{L4C.1} substituent group is substituted, the R^{L4C.1} substituent group is substituted with one or more second substituent groups denoted by R^{L4C.2} as explained in the definitions section above in the description of “first substituent group(s)”. In embodiments, when an R^{L4C.2} substituent group is substituted, the R^{L4C.2} substituent group is substituted with one or more third substituent groups denoted by R^{L4C.3} as explained in the definitions section above in the description of “first substituent group(s)”. In the above embodiments, L^{4C}, R^{L4C.1}, R^{L4C.2}, and R^{L4C.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{4C}, R^{L4C.1}, R^{L4C.2}, and R^{L4C.3}, respectively.

In embodiments, when L^{4F} is substituted, L^{4F} is substituted with one or more first substituent groups denoted by R^{L4F.1} as explained in the definitions section above in the description of “first substituent group(s)”. In embodiments, when an R^{L4F.1} substituent group is substituted, the R^{L4F.1} substituent group is substituted with one or more second substituent groups denoted by R^{L4F.2} as explained in the definitions section above in the description of “first substituent group(s)”. In embodiments, when an R^{L4F.2} substituent group is substituted, the R^{L4F.2} substituent group is substituted with one or more third substituent groups denoted by R^{L4F.3} as explained in the definitions section above in the description of “first substituent group(s)”. In the above embodiments, L^{4F}, R^{L4F.1}, R^{L4F.2}, and R^{L4F.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{4F}, R^{L4F.1}, R^{L4F.2}, and R^{L4F.3}, respectively.

In embodiments, when L^{5A} is substituted, L^{5A} is substituted with one or more first substituent groups denoted by R^{L5A.1} as explained in the definitions section above in the description of “first substituent group(s)”. In embodiments, when an R^{L5A.1} substituent group is substituted, the R^{L5A.1} substituent group is substituted with one or more second substituent groups denoted by R^{L5A.2} as explained in the definitions section above in the description of “first substituent group(s)”. In embodiments, when an R^{L5A.2} substituent group is substituted, the R^{L5A.2} substituent group is substituted with one or more third substituent groups denoted by R^{L5A.3} as explained in the definitions section above in the description of “first substituent group(s)”. In the above embodiments, L^{5A}, R^{L5A.1}, R^{L5A.2}, and R^{L5A.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{5A}, R^{L5A.1}, R^{L5A.2} and R^{L5A.3}, respectively.

In embodiments, when L^{5B} is substituted, L^{5B} is substituted with one or more first substituent groups denoted by R^{L5B.1} as explained in the definitions section above in the description of “first substituent group(s)”. In embodiments, when an R^{L5B.1} substituent group is substituted, the R^{L5B.1} substituent group is substituted with one or more second substituent groups denoted by R^{L5B.2} as explained in the definitions section above in the description of “first substituent group(s)”. In embodiments, when an R^{L5B.2} substituent group is substituted, the R^{L5B.2} substituent group is substituted with one or more third substituent groups denoted by R^{L5B.3} as explained in the definitions section above in the description of “first substituent group(s)”. In the above embodiments, L^{5B}, R^{L5B.1}, R^{L5B.2}, and R^{L5B.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{5B}, R^{L5B.1}, R^{L5B.2}, and R^{L5B.3}, respectively.

In embodiments, when L^{5C} is substituted, L^{5C} is substituted with one or more first substituent groups denoted by R^{L5C.1} as explained in the definitions section above in the description of “first substituent group(s)”. In embodiments, when an R^{L5C.1} substituent group is substituted, the R^{L5C.1} substituent group is substituted with one or more second substituent groups denoted by R^{L5C.2} as explained in the definitions section above in the description of “first substituent group(s)”. In embodiments, when an R^{L5C.2} substituent group is substituted, the R^{L5C.2} substituent group is substituted with one or more third substituent groups denoted by R^{L5C.3} as explained in the definitions section above in the description of “first substituent group(s)”. In the above embodiments, L^{5C}, R^{L5C.1}, R^{L5C.2}, and R^{L5C.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{5C}, R^{L5C.1}, R^{L5C.2}, and R^{L5C.3}, respectively.

In embodiments, when L^{5F} is substituted, L^{5F} is substituted with one or more first substituent groups denoted by R^{L5F.1} as explained in the definitions section above in the description of “first substituent group(s)”. In embodiments, when an R^{L5F.1} substituent group is substituted, the R^{L5F.1} substituent group is substituted with one or more second substituent groups denoted by R^{L5F.2} as explained in the definitions section above in the description of “first substituent group(s)”. In embodiments, when an R^{L5F.2} substituent group is substituted, the R^{L5F.2} substituent group is substituted with one or more third substituent groups denoted by R^{L5F.3} as explained in the definitions section above in the description of “first substituent group(s)”. In the above embodiments, L^{5F}, R^{L5F.1}, R^{L5F.2}, and R^{L5F.3} have values corresponding to the values of L^{WW}, R^{LWW.1}, R^{LWW.2} and R^{LWW.3}, respectively, as explained in the definitions section above in the description of “first substituent group(s)”, wherein L^{WW}, R^{LWW.1}, R^{LWW.2}, and R^{LWW.3} are L^{5F}, R^{L5F.1}, R^{L5F.2}, and R^{L5F.3}, respectively.

In embodiments, when L^{6A} is substituted, L^{6A} is substituted with one or more first substituent groups denoted by R^{L6A.1} as explained in the definitions section above in the description of “first substituent group(s)”. In embodiments, when an R^{L6A.1} substituent group is substituted, the R^{L6A.1} substituent group is substituted with one or more second substituent groups denoted by R^{L6A.2} as explained in the definitions section above in the description of “first substituent group(s)”. In embodiments, when an R^{L6A.2} substituent group is substituted, the R^{L6A.2} substituent group is substituted with one or more third substituent groups denoted by R^{L6A.3} as explained in the definitions section above in the description of “first substituent group(s)”. In the above embodiments, L^{6A}, R^{L6A.1}, R^{L6A.2}, and R^{L6A.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{6A}, R^{L6A.1}, R^{L6A.2}, and R^{L6A.3}, respectively.

In embodiments, when L^{6B} is substituted, L^{6B} is substituted with one or more first substituent groups denoted by R^{L6B.1} as explained in the definitions section above in the description of “first substituent group(s)”. In embodiments, when an R^{L6B.1} substituent group is substituted, the R^{L6B.1} substituent group is substituted with one or more second substituent groups denoted by R^{L6B.2} as explained in the definitions section above in the description of “first substituent group(s)”. In embodiments, when an R^{L6B.2} substituent group is substituted, the R^{L6B.2} substituent group is substituted with one or more third substituent groups denoted by R^{L6B.3} as explained in the definitions section above in the description of “first substituent group(s)”. In the above embodiments, L^{6B}, R^{L6B.1}, R^{L6B.2}, and R^{L6B.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{6B}, R^{L6B.1}, R^{L6B.2}, and R^{L6B.3}, respectively.

In embodiments, when L^{6C} is substituted, L^{6C} is substituted with one or more first substituent groups denoted by R^{L6C.1} as explained in the definitions section above in the description of “first substituent group(s)”. In embodiments, when an R^{L6C.1} substituent group is substituted, the R^{L6C.1} substituent group is substituted with one or more second substituent groups denoted by R^{L6C.2} as explained in the definitions section above in the description of “first substituent group(s)”. In embodiments, when an R^{L6C.2} substituent group is substituted, the R^{L6C.2} substituent group is substituted with one or more third substituent groups denoted by R^{L6C.3} as explained in the definitions section above in the description of “first substituent group(s)”. In the above embodiments, L^{6C}, R^{L6C.1}, R^{L6C.2}, and R^{L6C.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{6C}, R^{L6C.1}, R^{L6C.2}, and R^{L6C.3}, respectively.

In embodiments, when L^{6F} is substituted, L^{6F} is substituted with one or more first substituent groups denoted by R^{L6F.1} as explained in the definitions section above in the description of “first substituent group(s)”. In embodiments, when an R^{L6F.1} substituent group is substituted, the R^{L6F.1} substituent group is substituted with one or more second substituent groups denoted by R^{L6F.2} as explained in the definitions section above in the description of “first substituent group(s)”. In embodiments, when an R^{L6F.2} substituent group is substituted, the R^{L6F.2} substituent group is substituted with one or more third substituent groups denoted by R^{L6F.3} as explained in the definitions section above in the description of “first substituent group(s)”. In the above embodiments, L^{6F}, R^{L6F.1}, R^{L6F.2}, and R^{L6F.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{6F}, R^{L6F.1}, R^{L6F.2}, and R^{L6F.3}, respectively.

In embodiments, when L^{7A} is substituted, L^{7A} is substituted with one or more first substituent groups denoted by R^{L7A.1} as explained in the definitions section above in the description of “first substituent group(s)”. In embodiments, when an R^{L7A.1} substituent group is substituted, the R^{L7A.1} substituent group is substituted with one or more second substituent groups denoted by R^{L7A.2} as explained in the definitions section above in the description of “first substituent group(s)”. In embodiments, when an R^{L7A.2} substituent group is substituted, the R^{L7A.2} substituent group is substituted with one or more third substituent groups denoted by R^{L7A.3} as explained in the definitions section above in the description of “first substituent group(s)”. In the above embodiments, L^{7A}, R^{L7A.1}, R^{L7A.2}, and R^{L7A.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{7A}, R^{L7A.1}, R^{L7A.2} and R^{L7A.3}, respectively.

In embodiments, when L^{7B} is substituted, L^{7B} is substituted with one or more first substituent groups denoted by R^{L7B.1} as explained in the definitions section above in the description of “first substituent group(s)”. In embodiments, when an R^{L7B.1} substituent group is substituted, the R^{L7B.1} substituent group is substituted with one or more second substituent groups denoted by R^{L7B.2} as explained in the definitions section above in the description of “first substituent group(s)”. In embodiments, when an R^{L7B.2} substituent group is substituted, the R^{L7B.2} substituent group is substituted with one or more third substituent groups denoted by R^{L7B.3} as explained in the definitions section above in the description of “first substituent group(s)”. In the above embodiments, L^{7B}, R^{L7B.1}, R^{L7B.2}, and R^{L7B.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{7B}, R^{L7B.1}, R^{L7B.2}, and R^{L7B.3}, respectively.

In embodiments, when L^{7C} is substituted, L^{7C} is substituted with one or more first substituent groups denoted by R^{L7C.1} as explained in the definitions section above in the description of “first substituent group(s)”. In embodiments, when an R^{L7C.1} substituent group is substituted, the R^{L7C.1} substituent group is substituted with one or more second substituent groups denoted by R^{L7C.2} as explained in the definitions section above in the description of “first substituent group(s)”. In embodiments, when an R^{L7C.2} substituent group is substituted, the R^{L7C.2} substituent group is substituted with one or more third substituent groups denoted by R^{L7C.3} as explained in the definitions section above in the description of “first substituent group(s)”. In the above embodiments, L^{7C}, R^{L7C.1}, R^{L7C.2}, and R^{L7C.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{7C}, R^{L7C.1}, R^{L7C.2}, and R^{L7C.3}, respectively.

In embodiments, when L^{7F} is substituted, L^{7F} is substituted with one or more first substituent groups denoted by R^{L7F.1} as explained in the definitions section above in the description of “first substituent group(s)”. In embodiments, when an R^{L7F.1} substituent group is substituted, the R^{L7F.1} substituent group is substituted with one or more second substituent groups denoted by R^{L7F.2} as explained in the definitions section above in the description of “first substituent group(s)”. In embodiments, when an R^{L7F.2} substituent group is substituted, the R^{L7F.2} substituent group is substituted with one or more third substituent groups denoted by R^{L7F.3} as explained in the definitions section above in the description of “first substituent group(s)”. In the above embodiments, L^{7F}, R^{L7F.1}, R^{L7F.2}, and R^{L7F.3} have values corresponding to the values of L^{WW}, R^{LWW.1}, R^{LWW.2} and R^{LWW.3}, respectively, as explained in the definitions section above in the description of “first substituent group(s)”, wherein L^{WW}, R^{LWW.1}, R^{LWW.2}, and R^{LWW.3} are L^{7F}, R^{L7F.1}, R^{L7F.2}, and R^{L7F.3}, respectively.

In embodiments, when L^{8A} is substituted, L^{8A} is substituted with one or more first substituent groups denoted by R^{L8A.1} as explained in the definitions section above in the description of “first substituent group(s)”. In embodiments, when an R^{L8A.1} substituent group is substituted, the R^{L8A.1} substituent group is substituted with one or more second substituent groups denoted by R^{L8A.2} as explained in the definitions section above in the description of “first substituent group(s)”. In embodiments, when an R^{L8A.2} substituent group is substituted, the R^{L8A.2} substituent group is substituted with one or more third substituent groups denoted by R^{L8A.3} as explained in the definitions section above in the description of “first substituent group(s)”. In the above embodiments, L^{8A}, R^{L8A.1}, R^{L8A.2}, and R^{L8A.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{8A}, R^{L8A.1}, R^{L8A.2}, and R^{L8A.3}, respectively.

In embodiments, when L^{8B} is substituted, L^{8B} is substituted with one or more first substituent groups denoted by R^{L8B.1} as explained in the definitions section above in the description of “first substituent group(s)”. In embodiments, when an R^{L8B.1} substituent group is substituted, the R^{L8B.1} substituent group is substituted with one or more second substituent groups denoted by R^{L8B.2} as explained in the definitions section above in the description of “first substituent group(s)”. In embodiments, when an R^{L8B.2} substituent group is substituted, the R^{L8B.2} substituent group is substituted with one or more third substituent groups denoted by R^{L8B.3} as explained in the definitions section above in the description of “first substituent group(s)”. In the above embodiments, L^{8B}, R^{L8B.1}, R^{L8B.2}, and R^{L8B.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{8B}, R^{L8B.1}, R^{L8B.2}, and R^{L8B.3}, respectively.

In embodiments, when L^{8C} is substituted, L^{8C} is substituted with one or more first substituent groups denoted by R^{L8C.1} as explained in the definitions section above in the description of “first substituent group(s)”. In embodiments, when an R^{L1C.1} substituent group is substituted, the R^{L8C.1} substituent group is substituted with one or more second substituent groups denoted by R^{L8C.2} as explained in the definitions section above in the description of “first substituent group(s)”. In embodiments, when an R^{L8C.2} substituent group is substituted, the R^{L8C.2} substituent group is substituted with one or more third substituent groups denoted by R^{L8C.3} as explained in the definitions section above in the description of “first substituent group(s)”. In the above embodiments, L^{8C}, R^{L8C.1}, R^{L8C.2}, and R^{L8C.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{8C}, R^{L8C.1}, R^{L8C.2}, and R^{L8C.3}, respectively.

In embodiments, when L^{8F} is substituted, L^{8F} is substituted with one or more first substituent groups denoted by R^{L8F.1} as explained in the definitions section above in the description of “first substituent group(s)”. In embodiments, when an R^{L8F.1} substituent group is substituted, the R^{L8F.1} substituent group is substituted with one or more second substituent groups denoted by R^{L8F.2} as explained in the definitions section above in the description of “first substituent group(s)”. In embodiments, when an R^{L8F.2} substituent group is substituted, the R^{L8F.2} substituent group is substituted with one or more third substituent groups denoted by R^{L8F.3} as explained in the definitions section above in the description of “first substituent group(s)”. In the above embodiments, L^{8F}, R^{L8F.1}, R^{L8F.2}, and R^{L8F.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{8F}, R^{L8F.1}, R^{L8F.2}, and R^{L8F.3}, respectively.

In embodiments, when L^{9A} is substituted, L^{9A} is substituted with one or more first substituent groups denoted by R^{L9A.1} as explained in the definitions section above in the description of “first substituent group(s)”. In embodiments, when an R^{L9A.1} substituent group is substituted, the R^{L9A.1} substituent group is substituted with one or more second substituent groups denoted by R^{L9A.2} as explained in the definitions section above in the description of “first substituent group(s)”. In embodiments, when an R^{L9A.2} substituent group is substituted, the R^{L9A.2} substituent group is substituted with one or more third substituent groups denoted by R^{L9A.3} as explained in the definitions section above in the description of “first substituent group(s)”. In the above embodiments, L^{9A}, R^{L9A.1}, R^{L9A.2}, and R^{L9A.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{9A}, R^{L9A.1}, R^{L9A.2} and R^{L9A.3}, respectively.

In embodiments, when L^{9B} is substituted, L^{9B} is substituted with one or more first substituent groups denoted by R^{L9B.1} as explained in the definitions section above in the description of “first substituent group(s)”. In embodiments, when an R^{L9B.1} substituent group is substituted, the R^{L9B.1} substituent group is substituted with one or more second substituent groups denoted by R^{L9B.2} as explained in the definitions section above in the description of “first substituent group(s)”. In embodiments, when an R^{L9B.2} substituent group is substituted, the R^{L9B.2} substituent group is substituted with one or more third substituent groups denoted by R^{L9B.3} as explained in the definitions section above in the description of “first substituent group(s)”. In the above embodiments, L^{9B}, R^{L9B.1}, R^{L9B.2}, and R^{L9B.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{9B}, R^{L9B.1}, R^{L9B.2}, and R^{L9B.3}, respectively.

In embodiments, when L^{9C} is substituted, L^{9C} is substituted with one or more first substituent groups denoted by R^{L9C.1} as explained in the definitions section above in the description of “first substituent group(s)”. In embodiments, when an R^{L9C.1} substituent group is substituted, the R^{L9C.1} substituent group is substituted with one or more second substituent groups denoted by R^{L9C.2} as explained in the definitions section above in the description of “first substituent group(s)”. In embodiments, when an R^{L9C.2} substituent group is substituted, the R^{L9C.2} substituent group is substituted with one or more third substituent groups denoted by R^{L9C.3} as explained in the definitions section above in the description of “first substituent group(s)”. In the above embodiments, L^{9C}, R^{L9C.1}, R^{L9C.2}, and R^{L9C.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{9C}, R^{L9C.1}, R^{L9C.2}, and R^{L9C.3}, respectively.

In embodiments, when L^{9F} is substituted, L^{9F} is substituted with one or more first substituent groups denoted by R^{L9F.1} as explained in the definitions section above in the description of “first substituent group(s)”. In embodiments, when an R^{L9F.1} substituent group is substituted, the R^{L9F.1} substituent group is substituted with one or more second substituent groups denoted by R^{L9F.2} as explained in the definitions section above in the description of “first substituent group(s)”. In embodiments, when an R^{L9F.2} substituent group is substituted, the R^{L9F.2} substituent group is substituted with one or more third substituent groups denoted by R^{L9F.3} as explained in the definitions section above in the description of “first substituent group(s)”. In the above embodiments, L^{9F}, R^{L9F.1}, R^{L9F.2}, and R^{L9F.3} have values corresponding to the values of L^{WW}, R^{LWW.1}, R^{LWW.2} and R^{LWW.3}, respectively, as explained in the definitions section above in the description of “first substituent group(s)”, wherein L^{WW}, R^{LWW.1}, R^{LWW.2}, and R^{LWW.3} are L^{9F}, R^{L9F.1}, R^{L9F.2}, and R^{L9F.3}, respectively.

In embodiments, when L^{10A} is substituted, L^{10A} is substituted with one or more first substituent groups denoted by R^{L10A.1} as explained in the definitions section above in the description of “first substituent group(s)”. In embodiments, when an R^{L10A.1} substituent group is substituted, the R^{L10A.1} substituent group is substituted with one or more second substituent groups denoted by R^{L10A.2} as explained in the definitions section above in the description of “first substituent group(s)”. In embodiments, when an R^{L10A.2} substituent group is substituted, the R^{L10A.2} substituent group is substituted with one or more third substituent groups denoted by R^{L10A.3} as explained in the definitions section above in the description of “first substituent group(s)”. In the above embodiments, L^{10A}, R^{L10A.1}, R^{L10A.2}, and R^{L10A.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{10A}, R^{L10A.1}, R^{L10A.2}, and R^{L10A.3}, respectively.

In embodiments, when L^{10B} is substituted, L^{10B} is substituted with one or more first substituent groups denoted by R^{L10B.1} as explained in the definitions section above in the description of “first substituent group(s)”. In embodiments, when an R^{L10B.1} substituent group is substituted, the R^{L10B.1} substituent group is substituted with one or more second substituent groups denoted by R^{L10B.2} as explained in the definitions section above in the description of “first substituent group(s)”. In embodiments, when an R^{L10B.2} substituent group is substituted, the R^{L10B.2} substituent group is substituted with one or more third substituent groups denoted by R^{L10B.3} as explained in the definitions section above in the description of “first substituent group(s)”. In the above embodiments, L^{10B}, R^{L10B.1}, R^{L10B.2} and R^{L10B.3} have values corresponding to the values of L^{WW}, R^{LWW.1}, R^{LWW.2} and R^{LWW.3}, respectively, as explained in the definitions section above in the description of “first substituent group(s)”, wherein L^{WW}, R^{LWW.1}, R^{LWW.2}, and R^{LWW.3} are L^{10B}, R^{L10B.1}, R^{L10B.2}, and R^{L10B.3}, respectively.

In embodiments, when L^{10C} is substituted, L^{10C} is substituted with one or more first substituent groups denoted by R^{L10C.1} as explained in the definitions section above in the description of “first substituent group(s)”. In embodiments, when an R^{L10C.1} substituent group is substituted, the R^{L10C.1} substituent group is substituted with one or more second substituent groups denoted by R^{L10C.2} as explained in the definitions section above in the description of “first substituent group(s)”. In embodiments, when an R^{L10C.2} substituent group is substituted, the R^{L10C.2} substituent group is substituted with one or more third substituent groups denoted by R^{L10C.3} as explained in the definitions section above in the description of “first substituent group(s)”. In the above embodiments, L^{10C}, R^{L10C.1}, R^{L10C.2}, and R^{L10C.3} have values corresponding to the values of L^{WW}, R^{LWW.1}, R^{LWW.2} and R^{LWW.3}, respectively, as explained in the definitions section above in the description of “first substituent group(s)”, wherein L^{WW}, R^{LWW.1}, R^{LWW.2}, and R^{LWW.3} are L^{10C}, R^{L10C.1}, R^{L10C.2}, and R^{L10C.3}, respectively.

In embodiments, when L^{10D} is substituted, L^{10D} is substituted with one or more first substituent groups denoted by R^{L10D.1} as explained in the definitions section above in the description of “first substituent group(s)”. In embodiments, when an R^{L10D.1} substituent group is substituted, the R^{L10D.1} substituent group is substituted with one or more second substituent groups denoted by R^{L10D.2} as explained in the definitions section above in the description of “first substituent group(s)”. In embodiments, when an R^{L10D.2} substituent group is substituted, the R^{L10D.2} substituent group is substituted with one or more third substituent groups denoted by R^{L10D.3} as explained in the definitions section above in the description of “first substituent group(s)”. In the above embodiments, L^{10D}, R^{L10D.1}, R^{L10D.2}, and R^{L10D.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{10D}, R^{L10D.1}, R^{L10D.2}, and R^{L10D.3}, respectively.

In embodiments, when L^{10E} is substituted, L^{10E} is substituted with one or more first substituent groups denoted by R^{L10E.1} as explained in the definitions section above in the description of “first substituent group(s)”. In embodiments, when an R^{L10E.1} substituent group is substituted, the R^{L10E.1} substituent group is substituted with one or more second substituent groups denoted by R^{L10E.2} as explained in the definitions section above in the description of “first substituent group(s)”. In embodiments, when an R^{L10E.2} substituent group is substituted, the R^{L10E.2} substituent group is substituted with one or more third substituent groups denoted by R^{L10E.3} as explained in the definitions section above in the description of “first substituent group(s)”. In the above embodiments, L^{10E}, R^{L10E.1}, R^{L10E.2}, and R^{L10E.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{10E}, R^{L10E.1}, R^{L10E.2} and R^{L10E.3}, respectively.

In embodiments, when L^{10F} is substituted, L^{10F} is substituted with one or more first substituent groups denoted by R^{L10F.1} as explained in the definitions section above in the description of “first substituent group(s)”. In embodiments, when an R^{L10F.1} substituent group is substituted, the R^{L10F.1} substituent group is substituted with one or more second substituent groups denoted by R^{L10F.2} as explained in the definitions section above in the description of “first substituent group(s)”. In embodiments, when an R^{L10F.2} substituent group is substituted, the R^{L10F.2} substituent group is substituted with one or more third substituent groups denoted by R^{L10F.3} as explained in the definitions section above in the description of “first substituent group(s)”. In the above embodiments, L^{10F}, R^{L10F.1}, R^{L10F.2}, and R^{L10F.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{10F}, R^{L10F.1}, R^{L10F.2} and R^{L10F.3}, respectively.

In embodiments, when L^{11A} is substituted, L^{11A} is substituted with one or more first substituent groups denoted by R^{L11A.1} as explained in the definitions section above in the description of “first substituent group(s)”. In embodiments, when an R^{L11A.1} substituent group is substituted, the R^{L11A.1} substituent group is substituted with one or more second substituent groups denoted by R^{L11A.2} as explained in the definitions section above in the description of “first substituent group(s)”. In embodiments, when an R^{L11A.2} substituent group is substituted, the R^{L11A.2} substituent group is substituted with one or more third substituent groups denoted by R^{L11A.3} as explained in the definitions section above in the description of “first substituent group(s)”. In the above embodiments, L^{11A}, R^{L11A.1}, R^{L11A.2}, and R^{L11A.3} have values corresponding to the values of L^{WW}, R^{LWW.1}, R^{LWW.2} and R^{LWW.3}, respectively, as explained in the definitions section above in the description of “first substituent group(s)”, wherein L^{WW}, R^{LWW.1}, R^{LWW.2}, and R^{LWW.3} are L^{11A}, R^{L11A.1}, R^{L11A.2}, and R^{L11A.3}, respectively.

In embodiments, when L^{11B} is substituted, L^{11B} is substituted with one or more first substituent groups denoted by R^{L11B.1} as explained in the definitions section above in the description of “first substituent group(s)”. In embodiments, when an R^{L11B.1} substituent group is substituted, the R^{L11B.1} substituent group is substituted with one or more second substituent groups denoted by R^{L11B.2} as explained in the definitions section above in the description of “first substituent group(s)”. In embodiments, when an R^{L11B.2} substituent group is substituted, the R^{L11B.2} substituent group is substituted with one or more third substituent groups denoted by R^{L11B.3} as explained in the definitions section above in the description of “first substituent group(s)”. In the above embodiments, L^{11B}, R^{L11B.1}, R^{L11B.2}, and R^{L11B.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{11B}, R^{L11B.1}, R^{L11B.2}, and R^{L11B.3}, respectively.

In embodiments, when L^{11C} is substituted, L^{11C} is substituted with one or more first substituent groups denoted by R^{L11C.1} as explained in the definitions section above in the description of “first substituent group(s)”. In embodiments, when an R^{L11C.1} substituent group is substituted, the R^{L11C.1} substituent group is substituted with one or more second substituent groups denoted by R^{L11C.2} as explained in the definitions section above in the description of “first substituent group(s)”. In embodiments, when an R^{L11C.2} substituent group is substituted, the R^{L11C.2} substituent group is substituted with one or more third substituent groups denoted by R^{L11C.3} as explained in the definitions section above in the description of “first substituent group(s)”. In the above embodiments, L^{11C}, R^{L11C.1}, R^{L11C.2}, and R^{L11C.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{11C}, R^{11C.1}, R^{L11C.2} and R^{L11C.3}, respectively.

In embodiments, when L^{11F} is substituted, L^{11F} is substituted with one or more first substituent groups denoted by R^{L11F.1} as explained in the definitions section above in the description of “first substituent group(s)”. In embodiments, when an R^{L11F.1} substituent group is substituted, the R^{L11F.1} substituent group is substituted with one or more second substituent groups denoted by R^{L11F.2} as explained in the definitions section above in the description of “first substituent group(s)”. In embodiments, when an R^{L11F.2} substituent group is substituted, the R^{L11F.2} substituent group is substituted with one or more third substituent groups denoted by R^{L11F.3} as explained in the definitions section above in the description of “first substituent group(s)”. In the above embodiments, L^{11F}, R^{L11F.1}, R^{L11F.2}, and R^{L11F.3} have values corresponding to the values of L^{WW}, R^{LWW.1}, R^{LWW.2} and R^{LWW.3}, respectively, as explained in the definitions section above in the description of “first substituent group(s)”, wherein L^{WW}, R^{LWW.1}, R^{LWW.2}, and R^{LWW.3} are L^{11F}, R^{L11F.1}, R^{L11F.2}, and R^{L11F.3}, respectively.

In embodiments, when L^{12A} is substituted, L^{12A} is substituted with one or more first substituent groups denoted by R^{L12A.1} as explained in the definitions section above in the description of “first substituent group(s)”. In embodiments, when an R^{L12A.1} substituent group is substituted, the R^{L12A.1} substituent group is substituted with one or more second substituent groups denoted by R^{L12A.2} as explained in the definitions section above in the description of “first substituent group(s)”. In embodiments, when an R^{L12A.2} substituent group is substituted, the R^{L12A.2} substituent group is substituted with one or more third substituent groups denoted by R^{L12A.3} as explained in the definitions section above in the description of “first substituent group(s)”. In the above embodiments, L^{12A}, R^{L12A.1}, R^{L12A.2}, and R^{L12A.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{12A}, R^{L12A.1}, R^{L12A.2}, and R^{L12A.3}, respectively.

In embodiments, when L^{12B} is substituted, L^{12B} is substituted with one or more first substituent groups denoted by R^{L12B.1} as explained in the definitions section above in the description of “first substituent group(s)”. In embodiments, when an R^{L12B.1} substituent group is substituted, the R^{L12B.1} substituent group is substituted with one or more second substituent groups denoted by R^{L12B.2} as explained in the definitions section above in the description of “first substituent group(s)”. In embodiments, when an R^{L12B.2} substituent group is substituted, the R^{L12B.2} substituent group is substituted with one or more third substituent groups denoted by R^{L12B.3} as explained in the definitions section above in the description of “first substituent group(s)”. In the above embodiments, L^{12B}, R^{L12B.1}, R^{L12B.2}, and R^{L12B.3} have values corresponding to the values of L^{WW}, R^{LWW.1}, R^{LWW.2} and R^{LWW.3}, respectively, as explained in the definitions section above in the description of “first substituent group(s)”, wherein L^{WW}, R^{LWW.1}, R^{LWW.2}, and R^{LWW.3} are L^{12B}, R^{L12B.1}, R^{L12B.2}, and R^{L12B.3}, respectively.

In embodiments, when L^{12C} is substituted, L^{12C} is substituted with one or more first substituent groups denoted by R^{L12C.1} as explained in the definitions section above in the description of “first substituent group(s)”. In embodiments, when an R^{L12C.1} substituent group is substituted, the R^{L12C.1} substituent group is substituted with one or more second substituent groups denoted by R^{L12C.2} as explained in the definitions section above in the description of “first substituent group(s)”. In embodiments, when an R^{L12C.2} substituent group is substituted, the R^{L12C.2} substituent group is substituted with one or more third substituent groups denoted by R^{L12C.3} as explained in the definitions section above in the description of “first substituent group(s)”. In the above embodiments, L^{12C}, R^{L12C.1}, R^{L12C.2}, and R^{L12C.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L¹²c, R^{L12C.1}, R^{L12C.2}, and R^{L12C.3}, respectively.

In embodiments, when L^{12F} is substituted, L^{12F} is substituted with one or more first substituent groups denoted by R^{L12F.1} as explained in the definitions section above in the description of “first substituent group(s)”. In embodiments, when an R^{L12F.1} substituent group is substituted, the R^{L12F.1} substituent group is substituted with one or more second substituent groups denoted by R^{L12F.2} as explained in the definitions section above in the description of “first substituent group(s)”. In embodiments, when an R^{L12F.2} substituent group is substituted, the R^{L12F.2} substituent group is substituted with one or more third substituent groups denoted by R^{L12F.3} as explained in the definitions section above in the description of “first substituent group(s)”. In the above embodiments, L^{12F}, R^{L12F.1}, R^{L12F.2}, and R^{L12F.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{12F}, R^{L12F.1}, R^{L12F.2} and R^{L12F.3}, respectively.

In embodiments, when L^{13B} is substituted, L^{13B} is substituted with one or more first substituent groups denoted by R^{L13B.1} as explained in the definitions section above in the description of “first substituent group(s)”. In embodiments, when an R^{L13B.1} substituent group is substituted, the R^{L13B.1} substituent group is substituted with one or more second substituent groups denoted by R^{L13B.2} as explained in the definitions section above in the description of “first substituent group(s)”. In embodiments, when an R^{L13B.2} substituent group is substituted, the R^{L13B.2} substituent group is substituted with one or more third substituent groups denoted by R^{L13B.3} as explained in the definitions section above in the description of “first substituent group(s)”. In the above embodiments, L^{13B}, R^{L13B.1}, R^{L13B.2}, and R^{L13B.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{13B}, R^{L13B.1}, R^{L13B.2}, and R^{L13B.3}, respectively.

In embodiments, when L^{13C} is substituted, L^{13C} is substituted with one or more first substituent groups denoted by R^{L13C.1} as explained in the definitions section above in the description of “first substituent group(s)”. In embodiments, when an R^{L13C.1} substituent group is substituted, the R^{L13C.1} substituent group is substituted with one or more second substituent groups denoted by R^{L13C.2} as explained in the definitions section above in the description of “first substituent group(s)”. In embodiments, when an R^{L13C.2} substituent group is substituted, the R^{L13C.2} substituent group is substituted with one or more third substituent groups denoted by R^{L13C.3} as explained in the definitions section above in the description of “first substituent group(s)”. In the above embodiments, L^{13C}, R^{L13C.1}, R^{L13C.2}, and R^{L13C.3} have values corresponding to the values of L^{WW}, R^{LWW.1}, R^{LWW.2} and R^{LWW.3}, respectively, as explained in the definitions section above in the description of “first substituent group(s)”, wherein L^{WW}, R^{LWW.1}, R^{LWW.2}, and R^{LWW.3} are L^{13C}, R^{L13C.1}, R^{L13C.2}, and R^{L13C.3}, respectively.

In embodiments, when L^{13F} is substituted, L^{13F} is substituted with one or more first substituent groups denoted by R^{L13F.1} as explained in the definitions section above in the description of “first substituent group(s)”. In embodiments, when an R^{L13F.1} substituent group is substituted, the R^{L13F.1} substituent group is substituted with one or more second substituent groups denoted by R^{L13F.2} as explained in the definitions section above in the description of “first substituent group(s)”. In embodiments, when an R^{L13F.2} substituent group is substituted, the R^{L13F.2} substituent group is substituted with one or more third substituent groups denoted by R^{L13F.3} as explained in the definitions section above in the description of “first substituent group(s)”. In the above embodiments, L^{13F}, R^{L13F.1}, R^{L13F.2}, and R^{L13F.3} have values corresponding to the values of L^{WW}, R^{LWW.1}, R^{LWW.2} and R^{LWW.3}, respectively, as explained in the definitions section above in the description of “first substituent group(s)”, wherein L^{WW}, R^{LWW.1}, R^{LWW.2}, and R^{LWW.3} are L^{13F}, R^{L13F.1}, R^{L13F.2} and R^{L13F.3}, respectively.

In embodiments, when L^{14C} is substituted, L^{14C} is substituted with one or more first substituent groups denoted by R^{L14C.1} as explained in the definitions section above in the description of “first substituent group(s)”. In embodiments, when an R^{L14C.1} substituent group is substituted, the R^{L14C.1} substituent group is substituted with one or more second substituent groups denoted by R^{L14C.2} as explained in the definitions section above in the description of “first substituent group(s)”. In embodiments, when an R^{L14C.2} substituent group is substituted, the R^{L14C.2} substituent group is substituted with one or more third substituent groups denoted by R^{L14C.3} as explained in the definitions section above in the description of “first substituent group(s)”. In the above embodiments, L^{14C}, R^{L14C.1}, R^{L14C.2}, and R^{L14C.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{14C}, R^{L14C.1}, R^{L14C.2}, and R^{L14C.3}, respectively.

In embodiments, when L^{14F} is substituted, L^{14F} is substituted with one or more first substituent groups denoted by R^{L14F.1} as explained in the definitions section above in the description of “first substituent group(s)”. In embodiments, when an R^{L14F.1} substituent group is substituted, the R^{L14F.1} substituent group is substituted with one or more second substituent groups denoted by R^{L14F.2} as explained in the definitions section above in the description of “first substituent group(s)”. In embodiments, when an R^{L14F.2} substituent group is substituted, the R^{L14F.2} substituent group is substituted with one or more third substituent groups denoted by R^{L14F.3} as explained in the definitions section above in the description of “first substituent group(s)”. In the above embodiments, L^{14F}, R^{L14F.1}, R^{L14F.2}, and R^{L14F.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{14F}, R^{L14F.1}, R^{L14F.2}, and R^{L14F.3}, respectively.

In embodiments, when L^{15C} is substituted, L^{15C} is substituted with one or more first substituent groups denoted by R^{L15C.1} as explained in the definitions section above in the description of “first substituent group(s)”. In embodiments, when an R^{L15C.1} substituent group is substituted, the R^{L15C.1} substituent group is substituted with one or more second substituent groups denoted by R^{L15C.2} as explained in the definitions section above in the description of “first substituent group(s)”. In embodiments, when an R^{L15C.2} substituent group is substituted, the R^{L15C.2} substituent group is substituted with one or more third substituent groups denoted by R^{L15C.3} as explained in the definitions section above in the description of “first substituent group(s)”. In the above embodiments, L^{15C}, R^{L15C.1}, R^{L15C.2}, and R^{L15C.3} have values corresponding to the values of L^{WW}, R^{LWW.1}, R^{LWW.2} and R^{LWW.3}, respectively, as explained in the definitions section above in the description of “first substituent group(s)”, wherein L^{WW}, R^{LWW.1}, R^{LWW.2}, and R^{LWW.3} are L^{15C}, R^{L15C.1}, R^{L15C.2}, and R^{L15C.3}, respectively.

In embodiments, when L^{15F} is substituted, L^{15F} is substituted with one or more first substituent groups denoted by R^{L15F.1} as explained in the definitions section above in the description of “first substituent group(s)”. In embodiments, when an R^{L15F.1} substituent group is substituted, the R^{L15F.1} substituent group is substituted with one or more second substituent groups denoted by R^{L15F.2} as explained in the definitions section above in the description of “first substituent group(s)”. In embodiments, when an R^{L15F.2} substituent group is substituted, the R^{L15F.2} substituent group is substituted with one or more third substituent groups denoted by R^{L15F.3} as explained in the definitions section above in the description of “first substituent group(s)”. In the above embodiments, L^{15F}, R^{L15F.1}, R^{L15F.2}, and R^{L15F.3} have values corresponding to the values of L^{WW}, R^{LWW.1}, R^{LWW.2} and R^{LWW.3}, respectively, as explained in the definitions section above in the description of “first substituent group(s)”, wherein L^{WW}, R^{LWW.1}, R^{LWW.2}, and R^{LWW.3} are L^{15F}, R^{L15F.1}, R^{L15F.2} and R^{L15F.3}, respectively.

In embodiments, when L¹⁶ is substituted, L¹⁶ is substituted with one or more first substituent groups denoted by R^{L16.1} as explained in the definitions section above in the description of “first substituent group(s)”. In embodiments, when an R^{L16.1} substituent group is substituted, the R^{L16.1} substituent group is substituted with one or more second substituent groups denoted by R^{L16.2} as explained in the definitions section above in the description of “first substituent group(s)”. In embodiments, when an R^{L16.2} substituent group is substituted, the R^{L16.2} substituent group is substituted with one or more third substituent groups denoted by R^{L16.3} as explained in the definitions section above in the description of “first substituent group(s)”. In the above embodiments, L¹⁶, R^{L16.1}, R^{L16.2}, and R^{L16.3} have values corresponding to the values of L^{WW}, R^{LWW.1}, R^{LWW.2} and R^{LWW.3}, respectively, as explained in the definitions section above in the description of “first substituent group(s)”, wherein L^{WW}, R^{LWW.1}, R^{LWW.2}, and R^{LWW.3} are L¹⁶, R^{L16.1}, R^{L16.2}, and R^{L16.3}, respectively.

In embodiments, when L^{16A} is substituted, L^{16A} is substituted with one or more first substituent groups denoted by R^{L16A.1} as explained in the definitions section above in the description of “first substituent group(s)”. In embodiments, when an R^{L16A.1} substituent group is substituted, the R^{L16A.1} substituent group is substituted with one or more second substituent groups denoted by R^{L16A.2} as explained in the definitions section above in the description of “first substituent group(s)”. In embodiments, when an R^{L16A.2} substituent group is substituted, the R^{L16A.2} substituent group is substituted with one or more third substituent groups denoted by R^{L16A.3} as explained in the definitions section above in the description of “first substituent group(s)”. In the above embodiments, L^{16A}, R^{L16A.1}, R^{L16A.2}, and R^{L16A.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{16A}, R^{L16A.1}, R^{L16A.2}, and R^{L16A.3}, respectively.

In embodiments, when L^{16B} is substituted, L^{16B} is substituted with one or more first substituent groups denoted by R^{L16B.1} as explained in the definitions section above in the description of “first substituent group(s)”. In embodiments, when an R^{L16B.1} substituent group is substituted, the R^{L16B.1} substituent group is substituted with one or more second substituent groups denoted by R^{L16B.2} as explained in the definitions section above in the description of “first substituent group(s)”. In embodiments, when an R^{L16B.2} substituent group is substituted, the R^{L16B.2} substituent group is substituted with one or more third substituent groups denoted by R^{L16B.3} as explained in the definitions section above in the description of “first substituent group(s)”. In the above embodiments, L^{16B}, R^{L16B.1}, R^{L16B.2}, and R^{L16B.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{16B}, R^{L16B.1}, R^{L16B.2}, and R^{L16B.3}, respectively.

In embodiments, when L^{16C} is substituted, L^{16C} is substituted with one or more first substituent groups denoted by R^{L16C.1} as explained in the definitions section above in the description of “first substituent group(s)”. In embodiments, when an R^{L16C.1} substituent group is substituted, the R^{L16C.1} substituent group is substituted with one or more second substituent groups denoted by R^{L16C.2} as explained in the definitions section above in the description of “first substituent group(s)”. In embodiments, when an R^{L16C.2} substituent group is substituted, the R^{L16C.2} substituent group is substituted with one or more third substituent groups denoted by R^{L16C.3} as explained in the definitions section above in the description of “first substituent group(s)”. In the above embodiments, L^{16C}, R^{L16C.1}, R^{L16C.2}, and R^{L16C.3} have values corresponding to the values of L^{WW}, R^{LWW.1}, R^{LWW.2} and R^{LWW.3}, respectively, as explained in the definitions section above in the description of “first substituent group(s)”, wherein L^{WW}, R^{LWW.1}, R^{LWW.2}, and R^{LWW.3} are L¹⁶c, R^{L16C.1}, R^{L16C.2}, and R^{L16C.3}, respectively.

In embodiments, when L^{16D} is substituted, L^{16D} is substituted with one or more first substituent groups denoted by R^{L16D.1} as explained in the definitions section above in the description of “first substituent group(s)”. In embodiments, when an R^{L16D.1} substituent group is substituted, the R^{L16D.1} substituent group is substituted with one or more second substituent groups denoted by R^{L16D.2} as explained in the definitions section above in the description of “first substituent group(s)”. In embodiments, when an R^{L16D.2} substituent group is substituted, the R^{L16D.2} substituent group is substituted with one or more third substituent groups denoted by R^{L16D.3} as explained in the definitions section above in the description of “first substituent group(s)”. In the above embodiments, L^{16D}, R^{L16D.1}, R^{L16D.2}, and R^{L16D.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{16D}, R^{L16D.1}, R^{L16D.2}, and R^{L16D.3}, respectively.

In embodiments, when L^{16E} is substituted, L^{16E} is substituted with one or more first substituent groups denoted by R^{L16E.1} as explained in the definitions section above in the description of “first substituent group(s)”. In embodiments, when an R^{L16E.1} substituent group is substituted, the R^{L16E.1} substituent group is substituted with one or more second substituent groups denoted by R^{L16E.2} as explained in the definitions section above in the description of “first substituent group(s)”. In embodiments, when an R^{L16E.2} substituent group is substituted, the R^{L16E.2} substituent group is substituted with one or more third substituent groups denoted by R^{L16E.3} as explained in the definitions section above in the description of “first substituent group(s)”. In the above embodiments, L^{16E}, R^{L16E.1}, R^{L16E.2}, and R^{L16E.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{16E}, R^{L16E.1}, R^{L16E.2} and R^{L16E.3}, respectively.

In embodiments, when L^{16F} is substituted, L^{16F} is substituted with one or more first substituent groups denoted by R^{L16F.1} as explained in the definitions section above in the description of “first substituent group(s)”. In embodiments, when an R^{L16F.1} substituent group is substituted, the R^{L16F.1} substituent group is substituted with one or more second substituent groups denoted by R^{L16F.2} as explained in the definitions section above in the description of “first substituent group(s)”. In embodiments, when an R^{L16F.2} substituent group is substituted, the R^{L16F.2} substituent group is substituted with one or more third substituent groups denoted by R^{L16F.3} as explained in the definitions section above in the description of “first substituent group(s)”. In the above embodiments, L^{16F}, R^{L16F.1}, R^{L16F.2} and R^{L16F.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L¹⁶F, R^{L16F.1}, R^{L16F.2}, and R^{L16F.3}, respectively.

In embodiments, when L^{17A} is substituted, L^{17A} is substituted with one or more first substituent groups denoted by R^{L17A.1} as explained in the definitions section above in the description of “first substituent group(s)”. In embodiments, when an R^{L17A.1} substituent group is substituted, the R^{L17A.1} substituent group is substituted with one or more second substituent groups denoted by R^{L17A.2} as explained in the definitions section above in the description of “first substituent group(s)”. In embodiments, when an R^{L17A.2} substituent group is substituted, the R^{L17A.2} substituent group is substituted with one or more third substituent groups denoted by R^{L17A.3} as explained in the definitions section above in the description of “first substituent group(s)”. In the above embodiments, L^{17A}, R^{L17A.1}, R^{L17A.2}, and R^{L17A.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{17A}, R^{L17A.1}, R^{L17A.2}, and R^{L17A.3}, respectively.

In embodiments, when L^{17B} is substituted, L^{17B} is substituted with one or more first substituent groups denoted by R^{L17B.1} as explained in the definitions section above in the description of “first substituent group(s)”. In embodiments, when an R^{L17B.1} substituent group is substituted, the R^{L17B.1} substituent group is substituted with one or more second substituent groups denoted by R^{L17B.2} as explained in the definitions section above in the description of “first substituent group(s)”. In embodiments, when an R^{L17B.2} substituent group is substituted, the R^{L17B.2} substituent group is substituted with one or more third substituent groups denoted by R^{L17B.3} as explained in the definitions section above in the description of “first substituent group(s)”. In the above embodiments, L^{17B}, R^{L17B.1}, R^{L17B.2}, and R^{L17B.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{17B}, R^{L17B.1}, R^{L17B.2}, and R^{L17B.3}, respectively.

In embodiments, when L^{17C} is substituted, L^{17C} is substituted with one or more first substituent groups denoted by R^{L17C.1} as explained in the definitions section above in the description of “first substituent group(s)”. In embodiments, when an R^{L17C.1} substituent group is substituted, the R^{L17C.1} substituent group is substituted with one or more second substituent groups denoted by R^{L17C.2} as explained in the definitions section above in the description of “first substituent group(s)”. In embodiments, when an R^{L17C.2} substituent group is substituted, the R^{L17C.2} substituent group is substituted with one or more third substituent groups denoted by R^{L17C.3} as explained in the definitions section above in the description of “first substituent group(s)”. In the above embodiments, L^{17C}, R^{L17C.1}, R^{L17C.2}, and R^{L17C.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L¹⁷c, R^{L17C.1}, R^{L17C.2}, and R^{L17C.3}, respectively.

In embodiments, when L^{17D} is substituted, L^{17D} is substituted with one or more first substituent groups denoted by R^{L17D.1} as explained in the definitions section above in the description of “first substituent group(s)”. In embodiments, when an R^{L17D.1} substituent group is substituted, the R^{L17D.1} substituent group is substituted with one or more second substituent groups denoted by R^{L17D.2} as explained in the definitions section above in the description of “first substituent group(s)”. In embodiments, when an R^{L17D.2} substituent group is substituted, the R^{L17D.2} substituent group is substituted with one or more third substituent groups denoted by R^{L17D.3} as explained in the definitions section above in the description of “first substituent group(s)”. In the above embodiments, L^{17D}, R^{L17D.1}, R^{L17D.2}, and R^{L17D.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{17D}, R^{L17D.1}, R^{L17D.2}, and R^{L17D.3}, respectively.

In embodiments, when L^{17E} is substituted, L^{17E} is substituted with one or more first substituent groups denoted by R^{L17E.1} as explained in the definitions section above in the description of “first substituent group(s)”. In embodiments, when an R^{L17E.1} substituent group is substituted, the R^{L17E.1} substituent group is substituted with one or more second substituent groups denoted by R^{L17E.2} as explained in the definitions section above in the description of “first substituent group(s)”. In embodiments, when an R^{L17E.2} substituent group is substituted, the R^{L17E.2} substituent group is substituted with one or more third substituent groups denoted by R^{L17E.3} as explained in the definitions section above in the description of “first substituent group(s)”. In the above embodiments, L^{17E}, R^{L17E.1}, R^{L17E.2}, and R^{L17E.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{17E}, R^{L17E.1}, R^{L17E.2} and R^{L17E.3}, respectively.

In embodiments, when L^{17F} is substituted, L^{17F} is substituted with one or more first substituent groups denoted by R^{L17F.1} as explained in the definitions section above in the description of “first substituent group(s)”. In embodiments, when an R^{L17F.1} substituent group is substituted, the R^{L17F.1} substituent group is substituted with one or more second substituent groups denoted by R^{L17F.2} as explained in the definitions section above in the description of “first substituent group(s)”. In embodiments, when an R^{L17F.2} substituent group is substituted, the R^{L17F.2} substituent group is substituted with one or more third substituent groups denoted by R^{L17F.3} as explained in the definitions section above in the description of “first substituent group(s)”. In the above embodiments, L^{17F}, R^{L17F.1}, R^{L17F.2}, and R^{L17F.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{17F}, R^{L17F.1}, R^{L17F.2}, and R^{L17F.3}, respectively.

In embodiments, when L^{18A} is substituted, L^{18A} is substituted with one or more first substituent groups denoted by R^{L18A.1} as explained in the definitions section above in the description of “first substituent group(s)”. In embodiments, when an R^{L18A.1} substituent group is substituted, the R^{L18A.1} substituent group is substituted with one or more second substituent groups denoted by R^{L18A.2} as explained in the definitions section above in the description of “first substituent group(s)”. In embodiments, when an R^{L18A.2} substituent group is substituted, the R^{L1A.2} substituent group is substituted with one or more third substituent groups denoted by R^{L18A.3} as explained in the definitions section above in the description of “first substituent group(s)”. In the above embodiments, L^{18A}, R^{L18A.1}, R^{L18A.2}, and R^{L1A.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{18A}, R^{L18A.1}, R^{L18A.2}, and R^{L1A.3}, respectively.

In embodiments, when L^{18B} is substituted, L^{18B} is substituted with one or more first substituent groups denoted by R^{L18B.1} as explained in the definitions section above in the description of “first substituent group(s)”. In embodiments, when an R^{L18B.1} substituent group is substituted, the R^{L18B.1} substituent group is substituted with one or more second substituent groups denoted by R^{L18B.2} as explained in the definitions section above in the description of “first substituent group(s)”. In embodiments, when an R^{L18B.2} substituent group is substituted, the R^{L18B.2} substituent group is substituted with one or more third substituent groups denoted by R^{L18B.3} as explained in the definitions section above in the description of “first substituent group(s)”. In the above embodiments, L^{18B}, R^{L18B.1}, R^{L18B.2}, and R^{L18B.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{18B}, R^{L18B.1}, R^{L18B.2}, and R^{L18B.3}, respectively.

In embodiments, when L^{18C} is substituted, L^{18C} is substituted with one or more first substituent groups denoted by R^{L18C.1} as explained in the definitions section above in the description of “first substituent group(s)”. In embodiments, when an R^{L18C.1} substituent group is substituted, the R^{L18C.1} substituent group is substituted with one or more second substituent groups denoted by R^{L18C.2} as explained in the definitions section above in the description of “first substituent group(s)”. In embodiments, when an R^{L18C.2} substituent group is substituted, the R^{L18C.2} substituent group is substituted with one or more third substituent groups denoted by R^{L18C.3} as explained in the definitions section above in the description of “first substituent group(s)”. In the above embodiments, L^{18C}, R^{L18C.1}, R^{L18C.2}, and R^{L18C.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{18C}, R^{L18C.1}, R^{L18C.2}, and R^{L18C.3}, respectively.

In embodiments, when L^{18D} is substituted, L^{18D} is substituted with one or more first substituent groups denoted by R^{L18D.1} as explained in the definitions section above in the description of “first substituent group(s)”. In embodiments, when an R^{L18D.1} substituent group is substituted, the R^{L18D.1} substituent group is substituted with one or more second substituent groups denoted by R^{L18D.2} as explained in the definitions section above in the description of “first substituent group(s)”. In embodiments, when an R^{L18D.2} substituent group is substituted, the R^{L18D.2} substituent group is substituted with one or more third substituent groups denoted by R^{L18D.3} as explained in the definitions section above in the description of “first substituent group(s)”. In the above embodiments, L^{18D}, R^{L18D.1}, R^{L18D.2}, and R^{L18D.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{18D}, R^{L18D.1}, R^{L18D.2}, and R^{L18D.3}, respectively.

In embodiments, when L^{18E} is substituted, L^{18E} is substituted with one or more first substituent groups denoted by R^{L18E.1} as explained in the definitions section above in the description of “first substituent group(s)”. In embodiments, when an R^{L18E.1} substituent group is substituted, the R^{L18E.1} substituent group is substituted with one or more second substituent groups denoted by R^{L18E.2} as explained in the definitions section above in the description of “first substituent group(s)”. In embodiments, when an R^{L18E.2} substituent group is substituted, the R^{L18E.2} substituent group is substituted with one or more third substituent groups denoted by R^{L18E.3} as explained in the definitions section above in the description of “first substituent group(s)”. In the above embodiments, L^{18E}, R^{L18E.1}, R^{L18E.2}, and R^{L18E.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{18E}, R^{L18E.1}, R^{L18E.2}, and R^{L18E.3}, respectively.

In embodiments, when L^{18F} is substituted, L^{18F} is substituted with one or more first substituent groups denoted by R^{L18F.1} as explained in the definitions section above in the description of “first substituent group(s)”. In embodiments, when an R^{L18F.1} substituent group is substituted, the R^{L18F.1} substituent group is substituted with one or more second substituent groups denoted by R^{L18F.2} as explained in the definitions section above in the description of “first substituent group(s)”. In embodiments, when an R^{L18F.2} substituent group is substituted, the R^{L18F.2} substituent group is substituted with one or more third substituent groups denoted by R^{L18F.3} as explained in the definitions section above in the description of “first substituent group(s)”. In the above embodiments, L^{18F}, R^{L18F.1}, R^{L18F.2}, and R^{L18F.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L^{18F}, R^{L18F.1}, R^{L18F.2} and R^{L18F.3}, respectively.

In embodiments, the compound contacts the Switch 2 groove of human K-Ras protein. In embodiments, the compound contacts a Switch 2 groove amino acid corresponding to G60, Q61, D69, D92, H95, Y96, or Q99 of human K-Ras protein. In embodiments, the compound contacts a Switch 2 groove amino acid corresponding to G60 of human K-Ras protein. In embodiments, the compound contacts a Switch 2 groove amino acid corresponding to Q61 of human K-Ras protein. In embodiments, the compound contacts a Switch 2 groove amino acid corresponding to D69 of human K-Ras protein. In embodiments, the compound contacts a Switch 2 groove amino acid corresponding to D92 of human K-Ras protein. In embodiments, the compound contacts a Switch 2 groove amino acid corresponding to H95 of human K-Ras protein. In embodiments, the compound contacts a Switch 2 groove amino acid corresponding to Y96 of human K-Ras protein. In embodiments, the compound contacts a Switch 2 groove amino acid corresponding to Q99 of human K-Ras protein.

In embodiments, the compound contacts the Switch 2 groove of human H-Ras protein. In embodiments, the compound contacts a Switch 2 groove amino acid corresponding to G60, Q61, D69, D92, Q95, Y96, or Q99 of human H-Ras protein. In embodiments, the compound contacts a Switch 2 groove amino acid corresponding to G60 of human H-Ras protein. In embodiments, the compound contacts a Switch 2 groove amino acid corresponding to Q61 of human H-Ras protein. In embodiments, the compound contacts a Switch 2 groove amino acid corresponding to D69 of human H-Ras protein. In embodiments, the compound contacts a Switch 2 groove amino acid corresponding to D92 of human H-Ras protein. In embodiments, the compound contacts a Switch 2 groove amino acid corresponding to Q95 of human H-Ras protein. In embodiments, the compound contacts a Switch 2 groove amino acid corresponding to Y96 of human H-Ras protein. In embodiments, the compound contacts a Switch 2 groove amino acid corresponding to Q99 of human H-Ras protein.

In embodiments, the compound contacts the Switch 2 groove of human N-Ras protein. In embodiments, the compound contacts a Switch 2 groove amino acid corresponding to G60, Q61, D69, D92, L95, Y96, or Q99 of human N-Ras protein. In embodiments, the compound contacts a Switch 2 groove amino acid corresponding to G60 of human N-Ras protein. In embodiments, the compound contacts a Switch 2 groove amino acid corresponding to Q61 of human N-Ras protein. In embodiments, the compound contacts a Switch 2 groove amino acid corresponding to D69 of human N-Ras protein. In embodiments, the compound contacts a Switch 2 groove amino acid corresponding to D92 of human N-Ras protein. In embodiments, the compound contacts a Switch 2 groove amino acid corresponding to L95 of human N-Ras protein. In embodiments, the compound contacts a Switch 2 groove amino acid corresponding to Y96 of human N-Ras protein. In embodiments, the compound contacts a Switch 2 groove amino acid corresponding to Q99 of human N-Ras protein.

In embodiments, the compound binds a human K-Ras protein-GTP complex more strongly than the compound binds a human K-Ras protein-GDP complex under identical conditions. In embodiments, the compound binds a human K-Ras protein-GTP complex at least 2-fold stronger than the compound binds a human K-Ras protein-GDP complex under identical conditions. In embodiments, the compound binds a human K-Ras protein-GTP complex at least 5-fold stronger than the compound binds a human K-Ras protein-GDP complex under identical conditions. In embodiments, the compound binds a human K-Ras protein-GTP complex at least 10-fold stronger than the compound binds a human K-Ras protein-GDP complex under identical conditions. In embodiments, the compound binds a human K-Ras protein-GTP complex at least 20-fold stronger than the compound binds a human K-Ras protein-GDP complex under identical conditions. In embodiments, the compound binds a human K-Ras protein-GTP complex at least 40-fold stronger than said compound binds a human K-Ras protein-GDP complex under identical conditions. In embodiments, the compound binds a human K-Ras protein-GTP complex at least 60-fold stronger than the compound binds a human K-Ras protein-GDP complex under identical conditions. In embodiments, the compound binds a human K-Ras protein-GTP complex at least 80-fold stronger than the compound binds a human K-Ras protein-GDP complex under identical conditions. In embodiments, the compound binds a human K-Ras protein-GTP complex at least 100-fold stronger than said compound binds a human K-Ras protein-GDP complex under identical conditions. In embodiments, the compound binds a human K-Ras protein-GTP complex at least 500-fold stronger than the compound binds a human K-Ras protein-GDP complex under identical conditions.

In embodiments, the compound of formula (I), (II), or (III) binds a human K-Ras protein-GTP complex more strongly than the compound binds a human K-Ras protein-GDP complex under identical conditions. In embodiments, the compound of formula (I), (II), or (III) binds a human K-Ras protein-GTP complex at least 2-fold stronger than the compound binds a human K-Ras protein-GDP complex under identical conditions. In embodiments, the compound of formula (I), (II), or (III) binds a human K-Ras protein-GTP complex at least 5-fold stronger than the compound binds a human K-Ras protein-GDP complex under identical conditions. In embodiments, the compound of formula (I), (II), or (III) binds a human K-Ras protein-GTP complex at least 10-fold stronger than the compound binds a human K-Ras protein-GDP complex under identical conditions. In embodiments, the compound of formula (I), (II), or (III) binds a human K-Ras protein-GTP complex at least 20-fold stronger than the compound binds a human K-Ras protein-GDP complex under identical conditions. In embodiments, the compound of formula (I), (II), or (III) binds a human K-Ras protein-GTP complex at least 40-fold stronger than the compound binds a human K-Ras protein-GDP complex under identical conditions. In embodiments, the compound of formula (I), (II), or (III) binds a human K-Ras protein-GTP complex at least 60-fold stronger than the compound binds a human K-Ras protein-GDP complex under identical conditions. In embodiments, the compound of formula (I), (II), or (III) binds a human K-Ras protein-GTP complex at least 80-fold stronger than the compound binds a human K-Ras protein-GDP complex under identical conditions. In embodiments, the compound of formula (I), (II), or (III) binds a human K-Ras protein-GTP complex at least 100-fold stronger than the compound binds a human K-Ras protein-GDP complex under identical conditions. In embodiments, the compound of formula (I), (II), or (III) binds a human K-Ras protein-GTP complex at least 500-fold stronger than the compound binds a human K-Ras protein-GDP complex under identical conditions.

In embodiments, the compound of formula (IV) binds a human K-Ras protein-GDP complex more strongly than the compound binds a human K-Ras protein-GTP complex under identical conditions. In embodiments, the compound of formula (IV) binds a human K-Ras protein-GDP complex at least 2-fold stronger than the compound binds a human K-Ras protein-GTP complex under identical conditions. In embodiments, the compound of formula (IV) binds a human K-Ras protein-GDP complex at least 5-fold stronger than the compound binds a human K-Ras protein-GTP complex under identical conditions. In embodiments, the compound of formula (IV) binds a human K-Ras protein-GDP complex at least 10-fold stronger than the compound binds a human K-Ras protein-GTP complex under identical conditions. In embodiments, the compound of formula (IV) binds a human K-Ras protein-GDP complex at least 20-fold stronger than the compound binds a human K-Ras protein-GTP complex under identical conditions. In embodiments, the compound of formula (IV) binds a human K-Ras protein-GDP complex at least 40-fold stronger than the compound binds a human K-Ras protein-GTP complex under identical conditions. In embodiments, the compound of formula (IV) binds a human K-Ras protein-GDP complex at least 60-fold stronger than the compound binds a human K-Ras protein-GTP complex under identical conditions. In embodiments, the compound of formula (IV) binds a human K-Ras protein-GDP complex at least 80-fold stronger than the compound binds a human K-Ras protein-GTP complex under identical conditions. In embodiments, the compound of formula (IV) binds a human K-Ras protein-GDP complex at least 100-fold stronger than the compound binds a human K-Ras protein-GTP complex under identical conditions. In embodiments, the compound of formula (IV) binds a human K-Ras protein-GDP complex at least 500-fold stronger than the compound binds a human K-Ras protein-GTP complex under identical conditions.

In embodiments, the compound binds a human K-Ras G12D protein more strongly than the compound binds a human K-Ras wildtype protein under identical conditions. In embodiments, the compound binds a human K-Ras G12D protein at least 2-fold stronger than the compound binds a human K-Ras wildtype protein under identical conditions. In embodiments, the compound binds a human K-Ras G12D protein at least 5-fold stronger than the compound binds a human K-Ras wildtype protein under identical conditions. In embodiments, the compound binds a human K-Ras G12D protein at least 10-fold stronger than the compound binds a human K-Ras wildtype protein under identical conditions. In embodiments, the compound binds a human K-Ras G12D protein at least 20-fold stronger than the compound binds a human K-Ras wildtype protein under identical conditions. In embodiments, the compound binds a human K-Ras G12D protein at least 40-fold stronger than the compound binds a human K-Ras wildtype protein under identical conditions. In embodiments, the compound binds a human K-Ras G12D protein at least 60-fold stronger than the compound binds a human K-Ras wildtype protein under identical conditions. In embodiments, the compound binds a human K-Ras G12D protein at least 80-fold stronger than the compound binds a human K-Ras wildtype protein under identical conditions. In embodiments, the compound binds a human K-Ras G12D protein at least 100-fold stronger than the compound binds a human K-Ras wildtype protein under identical conditions. In embodiments, the compound binds a human K-Ras G12D protein at least 500-fold stronger than the compound binds a human K-Ras wildtype protein under identical conditions.

In embodiments, the compound is capable of binding a water molecule and wherein the water molecule simultaneously binds a D12 residue of a human K-Ras G12D protein.

In embodiments, the compound binds (e.g., covalently binds) a S12 residue of a human K-Ras G12S protein. In embodiments, the compound binds (e.g., covalently binds) a C12 residue of a human K-Ras G12C protein. In embodiments, the compound binds (e.g., covalently binds) a D12 residue of a human K-Ras G12D protein. In embodiments, the compound binds (e.g., covalently binds) a R12 residue of a human K-Ras G12R protein.

In embodiments, the compound binds (e.g., covalently binds) a K61 residue of a human K-Ras Q61K protein. In embodiments, the compound binds (e.g., covalently binds) a R61 residue of a human K-Ras Q61R protein. In embodiments, the compound binds (e.g., covalently binds) a H61 residue of a human K-Ras Q61H protein.

In embodiments, the compound binds (e.g., covalently binds) a K61 residue of a human H-Ras Q61K protein. In embodiments, the compound binds (e.g., covalently binds) a R61 residue of a human H-Ras Q61R protein. In embodiments, the compound binds (e.g., covalently binds) a H61 residue of a human H-Ras Q61H protein.

In embodiments, the compound binds (e.g., covalently binds) a K61 residue of a human N-Ras Q61K protein. In embodiments, the compound binds (e.g., covalently binds) a R61 residue of a human N-Ras Q61R protein. In embodiments, the compound binds (e.g., covalently binds) a H61 residue of a human N-Ras Q61H protein.

In embodiments, the compound binds a human K-Ras G12D protein and wherein the shortest average distance between the compound and the D12 residue of the human K-Ras protein is from 1.5 Å to 6.0 Å. In embodiments, the compound binds a human K-Ras G12D protein and wherein the shortest average distance between the compound and the D12 residue of the human K-Ras protein is from about 1.5 Å to about 6.0 Å. In embodiments, the compound binds a human K-Ras G12D protein and wherein the shortest average distance between the compound and the D12 residue of the human K-Ras protein is less than 1.5 Å. In embodiments, the compound binds a human K-Ras G12D protein and wherein the shortest average distance between the compound and the D12 residue of the human K-Ras protein is less than 2.0 Å. In embodiments, the compound binds a human K-Ras G12D protein and wherein the shortest average distance between the compound and the D12 residue of the human K-Ras protein is less than 3.0 Å. In embodiments, the compound binds a human K-Ras G12D protein and wherein the shortest average distance between the compound and the D12 residue of the human K-Ras protein is less than 4.0 Å. In embodiments, the compound binds a human K-Ras G12D protein and wherein the shortest average distance between the compound and the D12 residue of the human K-Ras protein is less than 5.0 Å. In embodiments, the compound binds a human K-Ras G12D protein and wherein the shortest average distance between the compound and the D12 residue of the human K-Ras protein is less than 6.0 Å.

In embodiments, the compound binds a human K-Ras protein-GTP complex and wherein the shortest average distance between the compound and terminal phosphate of the GTP of the human K-Ras protein-GTP complex is from 1.5 Å to 10.0 Å. In embodiments, the compound binds a human K-Ras protein-GTP complex and wherein the shortest average distance between the compound and terminal phosphate of the GTP of the human K-Ras protein-GTP complex is from about 1.5 Å to about 10.0 Å. In embodiments, the compound binds a human K-Ras protein-GTP complex and wherein the shortest average distance between the compound and terminal phosphate of the GTP of the human K-Ras protein-GTP complex is from 3.0 Å to 10.0 Å. In embodiments, the compound binds a human K-Ras protein-GTP complex and wherein the shortest average distance between the compound and terminal phosphate of the GTP of the human K-Ras protein-GTP complex is from about 3.0 Å to about 10.0 Å. In embodiments, the compound binds a human K-Ras protein-GTP complex and wherein the shortest average distance between the compound and terminal phosphate of the GTP of the human K-Ras protein-GTP complex is less than 1.5 Å. In embodiments, the compound binds a human K-Ras protein-GTP complex and wherein the shortest average distance between the compound and terminal phosphate of the GTP of the human K-Ras protein-GTP complex is less than 2.0 Å. In embodiments, the compound binds a human K-Ras protein-GTP complex and wherein the shortest average distance between the compound and terminal phosphate of the GTP of the human K-Ras protein-GTP complex is less than 3.0 Å. In embodiments, the compound binds a human K-Ras protein-GTP complex and wherein the shortest average distance between the compound and terminal phosphate of the GTP of the human K-Ras protein-GTP complex is less than 4.0 Å. In embodiments, the compound binds a human K-Ras protein-GTP complex and wherein the shortest average distance between the compound and terminal phosphate of the GTP of the human K-Ras protein-GTP complex is less than 5.0 Å. In embodiments, the compound binds a human K-Ras protein-GTP complex and wherein the shortest average distance between the compound and terminal phosphate of the GTP of the human K-Ras protein-GTP complex is less than 6.0 Å. In embodiments, the compound binds a human K-Ras protein-GTP complex and wherein the shortest average distance between the compound and terminal phosphate of the GTP of the human K-Ras protein-GTP complex is less than 7.0 Å. In embodiments, the compound binds a human K-Ras protein-GTP complex and wherein the shortest average distance between the compound and terminal phosphate of the GTP of the human K-Ras protein-GTP complex is less than 8.0 Å. In embodiments, the compound binds a human K-Ras protein-GTP complex and wherein the shortest average distance between the compound and terminal phosphate of the GTP of the human K-Ras protein-GTP complex is less than 9.0 Å. In embodiments, the compound binds a human K-Ras protein-GTP complex and wherein the shortest average distance between the compound and terminal phosphate of the GTP of the human K-Ras protein-GTP complex is less than 10.0 Å.

In embodiments, the compound binds a human H-Ras protein-GTP complex and wherein the shortest average distance between the compound and terminal phosphate of the GTP of the human H-Ras protein-GTP complex is from 1.5 Å to 10.0 Å. In embodiments, the compound binds a human H-Ras protein-GTP complex and wherein the shortest average distance between the compound and terminal phosphate of the GTP of the human H-Ras protein-GTP complex is from about 1.5 Å to about 10.0 Å. In embodiments, the compound binds a human H-Ras protein-GTP complex and wherein the shortest average distance between the compound and terminal phosphate of the GTP of the human H-Ras protein-GTP complex is from 3.0 Å to 10.0 Å. In embodiments, the compound binds a human H-Ras protein-GTP complex and wherein the shortest average distance between the compound and terminal phosphate of the GTP of the human H-Ras protein-GTP complex is from about 3.0 Å to about 10.0 Å. In embodiments, the compound binds a human H-Ras protein-GTP complex and wherein the shortest average distance between the compound and terminal phosphate of the GTP of the human H-Ras protein-GTP complex is less than 1.5 Å. In embodiments, the compound binds a human H-Ras protein-GTP complex and wherein the shortest average distance between the compound and terminal phosphate of the GTP of the human H-Ras protein-GTP complex is less than 2.0 Å. In embodiments, the compound binds a human H-Ras protein-GTP complex and wherein the shortest average distance between the compound and terminal phosphate of the GTP of the human H-Ras protein-GTP complex is less than 3.0 Å. In embodiments, the compound binds a human H-Ras protein-GTP complex and wherein the shortest average distance between the compound and terminal phosphate of the GTP of the human H-Ras protein-GTP complex is less than 5.0 Å. In embodiments, the compound binds a human H-Ras protein-GTP complex and wherein the shortest average distance between the compound and terminal phosphate of the GTP of the human H-Ras protein-GTP complex is less than 10.0 Å.

In embodiments, the compound binds a human N-Ras protein-GTP complex and wherein the shortest average distance between the compound and terminal phosphate of the GTP of the human N-Ras protein-GTP complex is from 1.5 Å to 10.0 Å. In embodiments, the compound binds a human N-Ras protein-GTP complex and wherein the shortest average distance between the compound and terminal phosphate of the GTP of the human N-Ras protein-GTP complex is from about 1.5 Å to about 10.0 Å. In embodiments, the compound binds a human N-Ras protein-GTP complex and wherein the shortest average distance between the compound and terminal phosphate of the GTP of the human N-Ras protein-GTP complex is from 3.0 Å to 10.0 Å. In embodiments, the compound binds a human N-Ras protein-GTP complex and wherein the shortest average distance between the compound and terminal phosphate of the GTP of the human N-Ras protein-GTP complex is from about 3.0 Å to about 10.0 Å. In embodiments, the compound binds a human N-Ras protein-GTP complex and wherein the shortest average distance between the compound and terminal phosphate of the GTP of the human N-Ras protein-GTP complex is less than 1.5 Å. In embodiments, the compound binds a human N-Ras protein-GTP complex and wherein the shortest average distance between the compound and terminal phosphate of the GTP of the human N-Ras protein-GTP complex is less than 2.0 Å. In embodiments, the compound binds a human N-Ras protein-GTP complex and wherein the shortest average distance between the compound and terminal phosphate of the GTP of the human N-Ras protein-GTP complex is less than 3.0 Å. In embodiments, the compound binds a human N-Ras protein-GTP complex and wherein the shortest average distance between the compound and terminal phosphate of the GTP of the human N-Ras protein-GTP complex is less than 5.0 Å. In embodiments, the compound binds a human N-Ras protein-GTP complex and wherein the shortest average distance between the compound and terminal phosphate of the GTP of the human N-Ras protein-GTP complex is less than 10.0 Å.

In embodiments, the compound is useful as a comparator compound. In embodiments, the comparator compound can be used to assess the activity of a test compound as set forth in an assay described herein (e.g., in the examples section, figures, or tables).

In embodiments, the compound is a compound as described herein, including in embodiments. In embodiments the compound is a compound described herein (e.g., in the examples section, figures, tables, or claims).

### III. Pharmaceutical Compositions

In an aspect is provided a pharmaceutical composition including a compound described herein, or a pharmaceutically acceptable salt thereof, and a pharmaceutically acceptable excipient.

In embodiments, the pharmaceutical composition includes an effective amount of the compound. In embodiments, the pharmaceutical composition includes a therapeutically effective amount of the compound.

In embodiments, the pharmaceutical composition includes an effective amount of a second agent, wherein the second agent is an anti-cancer agent. In embodiments, the anti-cancer agent is a MEK inhibitor (e.g., XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, or BAY 869766) or an EGFR inhibitor (e.g., gefitinib (Iressa™), erlotinib (Tarceva™), cetuximab (Erbitux™), lapatinib (Tykerb™) panitumumab (Vectibix™), vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, or BMS-599626).

### IV. Methods of Use

In an aspect is provided a method of treating a cancer in a patient in need of such treatment, the method including administering a therapeutically effective amount of a compound described herein, or a pharmaceutically acceptable salt thereof, to the patient.

In embodiments, the cancer is breast cancer, pancreatic cancer, prostate cancer, colorectal cancer, lung cancer, leukemia, bladder cancer, thyroid cancer, salivary duct carcinoma, epithelial carcinoma, or kidney cancer.

In an aspect is provided a method of modulating (e.g., reducing) the activity of a human Ras (e.g., K-Ras, H-Ras, or N-Ras) protein, the method including contacting the human Ras (e.g., K-Ras, H-Ras, or N-Ras) protein with an effective amount of a compound described herein, or a pharmaceutically acceptable salt thereof. In embodiments, the activity of Ras (e.g., K-Ras, H-Ras, or N-Ras) is reduced by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the activity of Ras (e.g., K-Ras, H-Ras, or N-Ras) is reduced by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold.

In embodiments, the human Ras protein is a human K-Ras protein. In embodiments, the activity of K-Ras is reduced by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the activity of K-Ras is reduced by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold.

In embodiments, the human Ras (e.g., K-Ras, H-Ras, or N-Ras) protein is a human Ras (e.g., K-Ras, H-Ras, or N-Ras) G12D protein, a human Ras (e.g., K-Ras, H-Ras, or N-Ras) G12C protein, a human Ras (e.g., K-Ras, H-Ras, or N-Ras) G12S protein, a human Ras (e.g., K-Ras, H-Ras, or N-Ras) G12V protein, or a human Ras (e.g., K-Ras, H-Ras, or N-Ras) G12R protein. In embodiments, the human Ras (e.g., K-Ras, H-Ras, or N-Ras) protein is a human Ras (e.g., K-Ras, H-Ras, or N-Ras) G12D protein. In embodiments, the human Ras (e.g., K-Ras, H-Ras, or N-Ras) protein is a human Ras (e.g., K-Ras, H-Ras, or N-Ras) G12C protein. In embodiments, the human Ras (e.g., K-Ras, H-Ras, or N-Ras) protein is a human Ras (e.g., K-Ras, H-Ras, or N-Ras) G12S protein. In embodiments, the human Ras (e.g., K-Ras, H-Ras, or N-Ras) protein is a human Ras (e.g., K-Ras, H-Ras, or N-Ras) G12V protein. In embodiments, the human Ras (e.g., K-Ras, H-Ras, or N-Ras) protein is a human Ras (e.g., K-Ras, H-Ras, or N-Ras) G12R protein.

In embodiments, the human Ras (e.g., K-Ras, H-Ras, or N-Ras) protein is a human Ras (e.g., K-Ras, H-Ras, or N-Ras) Q61L protein, a human Ras (e.g., K-Ras, H-Ras, or N-Ras) Q61K protein, a human Ras (e.g., K-Ras, H-Ras, or N-Ras) Q61R protein, or a human Ras (e.g., K-Ras, H-Ras, or N-Ras) Q61H protein. In embodiments, the human Ras (e.g., K-Ras, H-Ras, or N-Ras) protein is a human Ras (e.g., K-Ras, H-Ras, or N-Ras) Q61L protein. In embodiments, the human Ras (e.g., K-Ras, H-Ras, or N-Ras) protein is a human Ras (e.g., K-Ras, H-Ras, or N-Ras) Q61K protein. In embodiments, the human Ras (e.g., K-Ras, H-Ras, or N-Ras) protein is a human Ras (e.g., K-Ras, H-Ras, or N-Ras) Q61R protein. In embodiments, the human Ras (e.g., K-Ras, H-Ras, or N-Ras) protein is a human Ras (e.g., K-Ras, H-Ras, or N-Ras) Q61H protein.

In embodiments, the human Ras (e.g., K-Ras, H-Ras, or N-Ras) protein includes a Q61 mutation. In embodiments, the human Ras (e.g., K-Ras, H-Ras, or N-Ras) protein includes a Q61L mutation. In embodiments, the human Ras (e.g., K-Ras, H-Ras, or N-Ras) protein includes a Q61K mutation. In embodiments, the human Ras (e.g., K-Ras, H-Ras, or N-Ras) protein includes a Q61R mutation. In embodiments, the human Ras (e.g., K-Ras, H-Ras, or N-Ras) protein includes a Q61H mutation.

In embodiments, the activity of the human Ras (e.g., K-Ras, H-Ras, or N-Ras) protein is GTPase activity, nucleotide exchange, GDP binding, GTP binding, differential GDP or GTP binding, effector protein binding, Ras (e.g., K-Ras, H-Ras, or N-Ras) binding to Raf, effector protein activation, guanine exchange factor (GEF) binding, GEF-facilitated nucleotide exchange, phosphate release, nucleotide release, nucleotide binding, Ras (e.g., K-Ras, H-Ras, or N-Ras) subcellular localization, Ras (e.g., K-Ras, H-Ras, or N-Ras) post-translational processing, or Ras (e.g., K-Ras, H-Ras, or N-Ras) post-translational modifications. In embodiments, the activity of the human Ras (e.g., K-Ras, H-Ras, or N-Ras) protein is GTPase activity. In embodiments, the activity of the human Ras (e.g., K-Ras, H-Ras, or N-Ras) protein is nucleotide exchange. In embodiments, the activity of the human Ras (e.g., K-Ras, H-Ras, or N-Ras) protein is GDP binding. In embodiments, the activity of the human Ras (e.g., K-Ras, H-Ras, or N-Ras) protein is GTP binding. In embodiments, the activity of the human Ras (e.g., K-Ras, H-Ras, or N-Ras) protein is differential GDP or GTP binding. In embodiments, the activity of the human Ras (e.g., K-Ras, H-Ras, or N-Ras) protein is effector protein binding. In embodiments, the activity of the human Ras (e.g., K-Ras, H-Ras, or N-Ras) protein is Ras (e.g., K-Ras, H-Ras, or N-Ras) binding to Raf. In embodiments, the activity of the human Ras (e.g., K-Ras, H-Ras, or N-Ras) protein is effector protein activation. In embodiments, the activity of the human Ras (e.g., K-Ras, H-Ras, or N-Ras) protein is guanine exchange factor (GEF) binding. In embodiments, the activity of the human Ras (e.g., K-Ras, H-Ras, or N-Ras) protein is GEF-facilitated nucleotide exchange. In embodiments, the activity of the human Ras (e.g., K-Ras, H-Ras, or N-Ras) protein is phosphate release. In embodiments, the activity of the human Ras (e.g., K-Ras, H-Ras, or N-Ras) protein is nucleotide release. In embodiments, the activity of the human Ras (e.g., K-Ras, H-Ras, or N-Ras) protein is nucleotide binding. In embodiments, the activity of the human Ras (e.g., K-Ras, H-Ras, or N-Ras) protein is K-Ras subcellular localization. In embodiments, the activity of the human Ras (e.g., K-Ras, H-Ras, or N-Ras) protein is Ras (e.g., K-Ras, H-Ras, or N-Ras) post-translational processing. In embodiments, the activity of the human Ras (e.g., K-Ras, H-Ras, or N-Ras) protein is Ras (e.g., K-Ras, H-Ras, or N-Ras) post-translational modifications.

In embodiments, the human Ras protein is a human H-Ras protein. In embodiments, the activity of H-Ras is reduced by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the activity of H-Ras is reduced by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold.

In embodiments, the human Ras protein is a human N-Ras protein. In embodiments, the activity of N-Ras is reduced by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold. In embodiments, the activity of N-Ras is reduced by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold.

### V. Embodiments

Embodiment P1. A compound having the formula:

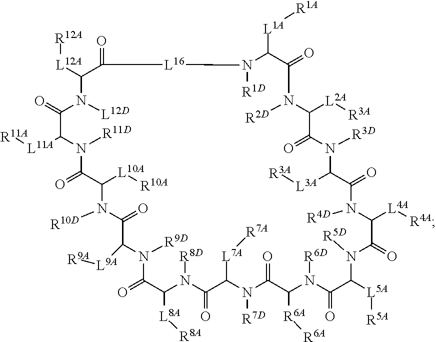

wherein
L^{1A}, L^{2A}, L^{3A}, L^{4A}, L^{5A}, L^{6A}, L^{7A}, L^{8A}, L^{9A}, L^{10A}, L^{11A}, and L^{12A} are independently a bond, substituted or unsubstituted alkylene, or substituted or unsubstituted heteroalkylene;
R^{1A}, R^{2A}, R^{2A}, R^{8A}, and R^{11A} are independently substituted or unsubstituted cycloalkyl, substituted or unsubstituted heterocycloalkyl, substituted or unsubstituted aryl, or substituted or unsubstituted heteroaryl;
R^{3A} is independently hydrogen, substituted or unsubstituted alkyl, substituted or unsubstituted cycloalkyl, or substituted or unsubstituted aryl;
R^{4A} and R^{7A} are independently hydrogen, —NH₂, —COOH, —CONH₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, substituted or unsubstituted alkyl, or substituted or unsubstituted heteroalkyl;
R^{6A}, R^{9A}, and R^{12A} are independently hydrogen, —CN, —NH₂, —CONH₂, —SO₂NH₂, —NHNH₂, —ONH₂, —NHC(O)NHNH₂, —NHC(O)NH₂, —NHC(NH)NH₂, —NHOH, substituted or unsubstituted alkyl, or substituted or unsubstituted heteroalkyl;
R^{3A} and R^{9A} may optionally be joined to form a covalent linker;
R^{10A} is independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —C(O)H, —COOH, —C(O)NH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, -L^{10D}L^{10E}-E, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L^{10D} is independently a bond, —S(O)₂—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L^{10E} is independently a bond, —S(O)₂—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted heteroalkylene, substituted or unsubstituted heterocycloalkylene, or substituted or unsubstituted heteroarylene;
E is an electrophilic moiety;
R^{1D}, R^{2D}, R^{3D}, R^{4D}, RSD, R^{6D}, R^{7D}, R^{8D}, R^{9D}, R^{10D}, R^{11D}, and R^{12D} are independently hydrogen or unsubstituted C₁-C₄ alkyl; and
L¹⁶ is a covalent linker.

Embodiment P2. The compound of embodiment P1, having the formula:

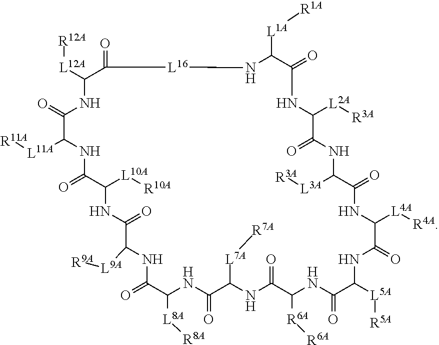

Embodiment P3. The compound of embodiment P1, having the formula:

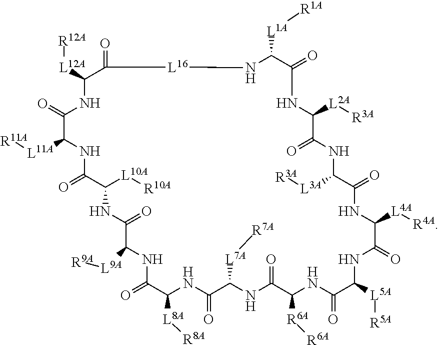

Embodiment P4. The compound of one of embodiments P1 to P3, wherein R^{10A} is independently hydrogen, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, or substituted or unsubstituted heteroalkyl.

Embodiment P5. The compound of one of embodiments P1 to P3, wherein R^{10A} is -L^{10D}-L^{10E}-E;

L^{10D} is independently a bond, —S(O)₂—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L^{10E} is independently a bond, —NH—, —O—, —C(O)—, —C(O)NH—, —NHC(O)NH—, —NHC(NH)NH—, substituted or unsubstituted heteroalkylene, substituted or unsubstituted heterocycloalkylene, or substituted or unsubstituted heteroarylene;
E is an electrophilic moiety capable of forming a covalent bond with a cysteine, aspartate, lysine, arginine, histidine, leucine, tyrosine, methionine, serine, or glutamate residue.

Embodiment P6. The compound of one of embodiments P1 to P3, wherein R^{10A} is -L^{10D}-L^{10E}-E;

L^{10D} is independently a bond, —S(O)₂—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L^{10E} is independently a bond, —S(O)₂—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; E is —SH, —SSR²⁶,

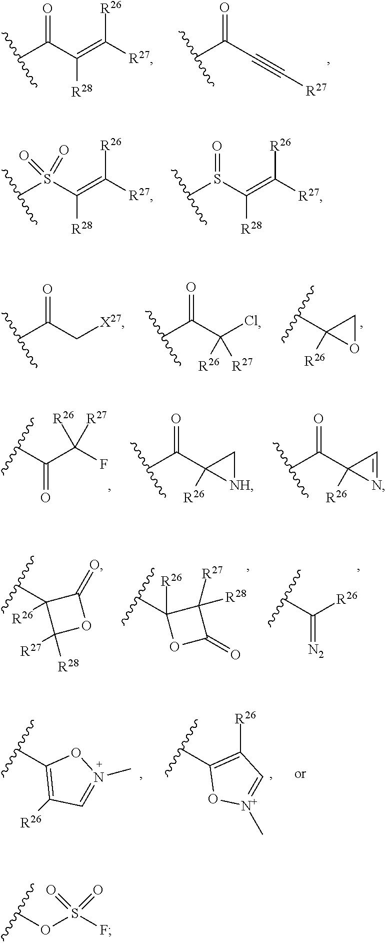

R²⁶, R²⁷, and R²⁸ are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —C(O)H, —C(O)OH, —C(O)NH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
X²⁷ is independently —F, —Cl, —Br, or —I.

Embodiment P7. The compound of one of embodiments P1 to P3, wherein R^{10A} is -L^{10D}-L^{10E}-E;

L^{10D} is independently a bond;
L^{10E} is independently —NH—; and

### E is

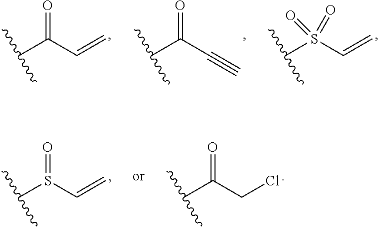

Embodiment P8. The compound of one of embodiments P1 to P3, wherein

-L^{10A}-R^{10A} is

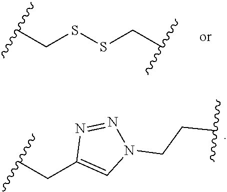 [SMILES: *CSSC* ; *CCn1cc(C*)nn1]

Embodiment P9. The compound of one of embodiments P1 to P8, wherein R^{3A} and R^{9A} are joined to form a bioconjugate linker.

Embodiment P10. The compound of one of embodiments P1 to P8, wherein R^{3A} and R^{9A} are joined to form a covalent linker having the formula -L^{18A}-L^{18B}-L^{18C}-L^{18D}-L^{18E}-L^{18F}-;

L^{18A}, L^{18B}, L^{18C}, L^{18D}, L^{18E}, and L^{18F} are independently bond, —SS—, —S(O)₂—, —OS(O)₂—, —S(O)₂O—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

Embodiment P11. The compound of one of embodiments P1 to P8, wherein R^{3A} and R^{9A} are joined to form

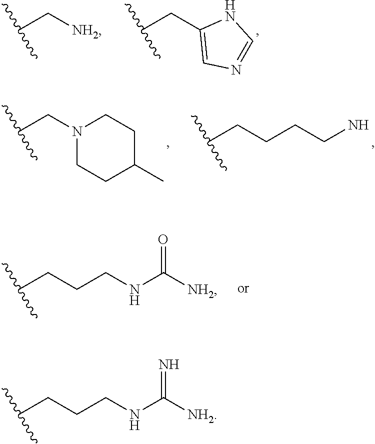

Embodiment P12. The compound of one of embodiments P1 to P3, having the formula:

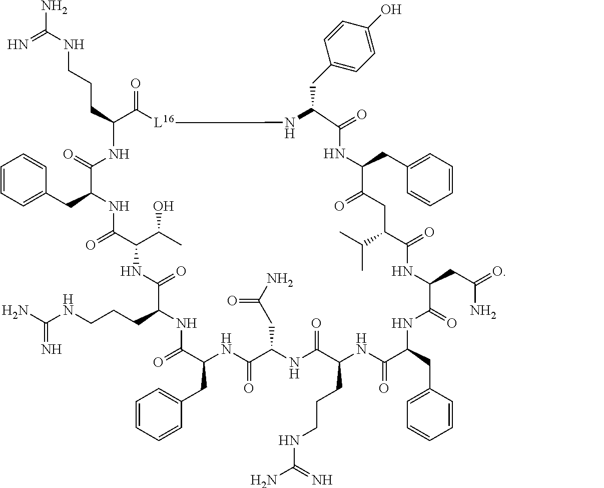

Embodiment P13. A compound having the formula:

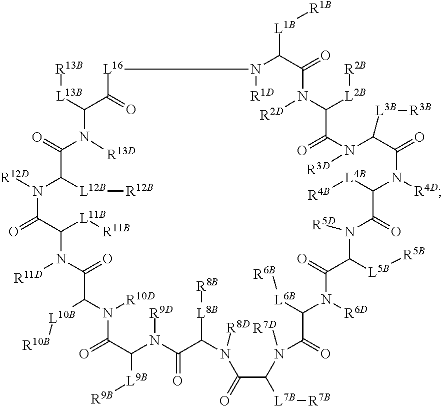

wherein
L^{1B}, L^{2B}, L^{3B}, L^{4B}, L^{5B}, L^{6B}, L^{7B}, L^{8B}, L^{9B}, L^{10B}, L^{11B}, L^{12B}, and L^{13B} are independently a bond, substituted or unsubstituted alkylene, or substituted or unsubstituted heteroalkylene;
R^{1B}, R^{8B}, and R^{10B} are independently substituted or unsubstituted cycloalkyl, substituted or unsubstituted heterocycloalkyl, substituted or unsubstituted aryl, or substituted or unsubstituted heteroaryl;
R^{2B}, R^{3B}, R^{4B}, R^{9B}, and R^{11B} are independently hydrogen, substituted or unsubstituted alkyl, substituted or unsubstituted cycloalkyl, or substituted or unsubstituted aryl;
R^{5B} is independently hydrogen, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, or substituted or unsubstituted heteroalkyl;
R^{6B} is independently hydrogen, —OH, —COOH, —NO₂, —SO₃H, —OSO₃H, substituted or unsubstituted alkyl, or substituted or unsubstituted heteroalkyl;
R^{7B}, R^{12B}, and R^{13B} are independently hydrogen, —NH₂, —CONH₂, —SO₂NH₂, —NHNH₂, —ONH₂, —NHC(O)NHNH₂, —NHC(O)NH₂, —NHC(NH)NH₂, substituted or unsubstituted alkyl, substituted or unsubstituted heteroalkyl, or substituted or unsubstituted heteroaryl; two substituents selected from R^{1B}, R^{2B}, R^{3B}, R^{4B}, R^{5B}, R^{6B}, L^{7B}, R^{8B}, R^{9B}, R^{10B}, R^{11B}, R^{12B}, and R^{13B} may optionally be joined to form a covalent linker;
R^{1D}, R^{2D}, R^{3D}, R^{4D}, RSD, R^{6D}, R^{7D}, R^{8D}, R^{9D}, R^{10D}, R^{11D}, R^{12D}, and R^{13D} are independently hydrogen or unsubstituted C₁-C₄ alkyl; and
L¹⁶ is a covalent linker.

Embodiment P14. The compound of embodiment P13, having the formula:

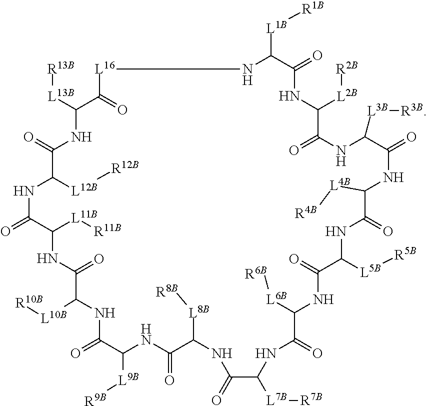

Embodiment P15. The compound of embodiment P13, having the formula:

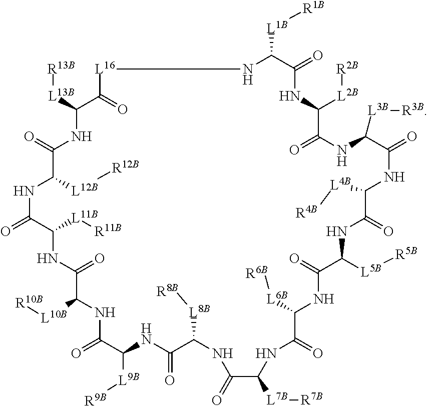

Embodiment P16. The compound of embodiment P13, having the formula:

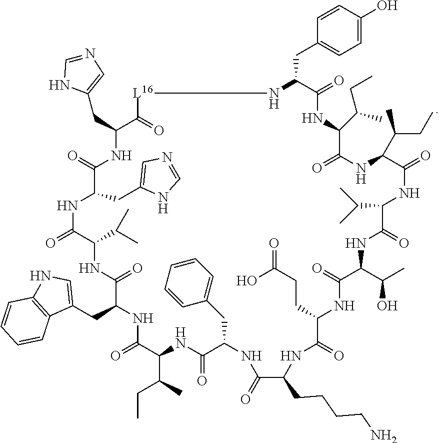

Embodiment P17. A compound having the formula:

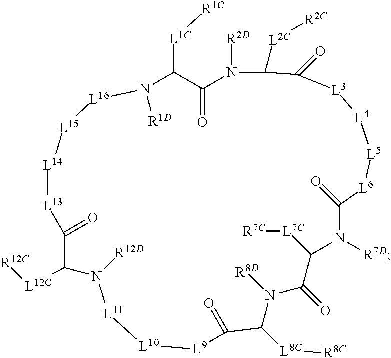

wherein
L^{1C}, L^{2C}, L^{3C}, L^{4C}, L^{5C}, L^{6C}, L^{7C}, L^{8C}, L^{9C}, L^{10C}, L^{11C}, L^{12C}, L^{13C}, L^{14C}, and L^{15C} are independently a bond, substituted or unsubstituted alkylene, or substituted or unsubstituted heteroalkylene;
R^{1C} is independently substituted or unsubstituted cycloalkyl, substituted or unsubstituted heterocycloalkyl, substituted or unsubstituted aryl, or substituted or unsubstituted heteroaryl;
R^{2C} is independently hydrogen, —OH, —NO₂, —CN, —NH₂, —C(O)OH, —C(O)NH₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, substituted or unsubstituted alkyl, or substituted or unsubstituted heteroalkyl;
L³ is independently a bond or

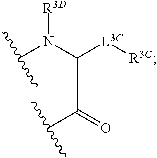

R^{3C} is independently hydrogen, —NH₂, —C(O)OH, —C(O)NH₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L⁴ is independently a bond or

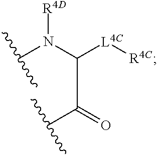

R^{4C} is independently hydrogen, substituted or unsubstituted alkyl, substituted or unsubstituted cycloalkyl, or substituted or unsubstituted aryl;
L⁵ is independently a bond or

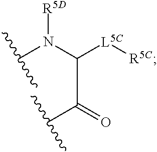

R^{5C} is independently substituted or unsubstituted cycloalkyl, substituted or unsubstituted heterocycloalkyl, substituted or unsubstituted aryl, or substituted or unsubstituted heteroaryl;
L⁶ is independently a bond,

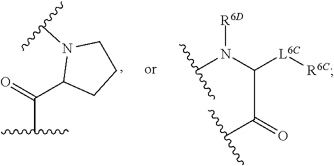

R^{6C} is independently hydrogen, —CN, —NH₂, —C(O)NH₂, —SO₂NH₂, —NHNH₂, —ONH₂, —NHC(O)NHNH₂, —NHC(O)NH₂, —NHC(NH)NH₂, —NHOH, substituted or unsubstituted alkyl, or substituted or unsubstituted heteroalkyl;
R^{7C} and R^{8C} are independently hydrogen, —CN, —NH₂, —C(O)NH₂, —NHNH₂, —ONH₂, —NHC(O)NHNH₂, —NHC(O)NH₂, —NHC(NH)NH₂, —NHOH, substituted or unsubstituted alkyl, or substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L⁹ is independently a bond,

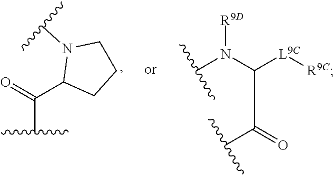

R^{9C} is independently hydrogen, substituted or unsubstituted alkyl, substituted or unsubstituted cycloalkyl, or substituted or unsubstituted aryl;
L¹⁰ is independently a bond,

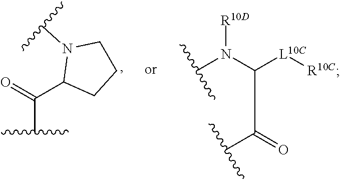

R^{10C} is independently hydrogen, substituted or unsubstituted alkyl, or substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L¹¹ is independently a bond or

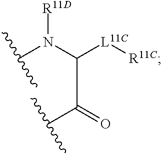

R^{11C} is independently hydrogen, —CN, —OH, —C(O)OH, —NO₂, —SO₃H, —OSO₃H, —NH₂, —C(O)NH₂, —SO₂NH₂, —NHNH₂, —ONH₂, —NHC(O)NHNH₂, —NHC(O)NH₂, —NHC(NH)NH₂, substituted or unsubstituted alkyl, or substituted or unsubstituted heteroalkyl;
R^{12C} is independently hydrogen, substituted or unsubstituted alkyl, or substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L¹³ is independently

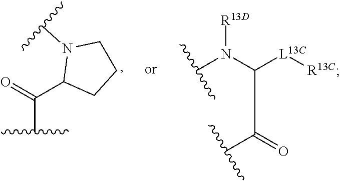

R^{13C} is independently hydrogen, —OH, —NH₂, —C(O)OH, —C(O)NH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, or substituted or unsubstituted heteroalkyl, substituted or unsubstituted cycloalkyl, or substituted or unsubstituted aryl;
L¹⁴ is independently a bond or

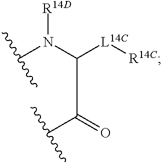

R^{14C} is independently hydrogen, —NH₂, —C(O)OH, —C(O)NH₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, substituted or unsubstituted alkyl, or substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L¹⁵ is independently a bond or

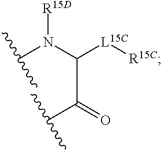

R^{15C} is independently hydrogen, —NH₂, —C(O)OH, —C(O)NH₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, substituted or unsubstituted alkyl, or substituted or unsubstituted heteroalkyl, substituted or unsubstituted cycloalkyl, substituted or unsubstituted heterocycloalkyl, substituted or unsubstituted aryl, or substituted or unsubstituted heteroaryl;
two substituents selected from R^{1C}, R^{2C}, R^{3C}, R^{4C}, R^{5C}, R^{6C}, R^{7C}, R^{8C}, R^{9C}, R^{10C}, R^{11C}, R^{12C}, R^{13C}, R^{14C}, and R^{15C} may optionally be joined to form a covalent linker;
R^{1D}, R^{2D}, R^{3D}, R^{4D}, R^{5D}, R^{6D}, R^{7D}, R^{8D}, R^{9D}, R^{10D}, R^{11D}, R^{12D}, R^{13D}, R^{14D}, and R^{15D} are independently hydrogen or unsubstituted C₁-C₄ alkyl; and
L¹⁶ is a covalent linker.

Embodiment P18. The compound of embodiment P17, having the formula:

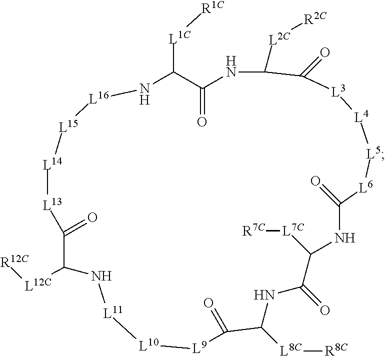

wherein
R^{3D}, R^{4D}, R^{5D}, R^{6D}, R^{9D}, R^{10D}, R^{11D}, R^{13D}, R^{14D}, and R^{15D} are independently hydrogen.

Embodiment P19. The compound of embodiment P18, having the formula:

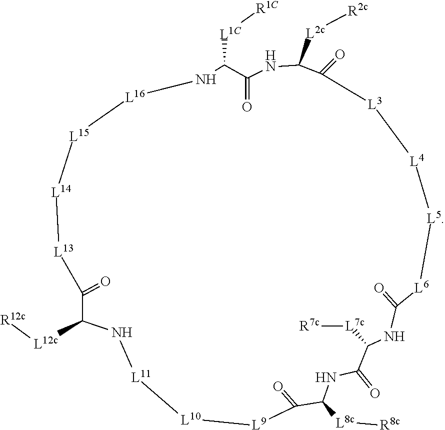

Embodiment P20. The compound of embodiment P18, having the formula:

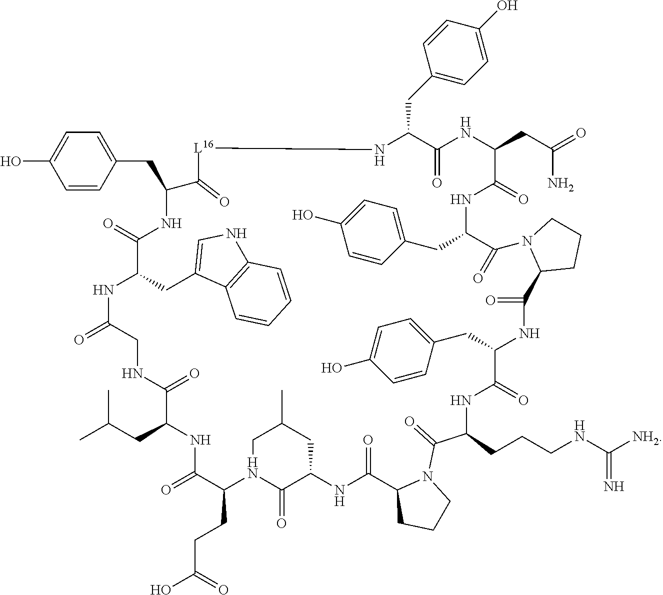

Embodiment P21. The compound of one of embodiments P1 to P20, wherein L¹⁶ is a bioconjugate linker.

Embodiment P22. The compound of one of embodiments P1 to P20, wherein L¹⁶ is a substituted or unsubstituted divalent amino acid.

Embodiment P23. The compound of embodiment P22, wherein L¹⁶ is a substituted or unsubstituted divalent δ-amino acid.

Embodiment P24. The compound of one of embodiments P1 to P20, wherein

L¹⁶ is -L^{16A}-L^{16B}-L^{16C}-L^{16D}-L^{16E}-L^{16F}-; and
L^{16A}, L^{16B}, L^{16C}, L^{16D}, L^{16E}, and L^{16F} are independently bond, —SS—, —S(O)₂—, —OS(O)₂—, —S(O)₂O—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

Embodiment P25. The compound of embodiment P24, wherein

L¹⁶ is —NH-L^{16B}-L^{16C}-L^{16D}-L^{16E}-C(O)—;

### L^{16B} is

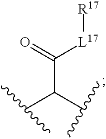

L^{16C}, L^{16D}, and L^{16E} are independently bond, —SS—, —S(O)₂—, —OS(O)₂—, —S(O)₂O—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁷ is -L^{17A}-L^{17B}-L^{17C}-L^{17D}-L^{17E}-L^{17F}-;
L^{17A}, L^{17B}, L^{17C}, L^{17D}, L^{17E}, and L^{17F} are independently bond, —SS—, —S(O)₂—, —OS(O)₂—, —S(O)₂O—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁷ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —C(O)H, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, a monovalent nucleic acid, a monovalent protein a detectable moiety, or a drug moiety.

Embodiment P26. The compound of embodiment P24, wherein L¹⁶ is a bond,

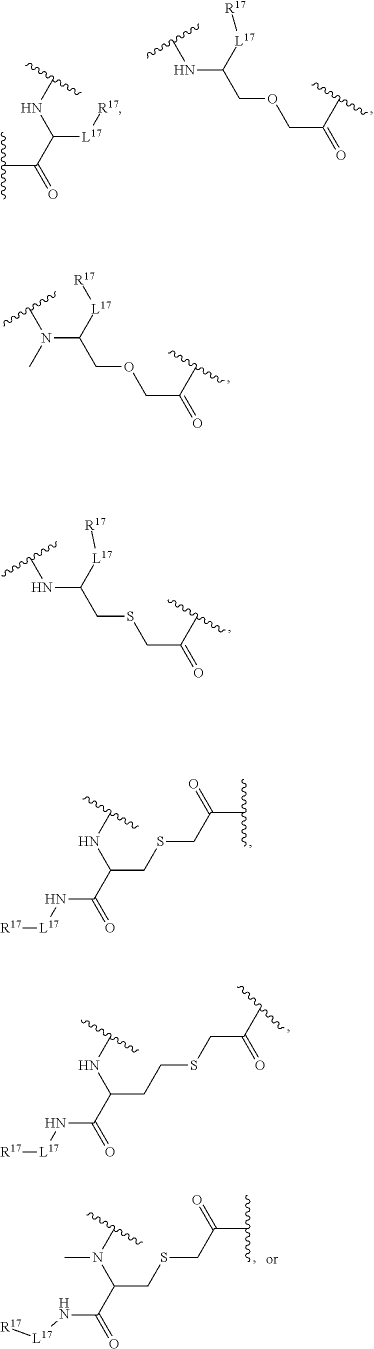

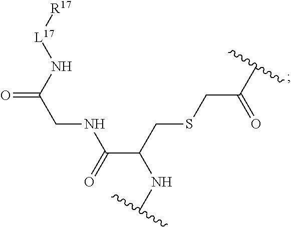

L¹⁷ is -L^{17A}-L^{17B}-L^{17C}-L^{17D}-L^{17E}-L^{17F}-;
L^{17A}, L^{17B}, L^{17C}, L^{17D}, L^{17E}, and L^{17F} are independently bond, —SS—, —S(O)₂—, —OS(O)₂—, —S(O)₂O—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁷ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —C(O)H, —C(O)OH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, a monovalent nucleic acid, a monovalent protein, a detectable moiety, or a drug moiety.

Embodiment P27. The compound of embodiment P24, wherein L¹⁶ is a bond,

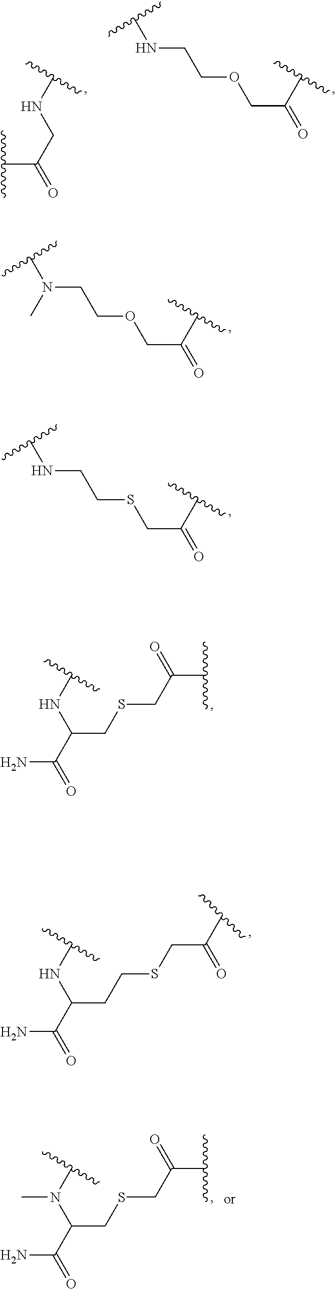

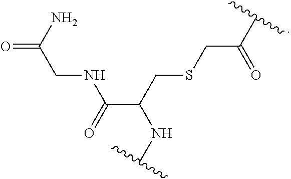 [SMILES: *NC(CSCC(*)=O)C(=O)NCC(N)=O]

Embodiment P28. A compound having the formula:

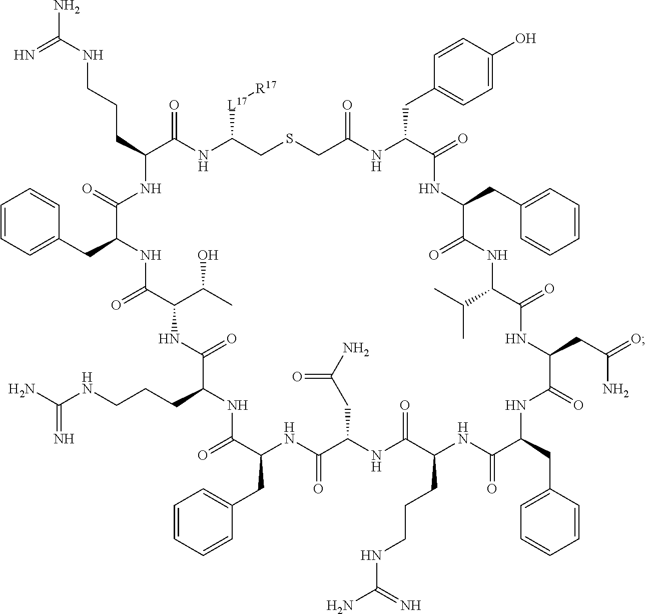

wherein
L¹⁷ is -L^{17A}-L^{17B}-L^{17C}-L^{17D}-L^{17E}-L^{17F}-;
L^{17A}, L^{17B}, L^{17C}, L^{17D}, L^{17E}, and L^{17F} are independently bond, —SS—, —S(O)₂—, —OS(O)₂—, —S(O)₂O—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁷ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —C(O)H, —C(O)OH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, a monovalent nucleic acid, a monovalent protein, a detectable moiety, or a drug moiety.

Embodiment P29. A compound having the formula:

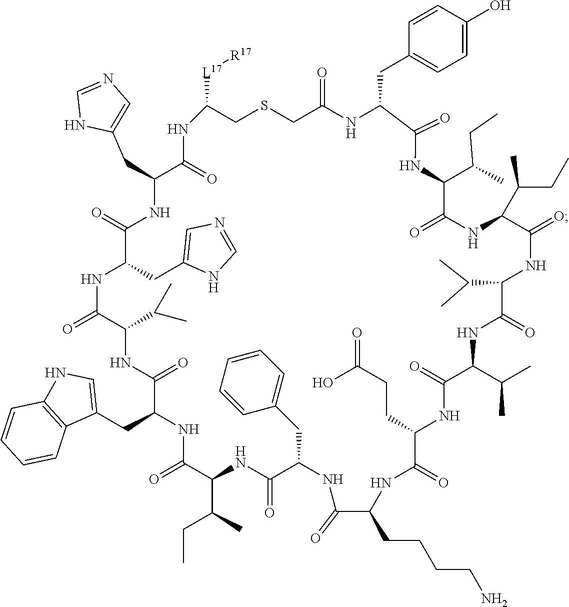

wherein
L¹⁷ is -L^{17A}-L^{17B}-L^{17C}-L^{17D}-L^{17E}-L^{17F}-;
L^{17A}, L^{17B}, L^{17C}, L^{17D}, L^{17E}, and L^{17F} are independently bond, —SS—, —S(O)₂—, —OS(O)₂—, —S(O)₂O—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁷ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —C(O)H, —C(O)OH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, a monovalent nucleic acid, a monovalent protein, a detectable moiety, or a drug moiety.

Embodiment P30. A compound having the formula:

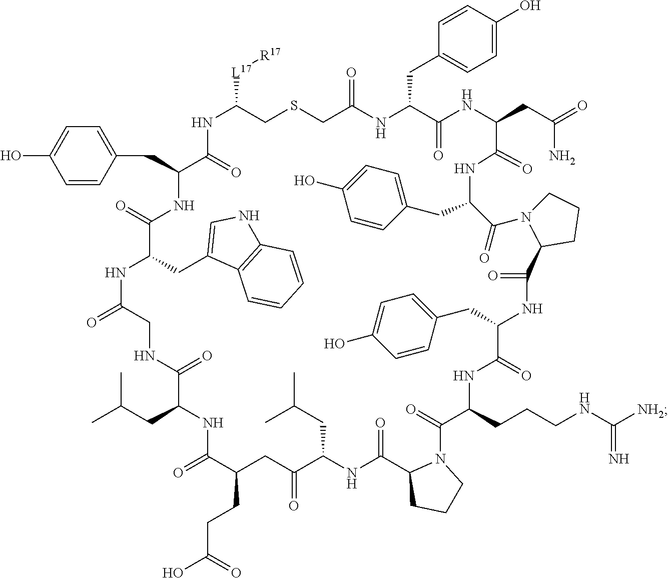

wherein
L¹⁷ is -L^{17A}-L^{17B}-L^{17C}-L^{17D}-L^{17E}-L^{17F}-.
L^{17A}, L^{17B}, L^{17C}, L^{17D}, L^{17E}, and L^{17F} are independently bond, —SS—, —S(O)₂—, —OS(O)₂—, —S(O)₂O—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹⁷ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —C(O)H, —C(O)OH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, a monovalent nucleic acid, a monovalent protein, a detectable moiety, or a drug moiety.

Embodiment P31. A compound having the formula:

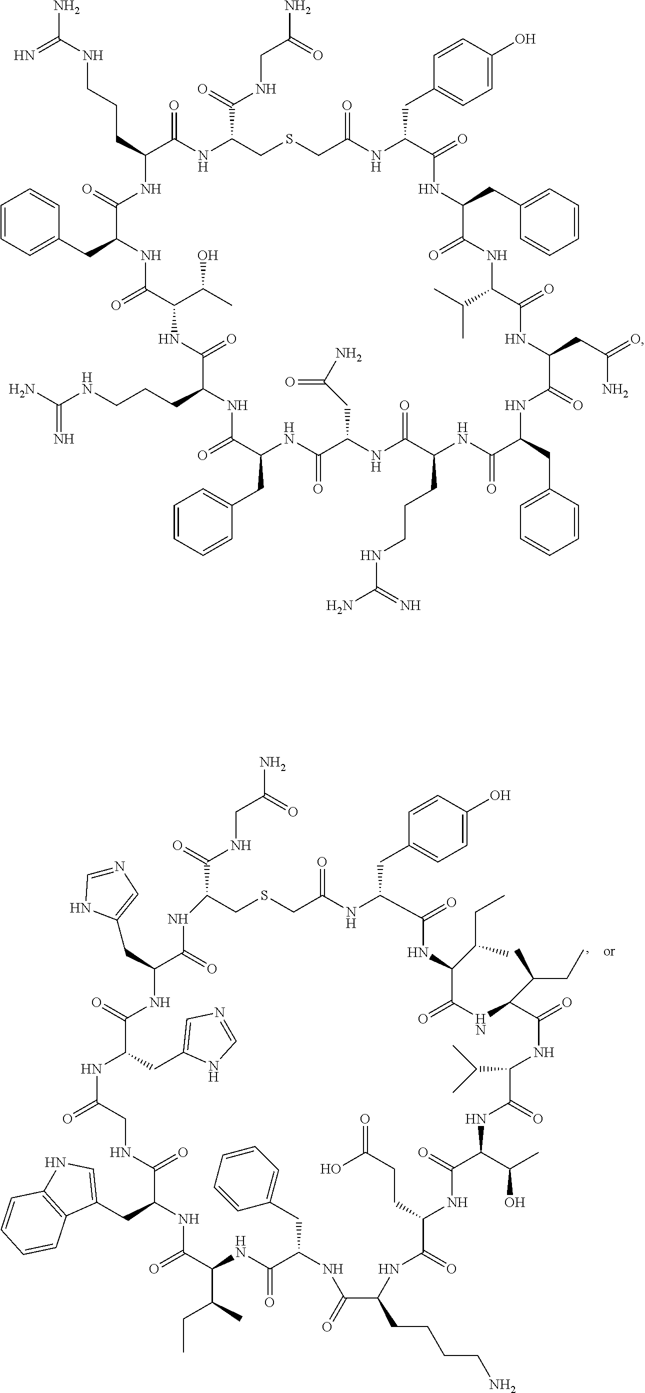

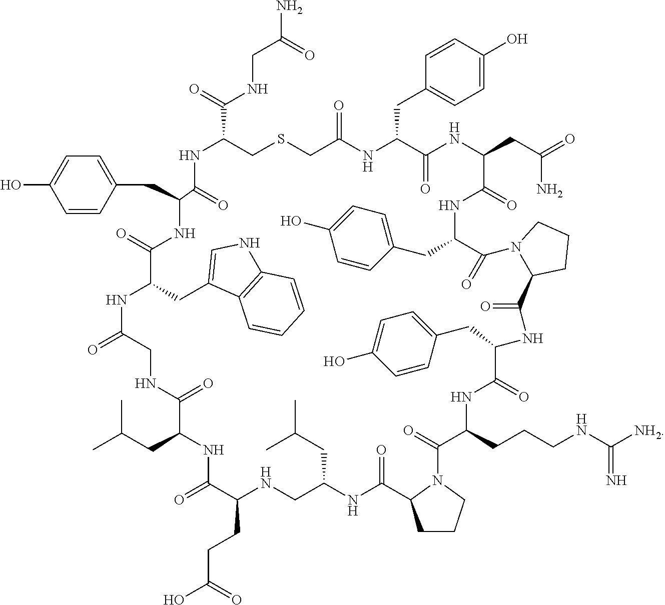 [SMILES: CC(C)C[C@H]1CN[C@@H](CCC(=O)O)C(=O)N[C@@H](CC(C)C)C(=O)NCC(=O)N[C@@H](Cc2c[nH]c3ccccc23)C(=O)N[C@@H](Cc2ccc(O)cc2)C(=O)N[C@H](C(=O)NCC(N)=O)CSCC(=O)N[C@H](Cc2ccc(O)cc2)C(=O)N[C@@H](CC(N)=O)C(=O)N[C@@H](Cc2ccc(O)cc2)C(=O)N2CCC[C@H]2C(=O)N[C@@H](Cc2ccc(O)cc2)C(=O)N[C@@H](CCCNC(=N)N)C(=O)N2CCC[C@H]2C(=O)N1]

Embodiment P32. The compound of one of embodiments P1 to P31, wherein said compound contacts the Switch 2 groove of human K-Ras protein.

Embodiment P33. The compound of one of embodiments P1 to P31, wherein said compound contacts a Switch 2 groove amino acid corresponding to G60, Q61, D69, D92, H95, Y96, or Q99 of human K-Ras protein.

Embodiment P34. The compound of one of embodiments P1 to P31, wherein said compound binds a human K-Ras protein-GTP complex more strongly than said compound binds a human K-Ras protein-GDP complex under identical conditions.

Embodiment P35. The compound of embodiment P34, wherein said compound binds a human K-Ras protein-GTP complex at least 2-fold stronger than said compound binds a human K-Ras protein-GDP complex under identical conditions.

Embodiment P36. The compound of embodiment P34, wherein said compound binds a human K-Ras protein-GTP complex at least 5-fold stronger than said compound binds a human K-Ras protein-GDP complex under identical conditions.

Embodiment P37. The compound of embodiment P34, wherein said compound binds a human K-Ras protein-GTP complex at least 40-fold stronger than said compound binds a human K-Ras protein-GDP complex under identical conditions.

Embodiment P38. The compound of embodiment P34, wherein said compound binds a human K-Ras protein-GTP complex at least 100-fold stronger than said compound binds a human K-Ras protein-GDP complex under identical conditions.

Embodiment P39. The compound of one of embodiments P1 to P38, wherein said compound binds a human K-Ras G12D protein more strongly than said compound binds a human K-Ras wildtype protein under identical conditions.

Embodiment P40. The compound of one of embodiments P1 to P38, wherein said compound binds a human K-Ras G12D protein at least 2-fold stronger than said compound binds a human K-Ras wildtype protein under identical conditions.

Embodiment P41. The compound of one of embodiments P1 to P38, wherein said compound binds a human K-Ras G12D protein at least 10-fold stronger than said compound binds a human K-Ras wildtype protein under identical conditions.

Embodiment P42. The compound of one of embodiments P1 to P38, wherein said compound binds a human K-Ras G12D protein at least 100-fold stronger than said compound binds a human K-Ras wildtype protein under identical conditions.

Embodiment P43. The compound of one of embodiments P1 to P42, wherein said compound is capable of binding a water molecule and wherein said water molecule simultaneously binds a D12 residue of a human K-Ras G12D protein.

Embodiment P44. The compound of one of embodiments P1 to P42, wherein said compound binds a human K-Ras G12D protein and wherein the shortest average distance between the compound and the D12 residue of said human K-Ras protein is from 1.5 Å to 6.0 Å.

Embodiment P45. The compound of one of embodiments P1 to P42, wherein said compound binds a human K-Ras protein-GTP complex and wherein the shortest average distance between the compound and terminal phosphate of the GTP of said human K-Ras protein-GTP complex is from 1.5 Å to 10.0 Å.

Embodiment P46. A pharmaceutical composition comprising the compound of any one of embodiments P1 to P45, or a pharmaceutically acceptable salt thereof, and a pharmaceutically acceptable excipient.

Embodiment P47. A method of treating a cancer in a patient in need of such treatment, said method comprising administering a therapeutically effective amount of a compound of any one of embodiments P1 to P45 to said patient.

Embodiment P48. A method of modulating the activity of a human K-Ras protein, said method comprising contacting said human K-Ras protein with an effective amount of a compound of any one of embodiments P1 to P45.

Embodiment P49. The method of embodiment P48, wherein said human K-Ras protein is a human K-Ras G12D protein, a human K-Ras G12C protein, a human K-Ras G12S protein, a human K-Ras G12V protein, or a human K-Ras G12R protein.

Embodiment P50. The method of embodiment P48, wherein said human K-Ras protein comprises a Q61 mutation.

Embodiment P51. The method of one of embodiments P48 to P50, wherein the activity of the human K-Ras protein is GTPase activity, nucleotide exchange, GDP binding, GTP binding, differential GDP or GTP binding, effector protein binding, K-Ras binding to Raf, effector protein activation, guanine exchange factor (GEF) binding, GEF-facilitated nucleotide exchange, phosphate release, nucleotide release, nucleotide binding, K-Ras subcellular localization, K-Ras post-translational processing, or K-Ras post-translational modifications.

Embodiment P52. The method of one of embodiments P48 to P50, wherein the activity of the human K-Ras protein is K-Ras binding to Raf.

Embodiment P53. A method of modulating the activity of a human H-Ras protein, said method comprising contacting said human H-Ras protein with an effective amount of a compound of any one of embodiments P1 to P45.

Embodiment P54. A method of modulating the activity of a human N-Ras protein, said method comprising contacting said human N-Ras protein with an effective amount of a compound of any one of embodiments P1 to P45.

### VI. Additional Embodiments

Embodiment 1. A compound having the formula:

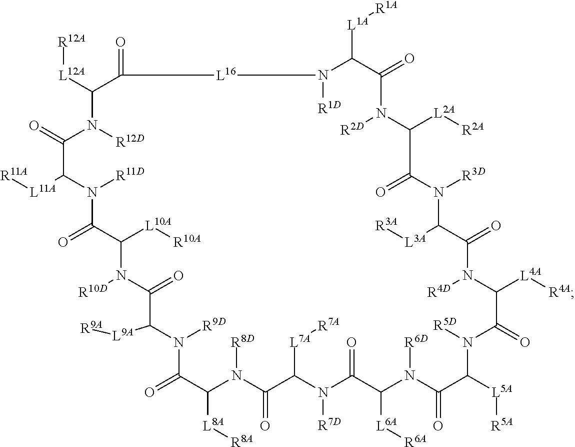

wherein
L^{1A}, L^{2A}, L^{3A}, L^{4A}, L^{5A}, L^{6A}, L^{7A}, L^{8A}, L^{9A}, L^{10A}, L^{11A}, and L^{12A} are independently a bond, substituted or unsubstituted alkylene, or substituted or unsubstituted heteroalkylene;
R^{1A}, R^{2A}, R^{5A}, R^{8A}, and R^{11A} are independently substituted or unsubstituted cycloalkyl, substituted or unsubstituted heterocycloalkyl, substituted or unsubstituted aryl, or substituted or unsubstituted heteroaryl;
R^{3A} is hydrogen, substituted or unsubstituted alkyl, substituted or unsubstituted cycloalkyl, or substituted or unsubstituted aryl;
R^{4A} and R^{7A} are independently hydrogen, —NH₂, —COOH, —CONH₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, substituted or unsubstituted alkyl, or substituted or unsubstituted heteroalkyl;
R^{6A}, R^{9A}, and R^{12A} are independently hydrogen, —CN, —NH₂, —CONH₂, —SO₂NH₂, —NHNH₂, —ONH₂, —NHC(O)NHNH₂, —NHC(O)NH₂, —NHC(NH)NH₂, —NHOH, substituted or unsubstituted alkyl, or substituted or unsubstituted heteroalkyl;
R^{3A} and R^{9A} may optionally be joined to form a covalent linker;
R^{10A} is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —C(O)H, —COOH, —C(O)NH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, -L^{10D}-L^{10E}-E, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L^{10D} is a bond, —S(O)₂—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L^{10E} is a bond, —S(O)₂—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted heteroalkylene, substituted or unsubstituted heterocycloalkylene, or substituted or unsubstituted heteroarylene;
E is an electrophilic moiety;
R^{1D}, R^{2D}, R^{3D}, R^{4D}, R^{5D}, R^{6D}, R^{7D}, R^{8D}, R^{9D}, R^{10D}, R^{11D}, and R^{12D} are independently hydrogen or unsubstituted C₁-C₄ alkyl; and
L¹⁶ is a covalent linker.

Embodiment 2. The compound of embodiment 1, having the formula:

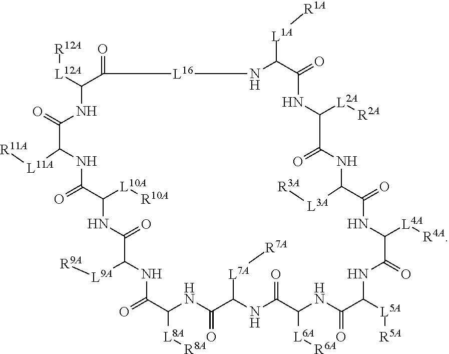

Embodiment 3. The compound of embodiment 1, having the formula:

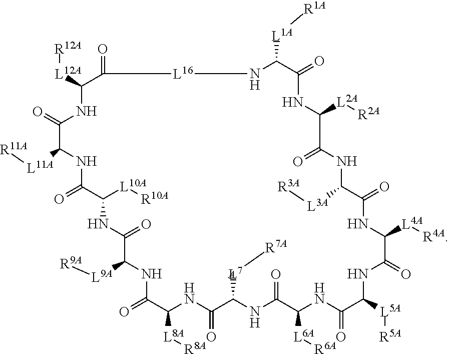

Embodiment 4. The compound of one of embodiments 1 to 3, wherein R^{10A} is hydrogen, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, or substituted or unsubstituted heteroalkyl.

Embodiment 5. The compound of one of embodiments 1 to 3, wherein R^{10A} is -L^{10D}-L^{10E}-E;

L^{10D} is a bond, —S(O)₂—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; L^{10E} is a bond, —NH—, —O—, —C(O)—, —C(O)NH—, —NHC(O)NH—, —NHC(NH)NH—, substituted or unsubstituted heteroalkylene, substituted or unsubstituted heterocycloalkylene, or substituted or unsubstituted heteroarylene; and
E is an electrophilic moiety capable of forming a covalent bond with a cysteine, aspartate, lysine, arginine, histidine, leucine, tyrosine, methionine, serine, or glutamate residue.

Embodiment 6. The compound of one of embodiments 1 to 3, wherein R^{10A} is -L^{10D}-L^{10E}-E;

L^{10D} is a bond, —S(O)₂—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L^{10E} is a bond, —S(O)₂—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;

### E is —SH, —SSR²⁶,

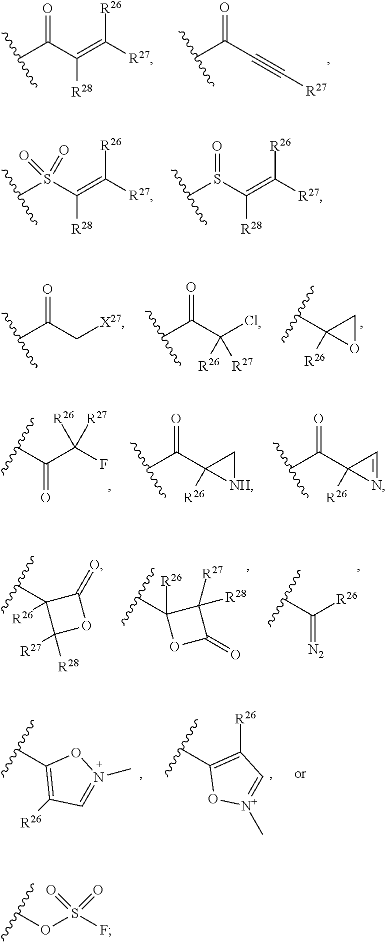

R²⁶, R²⁷, and R²⁸ are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —C(O)H, —C(O)OH, —C(O)NH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and

### X²⁷ is —F, —Cl, —Br, or —I.

Embodiment 7. The compound of one of embodiments 1 to 3, wherein

R^{10A} is -L^{10D}-L^{10E}-E;
L^{10D} is a bond;

### L^{10E} is —NH—; and

### E is

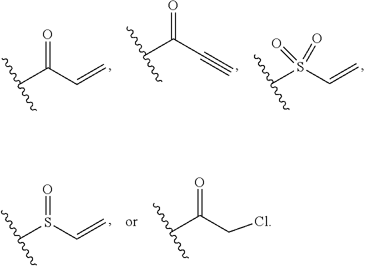

Embodiment 8. The compound of one of embodiments 1 to 3, wherein

-L^{10A}-R^{10A} is

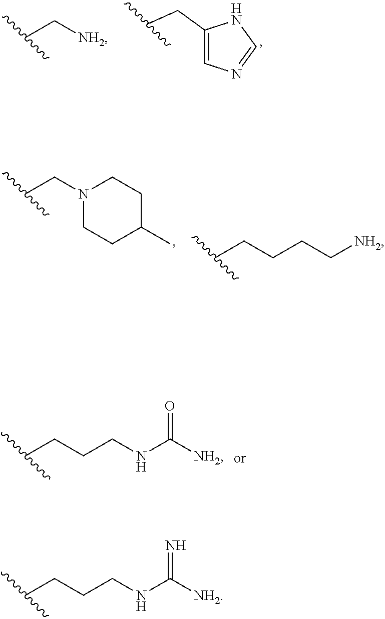

Embodiment 9. The compound of one of embodiments 1 to 8, wherein R^{3A} and R^{9A} are joined to form a bioconjugate linker.

Embodiment 10. The compound of one of embodiments 1 to 8, wherein R^{3A} and R^{9A} are joined to form a covalent linker having the formula -L^{IS}A-L^{18B}-L^{18C}-L^{18D}-L^{18E}-L^{18F}-; and

L^{18A}, L^{18B}, L^{18C}, L^{18D}, L^{18E}, and L^{18F} are independently bond, —SS—, —S(O)₂—, —OS(O)₂—, —S(O)₂O—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

Embodiment 11. The compound of one of embodiments 1 to 8, wherein R^{3A} and R^{9A} are joined to form

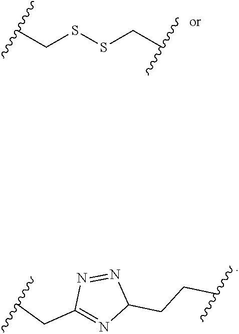 [SMILES: *CSSC* ; *CCC1N=NC(C*)=N1]

Embodiment 12. The compound of one of embodiments 1 to 3, having the formula:

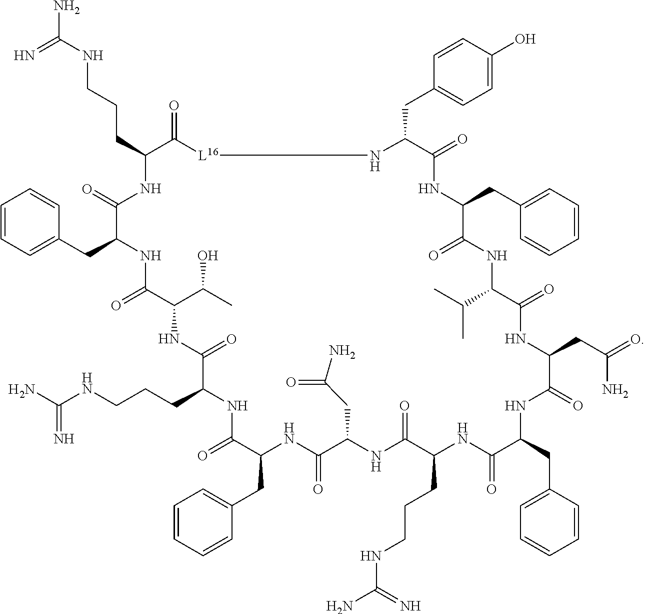

Embodiment 13. A compound having the formula:

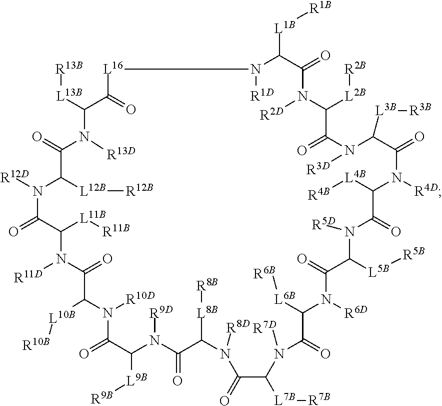

wherein
L^{1B}, L^{2B}, L^{3B}, L^{4B}, L^{5B}, L^{6B}, L^{7B}, L^{8B}, L^{9B}, L^{10B}, L^{11B}, L^{12B}, and L^{13B} are independently a bond, substituted or unsubstituted alkylene, or substituted or unsubstituted heteroalkylene;
R^{1B}, R^{8B}, and R^{10B} are independently substituted or unsubstituted cycloalkyl, substituted or unsubstituted heterocycloalkyl, substituted or unsubstituted aryl, or substituted or unsubstituted heteroaryl;
R^{2B}, R^{3B}, R^{4B}, R^{9B}, and R^{11B} are independently hydrogen, substituted or unsubstituted alkyl, substituted or unsubstituted cycloalkyl, or substituted or unsubstituted aryl;
R^{5B} is hydrogen, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, or substituted or unsubstituted heteroalkyl;
R^{6B} is hydrogen, —OH, —COOH, —NO₂, —SO₃H, —OSO₃H, substituted or unsubstituted alkyl, or substituted or unsubstituted heteroalkyl;
R^{7B}, R^{12B}, and R^{13B} are independently hydrogen, —NH₂, —CONH₂, —SO₂NH₂, —NHNH₂, —ONH₂, —NHC(O)NHNH₂, —NHC(O)NH₂, —NHC(NH)NH₂, substituted or unsubstituted alkyl, substituted or unsubstituted heteroalkyl, or substituted or unsubstituted heteroaryl;
two substituents selected from R^{1B}, R^{2B}, R^{3B}, R^{4B}, R^{5B}, R^{6B}, L^{7B}, R^{8B}, R^{9B}, R^{10B}, R^{11B}, R^{12B}, and R^{13B} may optionally be joined to form a covalent linker;
R^{1D}, R^{2D}, R^{3D}, R^{4D}, R^{5D}, R^{6D}, R^{7D}, R^{8D}, R^{9D}, R^{10D}, R¹HD, R^{12D}, and R^{13D} are independently hydrogen or unsubstituted C₁-C₄ alkyl; and
L¹⁶ is a covalent linker.

Embodiment 14. The compound of embodiment 13, having the formula:

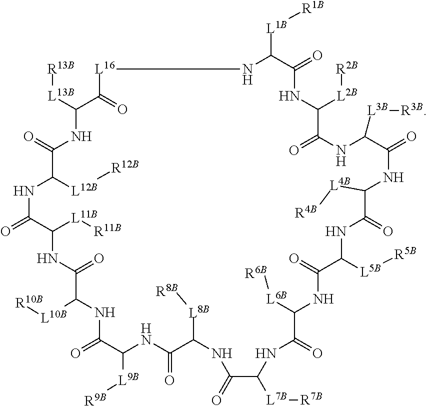

Embodiment 15. The compound of embodiment 13, having the formula:

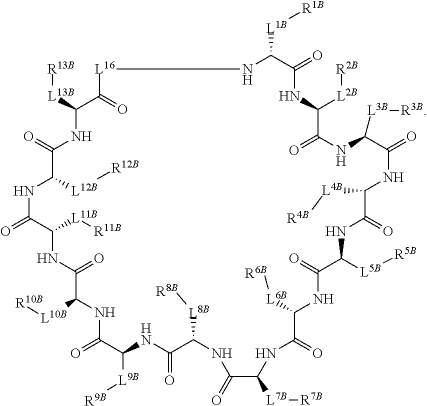

Embodiment 16. The compound of embodiment 13, having the formula:

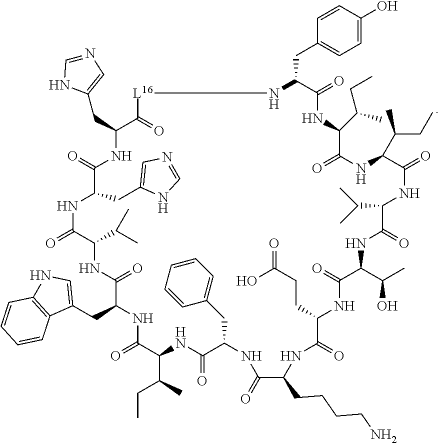

Embodiment 17. A compound having the formula:

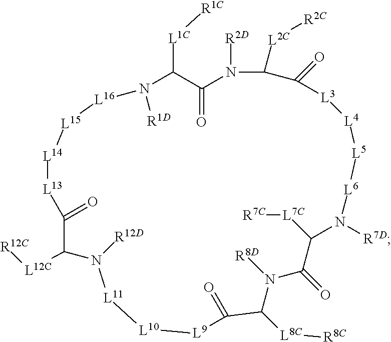

wherein
L^{1C}, L^{2C}, L^{3C}, L^{4C}, L^{5C}, L^{6C}, L^{7C}, L^{8C}, L^{9C}, L^{10C}, L^{11C}, L^{12C}, L^{13C}, L^{14C}, and L^{15C} are independently a bond, substituted or unsubstituted alkylene, or substituted or unsubstituted heteroalkylene;
R^{1C} is substituted or unsubstituted cycloalkyl, substituted or unsubstituted heterocycloalkyl, substituted or unsubstituted aryl, or substituted or unsubstituted heteroaryl;
R^{2C} is hydrogen, —OH, —NO₂, —CN, —NH₂, —C(O)OH, —C(O)NH₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, substituted or unsubstituted alkyl, or substituted or unsubstituted heteroalkyl;
L³ is a bond or

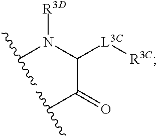

R^{3C} is hydrogen, —NH₂, —C(O)OH, —C(O)NH₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L⁴ is a bond or

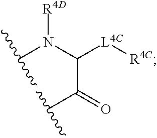

R^{4C} is hydrogen, substituted or unsubstituted alkyl, substituted or unsubstituted cycloalkyl, or substituted or unsubstituted aryl;
L⁵ is a bond or

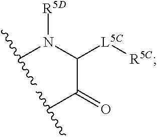

R^{5C} is substituted or unsubstituted cycloalkyl, substituted or unsubstituted heterocycloalkyl, substituted or unsubstituted aryl, or substituted or unsubstituted heteroaryl;
L⁶ is a bond,

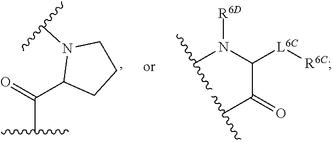

R^{6C} is hydrogen, —CN, —NH₂, —C(O)NH₂, —SO₂NH₂, —NHNH₂, —ONH₂, —NHC(O)NHNH₂, —NHC(O)NH₂, —NHC(NH)NH₂, —NHOH, substituted or unsubstituted alkyl, or substituted or unsubstituted heteroalkyl;
R^{7C} and R^{8C} are independently hydrogen, —CN, —NH₂, —C(O)NH₂, —NHNH₂, —ONH₂, —NHC(O)NHNH₂, —NHC(O)NH₂, —NHC(NH)NH₂, —NHOH, substituted or unsubstituted alkyl, or substituted or unsubstituted heteroalkyl, substituted or unsubstituted cycloalkyl, substituted or unsubstituted heterocycloalkyl, substituted or unsubstituted aryl, or substituted or unsubstituted heteroaryl;

L⁹ is a bond,

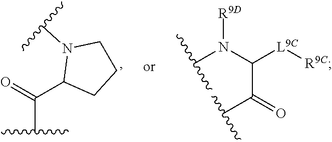

R^{9C} is hydrogen, substituted or unsubstituted alkyl, substituted or unsubstituted cycloalkyl, or substituted or unsubstituted aryl;
L¹⁰ is a bond,

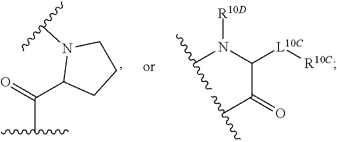

R^{10C} is hydrogen, substituted or unsubstituted alkyl, or substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L¹¹ is a bond or

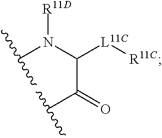

R^{11C} is hydrogen, —CN, —OH, —C(O)OH, —NO₂, —SO₃H, —OSO₃H, —NH₂, —C(O)NH₂, —SO₂NH₂, —NHNH₂, —ONH₂, —NHC(O)NHNH₂, —NHC(O)NH₂, —NHC(NH)NH₂, substituted or unsubstituted alkyl, or substituted or unsubstituted heteroalkyl;
R^{12C} is hydrogen, substituted or unsubstituted alkyl, or substituted or unsubstituted heteroalkyl, substituted or unsubstituted cycloalkyl, substituted or unsubstituted heterocycloalkyl, substituted or unsubstituted aryl, or substituted or unsubstituted heteroaryl;

### L¹ is

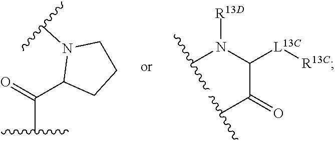

R^{13C} is hydrogen, —OH, —NH₂, —C(O)OH, —C(O)NH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, or substituted or unsubstituted heteroalkyl, substituted or unsubstituted cycloalkyl, or substituted or unsubstituted aryl;
L¹⁴ is a bond or

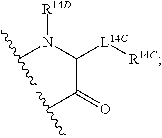

R^{14C} is hydrogen, —NH₂, —C(O)OH, —C(O)NH₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, substituted or unsubstituted alkyl, or substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L is a bond or

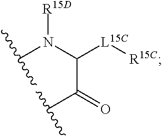

R^{15C} is hydrogen, —NH₂, —C(O)OH, —C(O)NH₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, substituted or unsubstituted alkyl, or substituted or unsubstituted heteroalkyl, substituted or unsubstituted cycloalkyl, substituted or unsubstituted heterocycloalkyl, substituted or unsubstituted aryl, or substituted or unsubstituted heteroaryl; two substituents selected from R^{1C}, R^{2C}, R^{3C}, R^{4C}, R^{5C}, R^{6C}, R^{7C}, R^{8C}, R^{9C}, R^{10C}, R^{11C}, R^{12C}, R^{13C}, R^{14C}, and R^{15C} may optionally be joined to form a covalent linker;
R^{1D}, R^{2D}, R^{3D}, R^{4D}, R^{5D}, R^{6D}, R^{7D}, R^{8D}, R^{9D}, R^{10D}, R^{11D}, R^{12D}, R^{13D}, R^{14D}, and R^{15D} are independently hydrogen or unsubstituted C₁-C₄ alkyl; and
L¹⁶ is a covalent linker.

Embodiment 18. The compound of embodiment 17, having the formula:

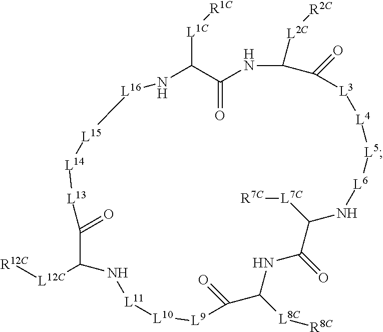

wherein
R^{3D}, R^{4D}, R^{5D}, R^{6D}, R^{9D}, R^{10D}, R^{11D}, R^{13D}, R^{14D}, and R^{15D} are hydrogen.

Embodiment 19. The compound of embodiment 18, having the formula:

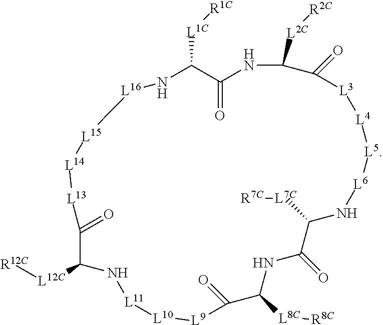

Embodiment 20. The compound of embodiment 18, having the formula:

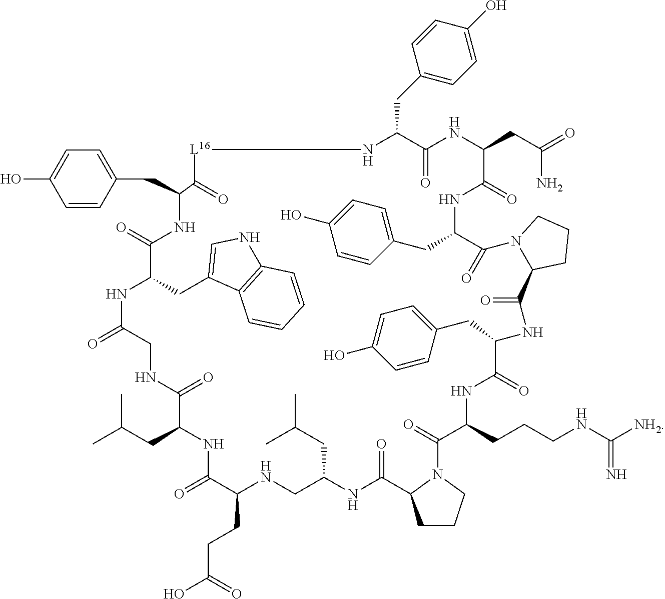

Embodiment 21. The compound of one of embodiments 1 to 20, wherein L¹⁶ is a bioconjugate linker.

Embodiment 22. The compound of one of embodiments 1 to 20, wherein L¹⁶ is a substituted or unsubstituted divalent amino acid.

Embodiment 23. The compound of embodiment 22, wherein L¹⁶ is a substituted or unsubstituted divalent δ-amino acid.

Embodiment 24. The compound of one of embodiments 1 to 20, wherein

L¹⁶ is -L^{16A}-L^{16B}-L^{16C}-L^{16D}-L^{16E}-L^{16F}-; and
L^{16A}, L^{16B}, L^{16C}, L^{16D}, L¹⁶E, and L^{16F} are independently bond, —SS—, S(O)₂—, —OS(O)₂—, —S(O)₂O—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

Embodiment 25. The compound of embodiment 24, wherein

L¹⁶ is —NH-L^{16B}-L^{16C}-L^{16D}-L^{16E}-C(O)—;

### L^{16B} is

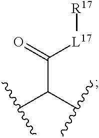

L^{16C}, L^{16D}, and L^{16E} are independently bond, —SS—, —S(O)₂—, —OS(O)₂—, —S(O)₂O—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹⁷ is -L^{17A}-L^{17B}-L^{17C}-L^{17D}-L^{17E}-L^{17F}-;
L^{17A}, L^{17B}, L^{17C}, L^{17D}, L^{17E}, and L^{17F} are independently bond, —SS—, —S(O)₂—, —OS(O)₂—, —S(O)₂O—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
R¹⁷ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —C(O)H, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, a monovalent nucleic acid, a monovalent protein a detectable moiety, or a drug moiety.

Embodiment 26. The compound of embodiment 24, wherein L¹⁶ is a bond,

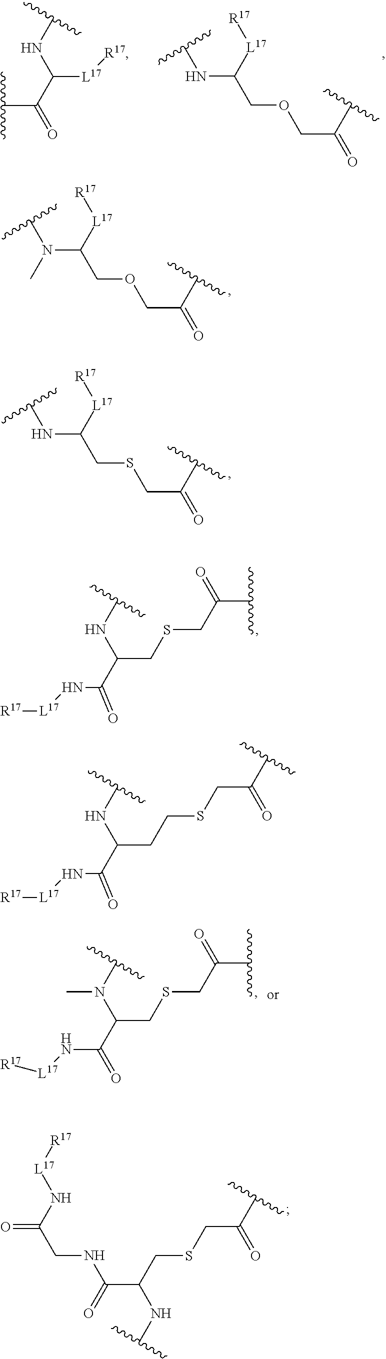

L¹⁷ is -L^{17A}-L^{17B}-L^{17C}-L^{17D}-L^{17E}-L^{17F}-;
L^{17A}, L^{17B}, L^{17C}, L^{17D}, L^{17E}, and L^{17F} are independently a bond, —SS—, —S(O)₂—, —OS(O)₂—, —S(O)₂O—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
R¹⁷ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —C(O)H, —C(O)OH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, a monovalent nucleic acid, a monovalent protein, a detectable moiety, or a drug moiety.

Embodiment 27. The compound of embodiment 24, wherein L¹⁶ is a bond,

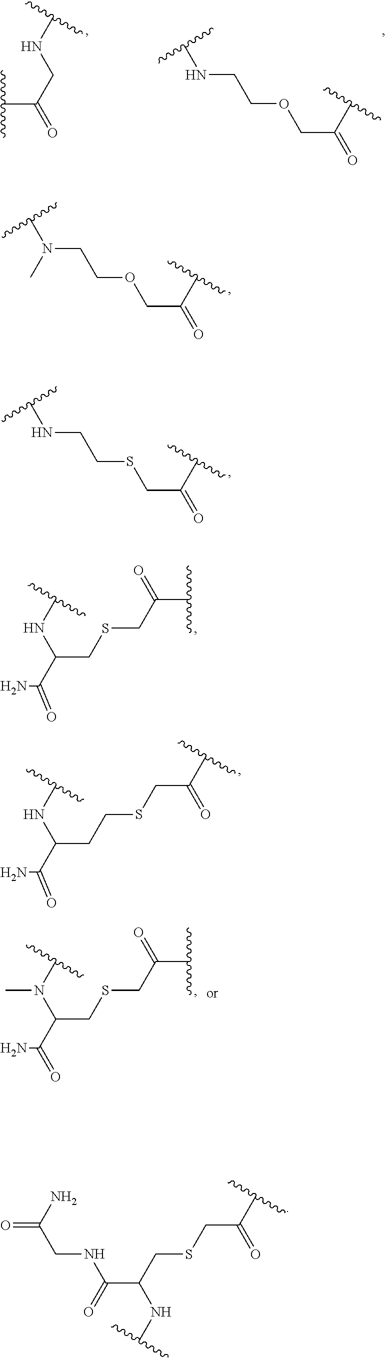

Embodiment 28. A compound having the formula:

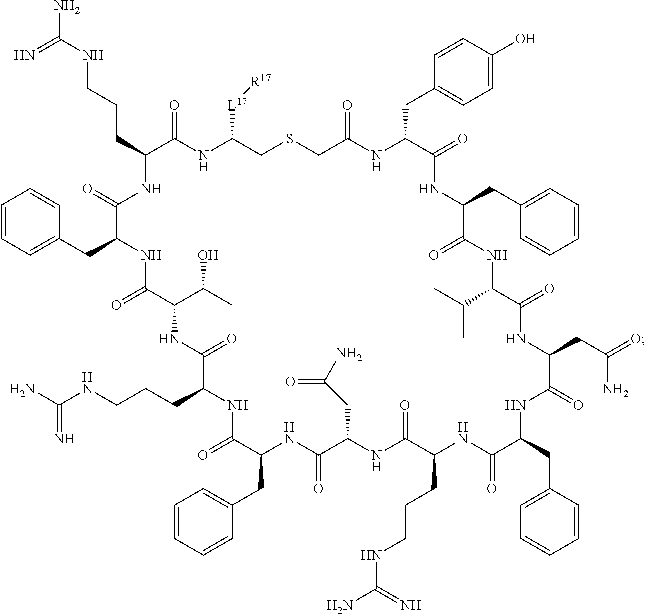

wherein
L¹⁷ is -L^{17A}-L^{17B}-L^{17C}-L^{17D}-L^{17E}-L^{17F}-;
L^{17A}, L^{17B}, L^{17C}, L^{17D}, L^{17E}, and L^{17F} are independently bond, —SS—, —S(O)₂—, —OS(O)₂—, —S(O)₂O—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
R¹⁷ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —C(O)H, —C(O)OH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, a monovalent nucleic acid, a monovalent protein, a detectable moiety, or a drug moiety.

Embodiment 29. A compound having the formula:

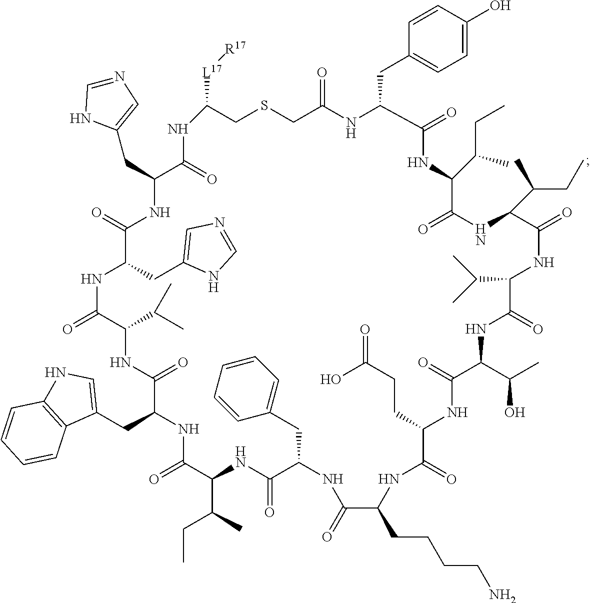

wherein
L¹⁷ is -L^{17A}-L^{17B}-L^{17C}-L^{17D}-L^{17E}-L^{17F}-;
L^{17A}, L^{17B}, L^{17C}, L^{17D}, L^{17E}, and L^{17F} are independently bond, —SS—, —S(O)₂—, —OS(O)₂—, —S(O)₂O—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
R¹⁷ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —C(O)H, —C(O)OH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, a monovalent nucleic acid, a monovalent protein, a detectable moiety, or a drug moiety.

Embodiment 30. A compound having the formula:

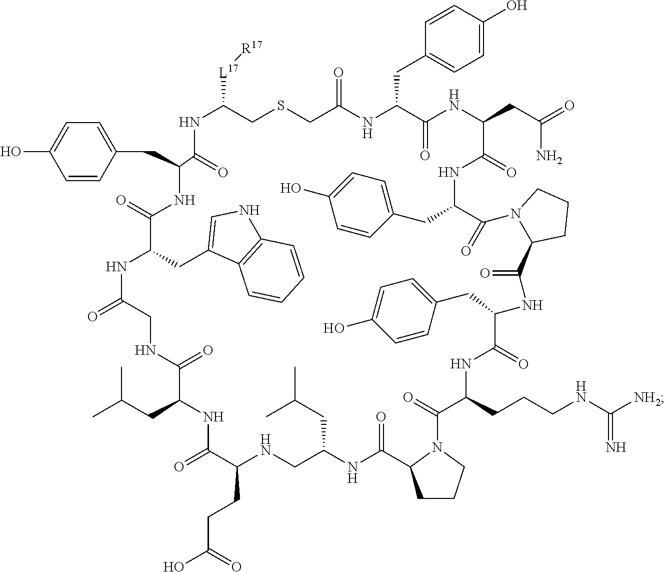

wherein
L¹⁷ is -L^{17A}-L^{17B}-L^{17C}-L^{17D}-L^{17E}-L^{17F}-;
L^{17A}, L^{17B}, L^{17C}, L^{17D}, L^{17E}, and L^{17F} are independently bond, —SS—, —S(O)₂—, —OS(O)₂—, —S(O)₂O—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
R¹⁷ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —C(O)H, —C(O)OH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, a monovalent nucleic acid, a monovalent protein, a detectable moiety, or a drug moiety.

Embodiment 31. A compound having the formula:

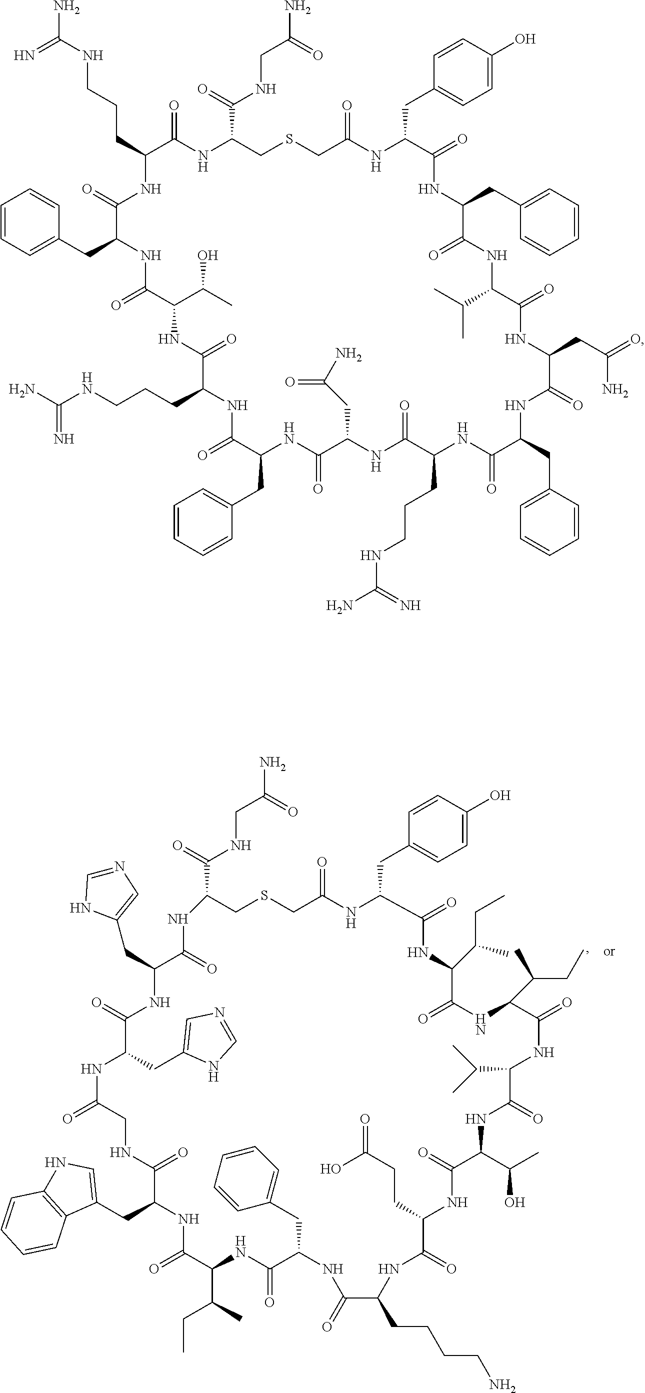

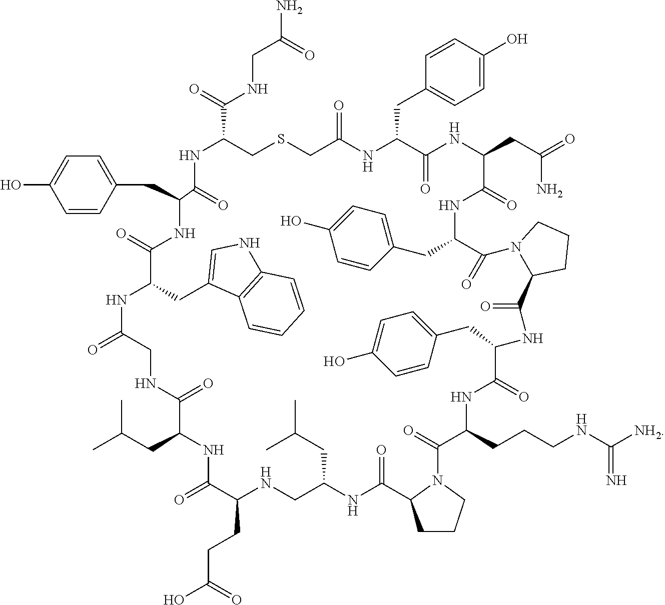 [SMILES: CC(C)C[C@H]1CN[C@@H](CCC(=O)O)C(=O)N[C@@H](CC(C)C)C(=O)NCC(=O)N[C@@H](Cc2c[nH]c3ccccc23)C(=O)N[C@@H](Cc2ccc(O)cc2)C(=O)N[C@H](C(=O)NCC(N)=O)CSCC(=O)N[C@H](Cc2ccc(O)cc2)C(=O)N[C@@H](CC(N)=O)C(=O)N[C@@H](Cc2ccc(O)cc2)C(=O)N2CCC[C@H]2C(=O)N[C@@H](Cc2ccc(O)cc2)C(=O)N[C@@H](CCCNC(=N)N)C(=O)N2CCC[C@H]2C(=O)N1]

Embodiment 32. The compound of one of embodiments 1 to 31, wherein said compound contacts the Switch 2 groove of human K-Ras protein.

Embodiment 33. The compound of one of embodiments 1 to 31, wherein said compound contacts a Switch 2 groove amino acid corresponding to G60, Q61, D69, D92, H95, Y96, or Q99 of human K-Ras protein.

Embodiment 34. The compound of one of embodiments 1 to 31, wherein said compound binds a human K-Ras protein-GTP complex more strongly than said compound binds a human K-Ras protein-GDP complex under identical conditions.

Embodiment 35. The compound of embodiment 34, wherein said compound binds a human K-Ras protein-GTP complex at least 2-fold stronger than said compound binds a human K-Ras protein-GDP complex under identical conditions.

Embodiment 36. The compound of embodiment 34, wherein said compound binds a human K-Ras protein-GTP complex at least 5-fold stronger than said compound binds a human K-Ras protein-GDP complex under identical conditions.

Embodiment 37. The compound of embodiment 34, wherein said compound binds a human K-Ras protein-GTP complex at least 40-fold stronger than said compound binds a human K-Ras protein-GDP complex under identical conditions.

Embodiment 38. The compound of embodiment 34, wherein said compound binds a human K-Ras protein-GTP complex at least 100-fold stronger than said compound binds a human K-Ras protein-GDP complex under identical conditions.

Embodiment 39. The compound of one of embodiments 1 to 38, wherein said compound binds a human K-Ras G12D protein more strongly than said compound binds a human K-Ras wildtype protein under identical conditions.

Embodiment 40. The compound of one of embodiments 1 to 38, wherein said compound binds a human K-Ras G12D protein at least 2-fold stronger than said compound binds a human K-Ras wildtype protein under identical conditions.

Embodiment 41. The compound of one of embodiments 1 to 38, wherein said compound binds a human K-Ras G12D protein at least 10-fold stronger than said compound binds a human K-Ras wildtype protein under identical conditions.

Embodiment 42. The compound of one of embodiments 1 to 38, wherein said compound binds a human K-Ras G12D protein at least 100-fold stronger than said compound binds a human K-Ras wildtype protein under identical conditions.

Embodiment 43. The compound of one of embodiments 1 to 42, wherein said compound is capable of binding a water molecule and wherein said water molecule simultaneously binds a D12 residue of a human K-Ras G12D protein.

Embodiment 44. The compound of one of embodiments 1 to 42, wherein said compound binds a human K-Ras G12D protein and wherein the shortest average distance between the compound and the D12 residue of said human K-Ras protein is from 1.5 Å to 6.0 Å.

Embodiment 45. The compound of one of embodiments 1 to 42, wherein said compound binds a human K-Ras protein-GTP complex and wherein the shortest average distance between the compound and terminal phosphate of the GTP of said human K-Ras protein-GTP complex is from 1.5 Å to 10.0 Å.

Embodiment 46. A pharmaceutical composition comprising the compound of any one of embodiments 1 to 45, or a pharmaceutically acceptable salt thereof, and a pharmaceutically acceptable excipient.

Embodiment 47. A method of treating a cancer in a patient in need of such treatment, said method comprising administering a therapeutically effective amount of a compound of any one of embodiments 1 to 45 to said patient.

Embodiment 48. A method of modulating the activity of a human K-Ras protein, said method comprising contacting said human K-Ras protein with an effective amount of a compound of any one of embodiments 1 to 45.

Embodiment 49. The method of embodiment 48, wherein said human K-Ras protein is a human K-Ras G12D protein, a human K-Ras G12C protein, a human K-Ras G12S protein, a human K-Ras G12V protein, or a human K-Ras G12R protein.

Embodiment 50. The method of embodiment 48, wherein said human K-Ras protein comprises a Q61 mutation.

Embodiment 51. The method of one of embodiments 48 to 50, wherein the activity of the human K-Ras protein is GTPase activity, nucleotide exchange, GDP binding, GTP binding, differential GDP or GTP binding, effector protein binding, K-Ras binding to Raf, effector protein activation, guanine exchange factor (GEF) binding, GEF-facilitated nucleotide exchange, phosphate release, nucleotide release, nucleotide binding, K-Ras subcellular localization, K-Ras post-translational processing, or K-Ras post-translational modifications.

Embodiment 52. The method of one of embodiments 48 to 50, wherein the activity of the human K-Ras protein is K-Ras binding to Raf.

Embodiment 53. A method of modulating the activity of a human H-Ras protein, said method comprising contacting said human H-Ras protein with an effective amount of a compound of any one of embodiments 1 to 45.

Embodiment 54. A method of modulating the activity of a human N-Ras protein, said method comprising contacting said human N-Ras protein with an effective amount of a compound of any one of embodiments 1 to 45.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1: GTP State-Selective Cyclic Peptide Ligands of K-Ras(G12D) Block its Interaction with Raf

We report herein, inter alia, the identification of three cyclic peptide ligands of K-Ras(G12D) using an integrated in vitro translation-mRNA display selection platform. These cyclic peptides show preferential binding to the GTP-bound state of K-Ras(G12D) over the GDP-bound state and block Ras-Raf interaction. A co-crystal structure of peptide KD2 with K-Ras(G12D)·GppNHp reveals that this peptide binds in the Switch II Groove region with concomitant opening of the Switch II loop and a 40° rotation of the α2 helix, and that a threonine residue (Thr10) on KD2 has direct access to the mutant aspartate (Asp12) on K-Ras. Replacing this threonine with non-natural amino acids afforded peptides with improved potency at inhibiting the interaction between Raf1-RBD and K-Ras(G12D) but not wildtype K-Ras. The union of G12D over wildtype selectivity and GTP-state/GDP-state selectivity is particularly desirable, considering that oncogenic K-Ras(G12D) exists predominantly in the GTP state in cancer cells, and wildtype K-Ras signaling is important for the maintenance of healthy cells.

Missense mutations of the RAS genes (KRAS, HRAS, and NRAS) occur frequently in human cancer and drive oncogenic transformation (1). Among these, KRAS G12D is the most prevalent point mutation associated with poor clinical outcome. The G12D mutation impairs both intrinsic and GTPase-accelerating protein (GAP)-mediated GTP hydrolysis and liberates K-Ras protein from functional control by GTPase activity (2,3). As a result, K-Ras(G12D) is enriched in its GTP-bound, signaling-competent state, given the near 10-fold higher concentration of GTP than GDP inside the cell (4).

Though Ras proteins were historically considered “undruggable” due to their picomolar affinity for guanine nucleotides and the lack of deep accessible pockets, recent efforts have fueled the discovery of both small molecule and macromolecule direct binders of Ras (5,6,15-24, 7-14). Earlier work from our laboratory identified a class of compounds that bind to Ras in a highly dynamic pocket near the Switch II region (SII-P) and leverage the nucleophilicity of the acquired cysteine in K-Ras(G12C) to covalently capture K-Ras in its inactive, GDP-bound state (18). Further chemical optimization has yielded ligands with potent K-Ras(G12C) dependent antitumor effects (25-27), and three compounds (AMG510 (28,29), MRTX849 (30), JNJ-74699157 (31)) have entered clinical trials in patients with K-Ras(G12C) mutant tumors (32). This strategy is uniquely suited for the G12C mutant because intrinsic GTPase activity is not affected by the G12C mutation (3), allowing the conversion from GTP-bound state to the compound-accessible GDP-bound state at a clinically relevant rate. Efforts to develop K-Ras(G12C) inhibitors capable of binding to the GTP-state have thus far been unsuccessful. The SIIP is occluded in the GTP-state of K-Ras effectively blocking the drug binding pocket in this protein state.

The loss of intrinsic GTPase activity in K-Ras(G12D) presents an additional challenge—in cells with the G12D mutation, only a minor fraction of the K-Ras protein will be GDP-bound, and conversion from GTP-bound state to GDP-bound state is extremely slow. To target K-Ras(G12D), we envision that a GTP-state selective K-Ras ligand will be advantageous because 1) it will bind to the predominant K-Ras population in G12D-mutant cells, and 2) it will be less likely to affect healthy cells, which express wildtype K-Ras with a significant GDP-bound population. Previous work has demonstrated that targeting the GTP-state of Ras is feasible. For example, Gentile et al. discovered Switch II Groove (SII-G) ligands that can recognize both GDP- and GTP-states of Ras, albeit with a preference for the GDP-state (20). Sakamoto et al. reported K-Ras(G12D)-targeting cyclic peptides generated from a random T7 phage display library that are weakly selective for the GDP-state and inhibit Sos-mediated nucleotide exchange (9, 33, 34). Wu et al. identified artificial cyclic peptides that block K-Ras(G12V)/Raf1-RBD interaction from a bead-display library but did not study the nucleotide state preference (35). To the best of our knowledge, GTP-state selective ligands have not been documented. Here we present the discovery of GTP-state selective and mutant-selective cyclic peptide ligands of K-Ras(G12D) using the random nonstandard peptides integrated discovery (RaPID) platform (36), an in vitro translation-mRNA-display technology encoding >10¹² macrocyclic peptides.

To target the GTP-state of K-Ras(G12D) we first considered a potential challenge: a ligand identified from a binding rather than a functional screen may not antagonize K-Ras oncogenic function. For example, an allosteric ligand that stabilizes the active conformation K-Ras(G12D)·GTP, but still allows effector binding, will render the protein constitutively active. To minimize this possibility, we took advantage of the two conformational states of Ras·GTP with distinct properties: State 1 (effector binding incompetent) and State 2 (effector binding competent) (37). While most Ras proteins exist in an equilibrium of these two interconverting states, a mutation of Thr35 to Ser locks the protein in State 1 (38). We therefore elected to use the double mutant K-Ras(G12D/T35S) for our initial selection, with the GppNHp-loaded protein as the selection target, and the GDP-loaded protein as the counter-target (FIG. 1A).

Starting from a high-diversity cDNA library (>10¹² encoded compounds), we performed five rounds of selection, and examined the output cDNA libraries by next generation sequencing (NGS). Each round of selection included in vitro transcription, puromycin ligation, N-chloroacetyl-D-tyrosine-initiated in vitro translation with in situ cyclization, reverse transcription, negative and positive selection with target protein (K-Ras(G12D)) immobilized on streptavidin beads, and PCR amplification of the selected cDNA libraries. At the end of the fifth round of selection, we subjected the output library to an additional round of selection for quantitative comparison of the binders to empty beads, immobilized K-Ras(G12D)·GDP, or immobilized K-Ras(G12D)·GppNHp using PCR and NGS. This allowed us to calculate the GTP/GDP selectivity index (to evaluate GTP-state selectivity) and the GTP/beads selectivity index (to assess the level of non-specific binding). The top 20 binders (ranked by number of NGS reads after round 5) contained 16 cyclic peptides with GTP/GDP selectivity index of >1 (FIG. 1B). These 16 peptides clustered into three distinct scaffolds, and members in each cluster displayed surprisingly high sequence homology. We chose one representative member from each cluster (namely, KD1, KD2, and KD17) and chemically synthesized linker-free cyclic peptides in multi-milligram quantities for further study.

Interestingly, when we performed a separate GTP-state positive selection using empty beads in lieu of GDP-loaded protein for negative selection, we obtained predominantly GDP-state selective binders (FIGS. 2A-2B), despite that the positive selection target was the same GppNHp-loaded protein.

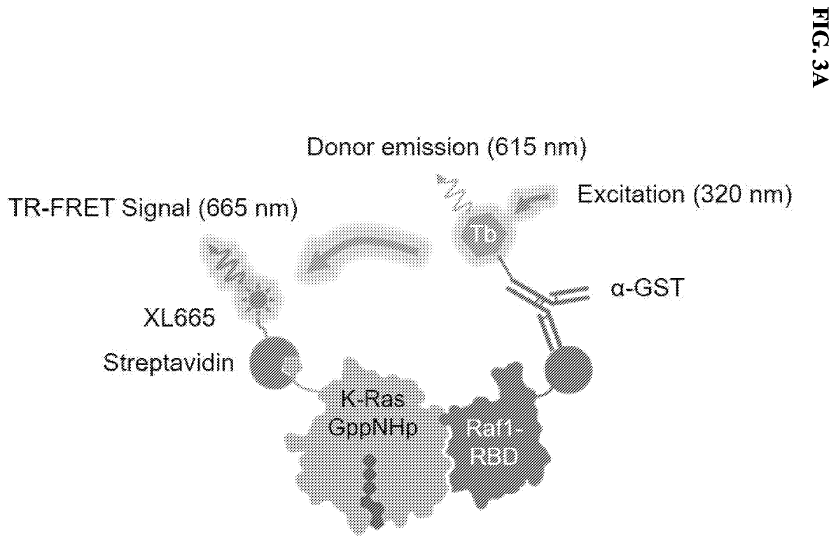

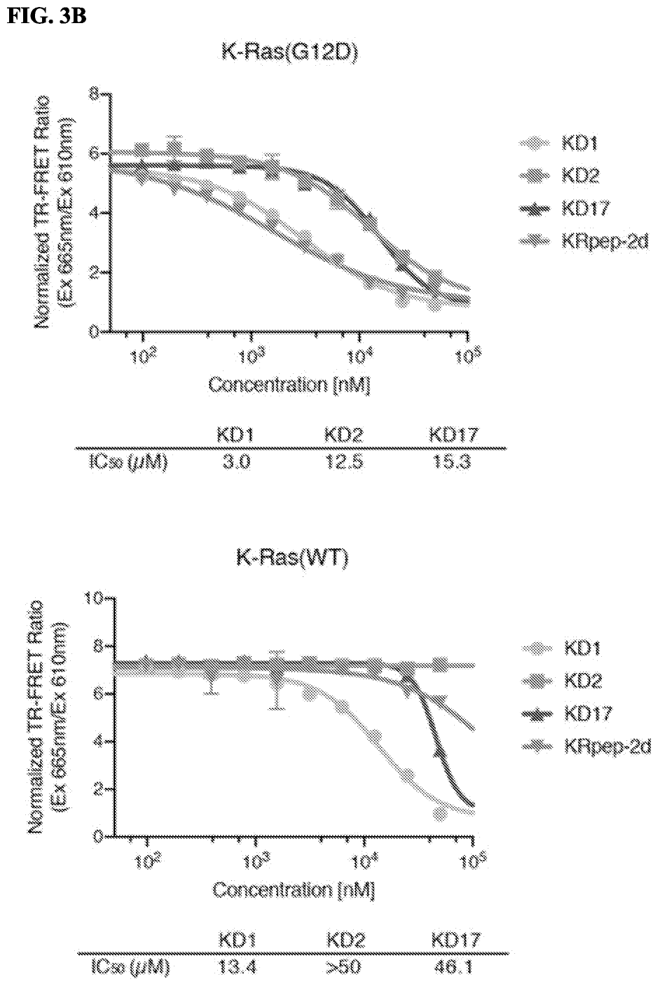

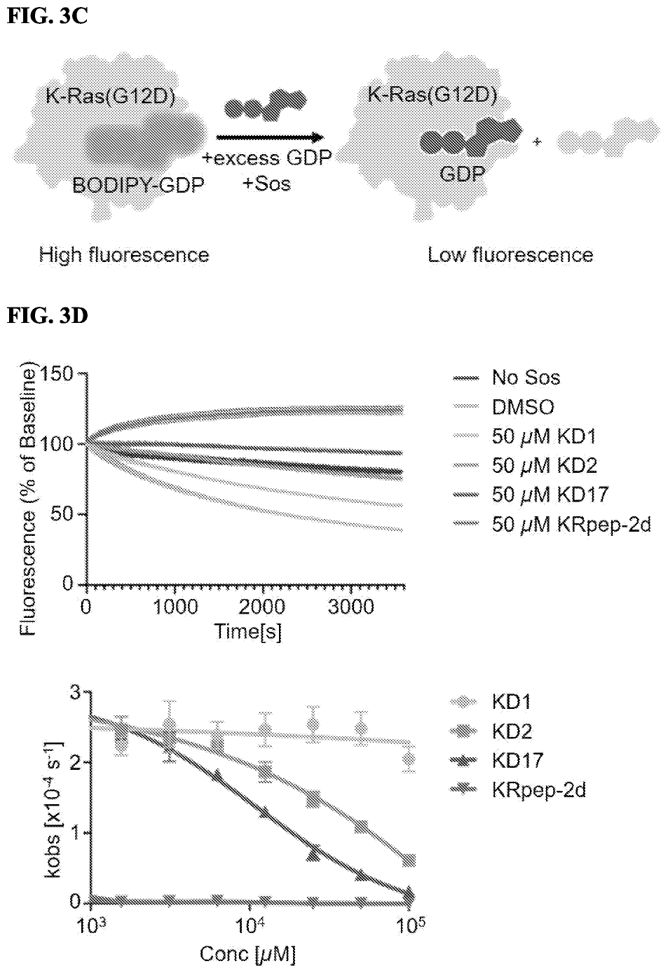

With these three GTP-state selective cyclic peptide ligands, we assessed their impact on effector binding to K-Ras(G12D). We used a time-resolved fluorescence energy transfer (TR-FRET) assay which allows the quantitation of the Ras·Raf complex formation (FIG. 3A). All three peptides inhibited the interaction between K-Ras(G12D)·GppNHp and Raf1-RBD at micromolar concentrations. Meanwhile, all three peptides were less potent at inhibiting wildtype K-Ras·GppNHp interaction with Raf1-RBD, with different levels of selectivity. Surprisingly, KD2 did not exhibit an inhibitory effect against wildtype K-Ras at the highest solubility-permitting concentration. We reasoned that the observed selectivity could come from two sources: 1) K-Ras(G12D) is known to have a weaker affinity for Raf RBD than wildtype K-Ras (K_{d}'s: 270±46 nM and 56±6 nM, respectively) (3); 2) the cyclic peptide may exhibit preferential recognition for the aspartate-12 residue.

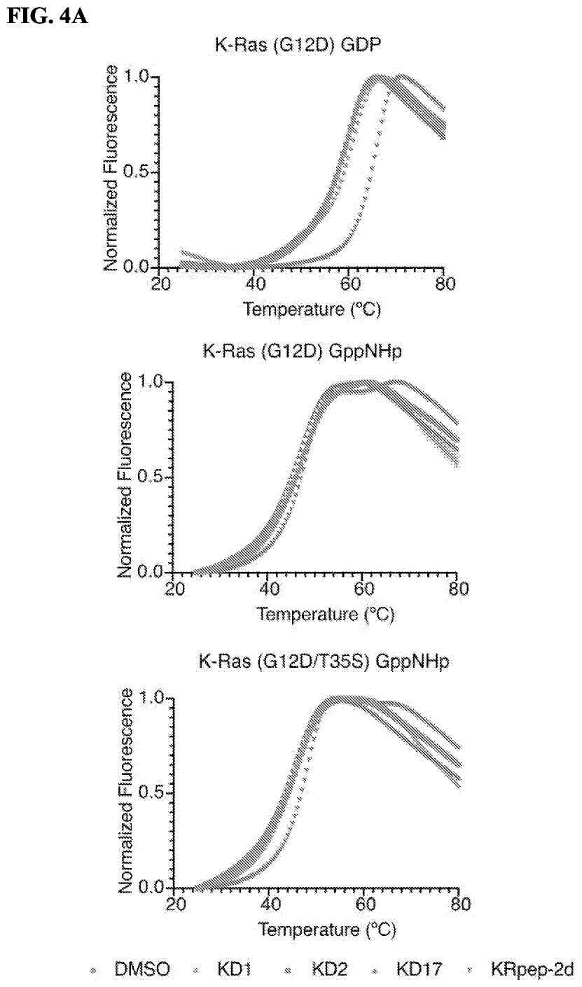

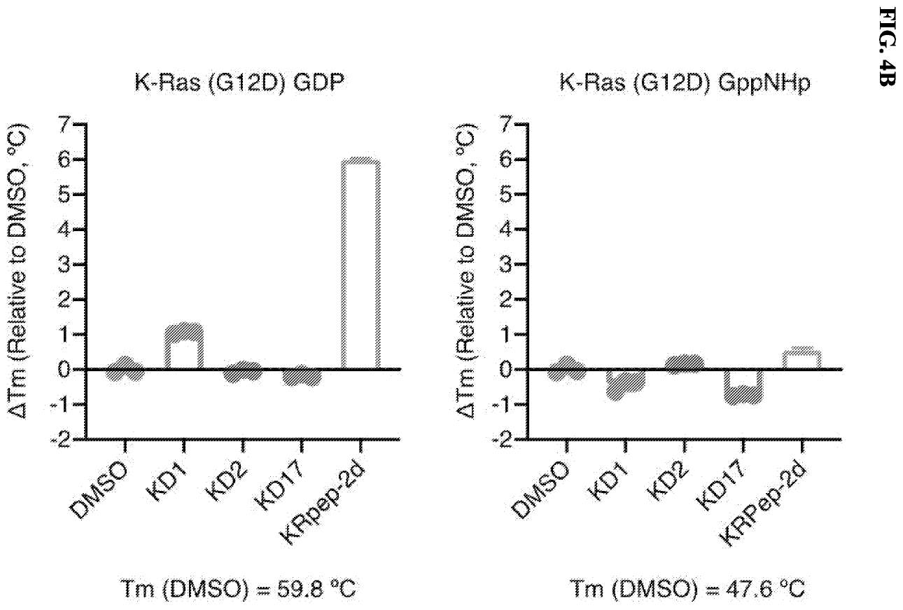

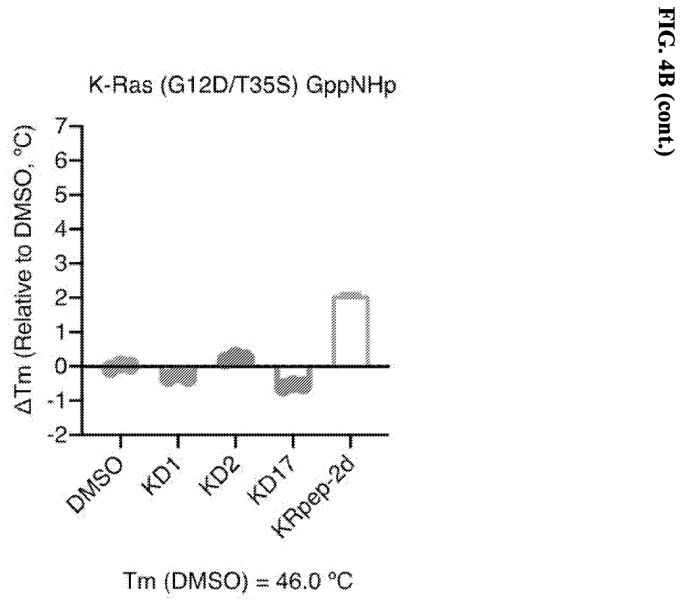

We next determined whether these cyclic peptides affect Sos-mediated nucleotide exchange, which takes GDP-state Ras as substrate. Inhibition of Sos-mediated nucleotide exchange required high concentrations of KD2 and KD17, and even near the solubility limit of 50 μM, KD1 only had a small effect on the rate of nucleotide exchange. By contrast, KRpep2d (a cyclic peptide ligand of K-Ras reported by Takeda (9)) is a highly potent inhibitor of nucleotide exchange, with an IC₅₀ below the lower assay limit of the current assay format. None of these three peptides stabilized K-Ras(G12D) against thermal denaturation either in their GDP-bound or GppNHp-bound state (ΔTm<1.0° C., FIGS. 4A-4B). By contrast, KRpep2d increased the melting temperature of K-Ras(G12D)·GDP by 6.0° C., but had little stabilization effect on K-Ras(G12D)·GppNHp.

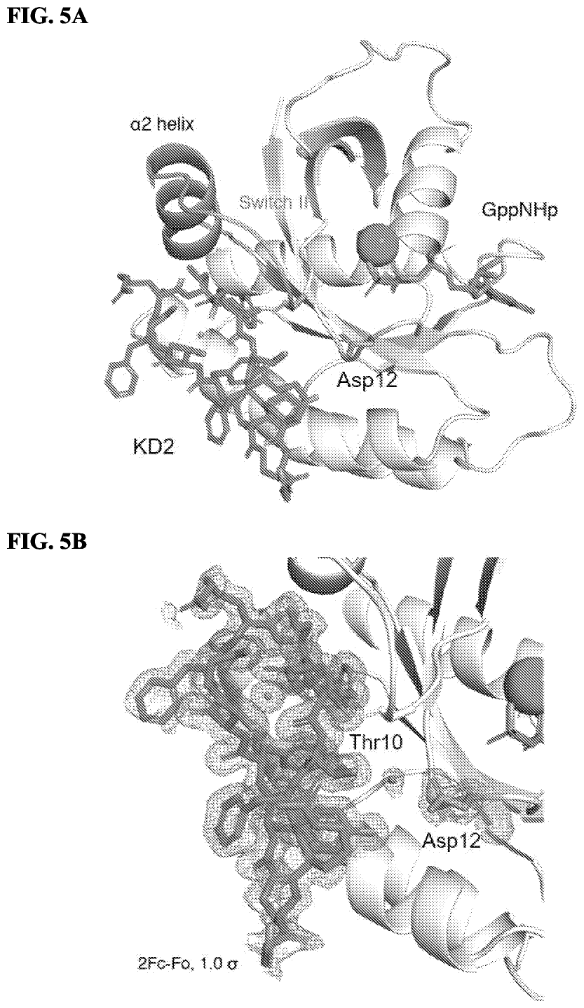

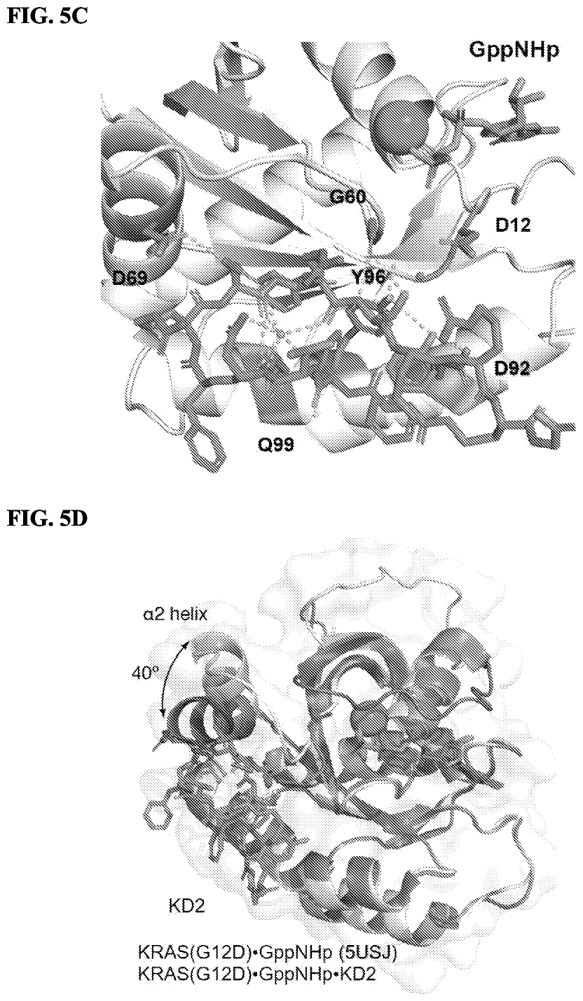

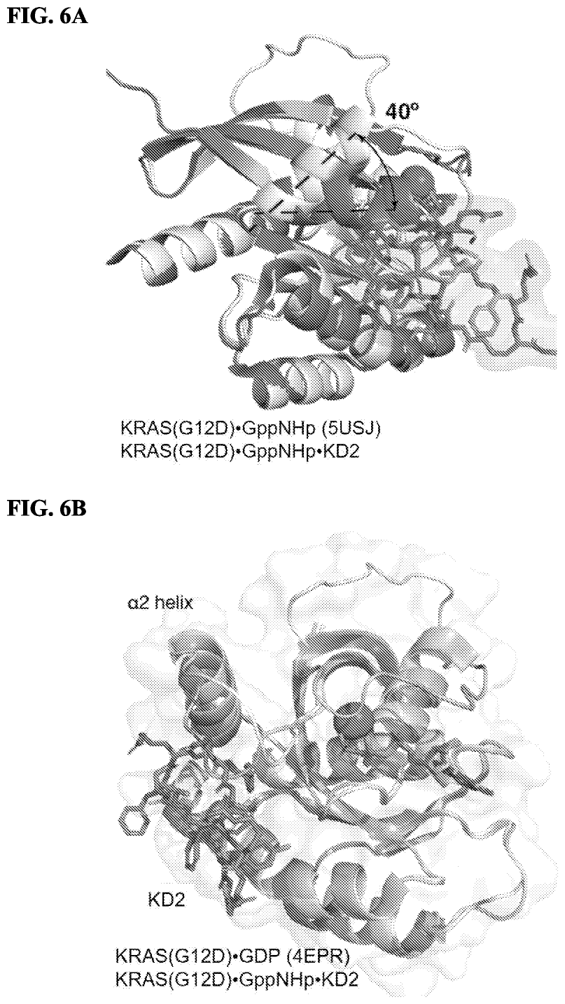

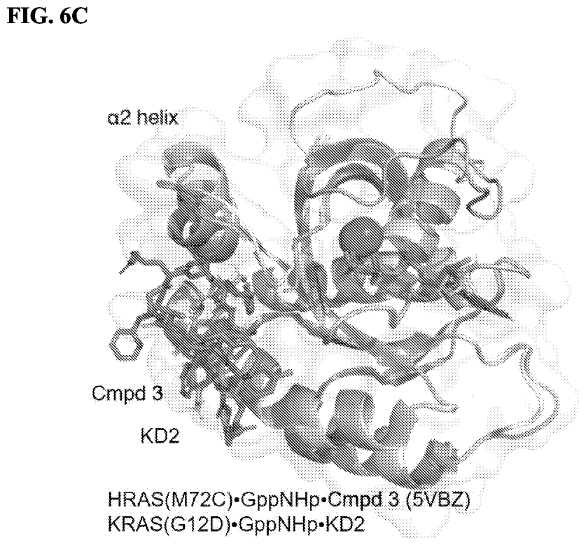

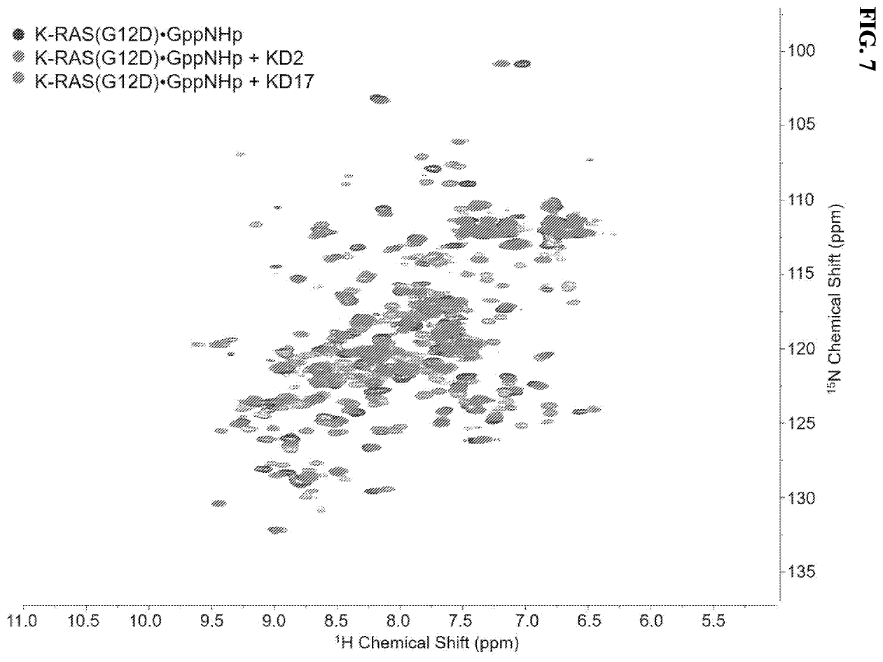

Owing to its GTP/GDP state selectivity (as revealed by selection NGS reads) and G12D/wildtype selectivity (as revealed by the Ras·Raf interaction assay), we focused our additional efforts on KD2. We successfully obtained co-crystals of KD2 and K-Ras(G12D) Cyslight·GppNHp (Cyslight) in a construct where all cysteines have been mutated to serine or leucine and which we previously found to have improved crystallization properties), and the crystal structure was determined by molecular replacement and refined to 1.6 Å (Table 1). The overall complex structure is shown in FIG. 5A, with the mutant aspartate residue (Asp12) highlighted, and KD2 and GppNHp shown in stick models. KD2 binds in the Switch II Groove region, below the α2 helix and the Switch II loop, the same pocket as previously reported covalent ligands targeting the engineering the cysteine in H-Ras(M72C)·GppNHp (20). This pocket is not observable in any published structures of the non-liganded GTP-state K-Ras. For example, examination of the structure of K-Ras(G12D)·GppNHp (PDB:5USJ) reveals a closed surface between Switch II and 3 helix. However, KD2 appears to have expanded the pocket by inducing a large shift of the α2 helix and the switch II loop (FIG. 5D, FIG. 6A).

KD2 forms an intricate hydrogen bond network both within the macrocycle and with residues on K-Ras(G12D). One intriguing observation was an ordered water molecule in the center of the macrocycle, forming hydrogen bonds with both side chain and backbone elements of cyclic KD2. We surmise that this water molecule may be critical to maintaining the conformation of the macrocycle. KD2 interacts with K-Ras(G12D) through residues on various domains (FIG. 5C), including G60 (Switch II), D69 (α2 helix), D92 (α3 helix), Y96 (α3 helix) and Q99 (α3 helix). Particularly remarkable is that the mutant aspartate residue (Asp12) is directly accessible from Thr10 on the cyclic peptide. We observed a low occupancy water molecule bridging these two amino acids in the crystal structure. To the best of our knowledge, this is the first ligand-bound crystal structure of K-Ras(G12D) in its GTP-state, where the ligand makes direct contact with the mutant residue at position 12.

To probe the structural perturbation of K-Ras(G12D) by all three cyclic peptides, we performed ¹H-¹⁵N heteronuclear single quantum coherence (HSQC) experiments with 100 μM K-Ras(G12D)·GppNHp in the presence of 200 μM cyclic peptide. The low solubility of KD1 in aqueous buffer (˜50 μM) precluded the acquisition of the HSQC spectrum for this peptide. Both KD2 and KD17 caused drastic perturbations of nearly all peaks in the HSQC spectrum of K-Ras(G12D)·GppNHp, with additional new peaks that are not present in the spectrum of unliganded K-Ras(G12D)·GppNHp (FIG. 7). These cyclic peptides seem to have slow dissociation rates and titration of the ligand did not allow us to trace the chemical shift change of each peak (instead, we observed two distinct populations). Among the easily identifiable peaks, G77 showed a +0.10 ppm ¹H chemical shift change upon the binding of either KD2 or KD17.

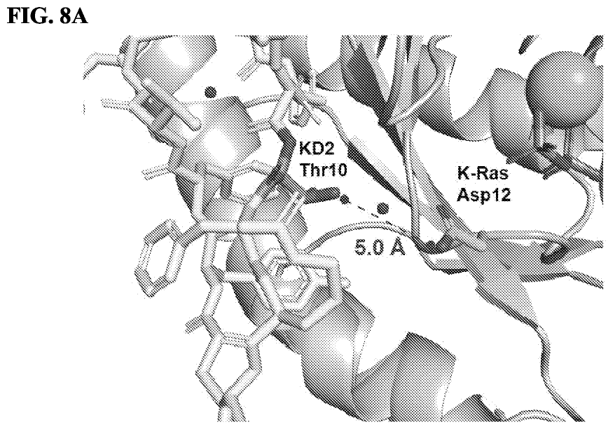

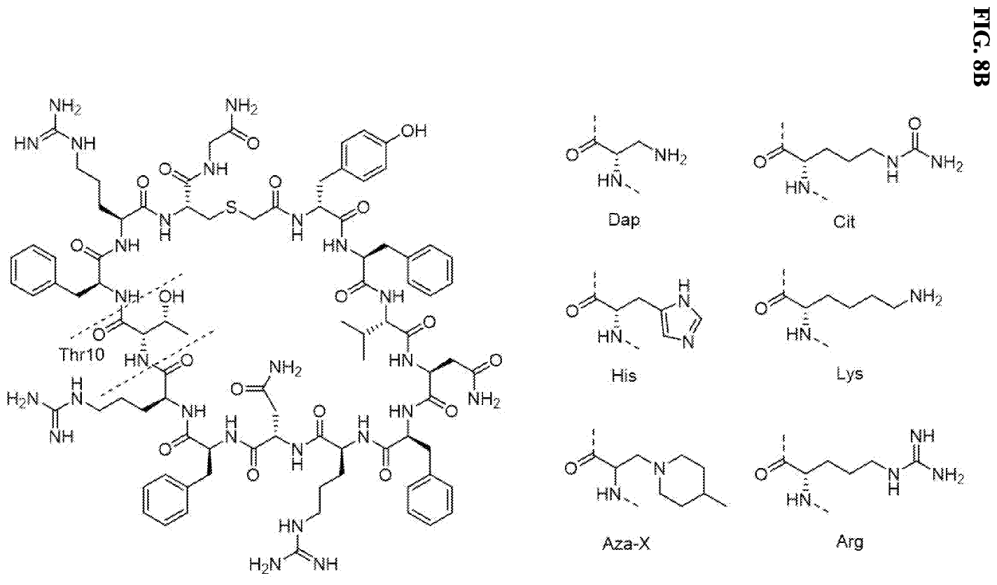

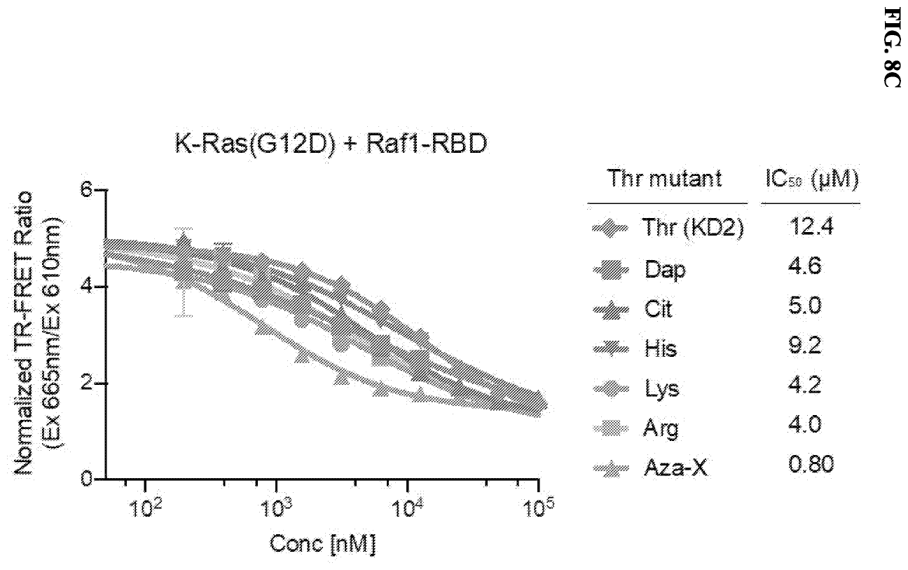

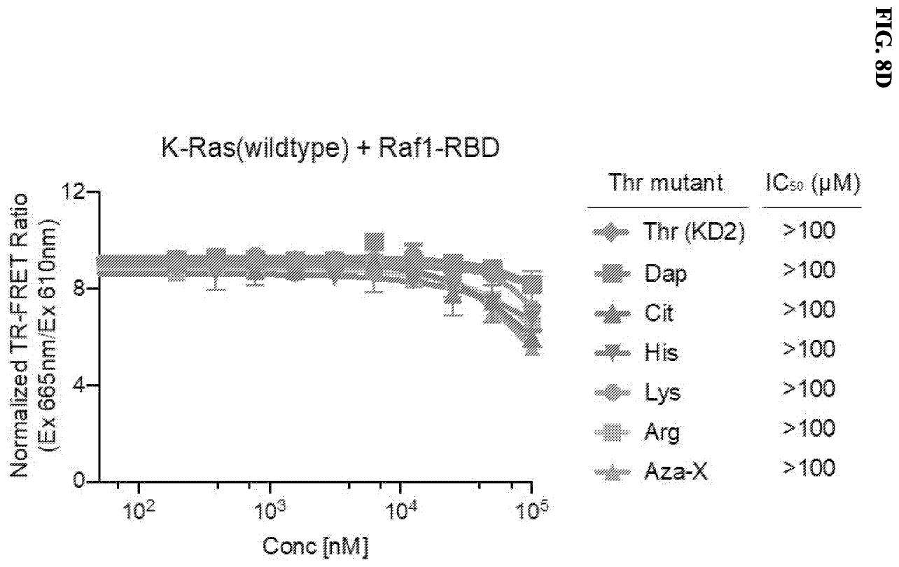

We next sought to improve the affinity and mutant/wildtype selectivity of KD2 to K-Ras(G12D) through structure-guided chemical modification. Asking whether we could enhance side-chain interaction between the cyclic peptide and Asp12 of K-Ras, we synthesized a set of KD2 analogs varying at the Thr10 position (FIGS. 8A-8B). These included the His, Lys and Arg mutants, as well as a few non-protogenic amino acids such as L-1,2-diaminopropanoic acid (Dap), L-citrulline (Cit), and L-β-azidoalanine (Aza). During cyclic peptide synthesis, an unexpected side reaction led the conversion of the azidoalanine residue into 4-methylpiperidinylalanine (labeled Aza-X). This transformation likely occurred during a Fmoc deprotection step where 40% 4-methylpiperidine was used.

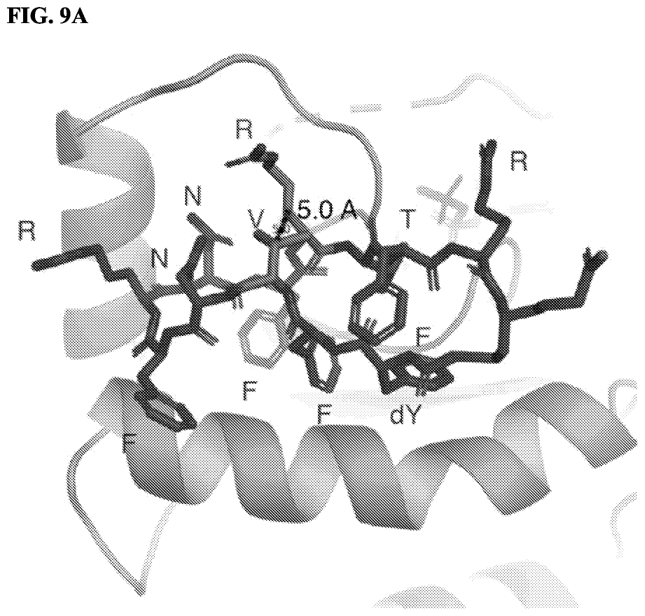

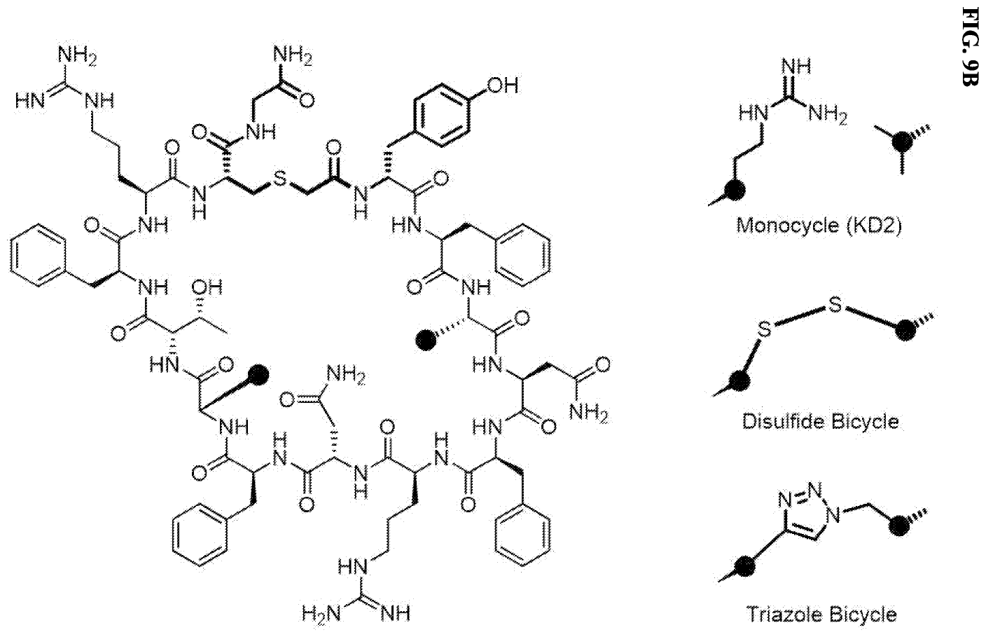

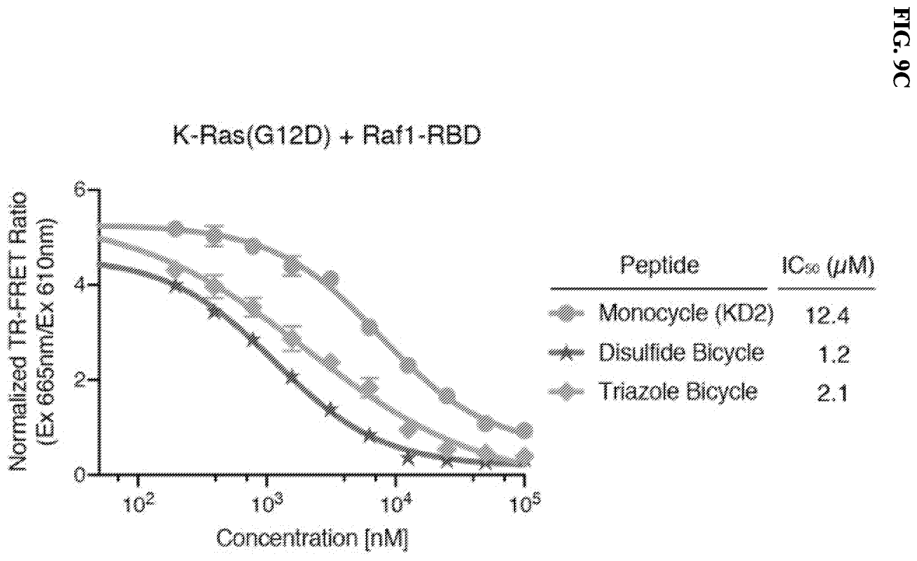

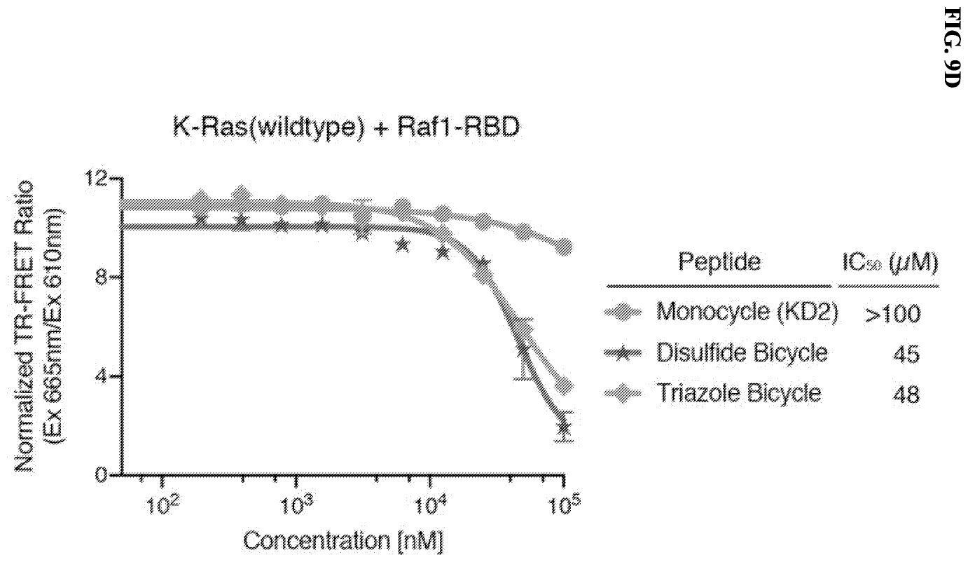

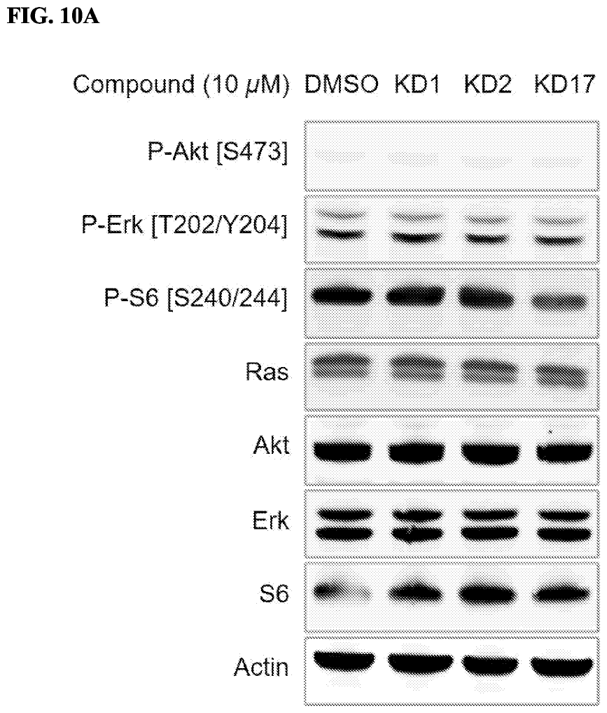

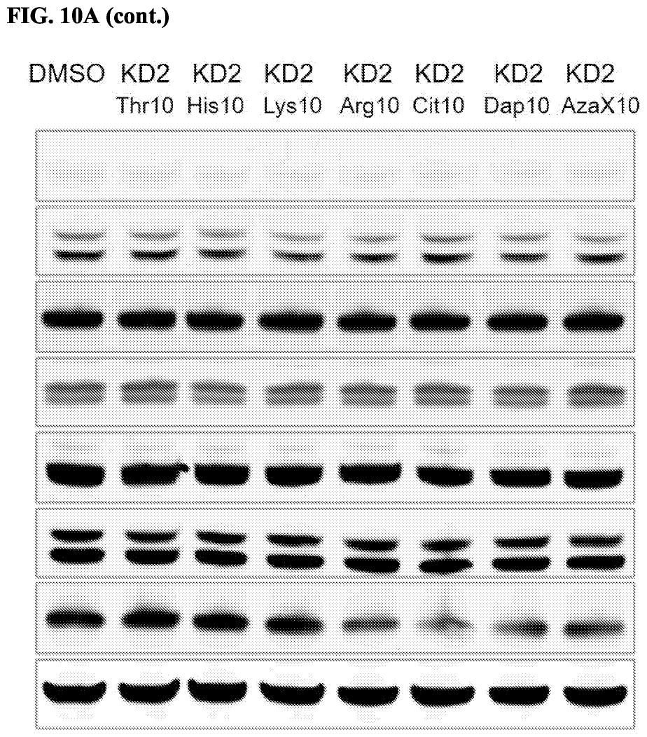

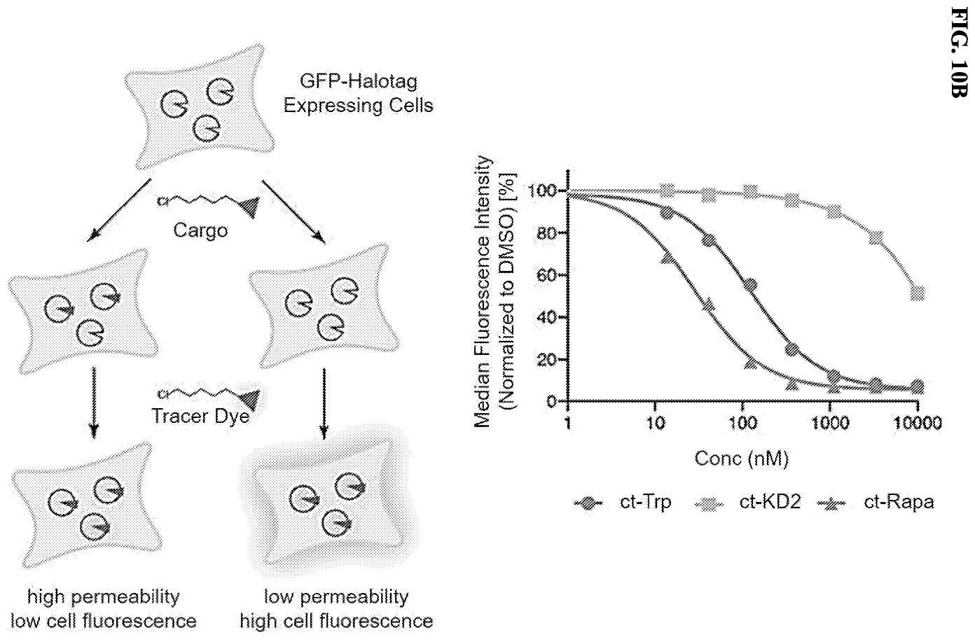

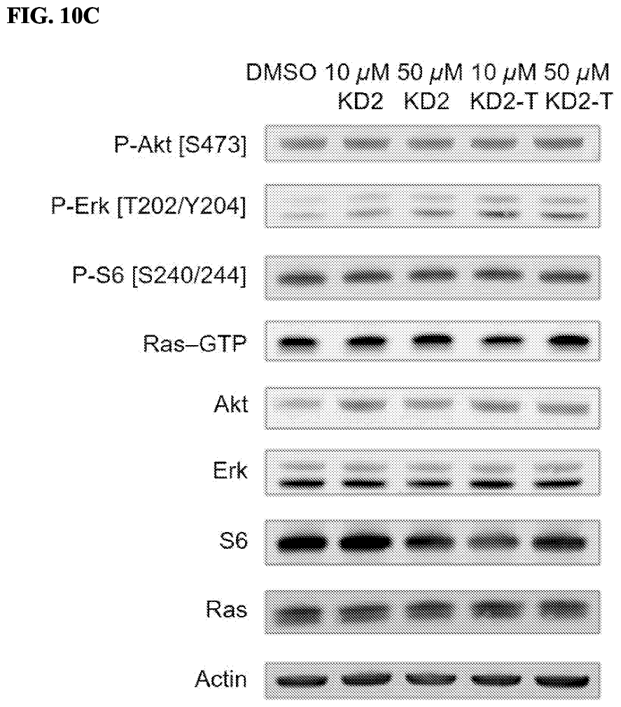

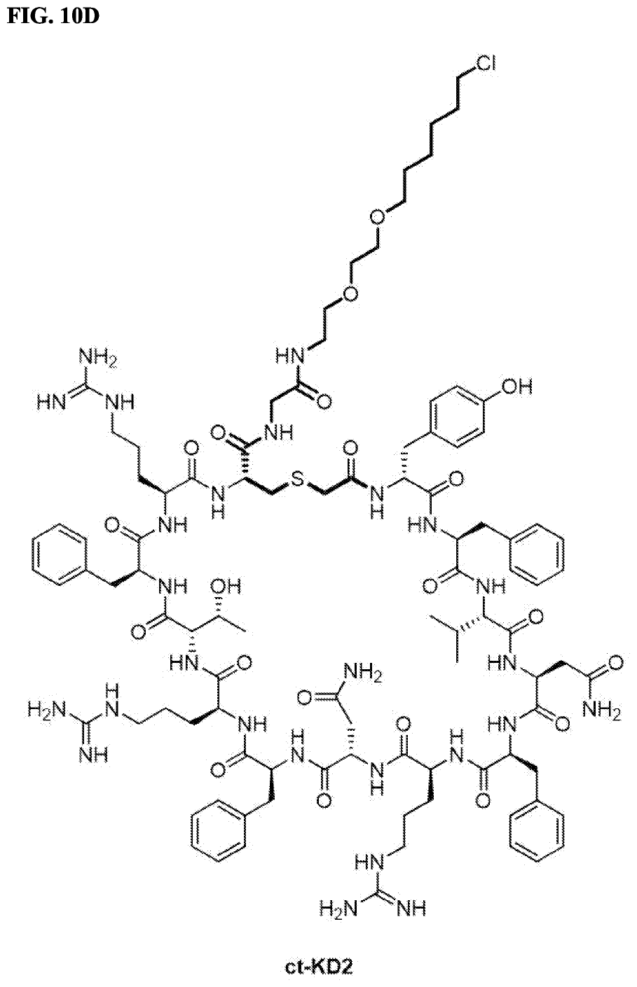

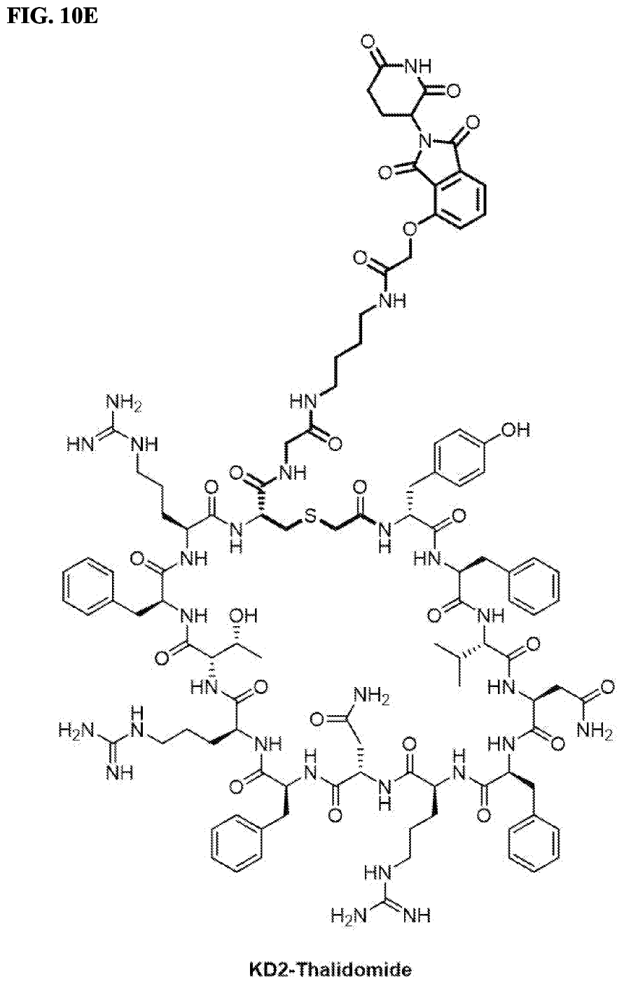

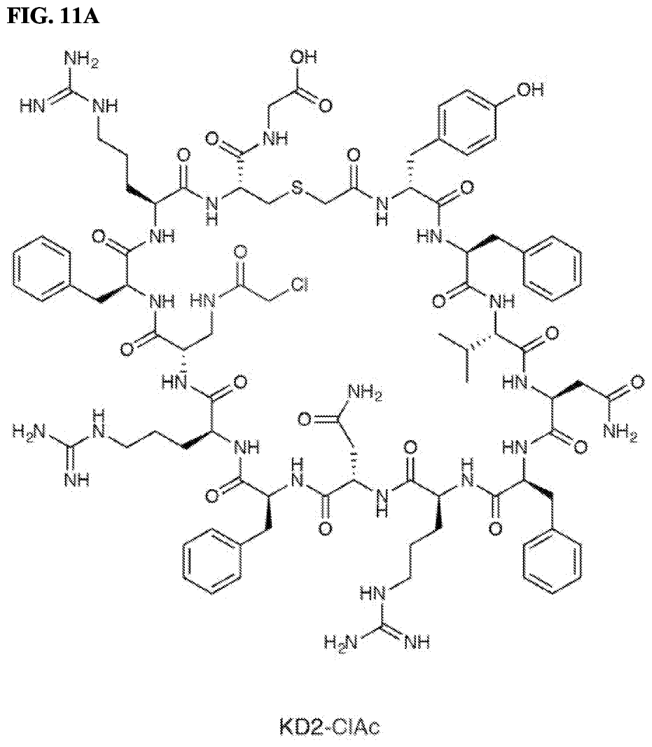

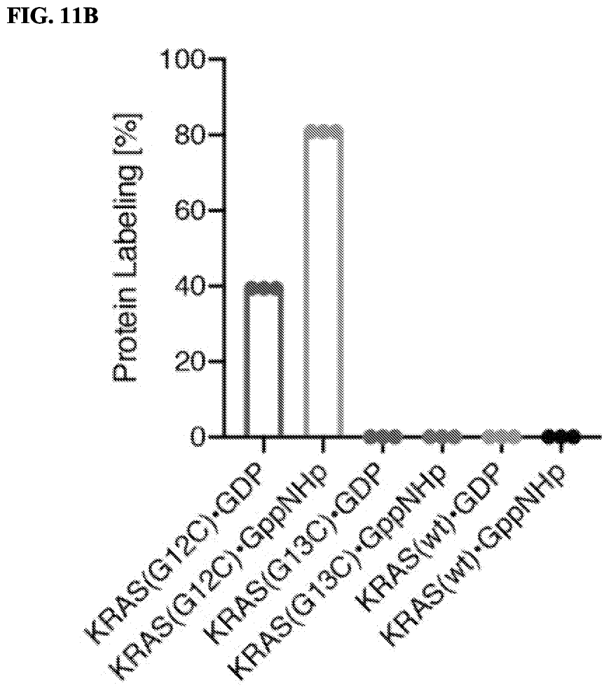

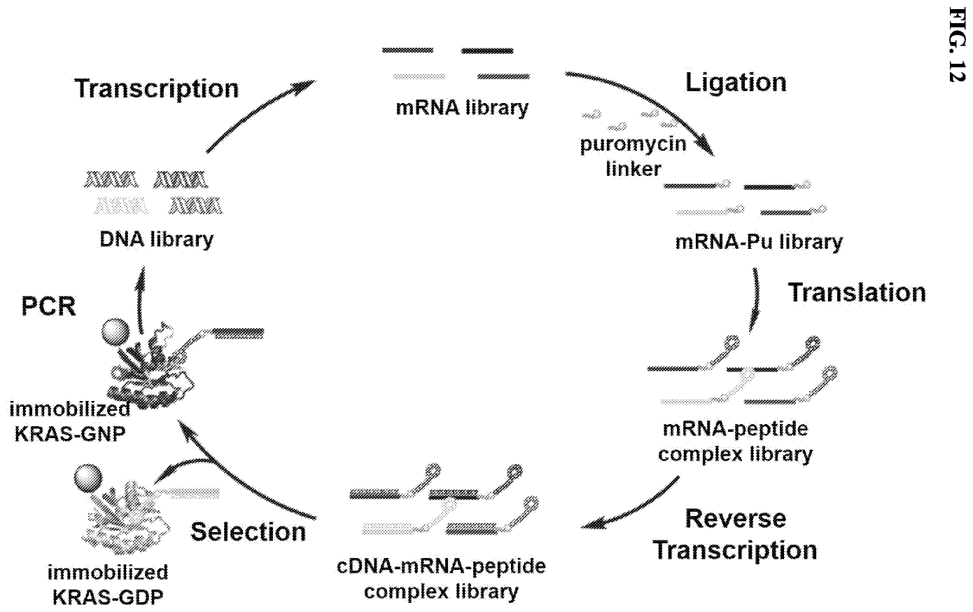

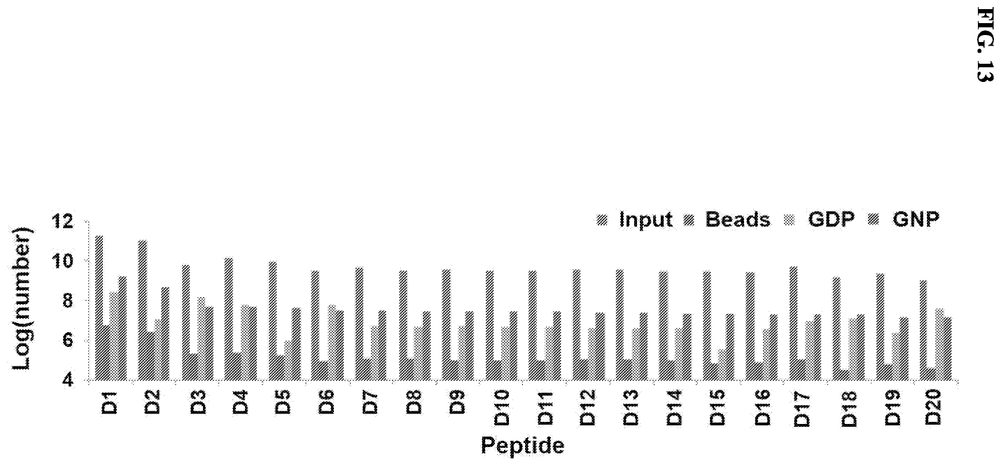

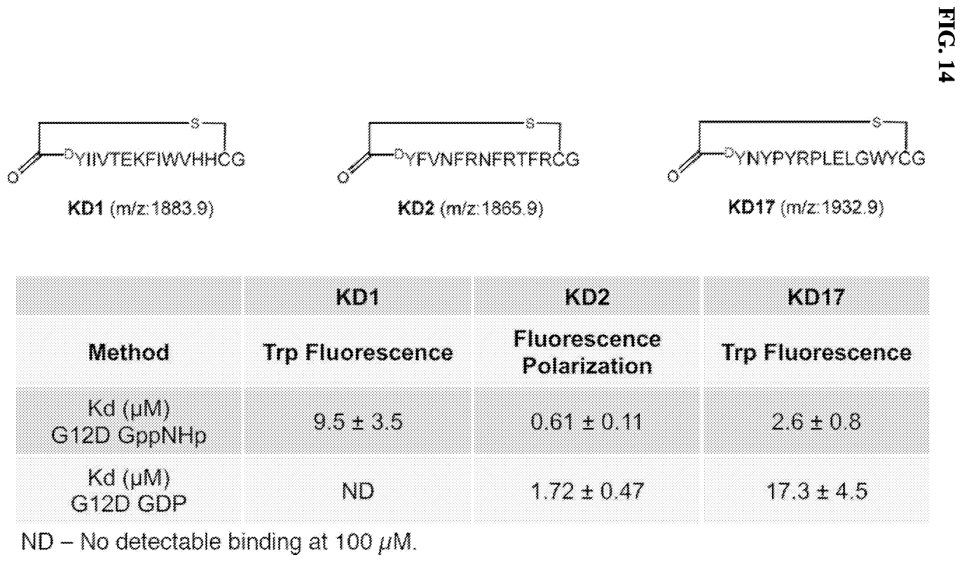

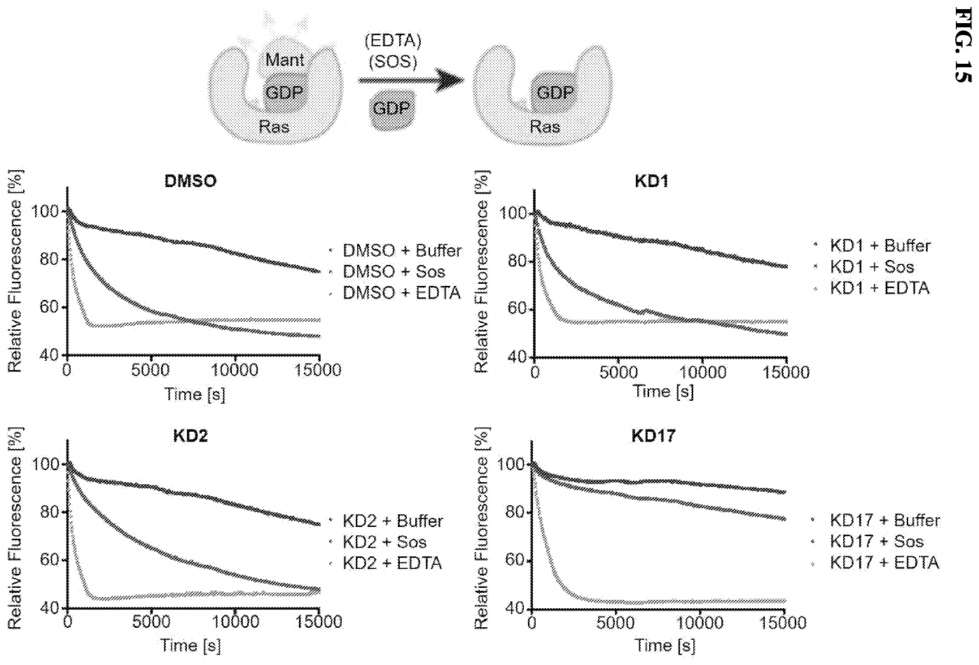

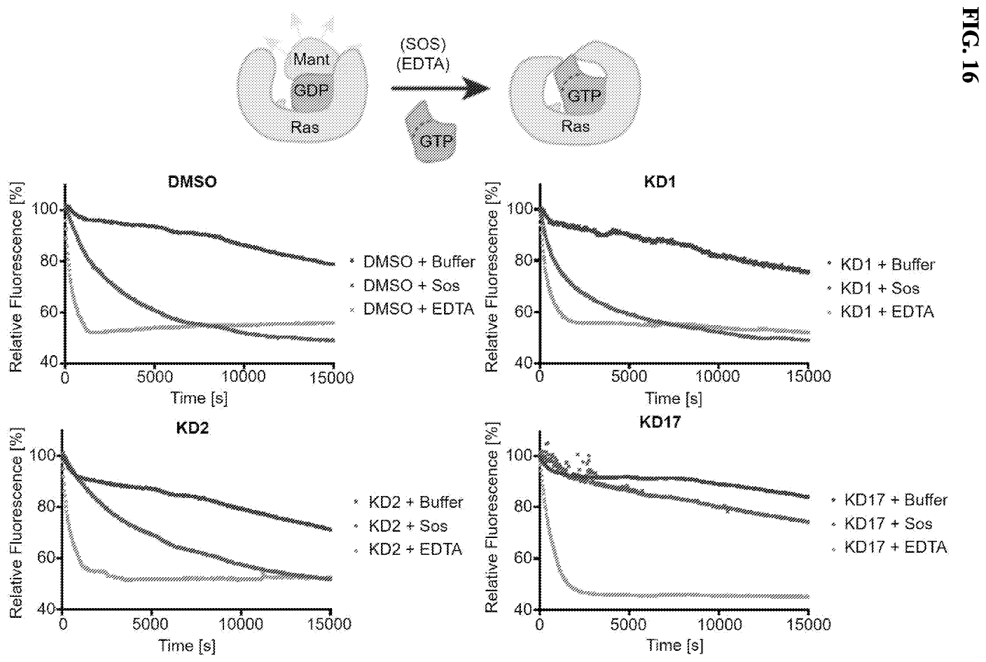

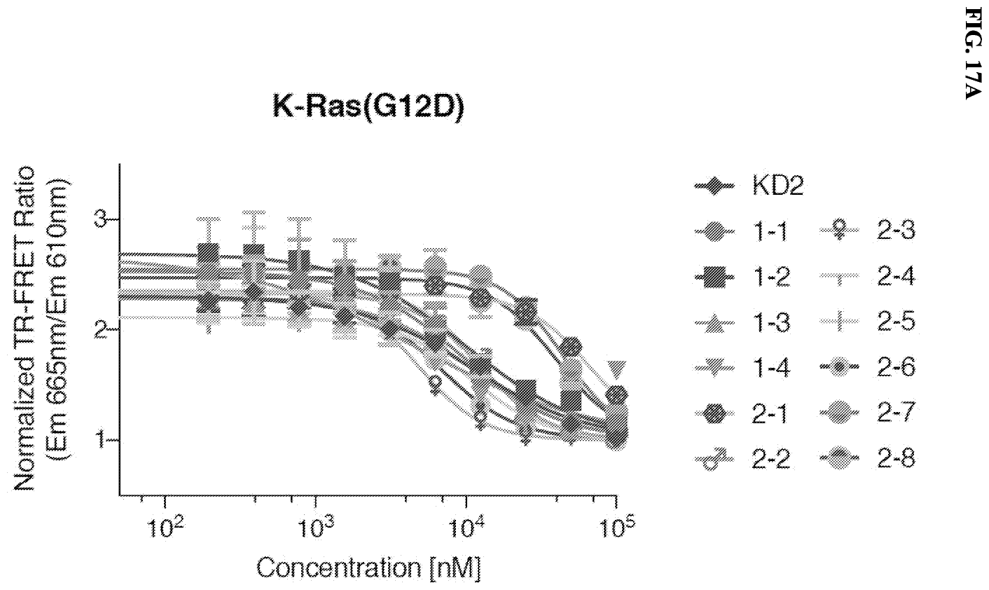

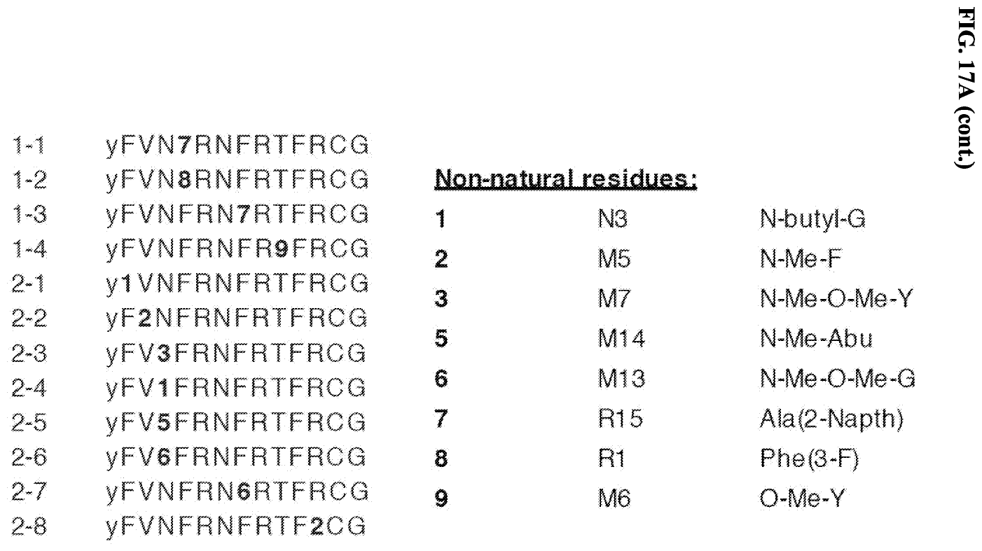

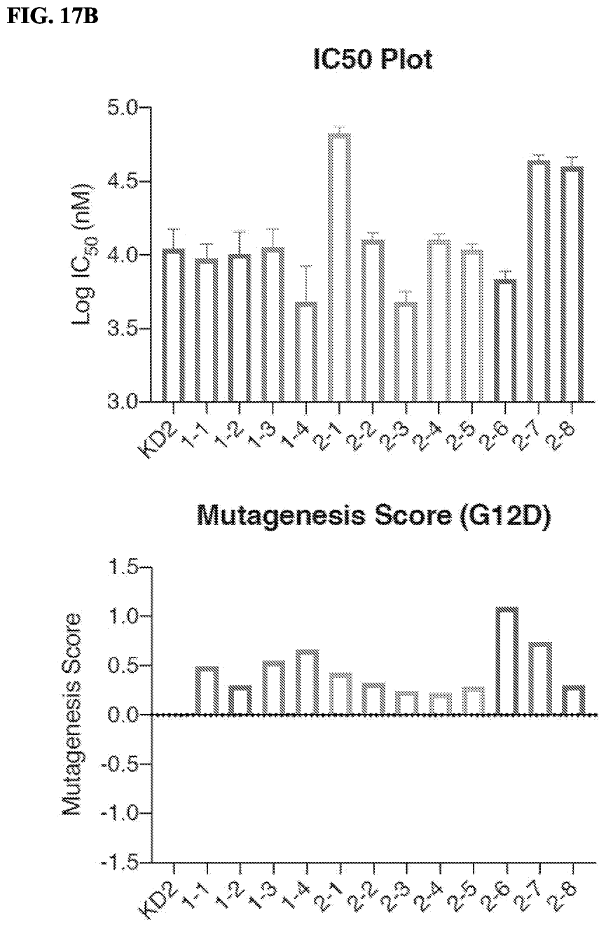

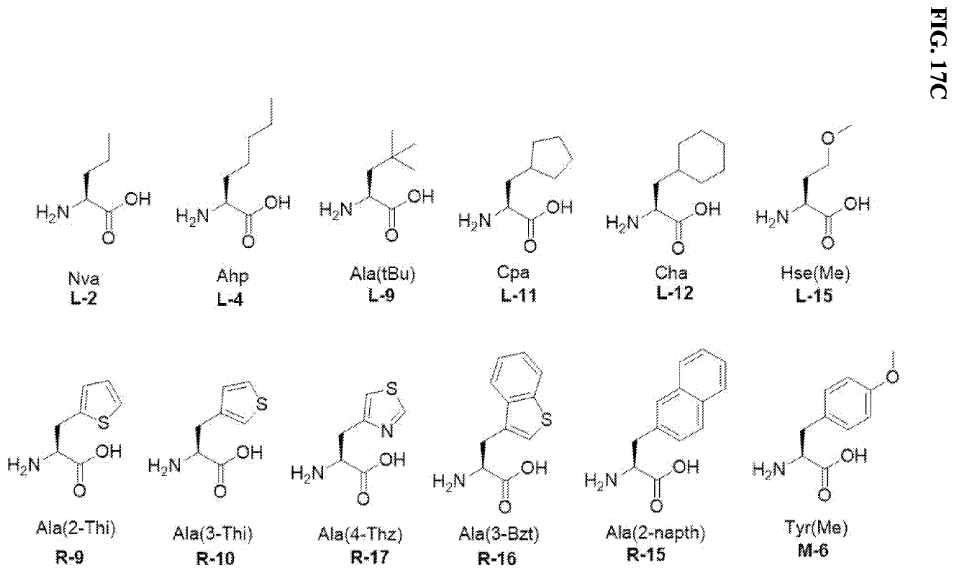

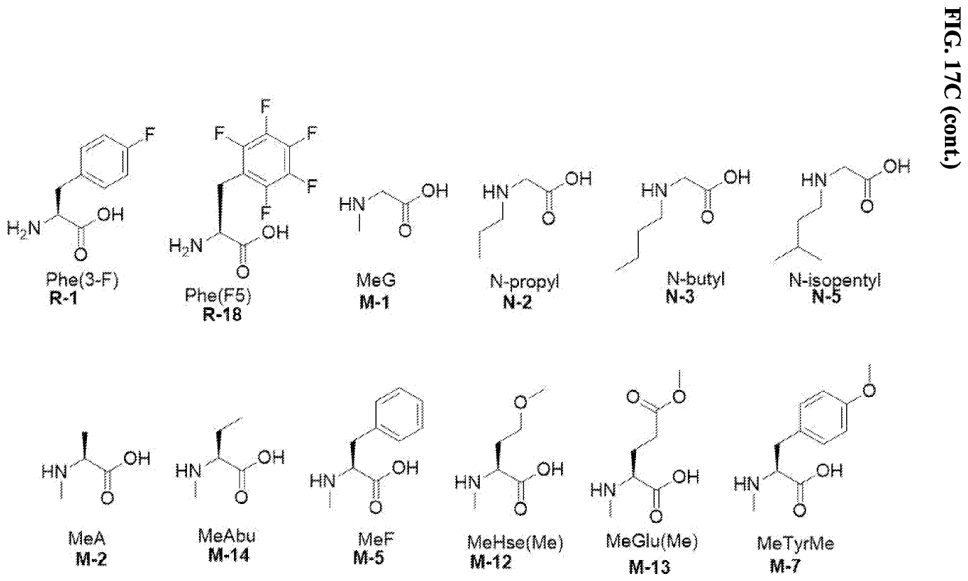

We tested these KD2 analogs in the TR-FRET-based Ras·Raf interaction assay (FIGS. 9C-9D). The measured IC₅₀ values closely matched the affinity change predicted by the saturation mutagenesis experiment, with the Lys and Arg mutants being about 3-fold more potent than KD2 (however, the solubility of these two mutants were significantly lower than the parent peptide KD2, preventing their testing at concentrations greater than 11.1 μM). Most strikingly, the byproduct (Aza-X) from the failed attempt to incorporate azidoalanine was the most potent among all compounds tested, with an IC₅₀ of 0.80 μM, a >10-fold improvement compared to KD2. Meanwhile, these analogs appeared to have maintained the G12D mutant selectivity: none of these KD2 mutants inhibited the interaction between Raf1-RBD and wildtype K-Ras·GppNHp by more than >50% at the highest concentrations tested (11.1 μM for the Lys, Arg, His and Dap mutants due to limited solubility, 100 μM for all others).

We also considered rigidification of the cyclic peptide scaffold through the formation of a transannular bridge, a structural feature that has been found both in natural and synthetic peptides to improve their activity (39-41). To achieve this, we first identified two amino acids in KD2, Val3 and Arg9, that do not participate in intramolecular interactions or binding interaction with K-Ras (FIG. 9A). By replacing these two amino acids with two cysteines or a combination of azidoalanine and propargylglycine, we synthesized two bicyclic variants of KD2 (FIG. 9B). These peptides were more potent inhibitors of the interaction between K-Ras(G12D) and Raf1-RBD (FIG. 9C). Although these two bicyclic peptide also inhibited the interaction between wildtype K-Ras and Raf1-RBD, the IC₅₀ values were more than 20-fold higher than that for K-Ras(G12D).

It is conceivable that both the potency and the mutant selectivity of these peptides can benefit from further structural optimization with two distinct approaches—Thr10 modification and scaffold rigidification. Importantly, the data here indicates that substitution of Thr10 is well tolerated and may be tailored to target the GTP-state of other G12 mutants of K-Ras.

To demonstrate this, we wondered if we could repurpose KD2 to target the GTP-state of K-Ras(G12C) by incorporating an electrophile at the Thr10 position on KD2. Using whole protein mass spectrometry, we found that a chloroacetamide derivative of KD2, but not an acrylamide derivative, covalently engaged both K-Ras(G12C)·GppNHp and K-Ras(G12C)·GDP. While the covalent labeling was relatively slow and required a high concentration of the peptide (100 μM), we observed that the labeling of K-Ras(G12C)·GppNHp was twice as fast as that of K-Ras(G12C)·GDP, achieving 80% and 40% covalent modification after 24 h incubation at 23° C., respectively. Whether this difference originates from higher binding affinity (lower Kᵢ) or faster covalent reaction (higher k_{inact}) will require further kinetic investigation with more soluble analogs. It is worth noting that the same ligand did not covalently label K-Ras(G13C) or K-Ras(wildtype) in either GppNHp- or GDP-bound state. To our knowledge, this is the first mutant-selective, GTP-state selective covalent ligand for K-Ras(G12C).

We studied the cellular activity of these cyclic peptides in SW1990, a cell line with homozygous G12D mutation at the KRAS locus. When cells were treated with 10 μM KD1, KD2, or KD17 for 24 h, we did not observe any change in p-Akt or p-Erk levels. This was true for all the KD2 Thr10 variants we had synthesized. To understand the discrepancy between biochemical inhibitory activity and the lack of effect on K-Ras signaling output, we asked whether these peptides entered cells. We utilized the chloroalkane cell penetration assay (CAPA)⁴², which employs a cell line expressing HaloTag proteins on the outer membrane of mitochondria and monitors the cellular intake of chloroalkyl-tagged (ct) cargo compounds by quantifying the remaining unreacted HaloTag protein after exposure of cells to ct-cargo for a fixed amount of time. Whereas two control compounds, ct-tryptophan (ct-W) and ct-rapamycin (ct-Rapa), were shown to be highly cell permeable (CP₅₀=125 nM and 28.5 nM, respectively), ct-KD2 did not readily enter cells under the assay conditions (CP₅₀>10 μM). We questioned whether this limited cell permeability could be sufficient for proteolysis-targeting chimeras (PROTACs), as this class of compounds have a catalytic mechanism of action (43) and may be less restricted by low cellular permeability (44). We synthesized KD2-thalidomide and used it to treat SW1990 cells at various concentrations. However, under no conditions was reduction of K-Ras protein level observed. Treatment with either KD2 or KD2-thalidomide also did not alter the level of Ras-GTP inside the cell. All the current evidence suggests that cell permeability is a key limiting factor for cellular activity of KD2, and that improving permeability should be a critical task in future compound optimization.

Employing a high-throughput selection platform (the RaPID system), we have identified three distinct cyclic peptide scaffolds that preferentially bind to K-Ras(G12D) in its GTP state from an initial library of 10¹² members. These cyclic peptides inhibit the interaction between K-Ras(G12D) and Raf1-RBD but are less effective for wildtype K-Ras protein. X-ray crystallography showed that one of these peptides binds to K-Ras in the Switch II Groove region previously discovered in a fragment screen. Structure-guided chemical diversification allowed rapid optimization of one initial hit into a compound with sub-micromolar potency at inhibiting Ras-Raf interaction. One remaining challenge is that these cyclic peptides do not readily enter cells, impeding their utility in a cellular setting. Cellular permeability of cyclic peptides is a complex problem under active research (45, 46). With guidance from abundant empirical rules and contemporary computational modeling, we are optimistic that this will be a surmountable problem after sufficient experimentation.

Our study has overturned the current understanding of the Switch II Pocket (SIIP) first revealed by the discovery of covalent ligands for K-Ras (G12C) and now extended by many more analogs which have advanced to the clinic. The K-Ras (G12C) ligands do not bind to the GTP state of K-Ras(G12C) and only bind in its GDP state, when switch II of K-Ras is open, exposing the SIIP. This understanding has dominated clinical trials for K-Ras (G12C) ligands because of the inability to identify ligands for the GTP state of K-Ras (G12C). This study set out to identify what we initially hypothesized would have to be a distinct pocket on K-Ras (G12D) in the GTP state since previous studies appeared to suggest SIIP was inaccessible. The co-crystal structure of KD2 bound to K-Ras(G12D)/GMPPNP was surprising because KD2 binds to the SIIP. This result suggests a previously unappreciated dynamic aspect of the switch II loop of K-Ras (G12D) in the GTP state exposing the SIIP for drug access. This is a promising therapeutic approach for many oncogenic K-Ras mutants that are devoid of GTPase activity and enriched in their GTP-bound state.

Our study shows that the dynamic Switch II Groove region is not only a viable drug pocket but can accommodate ligands of great size (in our example, 1800 Da). In addition, it provides possible direct access to mutant amino acid residues on the P-loop. The present work also demonstrates for the first time that GTP-state selective direct Ras ligands are present when exceptionally large libraries are accessed. This is a promising therapeutic approach for many oncogenic K-Ras mutants that are devoid of GTPase activity and enriched in their GTP-bound state.

### REFERENCES FOR EXAMPLE 1

1. Prior, I. A., Lewis, P. D. & Mattos, C. Cancer Res. 72, 2457-2467 (2012). 2. Ostrem, J. M. L. & Shokat, K. M. Nat. Rev. Drug Discov. 15, 771-785 (2016). 3. Hunter, J. C. et al. Mol. Cancer Res. 13, 1325-1335 (2015). 4. Traut, T. W. Physiological concentrations of purines and pyrimidines. Mol. Cell. Biochem. 140, 1-22 (1994). 5. Nickerson, S., Joy, S. T., Arora, P. S. & Bar-Sagi, D. Nat. Chem. Biol. 7, 585-587 (2011). 6. Hons, M., Niebel, B., Kamowski, N., Weiche, B. & Famulok, M. ChemBioChem 13, 1433-1437 (2012). 7. Trinh, T. B., Upadhyaya, P., Qian, Z. & Pei, D. ACS Comb. Sci. 18, 75-85 (2016). 8. Spencer-Smith, R. et al. Nat. Chem. Biol. 13, 62-68 (2017). 9. Sakamoto, K. et al. Biochem. Biophys. Res. Commun. 484, 605-611 (2017). 10. Martin-Gago, P. et al. Angew. Chem. Int. Ed. 56, 2423-2428 (2017). 11. Guillard, S. et al. Nat. Commun. 8, 1-11 (2017). 12. McGee, J. H. et al. J Biol. Chem. 293, 3265-3280 (2018). 13. Taveras, A. G. et al. Bioorganic Med. Chem. 5, 125-133 (1997). 14. D'Incalci, M. & Galmarini, C. M. Mol. Cancer Ther. 9, 2157-2163 (2010). 15. Maurer, T. et al. Proc. Natl. Acad. Sci. U.S.A 109, 5299-304 (2012). 16. Sun, Q. et al. Angew. Chem. Int. Ed. 51, 6140-6143 (2012). 17. Shima, F. et al. Proc. Natl. Acad. Sci. 110, 8182-8187 (2013). 18. Ostrem, J. M., Peters, U., Sos, M. L., Wells, J. A. & Shokat, K. M. Nature 503, 548-51 (2013). 19. Lim, S. M. et al. Angew. Chem. Int. Ed. 53, 199-204 (2014). 20. Gentile, D. R. et al. Cell Chem. Biol. 24, 1455-1466.e14 (2017). 21. Welsch, M. E. et al. Cell 168, 878-889.e29 (2017). 22. Feng, H. et al. Biochemistry 58, 2542-2554 (2019). 23. Kessler, D. et al. Proc. Natl. Acad. Sci. U.S.A. 116, 15823-15829 (2019). 24. Tran, T. H. et al. Proc. Natl. Acad. Sci. U.S.A 117, 3363-3364 (2020). 25. Patricelli, M. P. et al. Cancer Discov. 6, 316-329 (2016). 26. Janes, M. R. et al. Cell 172, 578-589 (2018). 27. Hansen, R. et al. Nat. Struct. Mol. Biol. 25, 454-462 (2018). 28. Fakih, M. et al. J. Clin. Oncol. 37, 3003 (2019). 29. Canon, J. et al. Nature 575, 217-223 (2019). 30. Papadopoulos, K. P. et al. J Clin. Oncol. 37, TPS3161-TPS3161 (2019). 31. Available at: https://clinicaltrials.gov/ct2/show/NCT04006301, accessed Jul. 27, 2019. 32. McCormick, F. Cancer Cell 37, 3-4 (2020). 33. Sogabe, S. et al. ACS Med. Chem. Lett. 8, 732-736 (2017). 34. Niida, A. et al. Bioorganic Med. Chem. Lett. 27, 2757-2761 (2017). 35. Wu, X. et al. ACS Comb. Sci. 13, 486-495 (2011). 36. Passioura, T. & Suga, H. 53, (2017). 37. Geyer, M. et al. Biochemistry 35, 10308-10320 (1996). 38. Spoemer, M., Herrmann, C., Vetter, I. R., Kalbitzer, H. R. & Wittinghofer, A. Proc. Natl. Acad. Sci. U.S.A. 98, 4944-9 (2001). 39. Sako, Y., Morimoto, J., Murakami, H. & Suga, H. J Am. Chem. Soc. 130, 7232-7234 (2008). 40. Rhodes, C. A. & Pei, D. Chem. —A Eur. J. 23, 12690-12703 (2017). 41. Thombare, V. J. & Hutton, C. A. Pept. Sci. 110, (2018). 42. Peraro, L. et al. J. Am. Chem. Soc. 140, 11360-11369 (2018). 43. Bondeson, D. P. et al. Nat. Chem. Biol. 11, 611-617 (2015). 44. Foley, C. A., Potjewyd, F., Lamb, K. N., James, L. I. & Frye, S. V. ACS Chem. Biol. 15, 290-295 (2020). 45. Dougherty, P. G., Sahni, A. & Pei, D. Chem. Rev. 119, 10241-10287 (2019). 46. Samson, M. et al. Future Med. Chem. 155, 3-12 (2017).

### Example 2: Materials and Methods

Cyclic Peptide Selection

Selection

Selections were performed with thioether-macrocyclic peptide library against GppNHp-bound K-Ras(G12D/T35S), using the GDP-bound K-Ras(G12D/T35S) as the negative selection (FIG. 1). Thioether-macrocyclic peptide libraries were constructed with N-chloroacetyl-D-tyrosine (ClAc^{D}Tyr) as an initiator by using the flexible in vitro translation (FIT) system (1). The mRNA libraries, ClAc^{D}Tyr-tRNA^{fMet}_{CAU} were prepared as reported (2-6). The mRNA library corresponding for the thioether-macrocyclic peptide library was designed to have an AUG initiator codon to incorporate N-chloroacetyl-D-tyrosine (ClAc^{D}Tyr) (1), followed by 8-12 NNK random codons (N=G, C, A or U; K=G or U) to code random proteinogenic amino acid, and then a fixed downstream UGC codon to assign Cys. After in vitro translation, a thioether bond formed spontaneously between the N-terminal ClAc group of the initiator^{D}Tyr residue and the sulfhydryl group of a downstream Cys residue.

In the first round of selection, the initial cyclic peptide library was formed by adding puromycin ligated mRNA library (225 pmol) to a 150 μL scale flexible in vitro translation system, in the presence of 30 μM of ClAc^{D}Tyr-tRNA^{fMet}_{CAU}. The translation was performed at 37° C. for 30 min, followed by an extra incubation at 25° C. for 12 min. After an addition of 15 μL of 200 mM EDTA (pH 8.0) solution, the reaction solution was incubated at 37° C. for 30 min to facilitate cyclization. Then the library was reversed transcribed by M-MLV reverse transcriptase at 42° C. for 1 h and subject to pre-washed Sephadex G-25 columns to remove salts. The desalted solution of peptide-mRNA/cDNA was applied to GppNHp-bound K-Ras(G12D/T35S) immobilized Dynabeads M280 streptavidin magnetic beads and rotated at 4° C. for 1 h in selection buffer (25 mM HEPES pH 7.5, 150 mM NaCl, 1 mM MgCl₂ and 0.05% Tween 20) containing 0.5 mM GppNHp and 0.1% acetylated BSA. Bead amounts were chosen that the final concentration of GppNHp-bound K-Ras was 200 nM. This process is referred to as positive selection. The selected peptide-mRNA/cDNAs were isolated from the beads by incubating in 1×PCR reaction buffer heated at 95° C. for 5 min. The amount of eluted cDNAs was measured by quantitative PCR (Roche LightCycler 96). The remaining cDNAs were amplified by PCR, purified and transcribed into mRNAs as a library for the next round of selection.

In the subsequent rounds of selection, ligated mRNA from previous round (7.5 pmol) was added to a 5 μL scale reprogrammed in vitro translation system. This was incubated at 37° C. for 30 min and at 25° C. for 12 min. Then 1 μL of 100 mM EDTA (pH 8.0) was added and incubated at 37° C. for 30 min. After reverse transcription and subject to pre-washed Sephadex G-25 columns to remove salts, negative selections was performed by adding the desalted solution of peptide-mRNA/cDNA to GDP-bound K-Ras(G12D/T35S) immobilized Dynabeads M280 streptavidin magnetic beads and rotated at 4° C. for 30 min in selection buffer containing 0.1% acetylated BSA. This process was repeated several times by removing the supernatant to fresh beads immobilized with GDP-bound K-Ras(G12D/T35S). The supernatant from the last negative selection was then added to beads immobilized with GppNHp-bound K-Ras(G12D/T35S) (final conc. 200 nM), and rotated at 4° C. for 30 min in selection buffer containing 0.5 mM GppNHp and 0.1% acetylated BSA. As described in first round of selection, the cDNA was quantified with qPCR, amplified with PCR, transcribed and ligated to puromycin. The subsequent selection was repeated for several rounds until a significant enrichment of cDNA was observed for GppNHp-bound K-Ras(G12D/T35S). The recovered cDNA was then identified by Miseq sequencing (Illumina).

Comparison Selection

In comparison selections, ligated mRNA (7.5 pmol) from last round selection was added to a 5 μL scale reprogrammed in vitro translation system. After translation, cyclization, reverse transcription and prewashed with Sephadex G-25 columns, the desalted solution of peptide-mRNA/cDNA library was split equally into three fractions, and perform three paralleled selections with the same amount of blank, GDP-bound K-Ras(G12D/T35S) or GppNHp-bound K-Ras(G12D/T35S) immobilized Dynabeads M280 streptavidin magnetic beads. For each of the paralleled selections, the beads were rotate at 4° C. for 30 min, washed three times with selection buffer. The remaining cDNAs were then eluted from the beads, quantified by qPCR, followed by Miseq sequencing. Finally, the identified sequences were compared after normalization of the Miseq abundance with its qPCR read for each paralleled selection.

### Cyclic Peptide Synthesis

General Workflow

Linear peptide precursors with 2-chloroacetylated N-termini were synthesized on NovaPEG Rink-Amide resin using standard Fmoc solid-phase synthesis technology. After cleavage of the peptides from the resin, macrocyclization (S_{N}2 between cysteine and chloroacetamide) was performed in solution under basic conditions, and the product was purified by preparative HPLC. Detailed protocols are provided below.

### Resin Preparation

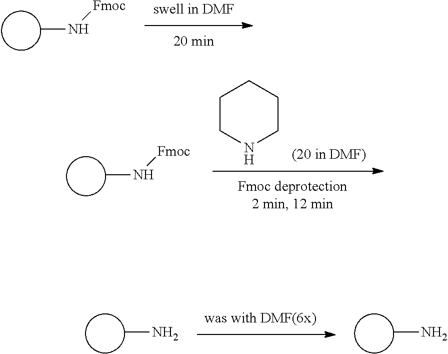

57 mg NovaPEG Rink-Amide resin (0.44 mmol/g, 25 μmol) was swelled with 4 mL of DMF for at least 20 min. The resin was washed three times with DMF (4 mL each wash). To remove the Fmoc group, 4 mL 20% Piperidine (in DMF) was added to the resin and the mixture was rotated at 23° C. for 2+12 min. The resin was washed sequentially with DMF (3×4 mL), DCM (3×4 mL) and DMF (3×4 mL).

Amino Acid Coupling

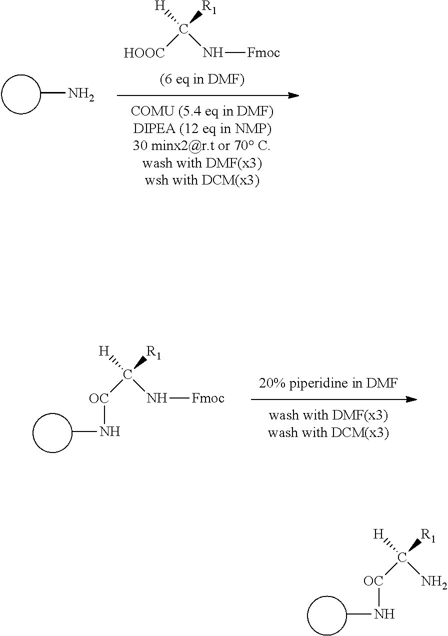

For each amino acid to be coupled to the polypeptide, a mixture of Fmoc-amino acid (600 μL of 0.5 M solution in DMF, 300 μmol), COMU (600 μL of 0.45 M solution in DMF, 270 μmol) and DIPEA (300 μL of 2.0 M solution in NMP, 600 μmol) was added to the resin and the mixture was vigorously shaken for 1 h. The resin was washed sequentially with DMF (3×4 mL) and DCM (3×4 mL). Chloranil test was performed to confirm complete coupling (a few resin beads mixed with 5 μL 2% acetaldehyde/DMF and 5 μL 2% chloranil/DMF; coupling is complete if resin is orange/yellow, incomplete if resin is blue/green/black). If coupling is incomplete, an additional round of coupling was performed using identical amounts of reagents. When coupling is complete, 4 mL of 20% piperidine (in DMF) was added and the mixture was rotated for 2+12 min to remove the Fmoc group. The resin was washed sequentially with DMF (3×4 mL), DCM (3×4 mL) and DMF (3×4 mL).

Note: In certain cases, Fmoc deprotection was performed with 4 mL 40% 4-methylpiperidine in DMF in lieu of 20% piperidine.

N-Terminal Capping with Chloroacetamide

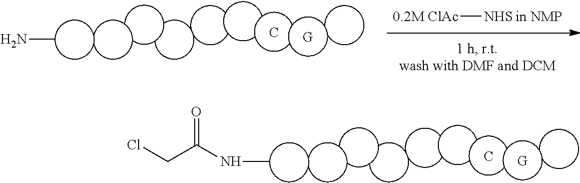

N-hydroxylsuccinmide chloroacetate (2 mL of 0.2 M solution in NMP) was added to peptide-conjugated resin and the mixture was rotated at 23° C. for 1 h. A few resin beads were taken for TNBS test. Briefly, the beads were mixed with 5 uL of 10% DIPEA/DMF+5 uL 1% 2,4,6-trinitrobenzenesulfonic acid (TNBS)/DMF and incubated at 23° C. for 10 min. Capping is complete ok if beads are colorless, but not if they are yellow/orange. The resin was washed by DMF (5×4 mL) followed by DCM (5×4 mL) and dried under reduced pressure overnight.

Deprotection and Cleavage

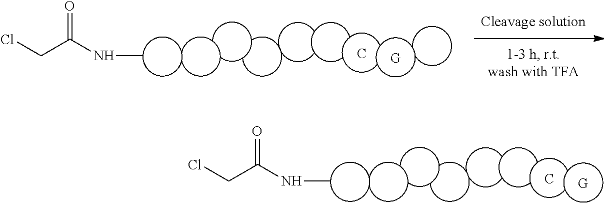

A cleavage solution was prepared as follows, chilled on ice and added to vacuum-dried resin (2 mL/25 μmol resin). The mixture was incubated on ice for 10 min, then at 23° C. for 3 h with gentle rotation. The supernatant was filtered into a 15 mL tube, and the resin was washed with trifluoroacetic acid (3×1 mL). The filtrates were combined. The bulk of TFA was removed under reduced pressure on a centrifugal evaporator to ˜1 mL volume. 20 mL ice-cold ether was added to the residue, giving rise to a while suspension, which was kept at −20° C. for 1 h. The precipitated peptide was pelleted by centrifugation and the supernatant was discarded. The pellet was washed with diethyl ether (5×5 mL) by resuspension and re-pelleting, then dried under vacuum.

| | | |
|---|---|---|
| Cleavage solution | TFA (trifluoroacetic acid) | 1850 μL |
| (25 μmol scale) | dH₂O | 50 μL |
| | TIS (triisopropyl silane) | 50 μL |
| | EDT (ethane dithiol) | 50 μL |
| Total | | 2000 μL |

Cyclization

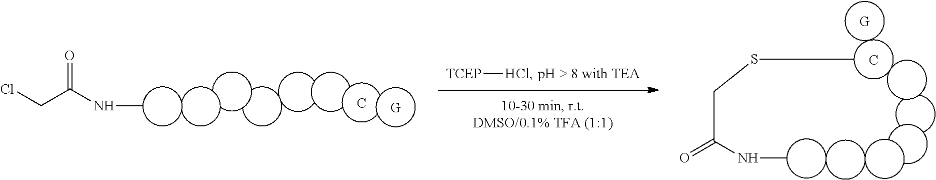

The precipitated peptide was dissolved in 5 mL DMSO. TCEP (200 μL of 500 mM solution in water) and triethylamine (100 μL) were added sequentially. The pH of the mixture should be 8. If not, adjust pH to 8 with additional triethylamine. The mixture was incubated at 23° C. Reaction progress was monitored by LC-MS every hour. When cyclization was complete, trifluoroacetic acid (60 μL) was added to the reaction mixture to quench the reaction. The mixture was diluted with 5 mL 0.1% TFA/water and filtered through a 0.45-μm PTFE filter. The filtered solution was purified by preparative reverse-phase HPLC (Waters XBridge C18 column 5 μm particle size 30×250 mm, 10-70% acetonitrile-water+0.1% formic acid, 40 min, 20 mL/min). Product containing fractions were concentrated under reduced pressure and lyophilized to afford the product peptide as a white powder. The counter ion was exchanged from formate to chloride by repeated (three times) dissolution of the peptide in 5 mM HCl in 50% acetonitrile/water and removal of solvent under vacuum.

### Synthesis of C-Terminally Modified Cyclic Peptides (Ct-KD2 and KD2-Thalidomide)

To prepare C-terminally modified cyclic peptides, the parent cyclic peptide (KD2) was prepared on Wang resin in lieu of Rink resin to afford the cyclic peptide with free carboxylic acid on the C-terminus (KD2-CO₂H). KD2-CO₂H (5 μmol) was dried by azeotropic distillation from of a suspension in benzene (three times), then was dissolved in DMF (100 μL). Amine coupling partner (100 μmol) was added as a solution in DMF (100 μL) to a solution of KD2-CO₂H (5 μmol) in DMF (100 μL). DIPEA (100 μL of 2.0 M solution in DMF, 200 μmol) and HATU (100 μL of 1.0 M solution in DMF, 100 μmol) were added sequentially, the reaction mixture was incubated at 23° C., and the reaction progress was monitored by LC-MS. Typically, the reaction did not achieve completion and the reaction was terminated after 16 h. The reaction mixture was diluted with 50% acetonitrile-water+0.1% formic acid to a volume to 5.0 mL, filtered through a 0.45-μm PTFE filter. The filtered solution was purified by preparative reverse-phase HPLC (Waters XBridge C18 column 5 μm particle size 30×250 mm, 10-70% acetonitrile-water+0.1% formic acid, 40 min, 20 mL/min). Product containing fractions were concentrated under reduced pressure and lyophilized to afford the product peptide as a white powder. The counter ion was exchanged from formate to chloride by repeated (three times) dissolution of the peptide in 5 mM HCl in 50% acetonitrile/water and removal of solvent under vacuum.

### Synthesis of Thr10 Variants of KD2

Except the example noted below, Thr10 variants were prepared following the general synthesis protocol using appropriate Fmoc-protected amino acids.

### Synthesis of KD2-ClAc

KD2-T10Dap (5 μmol), in which Thr10 of KD2 was replaced with L-diaminopropanoic acid was dried by azeotropic distillation from of a suspension in benzene (three times), then was dissolved in DMF (100 μL). A freshly prepared solution of N-hydroxysuccinimide chloroacetate (1.0 M in DMF, 50 μL, 50 μmol) was added to the cyclic peptide solution, the reaction mixture was incubated at 23° C., and the reaction progress was monitored by LC-MS. Typically the reaction was complete in 8 h. The reaction mixture was diluted with 50% acetonitrile-water+0.1% formic acid to a volume to 5.0 mL, filtered through a 0.45-μm PTFE filter. The filtered solution was purified by preparative reverse-phase HPLC (Waters XBridge C18 column 5 μm particle size 30×250 mm, 10-70% acetonitrile-water+0.1% formic acid, 40 min, 20 mL/min). Product containing fractions were concentrated under reduced pressure and lyophilized to afford the product peptide as a white powder. The counter ion was exchanged from formate to chloride by repeated (three times) dissolution of the peptide in 5 mM HCl in 50% acetonitrile/water and removal of solvent under vacuum.

Protein Expression and Purification

K-Ras(G12D) CysLight

K-Ras(G12D) CysLight (for crystallization only) was expressed and purified following previously reported protocols (7,8).

Biotinylated K-Ras Proteins

pET-Duet plasmids encoding BirA biotin-protein ligase and His-Avi-TEV-tagged K-Ras proteins were constructed using standard molecular biology techniques. Biotinylated K-Ras proteins were produced in BL21(DE3) E. coli strain. Briefly, chemically competent BL21(DE3) cells were transformed with the corresponding pET-Duet plasmid and grown on LB agar plates containing 100 μg/mL carbenicillin. A single colony was used to inoculate a culture at 37° C., 220 rpm in terrific broth containing 100 μg/mL carbenicillin. When the optical density reached 0.6, the culture temperature was reduced to 20° C., and protein expression was induced by the addition of IPTG to 1 mM and Biotin to 5 μM. After 16 h at 20° C., the cells were pelleted by centrifugation (6,500×g, 10 min) and lysed in lysis buffer [20 mM Tris 8.0, 500 mM NaCl, 5 mM imidazole] with a high-pressure homogenizer (Microfluidics, Westwood, Mass.). The lysate was clarified by high-speed centrifugation (19,000×g, 15 min) and the supernatant was used in subsequent purification by immobilized metal affinity chromatography (IMAC). His-Avi-TEV tagged K-Ras protein was captured with Co-TALON resin (Clonetech, Takara Bio USA, 2 mL slurry/liter culture) at 4° C. for 1 h with constant end-to-end mixing. The loaded beads were then washed with lysis buffer (50 mL/liter culture) and the protein was eluted with elution buffer [20 mM Tris 8.0, 500 mM NaCl, 300 mM imidazole]. The protein was further purified with anion exchange chromatography (HiTrapQ column, GE Healthcare Life Sciences) using a NaCl gradient of 50 mM to 500 mM in 20 mM Tris 8.0. Nucleotide loading was performed by mixing the ion exchange-purified protein with an excess of GDP (5 mg/liter culture) or GppNHp (5 mg/liter culture) and 5 mM EDTA at 23° C. for 30 min. The reaction was stopped by the addition of MgCl₂ to 10 mM. For GppNHp, an additional calf intestine phosphatase treatment was performed as follows to ensure high homogeneity of the loaded nucleotide. The protein buffer was exchanged into Phosphatase Buffer [32 mM Tris 8.0, 200 mM ammonium sulfate, 0.1 mM ZnCl₂] with a HiTrap Desalting Column (GE Healthcare Life Sciences). To the buffer-exchanged protein solutions, GppNHp was added to 5 mg/mL, and Calf Intestine Phosphatase (NEB) was added to 10 U/mL. The reaction mixture was incubated on ice for 1 h, and MgCl₂ was added to a final concentration of 20 mM. After nucleotide loading, the protein was concentrated using an 10K MWCO centrifugal concentrator (Amicon-15, Millipore) to 20 mg/mL and purified by size exclusion chromatography on a Superdex 75 10/300 GL column (GE Healthcare Life Sciences). Fractions containing pure biotinylated Ras protein were pooled and concentrated to 20 mg/mL and stored at −78° C. In our hands, this protocol gives a typical yield of 5-15 mg/liter culture.

GST-Raf1-RBD and Sos^{cat}

The GST-tagged RBD domain of Raf1 (residues 1-149, GST-Raf1-RBD) was expressed and purified following published protocol (9).

The catalytic domain of Sos (residues 466-1049, Sos^{cat}) was expressed and purified following published protocol (10).

Other Proteins

GST-tagged full-length B-Raf protein (GST-BRAF) was purchased from MRC PPU (University of Dundee).

Cell Culture

SW1990 cells were obtained from ATCC and maintained in DMEM (Gibco)+10% heat-inactivated FBS (Axenia Biologix) supplemented with penicillin and streptomycin (Gibco). When indicated, cells were treated with drugs at 60-80% confluency at a final DMSO concentration of 1%. At the end of treatment period, cells were placed on ice and washed once with PBS. The cells were scraped with a spatula, pelleted by centrifugation (500×g, 5 min) and lysed in RIPA buffer supplemented with protease and phosphatase inhibitors (cOmplete and phosSTOP, Roche) on ice for 10 min. Lysates were clarified by high-speed centrifugation. Concentrations of lysates were determined with protein BCA assay (Thermo Fisher) and adjusted to 2 mg/mL with additional RIPA buffer. Samples were mixed with 5×SDS Loading Dye and heated at 95° C. for 5 min.

Gel Electrophoresis and Western Blot

Unless otherwise noted, SDS-PAGE was run with Novex 4-12% Bis-Tris gel (Invitrogen) in MES running buffer (Invitrogen) at 200V for 40 min following the manufacturer's instructions. Protein bands were transferred onto 0.45-μm nitrocellulose membranes (Bio-Rad) using a wet-tank transfer apparatus (Bio-Rad Criterion Blotter) in 1×TOWBIN buffer with 10% methanol at 75V for 45 min. Membranes were blocked in 5% BSA-TBST for 1 h at 23° C. Primary antibody binding was performed with the indicated antibodies diluted in 5% BSA-TBST at 4° C. for at least 16 h. After washing the membrane three times with TBST (5 min each wash), secondary antibodies (goat anti-rabbit IgG-IRDye 800 and goat anti-mouse IgG-IRDye 680, Li-COR) were added as solutions in 5% skim milk-TBST at the dilutions recommended by the manufacturer. Secondary antibody binding was allowed to proceed for 1 h at 23° C. The membrane was washed three times with TBST (5 min each wash) and imaged on a Li-COR Odyssey fluorescence imager.

Differential Scanning Fluorimetry

The protein of interest was diluted with SEC Buffer [20 mM HEPES 7.5, 150 mM NaCl, 1 mM MgCl₂] to 8 μM and mixed with a 100×DMSO solution of the ligand of interest. When no ligand was added, DMSO was used. SYPRO Orange Dye (Invitrogen) was added as a 500× solution in DMSO to a final nominal concentration of 5×. The resulting mixture was dispensed into wells of a white 96-well PCR plate in triplicate (25 μL/well). Fluorescence was measured at 0.5° C. temperature intervals every 30 s from 25° C. to 95° C. on a Bio-Rad CFX96 qPCR system using the FRET setting. Each data set was normalized to the highest fluorescence and the normalized fluorescence reading was plotted against temperature in GraphPad Prism 6.0. Tm values were determined as the temperature(s) corresponding to the maximum(ma) of the first derivative of the curve.

Sos- or EDTA-Mediated Nucleotide Exchange Assay

This assay was performed as previously reported (7, 11-13) with slight modifications.

BODIPY-GDP-loaded Avi-KRas(G12D) was prepared freshly as follows. To a 10 μM solution of Avi-KRas(G12D)·GDP in SEC Buffer (1 mL) was added sequentially BODIPY-GDP (5 mM, 40 μL, Thermo Fisher, final concentration 200 μM) and Na-EDTA pH 8.0 (0.5 M, 5 μL, final concentration 2.5 mM). The mixture was incubated at 23° C. for 1 h, and a solution of MgCl₂ (1.0 M, 20 μL, final concentration 10 mM) was added to the reaction mixture. The protein solution was run through a PD-10 column to remove the excess nucleotide following the manufacturer's protocol. Briefly, sample (˜1.0 mL) and excess buffer (1.5 mL) were loaded onto the column (equilibrated with NucEx Buffer), and desalted protein was eluted with NucEx Buffer (3.5 mL). Protein concentration was measured with Bradford assay and adjusted to 1.25 μM with NucEx Buffer.

50 μL protein solution was mixed with 0.5 μL DMSO solution of the test compound and the mixture was incubated at 23° C. for 15 min. 12 μL of this solution (triplicate for each condition) was added to wells of a black 384-well low-volume assay plate (Corning 4514). 3 μL of either 1 mM GDP, 1 mM GDP+5 μM Sos, or 1 mM GDP+40 mM EDTA (all prepared in NucEx Buffer) was added via a multichannel pipet rapidly to the wells. This should take less than 15 s to finish. The plate was immediately placed in a TECAN Spark 20 M plate reader, and fluorescence for BODIPY (excitation 488 nm, emission 520 nm) was read every 30 s over 1 h. Fluorescence intensity was normalized to values at time 0 and plotted again time. Observed rate constant (k_{obs}) was derived by fitting the curve to first-order kinetic equation

F=(F₀-F_{∞})exp[−k_{obs}t]+F_{∞}

and plotted against time.

Time-Resolved Fluorescence Resonance Energy Transfer (TR-FRET) Assay

TR-FRET assay was performed using recommended conditions from Cisbio with slight modifications. For GST-Raf1-RBD assays, biotinylated Ras proteins were diluted in TR-FRET buffer to 25 nM, and GST-Raf1-RBD was in TR-FRET buffer to 25 nM. For GST-BRAF assays, biotinylated Ras proteins were diluted in TR-FRET buffer to 250 nM and GST-BRAF was diluted to 250 nM. These concentrations were determined previously with titrations of both reagents to allow the maximal assay window. The difference between the GST-Raf1-RBD assay and GST-BRAF likely reflects the lower affinity of full length BRAF for Ras (Fisher et al. J. Biol. Chem. 2007, 282, 26503-26516). Anti-GST-Tb (Cisbo) was diluted to 500 ng/mL. Streptavidin-XL665 (Cisbio) was diluted to 10 μg/mL. Compounds were prepared at 5×testing concentrations in TR-FRET assay buffer (5% DMSO). For each replicate of each assay condition, 4 μL compound solution, 4 μL of diluted Ras protein, 4 μL diluted GST-Raf1-RBD or GST-BRAF was mixed in a well of a black low-volume 384-well plate (Corning 4514), and the mixture was incubated at 23° C. for 1 h. 4 μL of Anti-GST-Tb and 4 μL of Streptavidin-XL665 were then added sequentially to each assay well, and the mixture was incubated at 23° C. for an additional 1 h. Time-resolved fluorescence was read on a TECAN Spark 20 M plate reader with the following parameters:

Lag time: 60 μs

Integration time: 500 μs

Read A: Excitation filter 320(25) nm, Emission filter 610(25) nm, Gain 130

Read B: Excitation filter 320(25) nm, Emission filter 665(8) nm, Gain 165

TR-FRET signal was calculated as the ratio fluorescence intensity [Read B]/[Read A]. The ratiometric signal was further normalized to a negative control containing GDP-bound Ras protein. Three replicates were performed for each assay condition.

Chloroalkane Cell Penetration (CAPA) Assay

This assay was performed following published protocols (Peraro et al. 2018).

Briefly, Hela cells stably expressing HaloTag-GFP-Mito were seeded in a 96-well plate the day before the experiment at a density of 4×10⁴ cells per well. The day of the experiment the media was aspirated, and 100 μL of Opti-MEM was added to the cells. Test compounds at 500× assay concentration were diluted in Opti-MEM to 5×, and serial dilutions of the peptides were performed in a separate 96-well plate (final DMSO: 1%). 25 μL of compound solution was added to each well, and the plate was incubated for 4 h at 37° C. with 5% CO₂. The contents of the wells were aspirated off, and wells were washed using fresh Opti-MEM for 15 min. The wash was aspirated off, and the cells were chased using 5 μM ct-TAMRA (Promega) for 15 min, except for the control wells, which were incubated with Opti-MEM alone. The contents of the wells were aspirated and washed with fresh Opti-MEM for 30 min. After aspiration, cells were rinsed once with phosphate-buffered saline (PBS). The cells were then trypsinized, resuspended in PBS, and analyzed using an Attune NxT flow cytometer (Thermo Fisher Scientific). Data analysis was performed on GFP positive cell population, and median fluorescence intensity (MFI) in the TAMRA channel, normalized to DMSO treatment control, was plotted against peptide concentration.

Crystallization

GppNHp-loaded K-Ras Cyslight (G12D/C51S/C80L/C118S) purified by size exclusion chromatography was concentrated to 20 mg/mL and 100 μL protein was mixed with 5 μL 30 mM cyclic peptide solution in DMSO and 5 μL 100 mM GppNHp in SEC buffer. The mixture was incubated at 23° C. for 24 h and centrifuged (21,000×g, 30 min) to remove particulates. The supernatant was transferred into new tubes. For crystallization, 0.1 μL of the protein was mixed with 0.1 μL well buffer containing 0.1 M Tris 9.0, 0.2 M lithium sulfate, 30% PEG4000. Crystals were grown at 20° C. in a 96-well plate using the hanging-drop vapor diffusion method. Maximal crystal growth was achieved after 25 days. The crystals were transferred to a cryoprotectant solution (0.1 M Tris 9.0, 0.2 M lithium sulfate, 30% PEG4000, 20% glycerol) and flash-frozen in liquid nitrogen.

X-Ray Data Collection and Structure Determination

Dataset was collected at the Advanced Light Source beamline 8.2.2 with X-ray at a wavelength of 0.999907 Å. The dataset was indexed and integrated using iMosfim (Battye et al., 2011), scaled with Scala (Evans, 2006) and solved by molecular replacement using Phaser (McCoy et al., 2007) in CCP4 software suite (Winn et al., 2011). The crystal structure of GppNHp-bound K-Ras(G12D) (PDB code: 5USJ) was used as the initial model. The structure was manually refined with Coot (Emsley et al., 2010) and PHENIX (Adams et al., 2010). Data collection and refinement statistics are listed in Table 1. In the Ramachandran plot of the final structure, 97.86% and 1.95% of the residues are in the favored regions and allowed regions, respectively. One residue, Arg41, directly adjacent to the partially disordered Switch I region (32-40), is calculated as an outlier.

TABLE 1
Data collection and refinement statistics.

| | K-Ras(G12D)•KD2 |
|---|---|
| Data Collection | |
| Space group | P 21 21 21 |
| Cell Dimensions | |
| a, b, c (Å) | 72.437, 79.155, 90.982 |
| α, β, γ (°) | 90, 90, 90 |
| Resolution (Å) | 50.00-1.60 (1.63-1.60) |
| Total reflections | 69379 |
| Unique reflections | 69342 |
| Redundancy | 9.6 (7.7) |
| Completeness (%) | 99.9 (100) |
| I/σ | 20.1 (1.6) |
| R_{merge} | 0.122 (0.921) |
| Rₘₑₐₛ | 0.116 (0.804) |
| Rₚᵢₘ | 0.037 (0.286) |
| CC_{1/2} | 0.998 (0.866) |
| CC* | 1.000 (0.963) |
| Refinement | |
| Resolution range (Å) | 34.73-1.601 (1.659-1.601) |
| Reflections used in refinement | 66446 (4750) |
| Reflections used for R-free | 3261 (234) |
| R_{work} | 0.1839 (0.1997) |
| R_{free} | 0.2137 (0.2362) |
| Number of non-hydrogen atoms | 5007 |
| macromolecules | 4334 |
| ligands | 99 |
| solvent | 574 |
| Protein residues | 539 |
| RMS(bonds) | 0.007 |
| RMS(angles) | 1.18 |
| Ramachandran favored (%) | 98.05 |
| Ramachandran allowed (%) | 1.95 |
| Ramachandran outliers (%) | 0.00 |
| Rotamer outliers (%) | 0.21 |
| Clashscore | 3.33 |
| Average B-factor | 19.61 |
| macromolecules | 18.46 |
| ligands | 12.72 |
| solvent | 29.47 |

*Statistics for the highest-resolution shell are shown in parentheses.

Heteronuclear Single-Quantum Coherence (HSQC)

Samples of ¹⁵N-labeled, GppNHp-bound K-Ras(G12D) for NMR spectroscopy were prepared as follows. A 30 μL aliquot of protein at 1.0 mM in storage buffer (40 mM HEPES pH 7.4, 150 mM NaCl, 4 mM MgCl₂, 5% v/v glycerol, 7% v/v D₂O) was diluted with 240 μL of buffer (40 mM HEPES pH 7.4, 150 mM NaCl, 4 mM MgCl2, 7% v/v D₂O) in a 1.2 mL Eppendorf tube on ice. Then, 30 μL of either dmso-d6, or a cyclic peptide at 4.0 mM in dmso-d6 were added, the sample was mixed, and the resulting solution was transferred to a 5 mM D₂O-matched Shigemi NMR tube (BMS-3). The final concentrations of protein and cyclic peptide were 100 and 400 μM, respectively.

¹H-¹⁵N HSQC spectra (fhsqcf3gpph) were acquired on an 800 MHz Bruker Neo spectrometer at 288 K with 1024 and 512 points, 8 scans, and GARP decoupling. Chemical shifts were referenced to the HDO signal at 4.70 ppm.

Materials

Guanosine 5′-[β,γ-imido]triphosphate trisodium salt hydrate (GppNHp) and Guanosine 5′-diphosphate sodium salt were ordered from Sigma (St. Louis, Mo., USA). Sephadex™ G-25 fine was ordered from GE healthcare (Uppsala, Sweden). Acetylated bovine serum albumin (nuclease and protease tested) were ordered from Nacalai Tesque, Inc. (Kyoto, Japan). M-MLV Reverse Transcriptase and RNasin Plus RNase inhibitor were obtained from Promega (Madison, Wis., USA). Dynabeads M280 streptavidin was purchased from Thermo Fisher Scientific (Baltics, USA).

TABLE 2
List of antibodies

| Target | Supplier | Identifier | Dilution |
|---|---|---|---|
| Pan-Ras | abcam | 108062 | 1:5000 |
| P-ERK [T202/Y204] | Cell Signaling Technology | 9101 | 1:1000 |
| Total ERK | Cell Signaling Technology | 4695 | 1:1000 |
| P-S6 [S240/S244] | Cell Signaling Technology | 5364 | 1:2000 |
| S6 | Cell Signaling Technology | 2217 | 1:1000 |
| P-AKT [S473] | Cell Signaling Technology | 4060 | 1:1000 |
| AKT | Cell Signaling Technology | 2920 | 1:1000 |
| Actin | Proteintech | 60008-1-Ig | 1:50000 |

TABLE 3
List of buffer composition

| Name | Composition |
|---|---|
| RIPA Buffer | 25 mM Tris 7.4 |
| | 150 mM NaCl |
| | 0.1% SDS |
| | 1% NP-40 |
| | 0.5% sodium deoxycholate |
| Lysis Buffer | 20 mM Tris 8.0 |
| | 500 mM NaCl |
| | 5 mM imidazole |
| Elution Buffer | 20 mM Tris 8.0 |
| | 300 mM NaCl |
| | 300 mM imidazole |
| Phosphatase Buffer | 32 mM Tris 8.0 |
| | 200 mM ammonium sulfate |
| | 0.1 mM ZnCl₂ |
| SEC Buffer | 20 mM HEPES 8.0 |
| | 150 mM NaCl |
| | 1 mM MgCl₂ |
| NucEx Buffer | 20 mM HEPES 7.5 |
| | 150 mM NaCl |
| | 1 mM MgCl₂ |
| | 1 mM DTT |
| TR-FRET Buffer | 20 mM HEPES 7.5 |
| | 150 mM NaCl |
| | 1 mM MgCl₂ |
| | 0.05% Tween-20 |
| | 0.1% BSA |
| | 0.5 mM DTT |

List of Protein Sequences Used in this Study

Underlined texts indicate the affinity tags that were cleaved during purification.

>KRAS CysLight (G12D)
(SEQ ID NO: 4)
MHHHHHHSSGRENLYFQGMTEYKLVVVGADGVGKSALTIQLIQNHFVDEY
DPTIEDSYRKQVVIDGETSLLDILDTAGQEEYSAMRDQYMRTGEGFLLVF
AINNTKSFEDIHHYREQIKRVKDSEDVPMVLVGNKSDLPSRTVDTKQAQD
LARSYGIPFIETSAKTRQGVDDAFYTLVREIRKHKEK
>His-Avi-TEV-KRAS (G12D)
(SEQ ID NO: 5)
MGSSHHHHHHSGMSGLNDIFEAQKIEWHESSGENLYFQGMTEYKLVVVGA
DGVGKSALTIQLIQNHFVDEYDPTIEDSYRKQVVIDGETCLLDILDTAGQ
EEYSAMRDQYMRTGEGFLCVFAINNTKSFEDIHHYREQIKRVKDSEDVPM
VLVGNKCDLPSRTVDTKQAQDLARSYGIPFIETSAKTRQGVDDAFYTLVR
EIRKHKEK
>His-Avi-TEV-KRAS (wildtype)
(SEQ ID NO: 6)
MGSSHHHHHHSGMSGLNDIFEAQKIEWHESSGENLYFQGMTEYKLVVVGA
GGVGKSALTIQLIQNHFVDEYDPTIEDSYRKQVVIDGETCLLDILDTAGQ
EEYSAMRDQYMRTGEGFLCVFAINNTKSFEDIHHYREQIKRVKDSEDVPM
VLVGNKCDLPSRTVDTKQAQDEARSYGIPFIETSAKTRQGVDDAFYTLVR
EIRKHKEK
>GST-Raf1-RBD
(SEQ ID NO: 7)
MSPILGYWKIKGLVQPTRLLLEYLEEKYEEHLYERDEGDKWRNKKFELGL
EFPNLPYYIDGDVKLTQSMAIIRYIADKHNMLGGCPKERAEISMLEGAVL
DIRYGVSRIAYSKDFETLKVDFLSKLPEMLKMFEDRLCHKTYLNGDHVTH
PDFMLYDALDVVLYMDPMCLDAFPKLVCFKKRIEAIPQIDKYLKSSKYIA
WPLQGWQATFGGGDHPPKSDLVPRGSPIHIMEHIQGAWKTISNGFGFKDA
VFDGSSCISPTIVQQFGYQRRASDDGKLTDPSKTSNTIRVFLPNKQRTVV
NVRNGMSLHDCLMKALKVRGLQPECCAVFRLLHEHKGKKARLDWNTDAAS
LIGEELQVDFLDHVPLTTHNFARKTFLKLGIHRD
>GST-BRAF
(SEQ ID NO: 8)
MSPILGYWKIKGLVQPTRLLLEYLEEKYEEHLYERDEGDKWRNKKFELGL
EFPNLPYYIDGDVKLTQSMAIIRYIADKHNMLGGCPKERAEISMLEGAVL
DIRYGVSRIAYSKDFETLKVDFLSKLPEMLKMFEDRLCHKTYLNGDHVTH
PDFMLYDALDVVLYMDPMCLDAFPKLVCFKKRIEAIPQIDKYLKSSKYIA
WPLQGWQATFGGGDHPPKSDLEVLFQGPLGSPNSRVDAALSGGGGGGAEP
GQALFNGDMEPEAGAGAGAAASSAADPAIPEEVWNIKQMIKLTQEHIEAL
LDKFGGEHNPPSIYLEAYEEYTSKLDALQQREQQLLESLGNGTDFSVSSS
ASMDTVTSSSSSSLSVLPSSLSVFQNPTDVARSNPKSPQKPIVRVFLPNK
QRTVVPARCGVTVRDSLKKALMMRGLIPECCAVYRIQDGEKKPIGWDTDI
SWLTGEELHVEVLENVPLTTHNFVRKTFFTLAFCDFCRKLLFQGFRCQTC
GYKFHQRCSTEVPLMCVNYDQLDLLFVSKFFEHHPIPQEEASLAETALTS
GSSPSAPASDSIGPQILTSPSPSKSIPIPQPFRPADEDHRNQFGQRDRSS
SAPNVHINTIEPVNIDDLIRDQGFRGDGGSTTGLSATPPASLPGSLTNVK
ALQKSPGPQRERKSSSSSEDRNRMKTLGRRDSSDDWEIPDGQITVGQRIG
SGSFGTVYKGKWHGDVAVKMLNVTAPTPQQLQAFKNEVGVLRKTRHVNIL
LFMGYSTKPQLAIVTQWCEGSSLYHHLHIIETKFEMIKLIDIARQTAQGM
DYLHAKSIIHRDLKSNNIFLHEDLTVKIGDFGLATVKSRWSGSHQFEQLS
GSILWMAPEVIRMQDKNPYSFQSDVYAFGIVLYELMTGQLPYSNINNRDQ
IIFMVGRGYLSPDLSKVRSNCPKAMKRLMAECLKKKRDERPLFPQILASI
ELLARSLPKIHRSASEPSLNRAGFQTEDFSLYACASPKTPIQAGGYGAFP
VH

Characterization Data for Cyclic Peptides

Mass Spectrometry

| | | m/z | m/z |
|---|---|---|---|
| | | (calc'd | (found |
| Name | Sequence | for 2+) | for 2+) |
| KD1 | ^{D}YIIVTEKFIWVHHCG | 942.4856 | 942.4811 |
| KD2 | ^{D}YFVNFRNFRTFRCG | 933.4552 | 933.4468 |
| KD17 | ^{D}YNYPYRPLELGWYCG | 966.9412 | 966.9338 |
| KD2-His | ^{D}YFVNFRNFRHFRCG | 951.9529 | 951.9568 |
| KD2-Lys | ^{D}YFVNFRNFRKFRCG | 947.4709 | 947.4713 |
| KD2-Arg | ^{D}YFVNFRNFRRFRCG | 961.4740 | 961.4735 |
| KD2-Dap | ^{D}YFVNFRNFR(Dap)FRCG | 926.4474 | 926.4488 |
| KD2-Cit | ^{D}YFVNFRNFR(Cit)FRCG | 961.9660 | 961.9687 |
| KD2-AzaX | ^{D}YFVNFRNFR(Pip)FRCG | 967.4865 | 967.4877 |
| KD2-ClAc | ^{D}YFVNFRNFR(ClAcDap)FRCG | 964.4331 | 964.4311 |
| KD2-COH | ^{D}YFVNFRNFRTFRCG-CO₂H | 933.9472 | 933.9476 |
| Ct-KD2 | ^{D}YFVNFRNFRTFRCG-Ct | 1036.5088 | 1036.5116 |
| KD2-Thal | ^{D}YFVNFRNFRTFRCG-Thal | 1126.0189 | 1126.0177 |

### REFERENCES FOR EXAMPLE 2

1. Goto, Y., Katoh, T. & Suga, H. Nat. Protoc. 6, 779-790 (2011). 2. Hayashi, Y., Morimoto, J. & Suga, H. ACS Chem. Biol. 7, 607-613 (2012). 3. Hipolito, C. J., Tanaka, Y., Katoh, T., Nureki, O. & Suga, H. Molecules 18, 10514-10530 (2013). 4. Morimoto, J., Hayashi, Y. & Suga, H. Angew. Chem. Int. Ed. 51, 3423-3427 (2012). 5. Yamagata, K. et al. Structure 22, 345-352 (2014). 6. Yamagishi, Y. et al. Chem. Biol. 18, 1562-1570 (2011). 7. Ostrem, J. M., Peters, U., Sos, M. L., Wells, J. A. & Shokat, K. M. Nature 503, 548-51 (2013). 8. Gentile, D. R. et al. Cell Chem. Biol. 24, 1455-1466.e14 (2017). 9. Brtva, T. R. et al. J. Biol. Chem. 270, 9809-9812 (1995). 10. Sondermann, H. et al. Cell 119, 393-405 (2004). 11. Ahmadian, M. R. et al. Proc. Natl. Acad. Sci. U.S.A 96, 7065-70 (1999). 12. Huehls, A. M., Coupet, T. A. & Sentman, C. L. Immunol. Cell Biol. 93, 290-296 (2014). 13. Maurer, T. et al. Proc. Natl. Acad. Sci. U.S.A 109, 5299-304 (2012).

1. A compound having the formula: 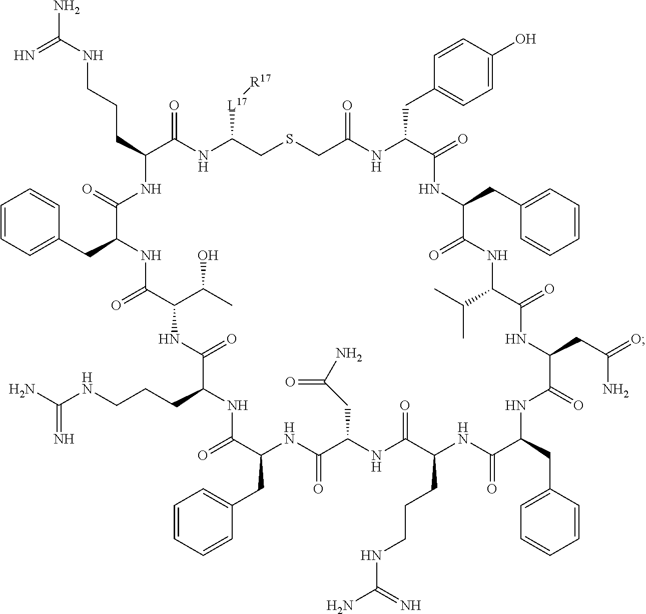
wherein
L¹⁷ is -L^{17A}-L^{17B}-L^{17C}-L^{17D}-L^{17E}-L^{17F}-;
L^{17A}, L^{17B}, L^{17C}, L^{17D}, L^{17E}, and L^{17F} are independently bond, —SS—, —S(O)₂—, —OS(O)₂—, —S(O)₂O—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
R¹⁷ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —C(O)H, —C(O)OH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, a monovalent nucleic acid, a monovalent protein, a detectable moiety, or a drug moiety.

2. A compound having the formula: 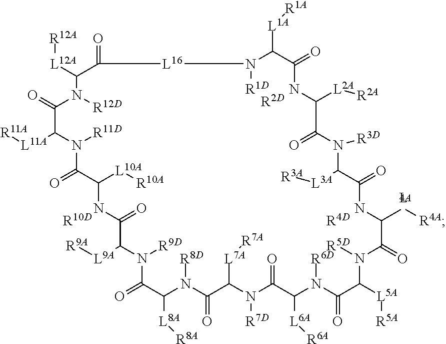
wherein
L^{1A}, L^{2A}, L^{3A}, L^{4A}, L^{5A}, L^{6A}, L^{7A}, L^{8A}, L^{9A}, L^{10A}, L^{11A}, and L^{12A} are independently a bond, substituted or unsubstituted alkylene, or substituted or unsubstituted heteroalkylene;
R^{1A}, R^{2A}, R^{5A}, R^{8A}, and R^{11A} are independently substituted or unsubstituted cycloalkyl, substituted or unsubstituted heterocycloalkyl, substituted or unsubstituted aryl, or substituted or unsubstituted heteroaryl;
R^{3A} is hydrogen, substituted or unsubstituted alkyl, substituted or unsubstituted cycloalkyl, or substituted or unsubstituted aryl;
R^{4A} and R^{7A} are independently hydrogen, —NH₂, —COOH, —CONH₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, substituted or unsubstituted alkyl, or substituted or unsubstituted heteroalkyl;
R^{6A}, R^{9A}, and R^{12A} are independently hydrogen, —CN, —NH₂, —CONH₂, —SO₂NH₂, —NHNH₂, —ONH₂, —NHC(O)NHNH₂, —NHC(O)NH₂, —NHC(NH)NH₂, —NHOH, substituted or unsubstituted alkyl, or substituted or unsubstituted heteroalkyl;
R^{3A} and R^{9A} may optionally be joined to form a covalent linker;
R^{10A} is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —C(O)H, —COOH, —C(O)NH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, -L^{10D}-L^{10E}-E, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L^{10D} is a bond, —S(O)₂—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L^{10E} is a bond, —S(O)₂—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted heteroalkylene, substituted or unsubstituted heterocycloalkylene, or substituted or unsubstituted heteroarylene;
E is an electrophilic moiety;
R^{1D}, R^{2D}, R^{3D}, R^{4D}, R^{5D}, R^{6D}, R^{7D}, R^{8D}, R^{9D}, R^{10D}, R^{11D}, and R^{12D} are independently hydrogen or unsubstituted C₁-C₄ alkyl; and
L¹⁶ is a covalent linker.

3.-6. (canceled)

7. The compound of claim 2, wherein R^{10A} is -L^{10D}-L^{10E}-E;
L^{10D} is a bond, —S(O)₂—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L^{10E} is a bond, —S(O)₂—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
E is —SH, —SSR²⁶, 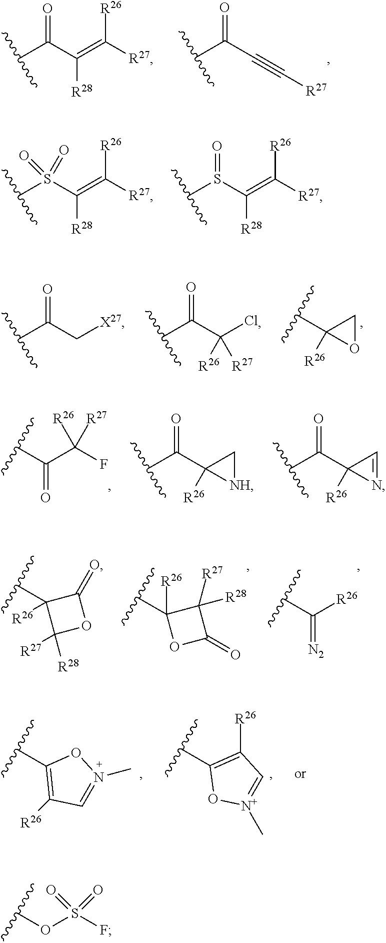
R²⁶, R²⁷, and R²⁸ are independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —C(O)H, —C(O)OH, —C(O)NH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
X²⁷ is —F, —Cl, —Br, or —I.

8.-10. (canceled)

11. The compound of claim 2, wherein R^{3A} and R^{9A} are joined to form a covalent linker having the formula -L^{18A}-L^{18B}-L^{18C}-L^{18D}-L^{18E}-L^{18F}-; and
L^{18A}, L^{18B}, L^{18C}, L^{18D}, L^{18E}, and L^{18F} are independently bond, —SS—, —S(O)₂—, —OS(O)₂—, —S(O)₂O—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

12. The compound of claim 2, wherein R^{3A} and R^{9A} are joined to form

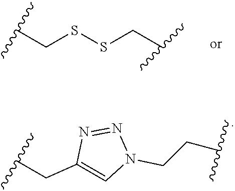 [SMILES: *CSSC* ; *CCn1cc(C*)nn1]

13. The compound of claim 2, having the formula:

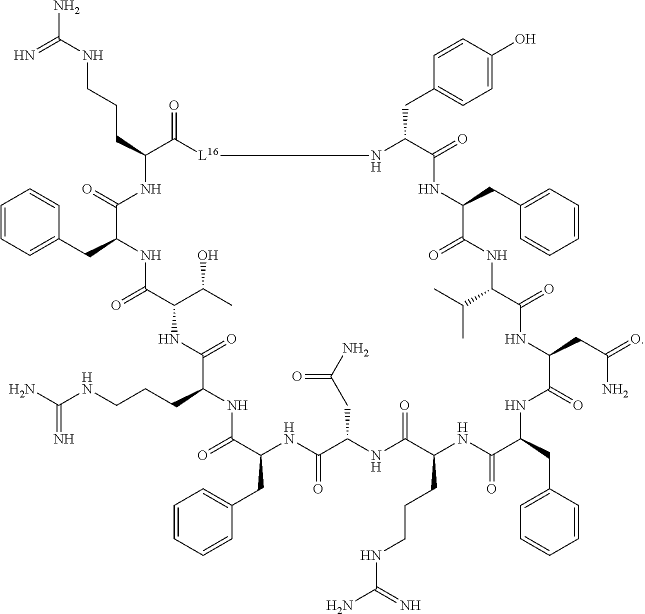

14.-16. (canceled)

17. The compound of claim 2, wherein
L¹⁶ is -L^{16A}-L^{16B}-L^{16C}-L^{16D}-L^{16E}-L^{16F}; and
L^{16A}, L^{16B}, L^{16C}, L^{16D}, L^{16E}, and L^{16F} are independently bond, —SS—, —S(O)₂—, —OS(O)₂—, —S(O)₂O—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

18. (canceled)

19. The compound of claim 17, wherein L¹⁶ is a bond, 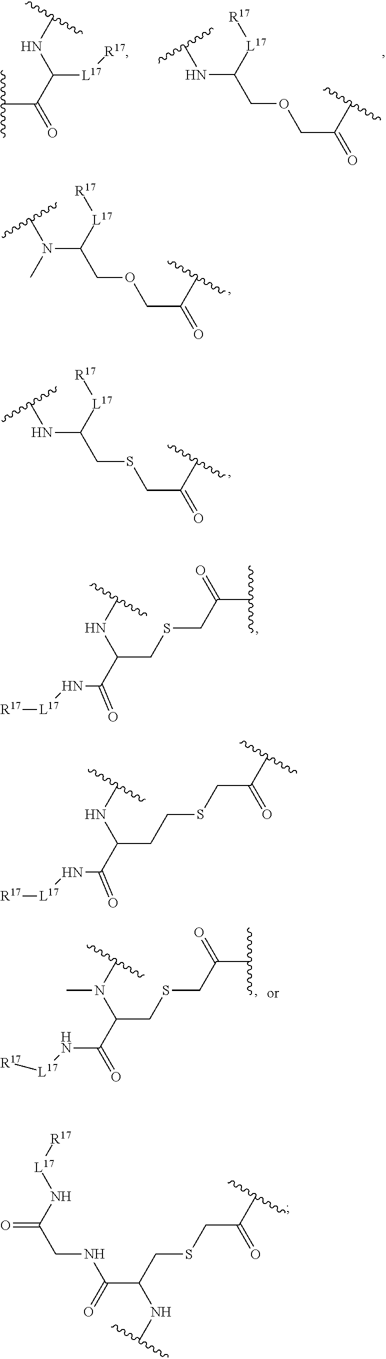
L¹⁷ is -L^{17A}-L^{17B}-L^{17C}-L^{17D}-L^{17E}-L^{17F}-;
L^{17A}, L^{17B}, L^{17C}, L^{17D}, L^{17E}, and L^{17F} are independently a bond, —SS—, —S(O)₂—, —OS(O)₂—, —S(O)₂O—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
R¹⁷ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —C(O)H, —C(O)OH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, a monovalent nucleic acid, a monovalent protein, a detectable moiety, or a drug moiety.

20. (canceled)

21. A compound having the formula: 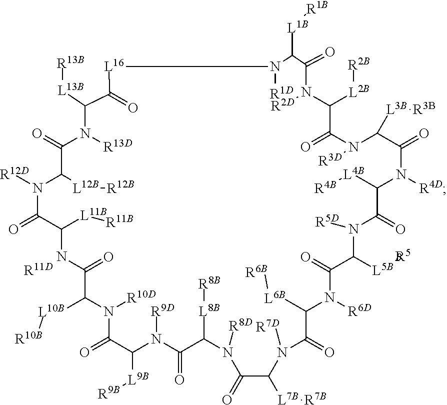
wherein
L^{1B}, L^{2B}, L^{3B}, L^{4B}, L^{5B}, L^{6B}, L^{7B}, L^{8B}, L^{9B}, L^{10B}, L^{11B}, L^{12B}, and L^{13B} are independently a bond, substituted or unsubstituted alkylene, or substituted or unsubstituted heteroalkylene;
R^{1B}, R^{8B}, and R^{10B} are independently substituted or unsubstituted cycloalkyl, substituted or unsubstituted heterocycloalkyl, substituted or unsubstituted aryl, or substituted or unsubstituted heteroaryl;
R^{2B}, R^{3B}, R^{4B}, R^{9B}, and R^{11B} are independently hydrogen, substituted or unsubstituted alkyl, substituted or unsubstituted cycloalkyl, or substituted or unsubstituted aryl;
R^{5B} is hydrogen, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, or substituted or unsubstituted heteroalkyl;
R^{6B} is hydrogen, —OH, —COOH, —NO₂, —SO₃H, —OSO₃H, substituted or unsubstituted alkyl, or substituted or unsubstituted heteroalkyl;
R^{7B}, R^{12B}, and R^{13B} are independently hydrogen, —NH₂, —CONH₂, —SO₂NH₂, —NHNH₂, —ONH₂, —NHC(O)NHNH₂, —NHC(O)NH₂, —NHC(NH)NH₂, substituted or unsubstituted alkyl, substituted or unsubstituted heteroalkyl, or substituted or unsubstituted heteroaryl;
two substituents selected from R^{1B}, R^{2B}, R^{3B}, R^{4B}, R^{5B}, R^{6B}, L^{7B}, R^{8B}, R^{9B}, R^{10B}, R^{11B}, R^{12B}, and R^{13B} may optionally be joined to form a covalent linker;
R^{1D}, R^{2D}, R^{3D}, R^{4D}, R^{5D}, R^{6D}, R^{7D}, R^{8D}, R^{9D}, R^{10D}, R^{11D}, R^{12D}, and R^{13D} are independently hydrogen or unsubstituted C₁-C₄ alkyl; and
L¹⁶ is a covalent linker.

22. (canceled)

23. (canceled)

24. The compound of claim 21, having the formula:

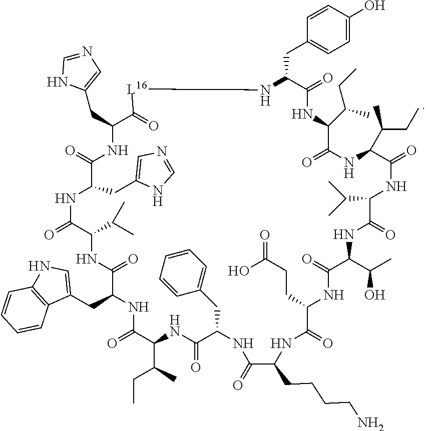

25.-31. (canceled)

32. A compound having the formula: 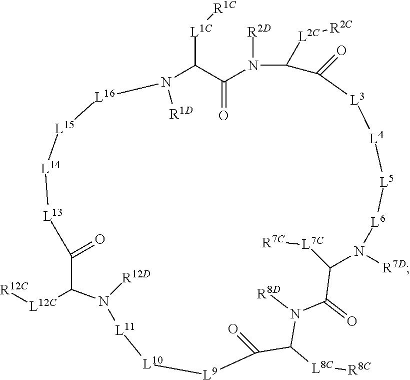
wherein
L^{1C}, L^{2C}, L^{3C}, L^{4C}, L^{5C}, L^{6C}, L^{7C}, L^{8C}, L^{9C}, L^{10C}, L^{11C}, L^{12C}, L^{13C}, L^{14C}, and L^{15C} are independently a bond, substituted or unsubstituted alkylene, or substituted or unsubstituted heteroalkylene;
R^{1C} is substituted or unsubstituted cycloalkyl, substituted or unsubstituted heterocycloalkyl, substituted or unsubstituted aryl, or substituted or unsubstituted heteroaryl;
R^{2C} is hydrogen, —OH, —NO₂, —CN, —NH₂, —C(O)OH, —C(O)NH₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, substituted or unsubstituted alkyl, or substituted or unsubstituted heteroalkyl;
L³ is a bond or 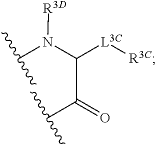
R^{3C} is hydrogen, —NH₂, —C(O)OH, —C(O)NH₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L⁴ is a bond or 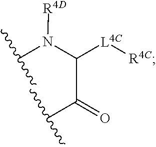
R^{4C} is hydrogen, substituted or unsubstituted alkyl, substituted or unsubstituted cycloalkyl, or substituted or unsubstituted aryl;
L⁵ is a bond or 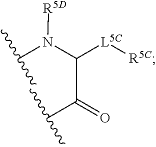
R^{5C} is substituted or unsubstituted cycloalkyl, substituted or unsubstituted heterocycloalkyl, substituted or unsubstituted aryl, or substituted or unsubstituted heteroaryl;
L⁶ is a bond, 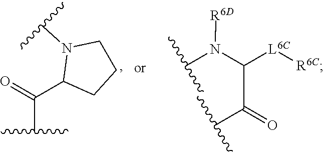
R^{6C} is hydrogen, —CN, —NH₂, —C(O)NH₂, —SO₂NH₂, —NHNH₂, —ONH₂, —NHC(O)NHNH₂, —NHC(O)NH₂, —NHC(NH)NH₂, —NHOH, substituted or unsubstituted alkyl, or substituted or unsubstituted heteroalkyl;
R^{7C} and R^{8C} are independently hydrogen, —CN, —NH₂, —C(O)NH₂, —NHNH₂, —ONH₂, —NHC(O)NHNH₂, —NHC(O)NH₂, —NHC(NH)NH₂, —NHOH, substituted or unsubstituted alkyl, or substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L⁹ is a bond, 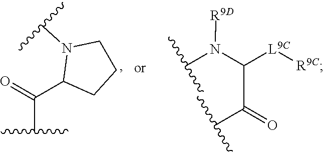
R^{9C} is hydrogen, substituted or unsubstituted alkyl, substituted or unsubstituted cycloalkyl, or substituted or unsubstituted aryl;
L¹⁰ is a bond, 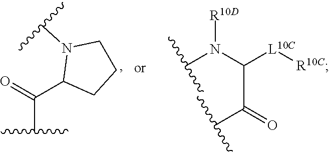
R^{10C} is hydrogen, substituted or unsubstituted alkyl, or substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L¹¹ is a bond or 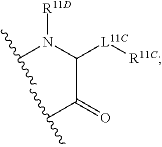
R^{11C} is hydrogen, —CN, —OH, —C(O)OH, —NO₂, —SO₃H, —OSO₃H, —NH₂, —C(O)NH₂, —SO₂NH₂, —NHNH₂, —ONH₂, —NHC(O)NHNH₂, —NHC(O)NH₂, —NHC(NH)NH₂, substituted or unsubstituted alkyl, or substituted or unsubstituted heteroalkyl;
R^{12C} is hydrogen, substituted or unsubstituted alkyl, or substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L¹³ is 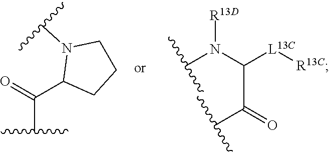
R^{13C} is hydrogen, —OH, —NH₂, —C(O)OH, —C(O)NH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, or substituted or unsubstituted heteroalkyl, substituted or unsubstituted cycloalkyl, or substituted or unsubstituted aryl;
L¹⁴ is a bond or 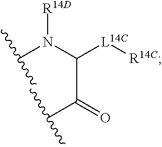
R^{14C} is hydrogen, —NH₂, —C(O)OH, —C(O)NH₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, substituted or unsubstituted alkyl, or substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L¹⁵ is a bond or 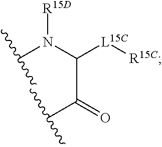
R^{15C} is hydrogen, —NH₂, —C(O)OH, —C(O)NH₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, substituted or unsubstituted alkyl, or substituted or unsubstituted heteroalkyl, substituted or unsubstituted cycloalkyl, substituted or unsubstituted heterocycloalkyl, substituted or unsubstituted aryl, or substituted or unsubstituted heteroaryl;
two substituents selected from R^{1C}, R^{2C}, R^{3C}, R^{4C}, R^{5C}, R^{6C}, R^{7C}, R^{8C}, R^{9C}, R^{10C}, R^{11C}, R^{12C}, R^{13C}, R^{14C}, and R^{15C} may optionally be joined to form a covalent linker;
R^{1D}, R^{2D}, R^{3D}, R^{4D}, R^{5D}, R^{6D}, R^{7D}, R^{8D}, R^{9D}, R^{10D}, R^{11D}, R^{12D}, R^{13D}, R^{14D}, and R^{15D} are independently hydrogen or unsubstituted C₁-C₄ alkyl; and
L¹⁶ is a covalent linker.

33. (canceled)

34. (canceled)

35. The compound of claim 32, having the formula:

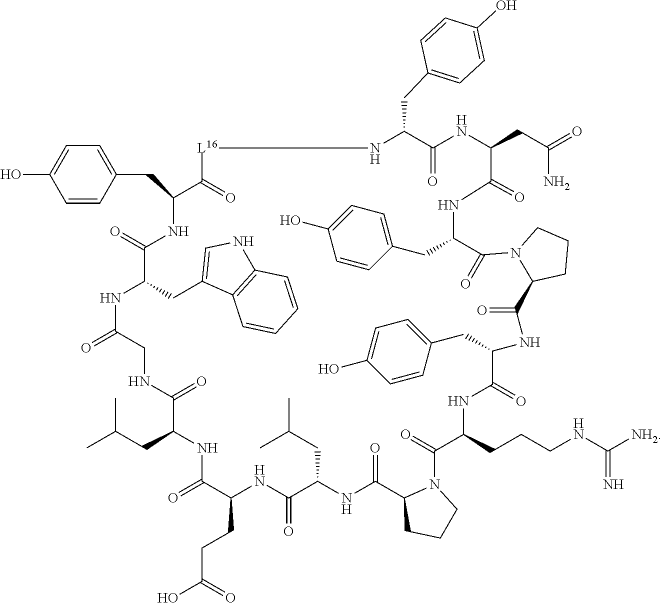

36.-42. (canceled)

43. A compound having the formula: 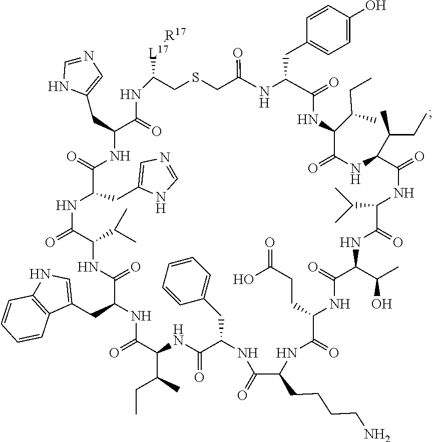
wherein
L¹⁷ is -L^{17A}-L^{17B}-L^{17C}-L^{17D}-L^{17E}-L^{17F}-;
L^{17A}, L^{17B}, L^{17C}, L^{17D}, L^{17E}, and L^{17F} are independently bond, —SS—, —S(O)₂—, —OS(O)₂—, —S(O)₂O—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
R¹⁷ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —C(O)H, —C(O)OH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, a monovalent nucleic acid, a monovalent protein, a detectable moiety, or a drug moiety.

44. A compound having the formula: 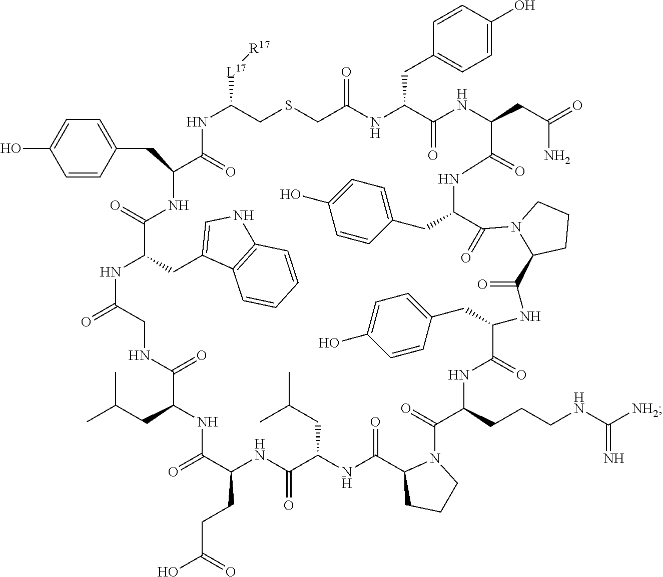
wherein
L¹⁷ is -L^{17A}-L^{17B}-L^{17C}-L^{17D}, L^{17E}-L^{17F}-;
L^{17A}, L^{17B}, L^{17C}, L^{17D}, L^{17E}, and L^{17F} are independently bond, —SS—, —S(O)₂—, —OS(O)₂—, —S(O)₂O—, —NH—, —O—, —S—, —C(O)—, —NHS(O)₂—, —S(O)₂NH—, —C(O)NH—, —NHC(O)—, —NHC(O)NH—, —NHC(NH)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; and
R¹⁷ is hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —C(O)H, —C(O)OH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, a monovalent nucleic acid, a monovalent protein, a detectable moiety, or a drug moiety.

45. A compound having the formula:

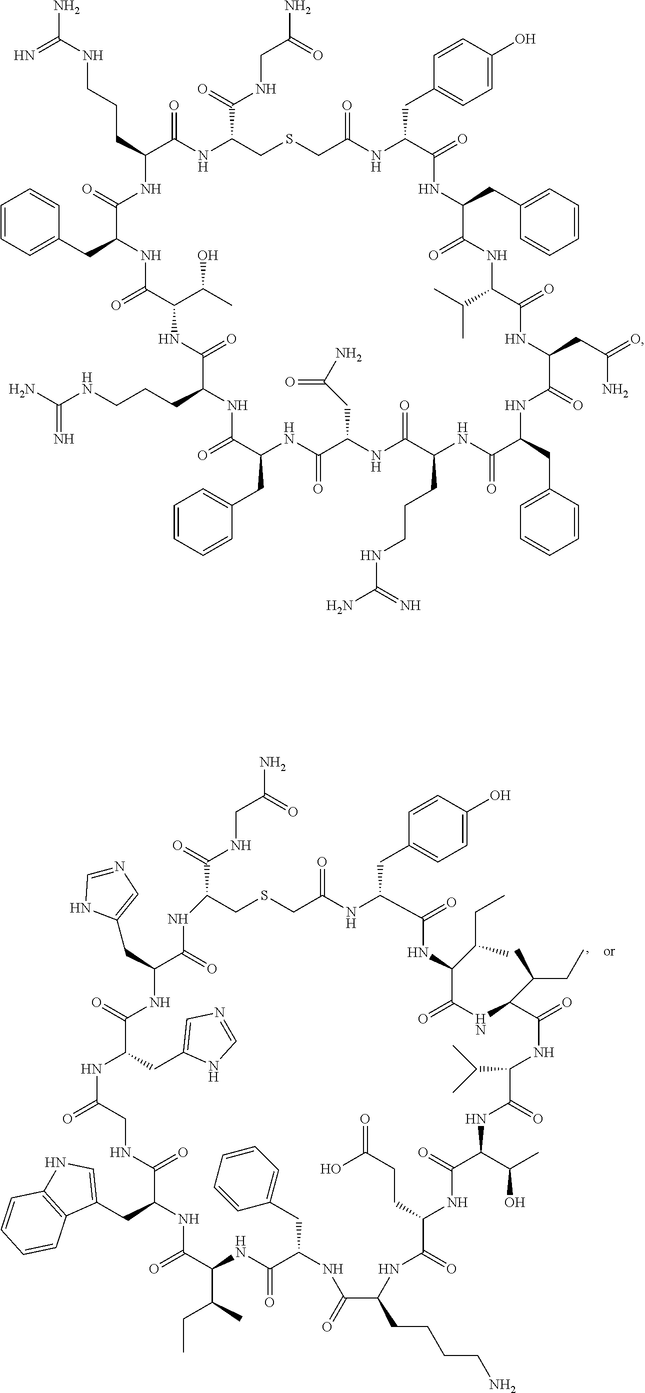

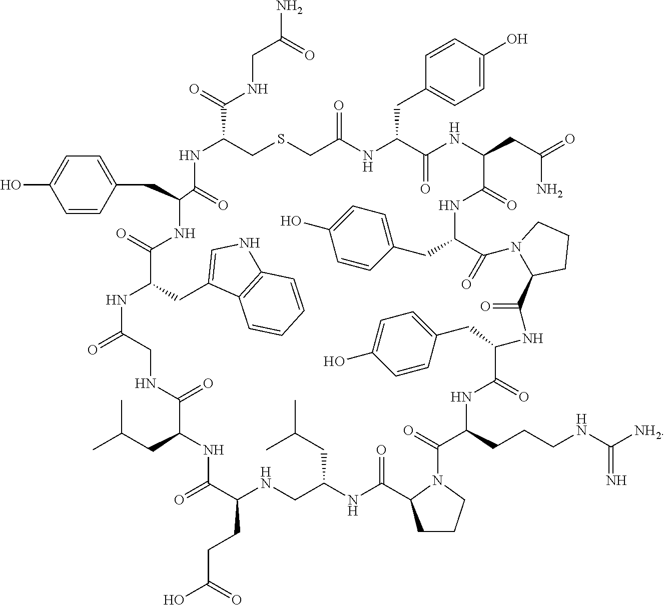 [SMILES: CC(C)C[C@H]1CN[C@@H](CCC(=O)O)C(=O)N[C@@H](CC(C)C)C(=O)NCC(=O)N[C@@H](Cc2c[nH]c3ccccc23)C(=O)N[C@@H](Cc2ccc(O)cc2)C(=O)N[C@H](C(=O)NCC(N)=O)CSCC(=O)N[C@H](Cc2ccc(O)cc2)C(=O)N[C@@H](CC(N)=O)C(=O)N[C@@H](Cc2ccc(O)cc2)C(=O)N2CCC[C@H]2C(=O)N[C@@H](Cc2ccc(O)cc2)C(=O)N[C@@H](CCCNC(=N)N)C(=O)N2CCC[C@H]2C(=O)N1]

46.-59. (canceled)

60. A pharmaceutical composition comprising the compound of claim 1, or a pharmaceutically acceptable salt thereof, and a pharmaceutically acceptable excipient.

61. A method of treating a cancer in a patient in need of such treatment, said method comprising administering a therapeutically effective amount of a compound of claim 1 to said patient.

62. A method of modulating the activity of a human K-Ras protein, said method comprising contacting said human K-Ras protein with an effective amount of a compound of claim 1.

63.-66. (canceled)

67. A method of modulating the activity of a human H-Ras protein, said method comprising contacting said human H-Ras protein with an effective amount of a compound of claim 1.

68. A method of modulating the activity of a human N-Ras protein, said method comprising contacting said human N-Ras protein with an effective amount of a compound of claim 1.


SEQUENCE LISTING (8 sequences)

SEQ ID NO: 1 - protein, 189 aa, Artificial Sequence [sequence omitted]

SEQ ID NO: 2 - protein, 189 aa, Artificial Sequence
  Synthetic polypeptide
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 3 - protein, 189 aa, Artificial Sequence
  Synthetic polypeptide
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

SEQ ID NO: 4 - protein, 187 aa, Artificial Sequence
  Synthetic polypeptide
MHHHHHHSSGRENLYFQGMTEYKLVVVGADGVGKSALTIQLIQNHFVDEYDPTIEDSYRKQVVIDGETSLLDILDTAGQEEYSAMRDQYMRTGEGFLLVFAINNTKSFEDIHHYREQIKRVKDSEDVPMVLVGNKSDLPSRTVDTKQAQDLARSYGIPFIETSAKTRQGVDDAFYTLVREIRKHKEK

SEQ ID NO: 5 - protein, 208 aa, Artificial Sequence
  Synthetic polypeptide
MGSSHHHHHHSGMSGLNDIFEAQKIEWHESSGENLYFQGMTEYKLVVVGADGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 6 - protein, 208 aa, Artificial Sequence
  Synthetic polypeptide
MGSSHHHHHHSGMSGLNDIFEAQKIEWHESSGENLYFQGMTEYKLVVVGAGGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 7 - protein, 384 aa, Artificial Sequence
  Synthetic polypeptide
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSPIHIMEHIQGAWKTISNGFGFKDAVFDGSSCISPTIVQQFGYQRRASDDGKLTDPSKTSNTIRVFLPNKQRTVVNVRNGMSLHDCLMKALKVRGLQPECCAVFRLLHEHKGKKARLDWNTDAASLIGEELQVDFLDHVPLTTHNFARKTFLKLGIHRD

SEQ ID NO: 8 - protein, 1002 aa, Artificial Sequence
  Synthetic polypeptide
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLGSPNSRVDAALSGGGGGGAEPGQALFNGDMEPEAGAGAGAAASSAADPAIPEEVWNIKQMIKLTQEHIEALLDKFGGEHNPPSIYLEAYEEYTSKLDALQQREQQLLESLGNGTDFSVSSSASMDTVTSSSSSSLSVLPSSLSVFQNPTDVARSNPKSPQKPIVRVFLPNKQRTVVPARCGVTVRDSLKKALMMRGLIPECCAVYRIQDGEKKPIGWDTDISWLTGEELHVEVLENVPLTTHNFVRKTFFTLAFCDFCRKLLFQGFRCQTCGYKFHQRCSTEVPLMCVNYDQLDLLFVSKFFEHHPIPQEEASLAETALTSGSSPSAPASDSIGPQILTSPSPSKSIPIPQPFRPADEDHRNQFGQRDRSSSAPNVHINTIEPVNIDDLIRDQGFRGDGGSTTGLSATPPASLPGSLTNVKALQKSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQILASIELLARSLPKIHRSASEPSLNRAGFQTEDFSLYACASPKTPIQAGGYGAFPVH